(19) United States
(12) Patent Application Publication
(10) Pub. No.: US 2021/0244941 A1
(43) Pub. Date: Aug. 12, 2021

(54) METHODS AND APPARATUS FOR A WEARABLE ELECTRONIC DIGITAL THERAPEUTIC DEVICE

(71) Applicants: John J. Daniels, Madison, CT (US); KINAPTIC, LLC, Madison, CT (US)

(72) Inventors: John J. Daniels, Madison, CT (US); Joseph Curcio, Gray, ME (US)

(21) Appl. No.: 17/201,091

(22) Filed: Mar. 15, 2021

Related U.S. Application Data
(63) Continuation of application No. PCT/US19/45429, filed on Aug. 7, 2019.
(60) Provisional application No. 62/780,288, filed on Dec. 16, 2018.
(60) Provisional application No. 62/771,117, filed on Nov. 25, 2018.
(60) Provisional application No. 62/742,284, filed on Oct. 6, 2018.

(51) Int. Cl. A61N 1/04; A61N 1/36

(52) U.S. Cl. CPC A61N 1/0456; A61N 1/0452; A61N 1/0428; A61N 1/36021; A61N 1/36031

(57) ABSTRACT

A wearable electronic therapeutic device has one or more biometric detectors each for detecting one or more biometric parameters. The biometric parameters are dependent on at least one physiological change to a patient in response to a therapeutic treatment. A microprocessor receives the one or more biometric parameters and applies probabilistic analysis to determine if at least one physiological change threshold has been exceeded dependent on the probabilistic analysis of the two or more biometric parameters. An activation circuit activates an action depending on the determined exceeded physiological change. The action that is activated can be applying an elcetroceutical treatment in addition or as an alternative to a pharmaceutical treatment.

### CROSS REFERENCE TO RELATED APPLICATIONS

This is a PCT application of U.S. Provisional Application No. 62/644,547, entitled Elastic Bandage and Dry Electrode System for Electro Stimulation Therapy, filed 18 Mar. 2018; and U.S. Provisional Application No. 62/742,284, entitled DIGITAL THERAPEUTIC FOR DETECTING AND REPORTING A BIOMETRIC AND AUTOMATICALLY ADJUSTING AN APPLIED THERAPY, filed 6 Oct. 2018; U.S. Provisional Application No. 62/771,117, entitled Digital Therapeutic Wearable Electronic Garments for Detecting and Reporting a Biometric and Automatically Adjusting an Applied Therapy, filed 25 Nov. 2018; and U.S. Provisional Application No. 62/780,288 entitled DIGITAL THERAPEUTIC FOR DETECTING AND REPORTING THE INGESTION OF A PHARMACEUTICAL SUCH AS A COAGULATION FACTOR INHIBITOR, filed 16 Dec. 2018

### TECHNICAL FIELD

The exemplary and non-limiting embodiments of this invention relate generally to digital therapeutic systems, methods, devices and computer programs and, more specifically, relate to digital therapeutic wearable electronic garments for detecting and reporting a biometric and automatically adjusting an applied therapy in response thereto.

The present invention pertains to a device architecture, specific-use applications, and computer algorithms used with for wearable electronics in the form of clothing and other wearable garments with the capability to detect biometric parameters for the treatment and monitoring of physiological conditions in humans and animals.

The present invention also relates to a method, apparatus and computer program code for detecting a biometric parameter, such as biomarkers, such as thrombin and/or d-dimer, for treatment and monitoring of physiological conditions, such as cardiovascular conditions, related to a physiological system, such as the contract system for coagulation and inflammation.

The present invention pertains to a device architecture, specific-use applications, and computer algorithms used with for wearable electronics in the form of clothing and other wearable garments with the capability to detect, analyze and apply electrical signals and other treatments and biometrics.

The present invention also relates to a novel pharmacological medicinal compound, method, apparatus and computer program code for providing a determination of patient adherence to a prescribe therapy and/or the ingestion or other delivery of medications taken by the patient.

The present invention pertains to a device architecture, specific-use applications, and computer algorithms used with for wearable electronics in the form of clothing and other wearable garments with the capability to detect and report the ingestion of a coagulation factor inhibitor.

The present invention also relates to a novel pharmacological medicinal compound, method, apparatus and computer program code for providing a determination of patient adherence to a prescribe therapy and/or the ingestion or other delivery of medications taken by the patient.

### BACKGROUND

This section is intended to provide a background or context to the exemplary embodiments of the invention as recited in the claims. The description herein may include concepts that could be pursued but are not necessarily ones that have been previously conceived, implemented or described. Therefore, unless otherwise indicated herein, what is described in this section is not prior art to the description and claims in this application and is not admitted to be prior art by inclusion in this section.

Electro stimulation therapy, or Transcutaneous electrical nerve stimulation (TENS), can be applied as a form of electroanalgesia. TENS has been conventionally used for a variety of conditions including low back pain, arthritis, neurogenic pain, visceral pain, and postsurgical pain.

The application of TENS results in neuromodulation through mechanisms such as presynaptic inhibition in the dorsal hom of the spinal cord, endogenous pain control (via endorphins, enkephalins, and dynorphins), direct inhibition of an abnormally excited nerve and the restoration of afferent input.

A typical TENS unit includes a battery powered electrical signal generator with long wires that connect with a set of gel electrodes. Self-adhesive electrodes for transcutaneous stimulation use a gel to make contact between a conductive member and the surface of the user's skin. The gel electrode is typically built in a multi-layer configuration sometimes including multiple layers of hydrogel. The skin interface layer may include an electrically conductive gel for removably contacting the user's skin. The conductive gel is made from co-polymers derived from polymerization, e.g. of acrylic acid and N-vinylpyrrolidone. In a multiple layer hydrogel, a second hydrogel layer connects a substrate conductive member (a low resistive material such carbon impregnated rubber or a wire mesh) with the skin hydrogel layer.

A typical TENS unit is able to generate signals with variable current strengths, pulse rates, and pulse widths. A preferred waveform is biphasic, to avoid the electrolytic and iontophoretic effects of a unidirectional current. The usual settings for the stimulus parameters used clinically include amplitude (signal intensity), pulse width (duration), and pulse rate (frequency).

When TENS is used analgesically, electrode positioning is an important consideration. The electrodes may be place on or near the painful area, or at other locations (for example, at cutaneous nerves, trigger points, acupuncture sites). Medical complications arising from use of TENS are rare. However, skin irritation often occur in due, at least in part, to drying out of the electrode gel and to the salt and other ingredients comprising the conductive hydrogel.

The conventional sticky gel electrode is a relatively expensive component that needs to be replaced often. Salts and other materials in the hydrogel can irritate the skin. The removal of the sticky gel electrode is often very discomforting, especially when applied over hair. Also, the sticky gel electrode become dirty and loses the ability to adhere to the skin very quickly.

The wires required to conduct the electrical signal from the TENS unit to the gel electrodes are cumbersome and often get entangled and either disconnect the gel electrode from the TENS unit, or pull the gel electrode off the user's skin. These wires are particularly inconvenient if the user wishes to have mobility while using the TENS treatment.

Accordingly, there is a need for a more convenient TENS system that avoids the drawbacks of the conventional sticky gel electrodes and avoids the need for long, loose wires to conduct the TENS signal from the TENS generator to the electrodes.

In the US, there are 900,000 people affected by deep vein thrombosis (DVT) and/or pulmonary embolism (PE). 1 out of 9 of these DVT/PE patients will the as a result of their condition. Each year, more people the of DVT/PE than the breast cancer, traffic accidents and HIV combined.

Clotting agents in blood, platelets (thromobcytes) and fibrin, are present to prevent blood loss. However, problems arise when blood clots lodged in blood vessels of the lower legs travel to the lungs. The treatment of deep vein thrombosis (DVT) is intended to prevent the clot from getting bigger and preventing it from breaking loose and causing a pulmonary embolism. Treatment options include blood thinners or anticoagulants that decrease the blood's ability to clot.

Clot buster drugs or thrombolytics may be prescribed to break up clots quickly but are generally reserved for severe cases of blood clots. A vena cava filter may be implanted to catch clots that break loose from lodging in the lungs, and compression stockings are typically worn to help prevent swelling associated with deep vein thrombosis, these are worn on the legs from the feet to about the level of the knees.

To heal an injury to a vein or artery, the body uses platelets (thrombocytes) and fibrin to clot the blood and prevent blood loss. Blood clots also can form within a blood vessel even when there is no injury. Thrombosis occurs when a blood clot formed inside a blood vessel obstructs the flow of blood through the circulatory system. If the clot is anchored in place within the blood vessel it may eventually dissolve without any issue. But, if the clot breaks free and begins to travel around the body, life threatening damage can occur. The dislodged clot, an embolus, can lodge within the circulatory system causing a type of embolism known as a thromboembolism.

DVT most commonly affects leg veins and occurs when a blood clot forms within a deep vein. A venous thromboembolism (VTE) can lodge in the lung causing a debilitating and often fatal pulmonary embolism. A PE occurs when a DVT blood clot dislodges from a blood vessel and becomes lodged in the lungs. A PE that blocks blood flow can be life threatening, damaging the lungs and other organs. The symptoms of PE include shortness of breath, pain with deep breathing, and coughing up blood. Some people experience these symptoms, unaware that they may have started as a deep vein blood clot. About 380 million people, or 5% of the world's population, is affected by DVT and VTE as some point in their lives.

The standard pharmacological treatment for thrombosis is anticoagulation to reduce the ability of platelets and fibrin to interact and cause blood clotting. Rivaroxaban, developed by Bayer and sold under the brand name Xarelto, is the first orally administered medication with a direct Factor Xa inhibitor. Factor Xa is chemical part of the body's coagulation mechanism.

In 2011, the US FDA approved rivaroxaban for stroke prevention in people with non-valvular atrial fibrillation. In 2012, the FDA approved Xarelto for treatment of deep vein thrombosis and pulmonary embolism.

A review of the patent literature shows the use of an electrical stimulator for the prevention of deep vein thrombosis, ankle edema, and venostasis. U.S. Pat. No. 5,653,331, entitled Method and device for prevention of deep vein thrombosis, issued Jul. 1, 1997 to Amiram Katz, shows the use of an anode and cathode electrode pair secured at or near the tibial nerve at the popliteal fossa on both legs of a patient. An electrical signal is applied to stimulate the nerve causing muscle contractions in the calf of the legs to prevent deep vein thrombosis, ankle edema, and venostasis.

PCT patent application PCT/US99/08450, entitled Neuromuscular electrical stimulation for preventing deep vein thrombosis, applied for by Stryker Instruments, International filing date 16 Apr. 1999 shows a neuromuscular electrical stimulation system that instigates muscle twitch to prevent DVT. The duration and duty cycle of the applied electrical pulses are controlled to instigate the muscle twitch without causing tetanic, or full and sustained, muscle contractions'.

Venography is the current standard for diagnosing DVT, where a special dye is injected into the bone marrow or veins. The dye has to be injected constantly via a catheter, making it an invasive procedure. Light Reflection Rheography (LRR) is a non-invasive technique that uses LEDs and a sensor to measure DVT with the LEDs and sensors at the skin surface. The intensity of the reflected light quantifies the venous function by measuring changes in microcirculation.

U.S. Pat. No. 5,282,467, entitled Non-invasive method for detecting deep venous thrombosis in the human body, issued Feb. 1, 1994 to Piantadosi et al. shows a non-invasive method for detecting deep venous thrombosis, a change in the amount of deoxyhemoglobin can be detected by trapping blood in a vein for a determined time period. Light sources are used to emit two selected wavelengths that penetrate into the deep venous system. The reflectance contribution of the selected wavelengths are used to measure changes in blood flow and amount of deoxyhemoglobin indicative of presence or absence of deep venous thrombosis.

The prior attempts at mitigating disease, such as DVT, all fall short of optimizing the cost, patient outcome, and general advantageous collection of biometric information. According there is a need for digital therapeutic systems, methods, devices and computer programs for detecting and reporting a biometric and automatically adjusting an applied therapy in response thereto.

The contact system is the pathway for coagulation and inflammation where a group of plasma proteins react to the presence of (patho)physiological materials and invasive pathogens. This system consists of three serine proteinases: coagulation factors XII (FXII) and XI (FXI), and plasma prekallikrein (PK), as well as the nonenzymatic cofactor high molecular weight kininogen (HK) (Colman R W, Schmaier A H. Contact system: a vascular biology modulator with anticoagulant, profibrinolytic, antiadhesive, and proinflammatory attributes. Blood. 1997; 90(10):3819-43).

Activation of the contact system results in blood coagulation and is also responsible for the generation of the proinflammatory products such as bradykinin. The contact system is also called the plasma kallikrein-kinin system, functioning in inflammatory conditions such as rheumatoid arthritis (RA) and inflammatory bowel disease (IBD).

The contact system proteins interact with a number of physiologic and pathophysiologic pathways, participating in the pathophysiological responses to injury, most notably in the processes of coagulation and inflammation. The activation of the contact system is involved in a wide variety of diseases, including septicemia and endotoxemia, ARDS, DIC, typhoid fever, Rocky Mountain spotted fever, Crohn's disease, transfusion reactions, renal allograft rejection, nephrotic syndrome, hereditary angioedema, and in extracorporeal circulation. Inhibitors of contact factor enzymes may be effective at modulating hypotension, inflammation or prolonged survival, possibly participating in the mechanisms for host defense and innate immunity.

The contact system is initiated by molecules that come from injured cells or that are present in pathogens. These molecules bind FXII and HK, and initiate a reciprocal activation system where FXII is activated to FXIIa through an autocatalytic reaction involving Zn2+. HK bridges FXI and PK together to bring them into proximity of FXII. A cycle begins where FXIIa activates HK-bound PK, resulting in kallikrein that activates additional FXII. FXIIa also activates FXI in a HK-dependent fashion, and the subsequent FXIa then feeds into the intrinsic pathway by activating FIX, leading to thrombin generation.

A missing link was discovered in the regulation of coagulation. Gailani and Broze reported in 1991 that thrombin activates FXI in a positive feedback reaction (Gailani D, Broze G J. Factor XI activation in a revised model of blood coagula-tion. Science (1991) 253:909-12. doi:10.1126/science.1652157). This positive feedback reaction seems to obviate the role of the contact system by providing an alternative means of activating FXI. However, it also identifies new levels of regulation of coagulation. The pathways for FXI activation are bidirectional, and FXI is important for maximizing thrombin generation. The inhibition of FXI activation could therefore be used in a new generation of anticoagulation drugs for treatment of a wide range of cardiovascular ailments.

Coagulation factor II (Gene symbol F2) is proteolytically cleaved to form thrombin in the first step of the coagulation cascade which ultimately results in the stemming of blood loss. F2 also plays a role in maintaining vascular integrity during development and postnatal life. Mutations in F2 leads to various forms of thrombosis and dysprothrombinemi.

The prior art fails to provide a mechanism for detecting a biometric parameter, such as biomarkers, such as thrombin and/or d-dimer, for treatment and monitoring of physiological conditions, such as cardiovascular conditions, related to a physiological system, such as the contract system for coagulation and inflammation.

D-dimer is one of the protein fragments produced when a blood clot gets dissolved in the body. It is normally undetectable or detectable at a very low level unless the body is forming and breaking down blood clots.

Venous thrombosis activates the coagulation and fibrinolytic systems, and results in elevated levels of serum markers collectively called fibrin spit products. During thrombus formation, fibrinogen is converted to fibrin monomers which are then cross linked into polymers and produce a biomarker called the D-dimer fibrin fragment. D-dimer antigen levels are elevated in the acute phase of clot formation as would occur in acute deep venous thrombosis, and also the fibrinolytic stage that would occur in the setting of acute pulmonary embolism. D-dimers also have a half-life of 4 to 6 hours with continued fibrinolysis that occurs in DVT and pulmonary embolism causing the D-Dimer level to remain elevated for about 7 days. D-dimer levels correlate with the presence of fibrin clots regardless of the location in the body where the clot is formed.

It has been stated that “the organism is an algorithm.” Therefore, subject to Moore's law and exponential growth, the implications are that information technology and digital health will play a surprisingly rapid and increasingly important role in global healthcare—for humans and animals. Accordingly, there is a need for a digital therapeutic device that is capable of the detection and analysis of biometric parameters, and the modification of treatments based on the biometric parameters, to enable digital healthcare option.

### SUMMARY

The below summary section is intended to be merely exemplary and non-limiting. The foregoing and other problems are overcome, and other advantages are realized, by the use of the exemplary embodiments of this invention.

In accordance with an aspect of the invention, an apparatus comprises an elastic support with at least one electrode supportable by the elastic support. The at least one electrode applies stimulation electrical signals to skin of a user. At least one urging member may be provided supportable by the elastic support adjacent to the at least one electrode for urging the at least one electrode towards the skin of the user.

In accordance with another aspect of the invention, a method comprises providing an elastic support substrate, and fixing at least one electrode to the elastic support substrate. The at least one electrode for applying stimulation electrical signals to skin of a user. At least one urging member is fixed to the elastic support substrate, wherein the at least one urging member is disposed adjacent to the at least one electrode for urging the at least one electrode towards the skin of the user.

In accordance with another aspect of the invention, an apparatus is provided for applying an electrical stimulation to skin of a user for at least one of mitigating pain, creating a haptic stimulation and for causing an involuntary muscle contraction. The apparatus comprising an elastic support, at least one electrode supportable by the elastic support and for applying stimulation electrical signals to skin of a user and at least one urging member supportable by the elastic support adjacent to the at least one electrode for urging the at least one electrode towards the skin of the user.

The at least one electrode may comprise a plurality of individually addressable electrodes supported by the elastic support. The individually addressable electrodes are for at least one of applying stimulation electrical signals to skin of a user and detecting biometric electrical signals from the skin of the user.

At least one of a signal detector for detecting the biometric electrical signals and a signal generator for generating the stimulation electrical signals may be provided. An electrode multiplex circuit may be provided for addressing the plurality of individually addressable electrodes by at least one of routing the biometric electrical signals from the skin of the user through more than one of the plurality of individually addressable electrodes to the signal detector and routing the stimulation electrical signals from the signal generator through more than one of the plurality of individually addressable electrode to the skin of the user; and a microprocessor for controlling at least one of the signal detector, the signal generator, the electrode multiplex circuit.

In accordance with an aspect of the invention, a method comprises applying a therapeutic treatment to a user. A biometric parameter indicative of a physiological change dependent on the applied therapeutic treatment is detected. The applied therapeutic treatment is modified dependent on the detected biometric signal.

In accordance with other aspect of the invention, an apparatus, comprises at least one processor; and at least one memory including computer program code. The at least one memory and the computer program code are configured, with the at least one processor, to cause the apparatus to perform at least the following: apply a therapeutic treatment to a user; detect a biometric parameter indicative of a physiological change dependent on the applied therapeutic treatment; and modify the applied therapeutic treatment dependent on the detected biometric signal.

In accordance with another aspect of the invention, a computer program product comprising a computer-readable medium bearing computer program code embodied therein for use with a computer. The computer program code comprising: code for applying a therapeutic treatment to a user; code for detecting a biometric parameter indicative of a physiological change dependent on the applied therapeutic treatment; and code for modifying the applied therapeutic treatment dependent on the detected biometric signal.

In accordance with another aspect of the invention, in a digital therapeutic, an electrical muscle stimulation signal is applied having at least one signal characteristic. The electrical muscle stimulation signal is applied to at least one muscle of a user. A biometric parameter indicative of a physiological change dependent on the applied electrical muscle stimulation signal is detected. The applied electrical muscle stimulation signal is modified dependent on the detected biometric signal.

In accordance with another aspect of the invention, a digital therapeutic device includes a wearable electronic garment for applying an electrical muscle stimulation signal through the skin to induce involuntary contractions in one or more muscles of a user. A biometric signal detector detects a biometric parameter indicative of a physiological change dependent on the applied electrical muscle stimulation signal. A microprocessor controls the application of the electrical signal dependent on the detected biometric signal.

In accordance with another aspect of the invention, in a digital therapeutic to treat and/or prevent an atherothrombotic event, an electrical muscle stimulation signal is applied having at least one signal characteristic. The electrical muscle stimulation signal is applied to at least one muscle adjacent to a blood vessel of a patient. The electrical muscle stimulation signals causes an involuntary contraction of the at least one muscle to impart a squeezing action on the blood vessel and promote a flow of blood through the blood vessel. A biometric parameter indicative of the flow of blood through the blood vessel is detected. The applied electrical muscle stimulation signal is modified dependent on the detected biometric signal.

In accordance with another aspect of the invention, a digital therapeutic device comprises a wearable electronic gait lent having at least one pair of electrodes for applying an electrical muscle stimulation signal through the skin to induce involuntary contractions in one or more muscles adjacent to a blood vessel. The involuntary muscle contractions induce a squeezing action on the blood vessel and promote a flow of blood through the blood vessel. A biometric signal detector detects a biometric parameter indicative of the flow of blood through the blood vessel. A microprocessor controls the application of the electrical signal dependent on the detected biometric signal. The biometric parameter may be dependent on a therapeutic action of a pharmaceutical medicinal compound. The applied electrical muscle stimulation signal may be modified in response to the therapeutic action of the pharmaceutical medicinal compound. The biometric signal may be dependent on at least one detectable biometric parameter, and wherein the biometric parameter is detectable dependent on at least one of skin temperature, skin color, blood flow, pulse, heartbeat, blood pressure, skin tightness, swelling, blood chemistry, sweat chemistry, an electronic biomarker, a chemical biomarker, and electromyography.

In accordance with another aspect of the invention, a wearable electronic uses electronically induced involuntary muscle contractions to pump blood through the blood vessels of the lower legs and prevent clots from forming. The wearable electronic includes a biometric parameter detector that generates a signal analyzed by an artificial intelligence agent embedded in the wearable electronic circuit to modify the applied electrical signal and optimize the involuntary muscle contractions.

In accordance with another aspect of the invention, a novel pharmaceutical medicinal compound includes a first compound having a determined therapeutic action on a patient and a second compound acting as a biometric indicator and having a chemical analyte detectable by a wearable electronic therapeutic device. The detection of the chemical analyte by the wearable electronic digital therapeutic device indicates the presence of the pharmaceutical medicinal compound in the patient. The chemical analyte may be detectable by the wearable electronic therapeutic device for positively indicating patient adherence to ingestion of the pharmaceutical medicinal compound.

In accordance with another aspect of the invention, a device for detecting the ingestion of a pharmaceutical medicinal compound comprises a wearable electronic digital therapeutic device including a biometric indicator detector for detecting a biometric indicator having a chemical analyte for positively indicating patient adherence to ingestion of the pharmaceutical medicinal compound. The pharmaceutical medicinal compound includes a first compound having a determined therapeutic action on the patient. A second compound acts as the biometric indicator and has the chemical analyte detectable by the wearable electronic digital therapeutic device. The detection of the chemical analyte by the wearable electronic digital therapeutic device indicates at least one of the absence and presence of the pharmaceutical medicinal compound ingested by the patient.

In accordance with the inventive apparatus, method and computer program product for the biometric detection of biomarkers, such as thrombin and/or D-dimer, new highly useful solutions are obtained for the treatment and monitoring of conditions related to coagulation and inflammation. The biometric detection of biomarkers, such as thrombin and/or D-dimer, is also disclosed for monitoring and treating a variety of cardiovascular, infectious, and inflammatory and autoimmune diseases.

In accordance with the inventive wearable electronic digital therapeutic device, a biomarker, such as thrombin or D-dimer, is detected, for example, through sweat chemistry analysis. The biomarkers can be used for the diagnosis and monitoring of cardiovascular conditions. These biomarkers can also be used to aid in modifying an administered pharmaceutical and/or electroceutical therapy. In accordance with the inventive wearable electronic digital therapeutic device, these biomarkers are used along with other automatically detected biometrics to enable the continuous and automatic monitoring of physiological changes in the patient with a higher degree of accuracy, convenience and accessibility than has been available with the conventional cardiovascular diagnostic and therapeutic techniques and apparatus.

In accordance with an aspect of the invention, a wearable electronic therapeutic device has one or more biometric detectors each for detecting one or more biometric parameters. The biometric parameters are dependent on at least one physiological change to a patient in response to a therapeutic treatment. A microprocessor receives the one or more biometric parameters and applies probabilistic analysis to determine if at least one physiological change threshold has been exceeded dependent on the probabilistic analysis of the two or more biometric parameters. An activation circuit activates an action depending on the determined exceeded physiological change. The action that is activated can be applying an electroceutical treatment in addition or as an alternative to a pharmaceutical treatment.

In accordance with an aspect of the invention, a method, comprises: detecting one or more biometric parameters, where the biometric parameters are dependent on at least one physiological change to a patient in response to a therapeutic treatment; receiving the one or more biometric parameters and applying probabilistic analysis to determine if at least one physiological change threshold has been exceeded dependent on the probabilistic analysis of the two or more biometric parameters; and activating an action depending on the determined exceeded said at least one physiological change.

In accordance with as aspect of the invention, an apparatus, comprises: at least one processor; and at least one memory including computer program code, the at least one memory and the computer program code configured, with the at least one processor, to cause the apparatus to perform at least the following: detect one or more biometric parameters, where the biometric parameters are dependent on at least one physiological change to a patient in response to a therapeutic treatment; receive the one or more biometric parameters by the at least one processor and apply probabilistic analysis to determine if at least one physiological change threshold has been exceeded dependent on the probabilistic analysis of the two or more biometric parameters; and activate an action depending on the determined exceeded said at least one physiological change.

In accordance with an aspect of the invention, a computer program product comprises a computer-readable medium bearing computer program code embodied therein for use with a computer, the computer program code comprising: code for detecting one or more biometric parameters, where the biometric parameters are dependent on at least one physiological change to a patient in response to a therapeutic treatment; code for receiving the one or more biometric parameters and applying probabilistic analysis to determine if at least one physiological change threshold has been exceeded dependent on the probabilistic analysis of the two ore more biometric parameters; and code for activating an action depending on the determined exceeded said at least one physiological change.

In accordance with an aspect of the invention, a wearable electronic garment has at least one pair of electrodes for applying an electrical muscle stimulation signal through the skin of a patient to induce involuntary contractions in one or more muscles adjacent to a deep vein blood vessel. The involuntary muscle contractions induce a squeezing action on the blood vessel and promote a flow of blood through the blood vessel in a direction towards a heart of the patient. A biometric signal detector detects a biometric parameter indicative of the flow of blood through the blood vessel. The biometric parameter is dependent on a therapeutic action of a pharmaceutical medicinal compound for inhibiting an initiation of coagulation of blood. A microprocessor modifies the application of the electrical signal dependent on the detected biometric signal. The applied electrical muscle stimulation signal is modified in response to the therapeutic action of the pharmaceutical medicinal compound.

In accordance with an aspect of the invention, a digital therapeutic device is provided for detecting a patient adherence to the ingestion of a medicinal compound. A wearable electronic digital therapeutic device includes a sweat chemistry sensor for sensing one or more water-soluble metabolites present in blood of a patient for positively indicating patient adherence to ingestion of the medicinal compound. The medicinal compound includes an initially ingested molecular structure that is insoluble in water that is metabolized after ingestion into the one or more water-soluble metabolites. The detection of the one or more water-soluble metabolites by the wearable electronic digital therapeutic device indicates an adherence by the patient to the ingestion of the medicinal compound. An on-demand sweat stimulator stimulates the production of sweat by the patient. The sweat is received by the sweat chemistry sensor for sensing the one or more water-soluble metabolites. A data transmitter is provided for transmitting data indicating the patient adherence to the ingestion of the pharmaceutical medicinal compound. The pharmaceutical medical compound may be, for example, a water-insoluble anticoagulant medicinal compound.

In accordance with another aspect of the invention, a digital therapeutic device is provided for detecting a patient adherence to the ingestion of a medicinal compound. A wearable electronic digital therapeutic device includes a chemistry sensor for sensing one or more water-soluble metabolites present in blood of a patient for positively indicating patient adherence to ingestion of the medicinal compound. The medicinal compound includes an initially ingested molecular structure that is insoluble in water that is metabolized after ingestion into the one or more water-soluble metabolites. The detection of the one or more water-soluble metabolites by the wearable electronic digital therapeutic device indicates an adherence by the patient to the ingestion of the medicinal compound.

In accordance with another aspect of the invention, a pharmaceutical medicinal compound comprises a first compound having a determined therapeutic action on a patient, and a second compound acting as a biometric indicator and having a metabolite acting as a chemical analyte detectable by a wearable electronic therapeutic device. The detection of the chemical analyte by the wearable electronic digital therapeutic device indicates the presence of the pharmaceutical medicinal compound in the body of the patient.

In accordance with another aspect of the invention, a device for detecting the ingestion of a pharmaceutical medicinal compound is provided comprising a wearable electronic digital therapeutic device. The wearable electronic digital therapeutic device includes a biometric indicator detector for detecting a biometric indicator having a chemical analyte detectable after ingestion of the pharmaceutical medicinal compound. The pharmaceutical medicinal compound includes a first compound having a determined therapeutic action on the patient as the first compound becomes bioactive within the body of a patient, and a second compound acting as the biometric indicator and having the chemical analyte detectable by the wearable electronic digital therapeutic device. The detection of the chemical analyte indicates the ingestion of the pharmaceutical medicinal compound.

In accordance with another aspect of the invention, a digital therapeutic device is provided comprising a wearable electronic therapeutic device having one or more biometric detectors each for detecting one or more biometric parameters. The biometric parameters are dependent on at least one physiological change to a patient. A microprocessor receives the one or more biometric parameters and determines if at least one physiological change threshold has been exceeded dependent on the one or more biometric parameters. An activation circuit activates an action depending on the determined exceeded physiological change. The action includes at least one of transmitting an alert, modifying a therapeutic treatment, and transmitting data dependent on at least one of the physiological changes, the one or more biometric parameters, and the therapeutic treatment.

In accordance with another aspect of the invention, a method comprises applying a therapeutic treatment to a patient. A biometric parameter indicative of a physiological change dependent on the applied therapeutic treatment is detected. The applied therapeutic treatment is modified dependent on the detected biometric signal.

In accordance with another aspect of the invention, a method and computer program product for the biometric detection of biomarkers, such as thrombin and/or D-dimer, new highly useful solutions are obtained for the treatment and monitoring of conditions related to coagulation and inflammation. The biometric detection of biomarkers, such as thrombin and/or D-dimer, is also disclosed for monitoring and treating a variety of cardiovascular, infectious, and inflammatory and autoimmune diseases.

In accordance with another aspect of the invention, a wearable electronic digital therapeutic device detects a biomarker, such as thrombin or D-dimer, for example, through sweat chemistry analysis. The biomarkers can be used for the diagnosis and monitoring of cardiovascular conditions. These biomarkers can also be used to aid in modifying an administered pharmaceutical and/or electroceutical therapy. In accordance with the inventive wearable electronic digital therapeutic device, these biomarkers are used along with other automatically detected biometrics to enable the continuous and automatic monitoring of physiological changes in the patient with a higher degree of accuracy, convenience and accessibility than has been available with the conventional cardiovascular diagnostic and therapeutic techniques and apparatus.

### BRIEF DESCRIPTION OF THE DRAWINGS

The foregoing and other aspects of exemplary embodiments of this invention are made more evident in the following Detailed Description, when read in conjunction with the attached Drawing Figures, wherein:

FIG. 1 shows a bare arm of a user;

FIG. 2 shows an embodiment of the inventive elastic bandage and dry electrode system being wrapped on the arm of the user;

FIG. 3 shows an end the inventive elastic bandage and dry electrode system being passed through a buckle and reversed in direction to facilitate wrapping on the arm of the user;

FIG. 4 shows the inventive elastic bandage and dry electrode system wrapped on the arm of the user;

FIG. 5 shows the inventive elastic bandage and dry electrode system being wrapped on the knee of the user;

FIG. 6 shows the inventive elastic bandage and dry electrode system wrapped on the knee of the user;

FIG. 7 shows the inventive elastic bandage and dry electrode system wrapped on the lower back of the user;

FIG. 8 shows the inventive elastic bandage and dry electrode system wrapped on the shoulder of the user;

FIG. 9(a) shows an embodiment of the inventive elastic bandage and dry electrode system comprised on a long elastic bandage, a number of dry electrodes integrally fixed on the elastic bandage, and an electronic TENS signal generator unit;

FIG. 9(b) shows the reverse side of the TENS unit, showing female connection snaps;

FIG. 10 shows the reverse side of the elastic bandage at the area of the dry electrodes showing male snap connectors that connect with the TENS unit with the connection indexable among the snap connectors to selectively conduct an electrical TENS signal to two of the dry electrodes;

FIG. 11 shows the constituent components that are assembled to form and embodiment of the inventive elastic bandage and dry electrode system for applying electro stimulation through the skin of the user;

FIG. 12 shows a jig and foam blocks used during the assembly of some of the constituent components into the inventive elastic bandage and dry electrode system through the application of heat and pressure;

FIG. 13 shows the jig with dry electrodes adhered to the foam blocks after the application of heat and pressure;

FIG. 14 shows the dry electrodes with the foam blocks adhered;

FIG. 15 show the complete dry electrode system adhered to the elastic bandage and connected to snap connectors for selective conduction of the TENS signal between two of the dry electrodes;

FIG. 16 shows another embodiment of the inventive elastic bandage and dry electrode system comprised on a long elastic bandage comprised of a neoprene middle layer with Velcro-compatible out layers, a number of dry electrodes removably fixable on the elastic bandage, and a stretchable fabric connection patch having adhered stretchable conductive fabric strips and connection snaps for mating with connection snaps on the removably fixable dry electrodes;

FIG. 17 shows the TENS unit connected to the neoprene elastic bandage;

FIG. 18 shows a fabric connection patch and stretchable conductive fabric strips prior to assembly on a electro stimulation wrist sleeve;

FIG. 19 shows the reverse side of an electro stimulation wrist sleeve;

FIG. 20 shows the obverse side of the electro stimulation wrist sleeve;

FIG. 21 shows a knee sleeve having removably fixable dry electrodes;

FIG. 22 shows a neoprene elastic bandage having nine individually addressable dry electrodes;

FIG. 23 shows the neoprene elastic bandage with nine snap connectors for individually addressing the nine individually addressable electrodes;

FIG. 24 shows an array of foam blocks disposed in a jig used for making the removably fixable dry electrodes;

FIG. 25 shows a first elastic fabric having an elastic TPU being applied to the array of foam blocks;

FIG. 26 shows the foam blocks adhered to the elastic fabric;

FIG. 27 shows the first elastic fabric and foam blocks flipped over and positioned on the jig;

FIG. 28 shows a second elastic fabric having a conductive surface protected by a release sheet being applied to the array of foam blocks adhered to the first elastic fabric;

FIG. 29 shows a completed sheet of uncut dry electrodes;

FIG. 30 shows the completed sheet of uncut dry electrodes being placed onto a steel rule the for cutting into individual dry electrodes;

FIG. 31 shows an embodiment of the removably fixable dry electrodes having a conductive surface comprised of Ag/AgCl elastic conductive screen-printed ink;

FIG. 32 shows an embodiment of the removably fixable dry electrodes having a conductive surface comprised of Ag/AgCl with an overprint of a carbon elastic conductive screen-printed ink;

FIG. 33 shows embodiments of the removably fixable dry electrodes having a conductive surface comprised of a TPU adhered conductive fabric;

FIG. 34 shows a splayed stretchable fabric wearable electronic sleeve having stretchable wiring leads disposed on a reverse surface;

FIG. 35 shows a splayed stretchable fabric wearable electronic sleeve having individually addressable electrodes on an obverse side;

FIG. 36 shows an assembled stretchable fabric wearable electronic sleeve formed using heat and pressure to adhere seams using an elastic TPU;

FIG. 37 shows a back support having an array of individually addressable removably fixed dry electrodes, where each dry electrode includes a bifurcated separately addressable conductive surface;

FIG. 38 shows the wiring connection plane of the back support shown in FIG. 37;

FIG. 39 shows a foam block having an embedded vibrator used for forming individually addressable electrodes that include a vibration feature;

FIG. 40 illustrates an exemplary embodiment showing electrical signals applied to a plurality of individually addressable electrodes routed through an electrode multiplex circuit and a signal multiplex circuit;

FIG. 41(a) is a top view of a removably fixable dry electrode;

FIG. 41(b) is an exploded cross-sectional side view of the removably fixable dry electrode having fluid permeable dry electrode fabric, a conductivity enhancing fluid charged foam and fluid absorbent holding material;

FIG. 41(c) is an assembly cross-sectional side view of the removably fixable dry electrode;

FIG. 42(a) shows a close-up of individually addressable dry electrode strips ganged on a stretchable fabric substrate constructed with fluid permeable dry electrode fabric, conductivity enhancing fluid charged foam and fluid absorbent holding material;

FIG. 42(b) shows a top view of the individually addressable dry electrode strips ganged on a stretchable fabric substrate;

FIG. 42(c) shows a top view of an assembled elastic wrap and the individually addressable dry electrode strips ganged on a stretchable fabric substrate;

FIG. 43 is a close-up view showing individually addressable dry electrode strips having a stretchable serpentine pattern ganged on a stretchable fabric substrate;

FIG. 44 shows an assembled elastic wrap having integrally fixed individually addressable dry electrode strips having a stretchable serpentine pattern;

FIG. 45(a) shows a first step to wrapping an embodiment of the individually addressable thy electrode strips assembled on an elastic bandage wrap on the calf of a patient;

FIG. 45(b) shows a second step to wrapping the embodiment of the individually addressable dry electrode strips assembled on an elastic bandage wrap;

FIG. 45(c) shows a third step to wrapping the embodiment of the individually addressable dry electrode strips assembled on an elastic bandage wrap;

FIG. 45(d) shows a fourth step to wrapping the embodiment of the individually addressable dry electrode strips assembled on an elastic bandage wrap;

FIG. 46(a) shows the inner side of a compression stocking having dry electrodes with fluid permeable dry electrode fabric, conductivity enhancing fluid charged foam and fluid absorbent holding material for applying electrical muscle stimulation signals to at least one of nerves and muscles of the patient;

FIG. 46(b) shows the outer side of the compression stocking with detectable electronics for generating the EMS signal;

FIG. 46(c) shows the electronics detached from snap connectors on the compression stocking and a remote control for user control of the electronics;

FIG. 47 illustrates a non-limiting embodiment of the inventive digital therapeutic device configured as a pair of leg stockings for applying an EMS therapy to the calf muscles of a patient;

FIG. 48 illustrates an embodiment of the inventive digital therapeutic worn on the calf of a patient;

FIG. 49 illustrates the location of EMS applying electrodes relative to muscles of the calf of a patient;

FIG. 50 illustrates the location of EMS applying electrodes relative to blood vessels of the calf of a patient;

FIG. 51 illustrates sequentially applied EMS signals applied via the EMS applying electrodes of the inventive digital therapeutic device;

FIG. 52 illustrates a deep vein showing the direction of blood flow towards the heart;

FIG. 53 illustrates biometric detecting, EMS/TENS applying, selective heat applying, electrodes in accordance with an embodiment of the inventive digital therapeutic device;

FIG. 54 illustrates the inventive digital therapeutic device configured as a leg stocking and having a multiple biometric sensors and multiple electrodes;

FIG. 55 shows a cross section of an inventive digital therapeutic device sweat chemistry sensor tuned to detect at least one biometric indicator associated with the presence of a therapeutic drug in the blood stream of a patient;

FIG. 56 shows a top view of the inventive digital therapeutic device sweat chemistry sensor tuned to detect at least one biometric indicator;

FIG. 57 is an isolated view showing a sweat collector of the inventive sweat chemistry sensor;

FIG. 58 shows a cross section of an inventive digital therapeutic device sensor patch with a suite of biometric detectors;

FIG. 59 shows a top view of the inventive digital therapeutic device sensor patch with a suite of biometric detectors;

FIG. 60 is a flow chart showing an algorithm for drug level and biometric parameter detection;

FIG. 61 is a flow chart showing an algorithm for detecting multiple biometric parameters used to determine when and in what quantity to deliver a drug and how to adjust an applied treatment signal;

FIG. 62 is a flow chart showing an algorithm for biometric parameter detection and analysis, and then the adjustment of an applied therapy dependent on that analysis;

FIG. 63 is a flow chart showing an algorithm for using a detected heartbeat biometric parameter to adjust an applied EMS therapy;

FIG. 64 is a flow chart showing an algorithm for using a detected blood flow biometric parameter to adjust an applied EMS therapy;

FIG. 65 is a flow chart showing an algorithm for using multiple detected biometric parameters to adjust an applied therapy;

FIG. 66 is a flow chart showing an algorithm for using multiple detected biometric parameters of heartbeat and blood flow to adjust an EMS applied therapy;

FIG. 67 illustrates an exemplary embodiment showing bi-directional electrical signals applied through a plurality of individually addressable electrodes routed through an electrode multiplex circuit and a signal multiplex circuit for applying a sequential EMS signal and detecting biometric feedback from the calf of a patient;

FIG. 68 is a flow chart showing an algorithm for detecting the blood level of a drug, detecting a biometric parameter related to a physiological effect of the drug, sending data related to the detected drug level and biometric parameter and using the detected blood level and biometric parameter to indicate or automatically adjust a dosage of the drug;

FIG. 69 is a flow chart showing an algorithm for detecting the blood level of a drug through sweat chemistry detection, detecting a biometric parameter related to a physiological effect of the drug, sending data related to the detected drug level and biometric parameter and using the detected blood level and biometric parameter to indicate or automatically adjust a dosage of the drug;

FIG. 70 illustrates the location of muscles of the calf of a patient targeted for sequential involuntary contractions in accordance with an embodiment of the inventive digital therapeutic;

FIG. 71 illustrates an embodiment of the inventive digital therapeutic having circumferential electrodes for applying a sequential EMS signal effective for causing simultaneous contractions in multiple targeted muscles synchronized with an expected and/or detected biometric parameter;

FIG. 72 illustrates an embodiment of the inventive digital therapeutic having multiple biometric detectors and multiple individually addressable electrodes to enable the use of multiple detected biometric parameters to adjust an applied therapy;

FIG. 73 illustrates the inventive digital therapeutic for selectively applying transcutaneous electrical muscle and/or nerve stimulation as an applied therapy and selectively detecting electromyography as a biometric parameter through the same electrodes and/or circuit components;

FIG. 74 is a flow chart showing an algorithm for detecting the taking of a target drug along with a biometric indicator incorporated in the same pill or capsule or otherwise imparted into the patient at the same time as the target drug, and using detection of the biometric indicator as a positive indication that the target drug has been taken by the patient;

FIG. 75 is a flow chart showing a Body-in-the-Loop™ algorithm for detecting the blood level of a drug, detecting a biometric parameter related to at least one of a biometric indicator taken along with the drug and/or a physiological effect of the drug, logging data related to the detected drug level and biometric parameter and using the detected blood level and biometric parameter to indicate or automatically adjust a dosage of the drug;

FIG. 76 is a cross section of a pharmaceutical pill having a target drug and a biometric indicator, where the biometric indicator is detectable by the inventive digital therapeutic for positively indicating patient adherence to the ingestion of the target drug;

FIG. 77 is a cross section of a pharmaceutical pill having a controlled release target drug and a fast release biometric indicator, where the fast release biometric indicator provides a relatively quicker detectable signal compared to the controlled release target drug for positively indicating patient adherence to the ingestion of the target drug;

FIG. 78 is a cross section of a capsule containing a time released target drug and a time released biometric indicator, where the biometric indicator remains detectable for a duration that relates to the time release of the target drug to provide an indication of the activity of the target drug from ingestion through to full or partial metabolism (or other activation/deactivation mechanism);

FIG. 79 is a cross section of a capsule containing a time released target drug and a time released biometric indicator, where the biometric indicator remains detectable for a duration that relates to the time release of the target drug, the capsule shell contains a fast release biometric indicator to provide a relatively quicker detectable signal compared to the slow release biometric indicator for positively indicating patient adherence to the ingestion of the target drug;

FIG. 80 is a flow chart showing an algorithm for detecting a patient's adherence to a scheduled drug ingestion of a target drug through the detection of the presence of a biometric indicator;

FIG. 81 is a flow chart showing an algorithm for detecting the taking of a target drug along with a biometric indicator incorporated in the same pill or capsule or otherwise imparted into the patient at the same time as the target drug and using detection of the biometric indicator as a positive indication that the target drug has been taken by the patient;

FIG. 82 shows the legs of a patient showing the location of popiliteal and tibial blood vessels at the back of the knee and saphenous and pedis blood vessels at the ankle;

FIG. 83 illustrates a sock showing a sweat stimulator/collector and block diagram of electronics;

FIG. 84 illustrates an embodiment of the inventive wearable electronic digital therapeutic device having blood vessel detectors and sweat chemistry sensors;

FIG. 85 illustrates an embodiment of the inventive wearable electronic digital therapeutic for the analysis of a therapeutic effect based on an activated physiological change and one or more detected biometric parameters;

FIG. 86 is a top view of components of a sweat chemistry sensor that includes an activatable physiological change in form of induced sweat stimulation;

FIG. 87 is a cross sectional view of an iontophoresis patch sweat chemistry sensor;

FIG. 88 is a flow chart illustrating an algorithm for analysis of a therapeutic effect based on an activated physiological change and one or more detected biometric parameters;

FIG. 89 illustrates an embodiment of the inventive wearable electronic digital therapeutic for the analysis and modification of pharmaceutical and/or electroceutical treatments based on an activated physiological change and detected biometric parameters;

FIG. 90 is a flow chart illustrating an algorithm for the analysis and modification of pharmaceutical and/or electroceutical treatments based on an activated physiological change and detected biometric parameters;

FIG. 91 is a flow chart illustrating an algorithm for analysis of an anticoagulant therapeutic effect based on an activated sweat stimulation detected biomarkers, such as thrombin and/or D-Dimer, and blood flow biometric parameters;

FIG. 92 is a cross section of a rodent tail showing the location of blood vessels and an optical detection system scaled for detecting biometric parameters on the rodent tail;

FIG. 93 is an isolated view of a foot of a rodent showing foot pad and sweat glands;

FIG. 94 is an isolated view of a foot of a rodent showing a sweat collection sock and iontophoresis sweat stimulation/chemistry detection patch;

FIG. 95 illustrates a rodent with a biometric detection system installed on the tail the rodent;

FIG. 96 illustrates an embodiment of the inventive wearable electronic digital therapeutic device configured as a pair of stockings for a combination thrombosis/PAD detection with muscle pump EMS therapy;

FIG. 97 is a flowchart of an algorithm for a combination thrombosis/PAD detector with muscle pump EMS activation system;

FIG. 98 illustrates an embodiment of the inventive wearable electronic digital therapeutic device configured as a pair of stockings for a combination thrombosis/PAD detection with muscle pump EMS therapy;

FIG. 99 illustrates a series of user interface screens for an inventive thrombosis/PAD detection stockings with muscle pump EMS therapy;

FIG. 100 shows a cross section of an inventive digital therapeutic device sweat chemistry sensor tuned to detect at least one biometric indicator associated, for example, with the presence of a therapeutic drug in the blood stream of a patient;

FIG. 101 shows a top view of the inventive digital therapeutic device sweat chemistry sensor tuned to detect at least one biometric indicator;

FIG. 102 is an isolated view showing a sweat collector of the inventive sweat chemistry sensor;

FIG. 103 shows a cross section of an inventive digital therapeutic device sensor patch with a suite of biometric detectors;

FIG. 104 shows a top view of the inventive digital therapeutic device sensor patch with a suite of biometric detectors;

FIG. 105 shows a first step in forming a sweat collector having a flow through structure;

FIG. 106 shows a second step in forming a sweat collector having a flow through structure;

FIG. 107 shows a third step in forming a sweat collector having a flow through structure;

FIG. 108 shows a fourth step in forming a sweat collector having a flow through structure;

FIG. 109 illustrates an embodiment of the inventive wearable electronic digital therapeutic for the analysis of a therapeutic effect based on an activated physiological change and multiple detected biometric parameters;

FIG. 110 is a top view of components of a sweat chemistry sensor that includes an activatable physiological change in form of induced sweat stimulation and a moisture barrier to retain the sweat induced from the sweat stimulation;

FIG. 111 is a cross sectional view of an iontophoresis patch sweat chemistry sensor with a moisture barrier;

FIG. 112 is a cross section of a pharmaceutical pill including a water insoluble target drug having a water soluble metabolite and a shell, where the water soluble metabolite is detectable by the inventive digital therapeutic for positively indicating patient adherence to the ingestion of the target drug;

FIG. 113 is a cross section of a pharmaceutical pill having a water insoluble target drug having a water soluble metabolite and a fast release biometric indicator, where the fast release biometric indicator provides a relatively quicker detectable signal compared to the metabolism of the target drug for positively indicating patient adherence to the ingestion of the target drug through detection of the biometric indicator and for determining therapeutic conditions of the target drug from the detection of the metabolite;

FIG. 114 is a cross section of a capsule containing a water insoluble target drug having a water soluble metabolite and a time released biometric indicator, where the biometric indicator remains detectable for a duration that relates to the time release of the target drug to provide an indication of the activity of the target drug from ingestion through to full or partial metabolism (or other activation/deactivation mechanism) for comparison with the detection of the water soluble metabolite;

FIG. 115 is a cross section of a capsule containing a time released water insoluble target drug having a water soluble metabolite and a time released biometric indicator, where the biometric indicator remains detectable for a duration that relates to the time release of the target drug, the capsule shell contains a fast release biometric indicator to provide a relatively quicker detectable signal compared to the slow release biometric indicator for positively indicating patient adherence to the ingestion of the target drug;

FIG. 116 is a flow chart showing an algorithm for detecting a patient's adherence to a scheduled drug ingestion of a target drug through the detection of the presence of a biometric indicator;

FIG. 117 is a flow chart showing an algorithm for detecting the taking of a target drug along with a biometric indicator incorporated in the same pill or capsule or otherwise imparted into the patient at the same time as the target drug and using detection of the biometric indicator as a positive indication that the target drug has been taken by the patient;

FIG. 118 shows a water-insoluble anticoagulant drug and molecular pathways to water-soluble metabolites, in this example, the water-insoluble anticoagulant drug is rivaroxaban;

FIG. 119(a) shows a water-insoluble molecule of a therapeutic medicinal compound;

FIG. 119(b) shows a water-soluble molecule that is a metabolite of the water-insoluble molecule;

FIG. 120 is a flow chart illustrating an algorithm for the formulation and delivery of patch delivered bioactive water-soluble and/or nanoparticle constituents of a therapeutic medicinal compound(s);

FIG. 121 illustrates an embodiment of the inventive wearable electronic digital therapeutic device;

FIG. 122 is a flow chart illustrating an algorithm for determining drug administration patient adherence;

FIG. 123 illustrates constituent parts of a system for remotely monitoring and controlling a wearable electronic digital therapeutic device;

FIG. 124 is a flow chart illustrating an algorithm for analyzing a therapeutic effect based on an activated physiological change and detected biometric parameters;

FIG. 125 illustrates an embodiment configured as a wristwatch, bracelet, sleeve or armband;

FIG. 126 is a flow chart illustrating an algorithm for modifying a combination of pharmaceutical and electroceutical treatments based on an activated physiological change and detected biometric parameters;

FIG. 127 is a flow chart illustrating an algorithm for using a Body-in-the-Loop™ digital therapeutic for dosage adjustment based on detected in-vivo drug levels and biometric parameters;

FIG. 128 illustrates a patch configuration of an embodiment;

FIG. 129 illustrates a multi-part configuration having a printed electronics flexible display with near-distance and mid/long-distance relayed wireless communications of an embodiment;

FIG. 130 illustrates a ring configuration of an embodiment;

FIG. 131 illustrates an anklet configuration of an embodiment;

FIG. 132 illustrates the location of various biometric detectors/sensors/transmitters/processors/actuators;

FIG. 133 illustrates an embodiment of a pharmaceutical/electroceutical combination treatment device for applying an electroceutical signal in combination with an administered pharmaceutical and detecting a biometric physiological response;

FIG. 134 illustrates an embodiment of a pharmaceutical/electroceutical combination treatment device for monitoring physiological changes in response to an administered pharmaceutical;

FIG. 135 is a flow chart illustrating an algorithm for applied probabilistic analysis to determine a concerning physiological change;

FIG. 136 is a flow chart illustrating an algorithm for an early warning system with applied probabilistic analysis of multiple biometric parameters;

FIG. 137 is a flow chart illustrating an algorithm for a single parameter early warning system;

FIG. 138 is a flow chart illustrating an algorithm for biometric fusion analysis of multiple biometrics to determine a physiological change;

FIG. 139 is a flow chart illustrating an algorithm of a single parameter thrombosis early warning system;

FIG. 140 is a flow chart illustrating an algorithm a multiple parameter thrombosis early warning system;

FIG. 141 illustrates the location of blood vessels in the lower leg;

FIG. 142 illustrates the location of blood pulse/vessels under the skin surface of the lower leg;

FIG. 143 illustrates a wearable electronic digital therapeutic device configured as a stocking for detecting biometric parameters including blood pressure using a blood pressure cuff and for using the pressure cuff for applying a compressive therapeutic action in combination with electroceutical and/or pharmaceutical therapies;

FIG. 144 is a flow chart illustrating an algorithm for extrapolating biometric parameters for detecting physiological change;

FIG. 145 illustrates an embodiment of a partial stocking with a drawstring closure for adjustably fitting the electrodes and sensors/detectors/actuators in face to face contact with the skin of the leg;

FIG. 146 illustrates the partial stocking with the drawstring closure shown with a snug fit to the lower leg;

FIG. 147 illustrates an embodiment of a partial stocking with a stirrup portion;

FIG. 148 shows a hand of a patient with upper limb contracture;

FIG. 149 shows a hand and forearm of a patient with upper limb contracture;

FIG. 150 shows the location of muscles and EMS electrodes for an embodiment of the inventive contracture sleeve;

FIG. 151 illustrates an embodiment of the inventive contracture sleeve showing the location of electrodes for applying EMS to the lower forearm muscles;

FIG. 152 shows the location of muscles and EMS electrodes for an embodiment of the inventive contracture sleeve;

FIG. 153 illustrates an embodiment of the inventive contracture sleeve showing the location of electrodes for applying EMS to the upper forearm muscles;

FIG. 154 shows the lower muscles of a forearm in a contracture position;

FIG. 155 shows the upper muscles of a forearm in a contracture position;

FIG. 156 shows the lower muscles of a forearm stretching a contracture;

FIG. 157 shows the upper muscles of a forearm stretching a contracture;

FIG. 158 illustrates constituent parts of a system for remotely monitoring and controlling a wearable electronic digital therapeutic device;

FIG. 159 is a flow chart of an algorithm for a detect, analyze-to-treat, and apply wearable electronic digital therapeutic;

FIG. 160 is a flow chart of an algorithm for a contracture sleeve;

FIG. 161 is a flow chart of an algorithm for a pinch and grasp sleeve;

FIG. 162 illustrates an exemplary embodiment showing bi-directional electrical signals applied through a plurality of individually addressable electrodes routed through an electrode multiplex circuit and a signal multiplex circuit for applying a sequential EMS signal and detecting biometric feedback from the muscles of a patient;

FIG. 163 illustrates the plurality of individually addressable electrodes showing the muscles and nerves underlying the skin of the patient;

FIG. 164 shows a configuration of a plurality of individually addressable electrodes having biometric signal detection electrodes disposed in pairs that approximately line up with the long axis of muscles in the forearm of a patient, along with reference electrodes disposed between the electrode pairs;

FIG. 165 shows a three-dimensional representation of a pattern of individually addressable electrodes for an Haptic Human/Machine Interface (HHMI) forearm sleeve;

FIG. 166 illustrates an electrode pattern for an HHMI forearm sleeve for detecting and applying electrical singles using a single signal detector and a single signal generator, with a multiplexor circuit system for routing the electrical signals;

FIG. 167 is an illustration showing the muscles of the arm of a patient;

FIG. 168 is an illustration showing an exemplary embodiment of the inventive HHMI configured as a sleeve disposed on the arm of the patient;

FIG. 169 illustrates an embodiment of the inventive HHMI configured as a sleeve having addressable electrodes connected via a grid of x and y electrodes;

FIG. 170 is a flow chart showing a calibration algorithm for calibrating the HHMI to an individual patient's body;

FIG. 171 is a flow chart showing a refinement algorithm for refining the calibration of the HIM;

FIG. 172 illustrates an electrode pattern for an HHMI forearm sleeve for detecting and applying electrical signals using a single signal detector and a single signal generator, with a multiplexor circuit system for routing the electrical signals;

FIG. 173 is a photograph showing an embodiment of a contracture sleeve having a compression sleeve portion, foam urging block adjacent to electrodes disposed in use in face to face electrical contact with the forearm skin, and an elastic bandage for applying an urging force against the foam block to press the electrodes against the skin;

FIG. 174 is a photograph showing an embodiment of a contracture sleeve having a compression sleeve portion and showing the elastic bandage for applying an urging force against the foam block to press the electrodes against the skin;

FIG. 175 is a photograph showing an embodiment of a contracture sleeve having a compression sleeve portion located on the upper forearm of the patient, with a foam urging block adjacent to electrodes disposed in use in face to face electrical contact with the forearm skin, and an elastic bandage wrapped around the forearm for applying an urging force against the foam block to press the electrodes against the skin; and

FIG. 176 is a photograph showing a configuration of a stocking having biometric detectors, microprocessor, battery and EMS signal generator.

### DETAILED DESCRIPTION

Below are provided further descriptions of various non-limiting, exemplary embodiments. The exemplary embodiments of the invention, such as those described immediately below, may be implemented, practiced or utilized in any combination (e.g., any combination that is suitable, practicable and/or feasible) and are not limited only to those combinations described herein and/or included in the appended claims. Throughout this application, an applied electrical signal is denoted as being a TENS, EMS, NMES, or other acronym. The difference among the applied electrical signal may be one of frequency or other signal characteristic and unless specified or otherwise inferable through the descriptive context, the terms and acronyms used for the applied electrical signal may be considered interchangeable with each other. For example, TENS will typically be used when describing an electrical signal applied for pain mitigation however, as used herein it might also be used when describing a signal that invokes an involuntary muscle contraction.

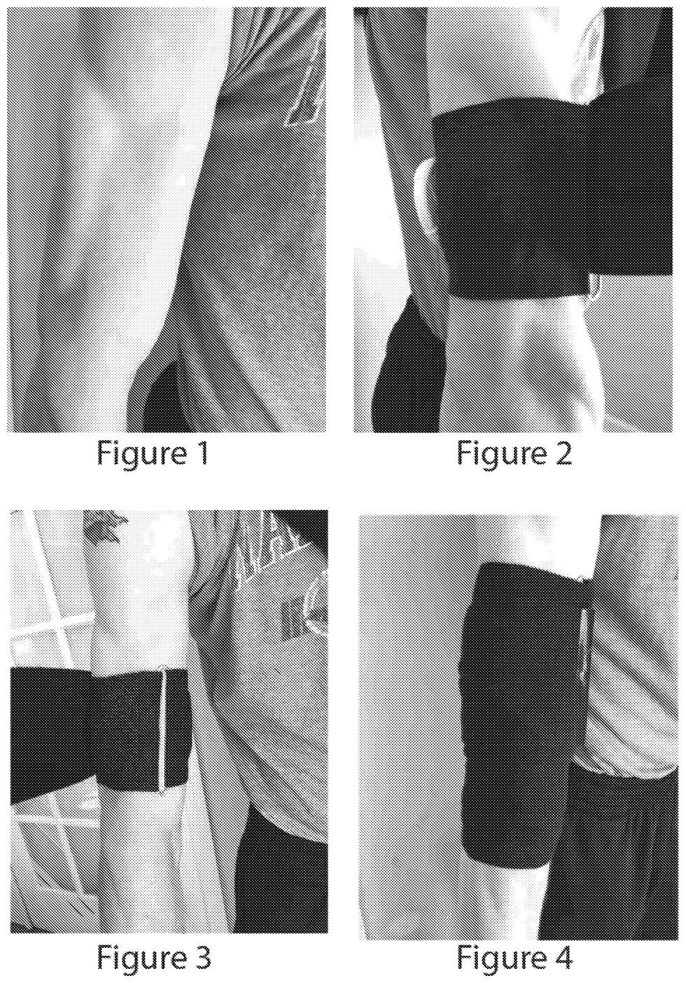

A non-limiting exemplary embodiment, FIGS. 1-176 shown, for example, an apparatus that comprises an elastic support with at least one electrode supportable by the elastic support. The at least one electrode for applying stimulation electrical signals to skin of a user. At least one urging member is supportable by the elastic support adjacent to the at least one electrode for urging the at least one electrode towards the skin of the user to ensure adequate surface area contact between the electrode and the skin surface. Many configurations, embodiments, methods of manufacture, algorithms, electronic circuits, microprocessor, memory and computer software product combinations, networking strategies, database structures and uses, and other aspects are disclosed herein for a wearable electronic digital therapeutic device and system that has a number of medical and non-medical uses.

A dry electrode can be provided supportable by the elastic support and separate from or integrated with the elastic support. That is, the dry electrode and the elastic support can be separate components allowing the insert to be positioned relative to the elastic support to benefit the ergonomics or other application factors for an individual user or specific application. The dry electrode includes the at least one electrode surface.

The dry electrode further can include the at least one urging member. The dry electrode and the elastic support interact to cooperatively hold the at least one electrode in electrical contact with the skin of the user. For example, in the exemplary embodiments shown herein, the dry electrode is held snug against the skin of the user by the elastic support. The elastic support can comprise an elastic fabric for applying a squeezing force against the dry electrode for cooperatively acting with the least one urging member for holding and urging the at least one electrode in electrical contact with the skin of the user.

The at least one electrode may comprise a conductive fabric electrode fixed to at least one of the elastic support and the dry electrode. The at least one electrode can comprise a dry electrode formed by at least one of digital inkjet printing, screen printing, doctor blading, stamping, dip coating and spray painting of a conductive ink. The dry electrode may be made from a conductive fabric, conductive yarn, rubber impregnated with a conductive particulate such as carbon, silver, silver chloride, or the like.

The dry electrode may be constructed to apply the electrical signal through capacitive stimulation using polarizable electrodes that have little or no direct skin contact. This this case, the capacitor skin surface electrode has a relatively uniform current distribution and may stimulate motor units and muscles with the less skin sensation. As an example, a glass tube with a metal coating inside the tube has been proposed can be used with a high voltage of up to 60 kV to deliver about 40 mA currents with a pulse duration of about 70 μs. Using a capacitive electrode, twitches and tetanic contractions may be produced with little skin sensation (see, for example, Electrodes for transcutaneous (surface) electrical stimulation, Thierry Keller and Andreas Kuhn, JOURNAL OF AUTOMATIC CONTROL, UNIVERSITY OF BELGRADE, VOL. 18(2):35-45, 2008).

The dry electrode may be constructed to apply the electrical signal through the transference of the electrical signal from the conductive surface to the skin of the user. In the case of a printed electrode, a robust exposed electrode printing (REEP™) process can be used as described in PCT/US17/62429, filed Nov. 17, 2017, the entirety of which is incorporated by reference herewith.

The urging member may comprise at least one of a pneumatic bladder, a foam block, a wire spring, and an elastic fabric. The volume (and hence the pressure applied as the urging force) of the pneumatic bladder can be adjustable, for example, using an air pump. Additional urging members can be included between the elastic support and the dry electrode to provide a custom fit for a particular user's body, preference, or a specific application of the inventive electrical signal detector and/or applier system.

The at least one electrode may comprise a dry electrode pre-printed on a print medium, such as a TPU, and the at least one electrode fixed to the elastic support substrate by adhering the print medium to the elastic support substrate. The at least one electrode may comprise the dry electrode fixed to the elastic support substrate and or by printing the dry electrode onto the elastic support substrate.

The urging member may a respective foam block configured and dimensioned for urging a corresponding electrode towards the skin of the user. A cavity can be formed in the elastic support substrate and or in the dry electrode. The cavity is positioned adjacent to a corresponding electrode. The at least one urging member may comprise a compressible foam block configured and dimensioned to be received in the cavity effective for urging the electrode into face to face contact with the skin.

In accordance with a non-limiting exemplary embodiment, an apparatus is provided for applying an electrical stimulation to skin of a user for invoking involuntary muscle contractions, providing a massaging sensation, invoking skin sensation and/or mitigating pain. The apparatus comprises an elastic support with at least one electrode supportable by the elastic support for applying stimulation electrical signals to skin of a user. At least one urging member is supportable by the elastic support adjacent to the at least one electrode for urging the at least one electrode towards the skin of the user.

FIG. 1 shows a bare arm of a user. FIG. 2 shows an embodiment of the inventive elastic bandage and dry electrode system being wrapped on the arm of the user. FIG. 3 shows an end the inventive elastic bandage and dry electrode system being passed through a buckle and reversed in direction to facilitate wrapping on the arm of the user. FIG. 4 shows the inventive elastic bandage and dry electrode system wrapped on the arm of the user. In accordance with an aspect of the invention, an apparatus comprises an elastic support with at least one electrode supportable by the elastic support. The at least one electrode applies stimulation electrical signals to skin of a user. At least one urging member may be provided supportable by the elastic support adjacent to the at least one electrode for urging the at least one electrode towards the skin of the user.

When wrapping the elastic bandage, the dry electrodes are placed at a suitable location to apply the TENS signal through the skin to the underlying nerves and muscles. In accordance with an embodiment, a buckle is provided at the electrode end to facilitate wrapping the elastic bandage so that a conductive surface of the dry electrodes is urged into face to face electrical contact with the skin of the user. During the wrapping, the distal end of the elastic bandage is passed through the buckle and the wrapping direction reversed so that elastic bandage can be cinched and held in place on the arm or other body part of the user. The elastic bandage may be suitably long enough so that the electrodes have an adequate urging force to maintain suitable face to face electrical contact, and so that the remaining portion of the elastic bandage can be wrapped to provide support and compression to a joint or other body part of the user.

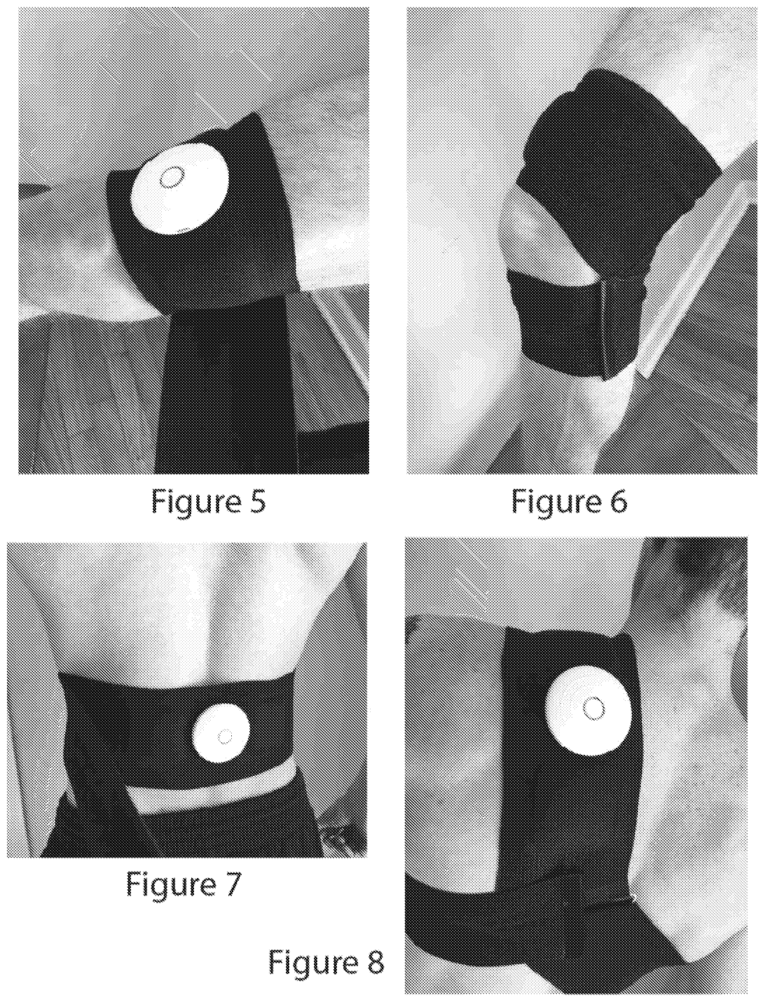

FIG. 5 shows the inventive elastic bandage and dry electrode system being wrapped on the knee of the user. FIG. 6 shows the inventive elastic bandage and dry electrode system wrapped on the knee of the user. The inventive elastic bandage and dry electrode system can be used on a wide range of body part of the user's body. For example, the knee of the user can be wrapped so that the dry electrodes are disposed at a suitable position to apply a TENS signal as desired. The elastic bandage is long enough to provide additional wrapping length so that the knee joint can be adequately supported in addition to the application of the TENS signal. The electrodes may be spaced and connected so that a desired spacing between the active electrodes can be selected to the when wrapped in a spiral, the active electrodes are spaced on the longitudinal length of the same muscle or spaced to span across two or more muscles, etc.

FIG. 7 shows the inventive elastic bandage and dry electrode system wrapped on the lower back of the user. FIG. 8 shows the inventive elastic bandage and dry electrode system wrapped on the shoulder of the user. Other body parts that can be wrapped to apply TENS signals and also compression and support include, but are not limited to, the wrist, hand, forearm, elbow, foot, ankle, calf, thigh, hips, stomach.

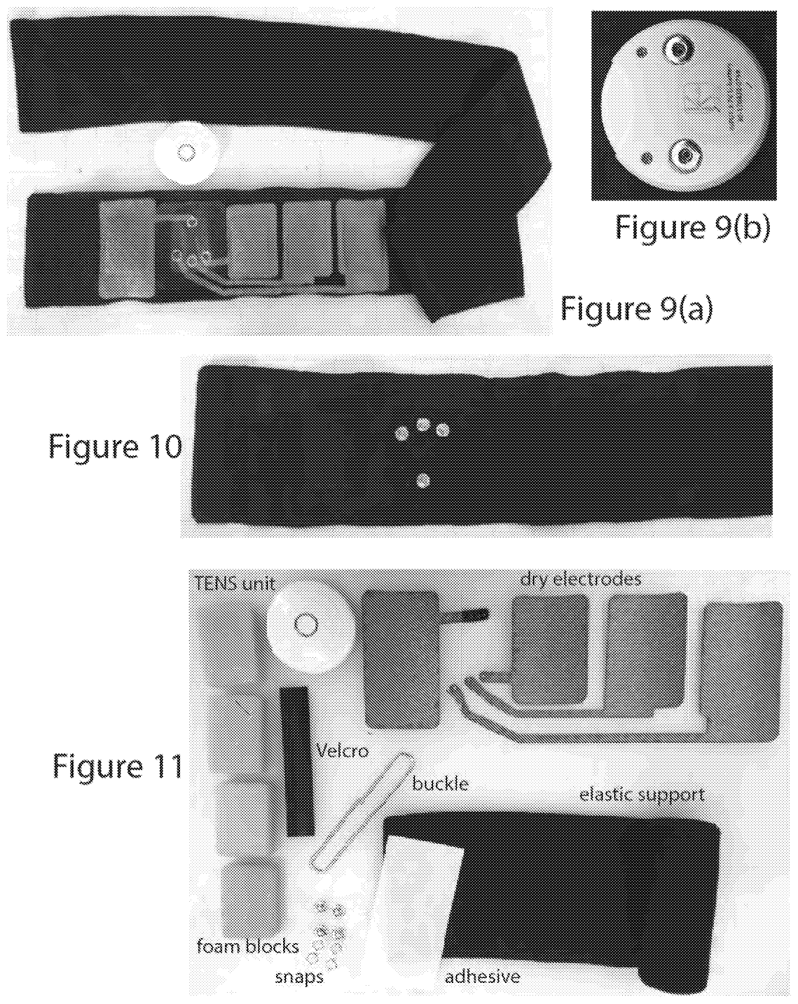

FIG. 9(a) shows an embodiment of the inventive elastic bandage and dry electrode system comprised on a long elastic bandage, a number of dry electrodes integrally fixed on the elastic bandage, and an electronic TENS signal generator unit. FIG. 9(b) shows the reverse side of the TENS unit, showing female connection snaps. The connecting side of the TENS unit has female snap connectors that receive the male snap connectors fixed on the elastic bandage. The TENS unit may have, for example, female snap connectors for mating with and electrically and mechanically connecting with male snaps fixed to the elastic bandage and connected with the dry electrodes.

FIG. 10 shows the reverse side of the elastic bandage at the area of the dry electrodes showing male snap connectors that connect with the TENS unit with the connection indexable among the snap connectors to selectively conduct an electrical TENS signal to two of the dry electrodes. As an example, the TENS unit may include a first female connector and a second female connect. A circuit for applying the TENS signal is completed when a first male connector of a first dry electrode is connected to the first female connect, and a second male connector of a second dry electrode is connected to the second female connector. The human body itself completes the electrical circuit and allows the TENS signal to be applied from the first and second female connectors of the TENS unit. As shown, two or more dry electrodes with respective male connectors may be provided so that the female connectors of the TENS unit can be indexed and select which pair of dry electrodes receive the TENS signal. By this construction, the spacing, geometry and location of the dry electrodes can be selected depending on the desired location on the user's body for applying the TENS signal.

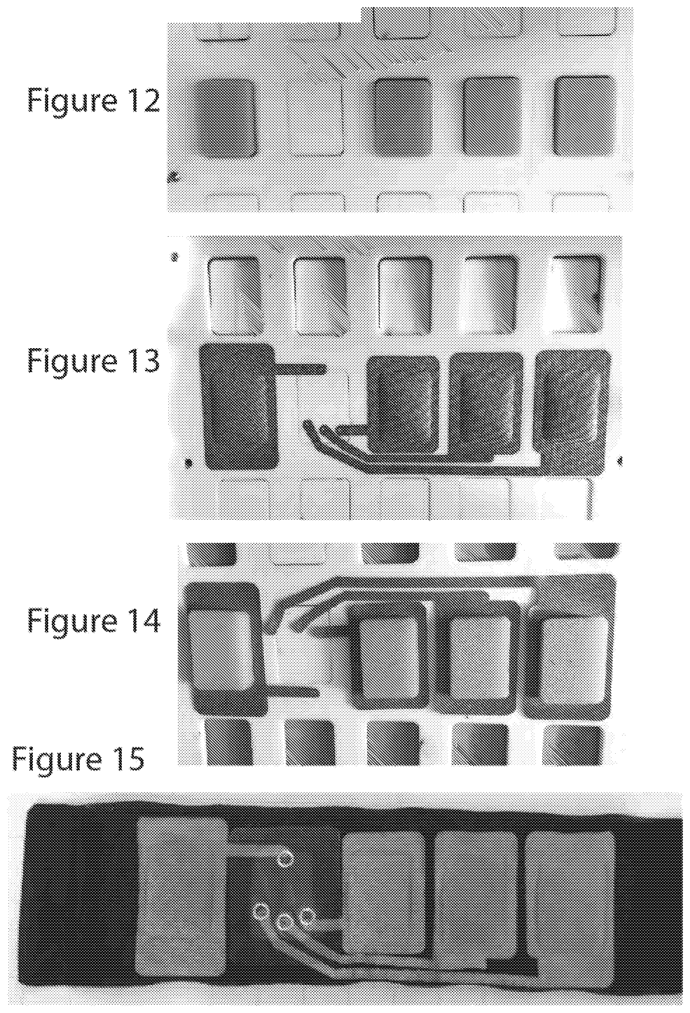

FIG. 11 shows the constituent components that are assembled to form an embodiment of the inventive elastic bandage and dry electrode system for applying electro stimulation through the skin of the user. The constituent parts include the TENS unit, dry electrodes, Velcro strip, buckle, foam blocks, elastic bandage support, adhesive and snaps.

FIG. 12 shows a jig with inserted foam blocks. The jig is used during the assembly of some of the constituent components into the inventive elastic bandage and dry electrode system through the application of heat and pressure. The at least one electrode comprises a conductive surface and a foam urging member disposed between the elastic support and the conductive surface. The jig is made from a heat resistance material, such as Teflon, and is able to withstand heat and pressure from a heated press. The heated press is used to apply heat and pressure to the materials held in the jig so that a TPU or adhesive sheet can be activated to bond the materials together. The jig holds the relative positions of the materials and accommodates, for example, the thickness of the foam blocks to improve the final dimensions and shape of the finished product.

FIG. 13 shows the jig with dry electrodes adhered to the foam blocks after the application of heat and pressure. FIG. 14 shows the dry electrodes with the foam blocks adhered. FIG. 15 shows the complete dry electrode system adhered to the elastic bandage and connected to snap connectors for selective conduction of the TENS signal between two of the dry electrodes. Each of the steps performed by the heat press and jig may be adapted to a scalable high-volume roll-to-roll manufacturing process. For example, heated pressure rollers can be used to apply the heat and pressure to a stack of materials to form a lamination package. The lamination package may comprise all or a portion of the constituent parts so that multiple passes through heated rollers can be used to successively build up the constituent parts as described herein. Also, a combination of flat presses and roller presses can be employed depending on the production line cost target, throughput and assembly methods employed.

The method for forming the inventive elastic bandage and dry electrode system may include providing an elastic support substrate. At least one electrode is fixed to the elastic support substrate. The at least one electrode is for applying stimulation electrical signals to skin of a user. At least one urging member can be fixed to the elastic support substrate, wherein the at least one urging member is disposed adjacent to the at least one electrode for urging the at least one electrode towards the skin of the user. The at least one electrode may comprise a dry electrode pre-printed on a print medium, such as a TPU or hotmelt sheet adhesive. The at least one electrode may be fixed to the elastic support substrate by adhering the print medium to the elastic support substrate. The at least one electrode may comprise a dry electrode fixed to the elastic support substrate by printing the dry electrode directly onto an elastic support substrate (e.g., the elastic bandage, stretchable fabric, or Velco-compatible neoprene) or a layer supported by the elastic support substrate. The urging member may comprise a respective foam block configured and dimensioned for urging a corresponding electrode towards the skin the user. A cavity may be formed in the elastic support substrate, for example, as a flap of material adhered or sewn to the elastic support substrate, or as a pocket formed in the elastic support substrate, that is positioned adjacent to a corresponding electrode. The at least one urging member may comprise the compressible block configured and dimensioned to be received in the cavity effective for urging the conductive surface against the skin of the user.

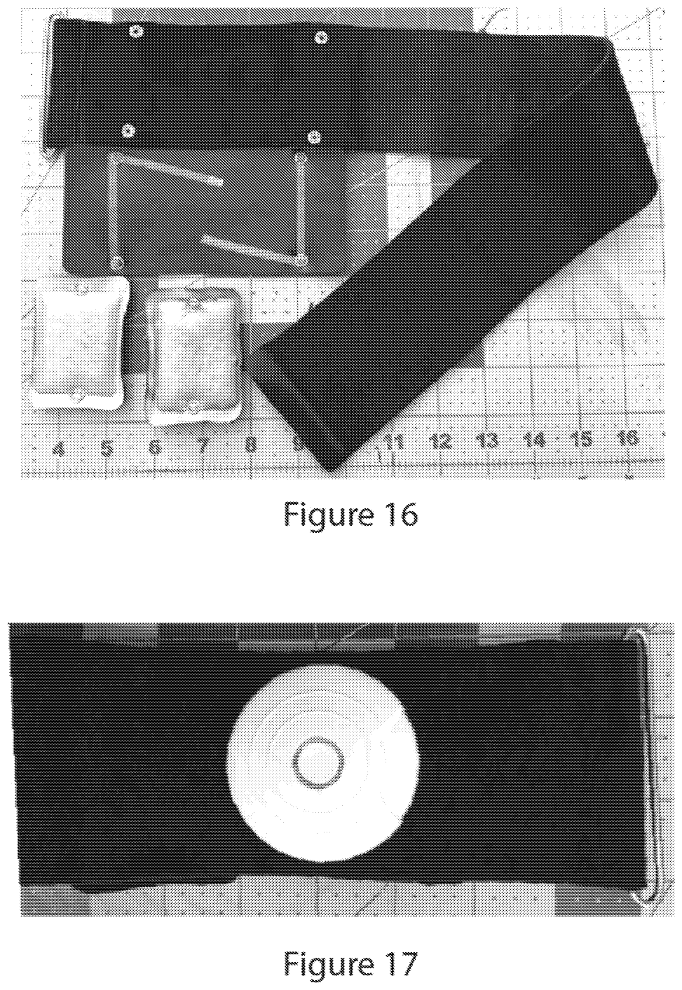

FIG. 16 shows another embodiment of the inventive elastic bandage and dry electrode system comprised on a long elastic bandage comprised of a neoprene middle layer with Velcro-compatible out layers, a number of dry electrodes removably fixable on the elastic bandage, and a stretchable fabric connection patch having adhered stretchable conductive fabric strips and connection snaps for mating with connection snaps on the removably fixable dry electrodes. The removably fixable dry electrode are supportable by the elastic support and separate from the elastic support, wherein the dry electrode includes the at least one electrode. The dry electrode may further include the at least one urging member, and the dry electrode and the elastic support interact to hold the at least one electrode in electrical contact with the skin of the user.

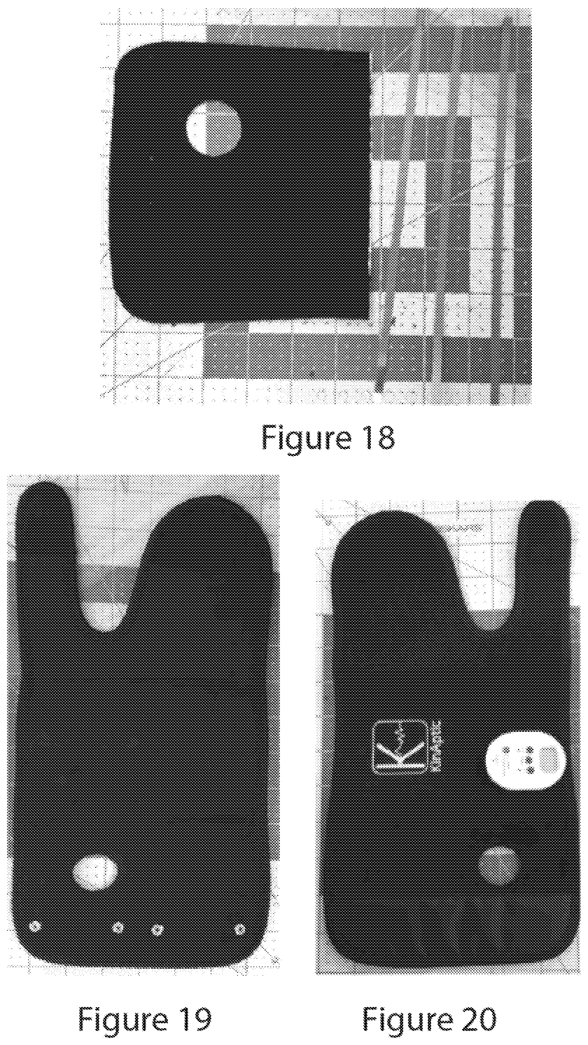

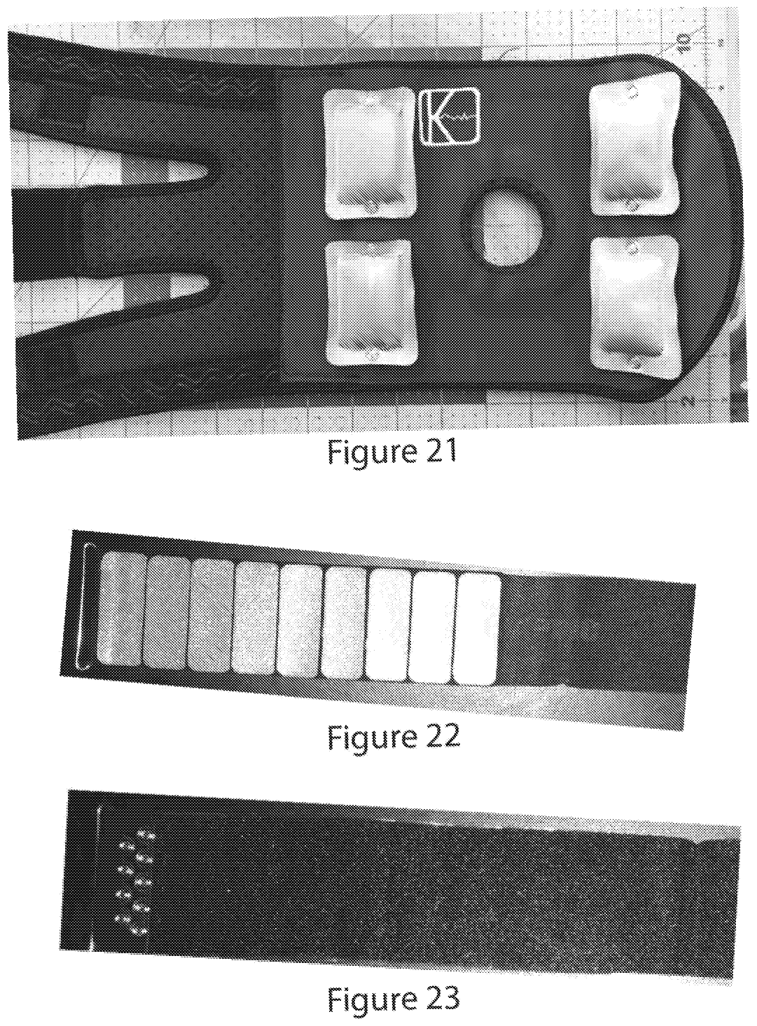

FIG. 17 shows the TENS unit connected to the neoprene elastic bandage. FIG. 18 shows a fabric connection patch and stretchable conductive fabric strips prior to assembly on an electro stimulation wrist sleeve. FIG. 19 shows the reverse side of an electro stimulation wrist sleeve. FIG. 20 shows the obverse side of the electro stimulation wrist sleeve. FIG. 21 shows a knee sleeve having removably fixable dry electrodes. FIG. 22 shows a neoprene elastic bandage having nine individually addressable dry electrodes. FIG. 23 shows the neoprene elastic bandage with nine snap connectors for individually addressing the nine individually addressable electrodes.

In accordance with the present invention, an elastic support substrate is provided and at least one electrode is fixed to the elastic support substrate. The at least one electrode is for applying stimulation electrical signals to skin of a user. At least one urging member is fixed to the elastic support substrate, wherein the at least one urging member is disposed adjacent to the at least one electrode for urging the at least one electrode towards the skin of the user.

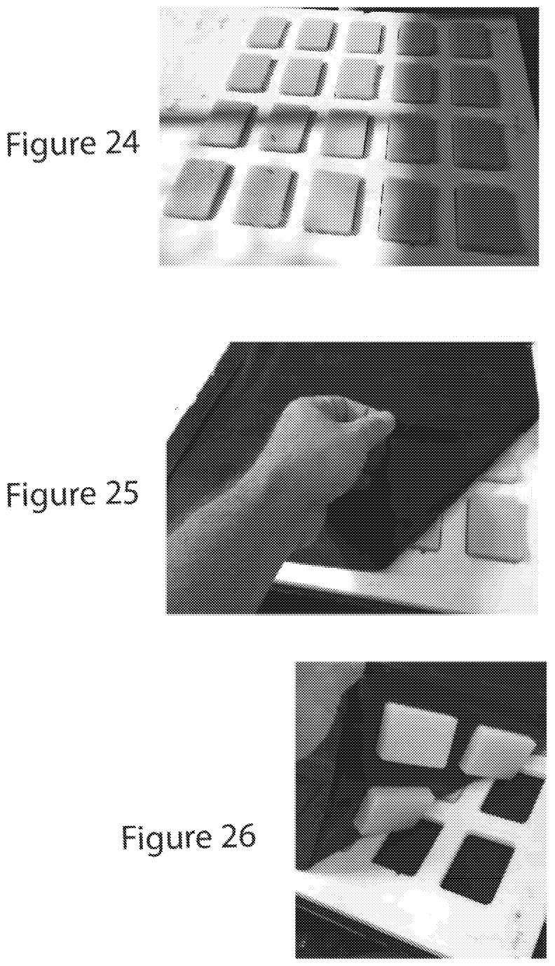

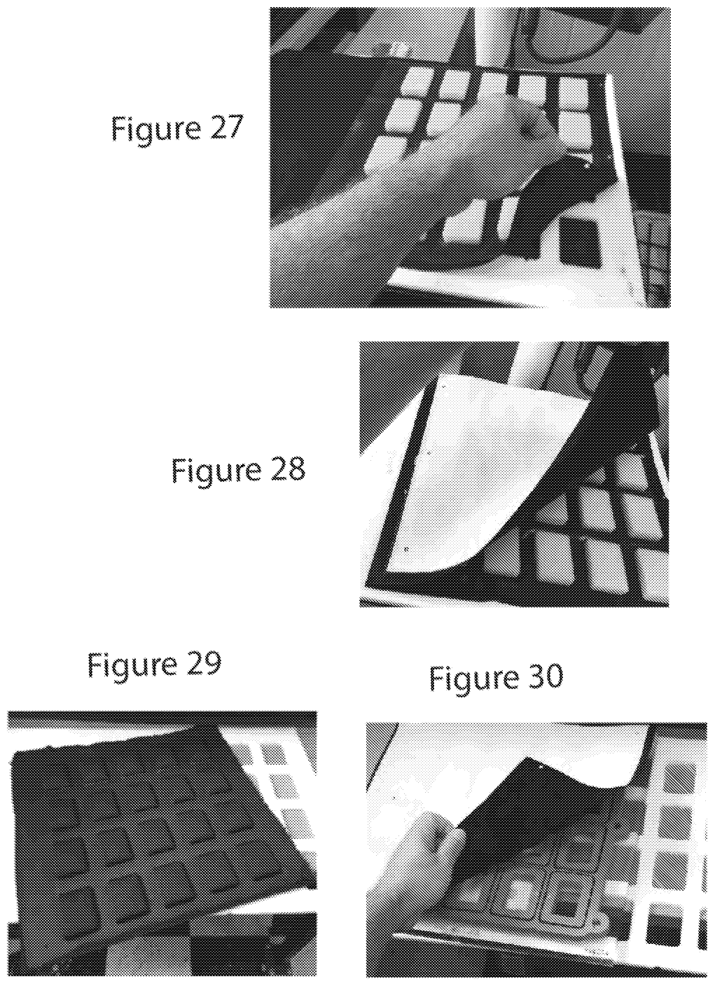

FIG. 24 shows an array of foam blocks disposed in a jig used for making the removably fixable dry electrodes. FIG. 25 shows a first elastic fabric having an elastic TPU being applied to the array of foam blocks. FIG. 26 shows the foam blocks adhered to the elastic fabric. FIG. 27 shows the first elastic fabric and foam blocks flipped over and positioned on the jig. FIG. 28 shows a second elastic fabric having a conductive surface protected by a release sheet being applied to the array of foam blocks adhered to the first elastic fabric. FIG. 29 shows a completed sheet of uncut dry electrodes. FIG. 30 shows the completed sheet of uncut dry electrodes being placed onto a steel rule the for cutting into individual dry electrodes.

The electrodes may be formed as removably fixed dry electrodes that can be snapped into place onto the elastic support substrate. As an alternative to snap connectors, another suitable electrical and mechanical connection system can be used, such as conductive Velcro. A jig for making the removably fixed dry electrodes is made from a heat resistance material, such as Teflon, and is able to withstand pressure from a heated press. The heated press is used to apply heat and pressure to the materials held in the jig so that a TPU or adhesive sheet can be activated to bond the materials together. The jig holds the relative positions of the materials and accommodates, for example, the thickness of the foam blocks to improve the final dimensions and shape of the finished product. Each of the steps performed by the heat press and jig may be adapted to a scalable high-volume roll-to-roll manufacturing process. For example, heated pressure rollers can be used to apply the heat and pressure to a stack of materials to form a lamination package. The lamination package may comprise all or a portion of the constituent parts so that multiple passes through heated rollers can be used to successively build up the constituent parts as described herein. Also, a combination of flat presses and roller presses can be employed depending on the production line cost target, throughput and assembly methods employed.

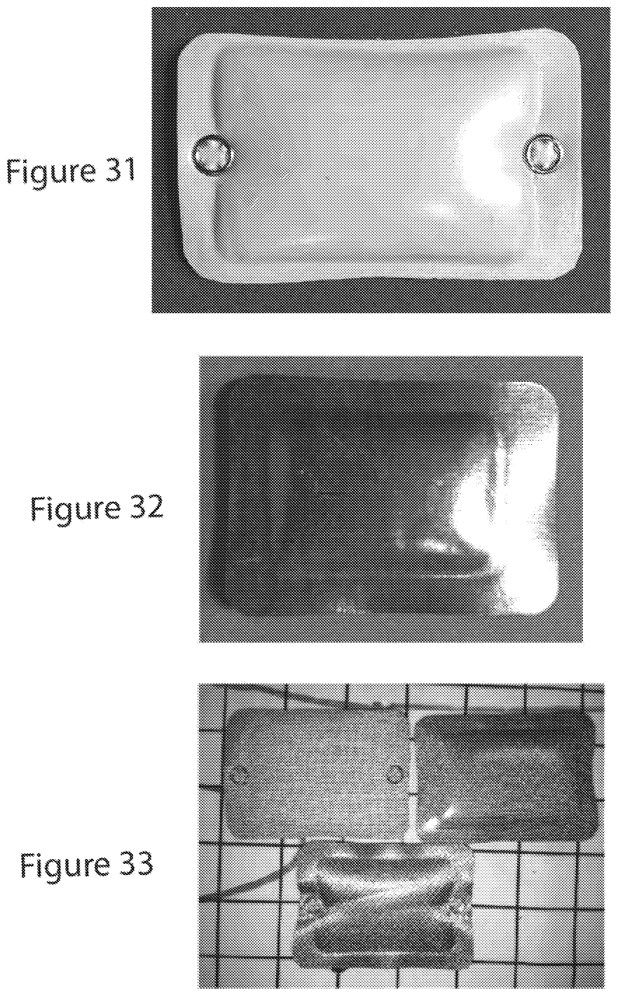

FIG. 31 shows an embodiment of the removably fixable dry electrodes having a conductive surface comprised of Ag/AgCl elastic conductive screen-printed ink. FIG. 32 shows an embodiment of the removably fixable dry electrodes having a conductive surface comprised of Ag/AgCl with an overprint of a carbon elastic conductive screen-printed ink. The Ag/AgCl may provide a good conductor for biometric signal detection, such as EMG, while also enabling the application of TENS through the same electrode. The overprint of carbon-based ink allows for an improved aesthetic and also protects the Ag/AgCl under print. If the conduction is suitable for the intended use, the Ag/AgCl layer may be avoided to reduce costs.

FIG. 33 shows embodiments of the removably fixable dry electrodes having a conductive surface comprised of a TPU that is adhered conductive fabric. In certain applications, conductive fabrics may provide easy fabrication and a more robust conductive surface. A combination of conductive fabric and printed conductive inks can be employed to create the electrodes as multilayer conductors and to provide lead lines for making electrical connections.

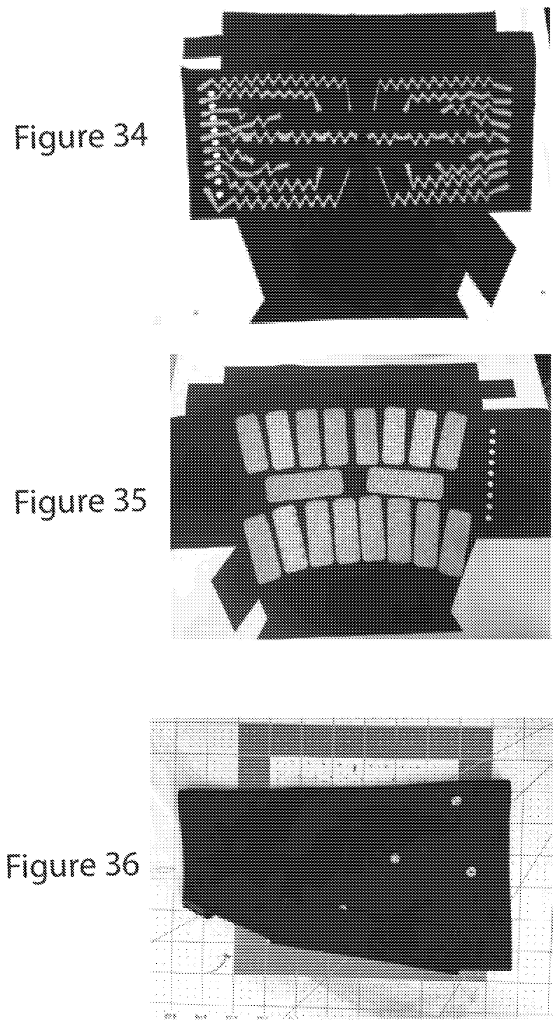

FIG. 34 shows a splayed stretchable fabric wearable electronic sleeve having stretchable wiring leads disposed on a reverse surface. FIG. 35 shows a splayed stretchable fabric wearable electronic sleeve having individually addressable electrodes on an obverse side. FIG. 36 shows an assembled stretchable fabric wearable electronic sleeve formed using heat and pressure to adhere seams using an elastic TPU.

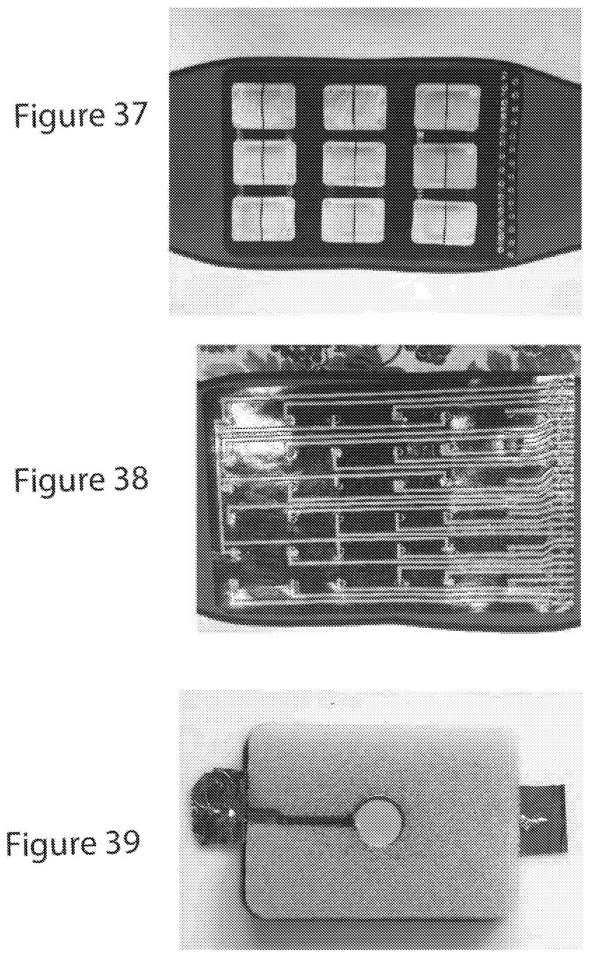

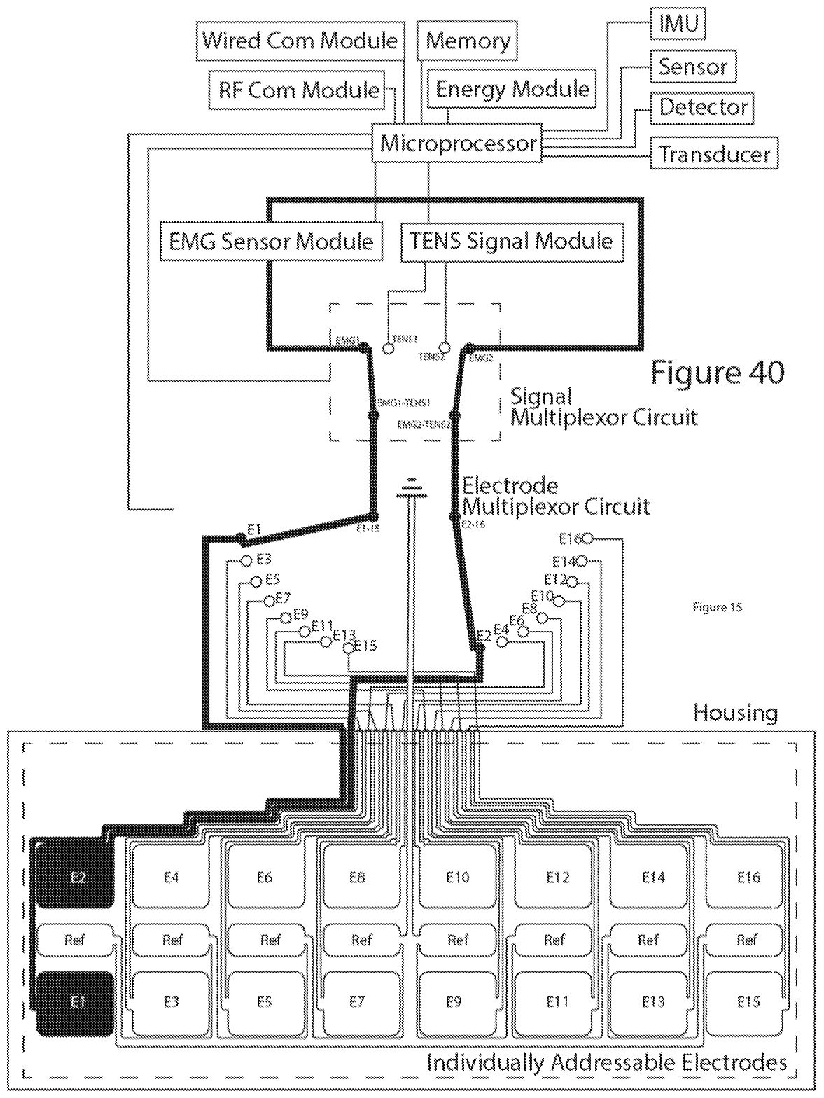

FIG. 37 shows a back support having an array of individually addressable removably fixed dry electrodes, where each dry electrode includes a bifurcated separately addressable conductive surface. FIG. 38 shows the wiring connection plane of the back support shown in FIG. 37. FIG. 39 shows a foam block having an embedded vibrator used for forming individually addressable electrodes that include a vibration feature. The bifurcated separately addressable conductive surfaces can be accessed as patterned groups to match the desired skin surface or underlying physiology of the user. The dry electrodes can be used for both detecting and applying electrical signals to/from the user's body, and the detection and application of electrical signals can be done in conjunction or separately from the application of vibration to the body.

FIG. 40 illustrates an exemplary embodiment showing electrical signals applied to a plurality of individually addressable electrodes routed through an electrode multiplex circuit and a signal multiplex circuit. An apparatus is provided for applying an electrical stimulation to skin of a user for at least one of mitigating pain, creating a haptic stimulation and for causing an involuntary muscle contraction. The apparatus comprising an elastic support, at least one electrode supportable by the elastic support and for applying stimulation electrical signals to skin of a user and at least one urging member supportable by the elastic support adjacent to the at least one electrode for urging the at least one electrode towards the skin of the user.

The at least one electrode may comprise a plurality of individually addressable electrodes supported by the elastic support. The individually addressable electrodes are for at least one of applying stimulation electrical signals to skin of a user and detecting biometric electrical signals from the skin of the user.

As shown in FIG. 40, in accordance with an aspect of the invention at least one of a signal detector for detecting the biometric electrical signals and a signal generator for generating the stimulation electrical signals may be provided. An electrode multiplex circuit may be provided for addressing the plurality of individually addressable electrodes by at least one of routing the biometric electrical signals from the skin of the user through one or more of the plurality of individually addressable electrodes to the signal detector and routing the stimulation electrical signals from the signal generator through one or more of the plurality of individually addressable electrode to the skin of the user. A microprocessor may be provided for controlling at least one of the signal detector, the signal generator, the electrode multiplex circuit enclosed within a housing is provided.

A plurality of individually addressable electrodes is supported by the elastic support (e.g., an elastic bandage, sleeve, etc.). The individually addressable electrodes are for at least one of applying stimulation electrical signals to skin of a user and detecting biometric electrical signals from the skin of the user. At least one of a signal detector for detecting the biometric electrical signals and a signal generator are provided for generating the stimulation electrical signals. An electrode multiplex circuit addresses the plurality of individually addressable electrodes by at least one of routing the biometric electrical signals from the skin of the user through the plurality of individually addressable electrodes to the signal detector and routing the stimulation electrical signals from the signal generator through the plurality of individually addressable electrode to the skin of the user. The microprocessor controls least one of the signal detector, the signal generator, the electrode multiplex circuit.

The microprocessor can control the electrode multiplex circuit to route the biometric electrical signals from the skin of the user sequentially through more than one of the plurality of individually addressable electrodes to the signal detector. The microprocessor can control the electrode multiplex circuit to route the biometric electrical signals from the skin of the user simultaneously through more than one of the plurality of individually addressable electrodes to the signal detector. The microprocessor can control the electrode multiplex circuit to route the stimulation electrical signals from the signal generator simultaneously through more than one of the plurality of individually addressable electrodes to the skin of the user.

The microprocessor can control the electrode multiplex circuit to route the stimulation electrical signals from the signal generator sequentially through more than one of the plurality of individually addressable electrodes to the skin of the user. The microprocessor can control the electrode multiplex circuit to route the stimulation electrical signals from the signal generator simultaneously through more than one of the plurality of individually addressable electrodes to the skin of the user.

A signal multiplex circuit controlled by the microprocessor can be provided for routing the electrical signals from the signal generator to skin of the user through the electrode multiplex circuit and to the signal detector from the skin of the user through the electrode multiplex circuit. A memory controlled by the microprocessor can be provided for storing data dependent on the biometric electrical signals, and a communication module can be provided for transmitting the stored data for analysis by a remote network device.

The same individually addressable electrode of the plurality of individually addressable electrodes can be used to both detect the biometric electrical signals from the skin and apply the stimulation electrical signals to the skin. The microprocessor can control the electrode multiplex circuit to address the plurality of electrodes for sampling the biometric electrical signals at a sampling rate effective for the detection by the signal detector of the biometric signals as electromyographic signals originating from subcutaneous motor units indicative of muscle contractions from two or more muscles of the user.

The microprocessor can control the electrode multiplex circuit to address the plurality of electrode for applying the stimulation electrical signals as application pulses at a pulse rate effective to cause involuntary contractions of the muscles of the user. The microprocessor can control the electrode multiplex circuit to address the plurality of individually addressable electrodes by at least one of sequentially and simultaneously routing both the biometric electrical signals from the skin of the user through more than one of the plurality of individually addressable electrodes to the signal detector and routing the stimulation electrical signals from the signal generator through more than one of the plurality of individually addressable electrode to the skin of the user. In addition to the components and features provide herein, an inertial measurement unit, accelerometer, GPS, another type of receiver, sensor or detector and another type of suitable receiver, transmitter, transducer may be supported by the elastic support.

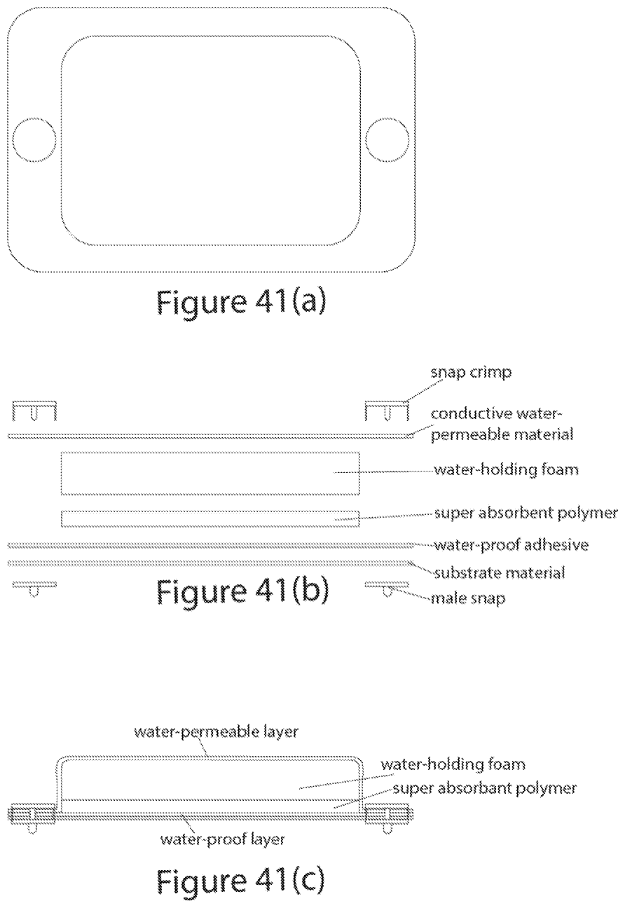

FIG. 41(a) is a top view of a removably fixable dry electrode. FIG. 41(b) is an exploded cross-sectional side view of the removably fixable dry electrode having fluid permeable dry electrode fabric, a conductivity enhancing fluid charged foam and fluid absorbent holding material. FIG. 41(c) is an assembled cross-sectional side view of the removably fixable dry electrode. A conductive fabric provides a water-permeable (or other fluid-permeable) material that allows conductivity enhancing fluid, which may be water soluble, to flow through the fluid- or water-holding foam. A super absorbent polymer, such as a hydrogel or other suitable material, may be provided for holding a considerable amount of water-soluble conductivity enhancing fluid to provide a long usage period of the wearable electronic digital therapeutic device.

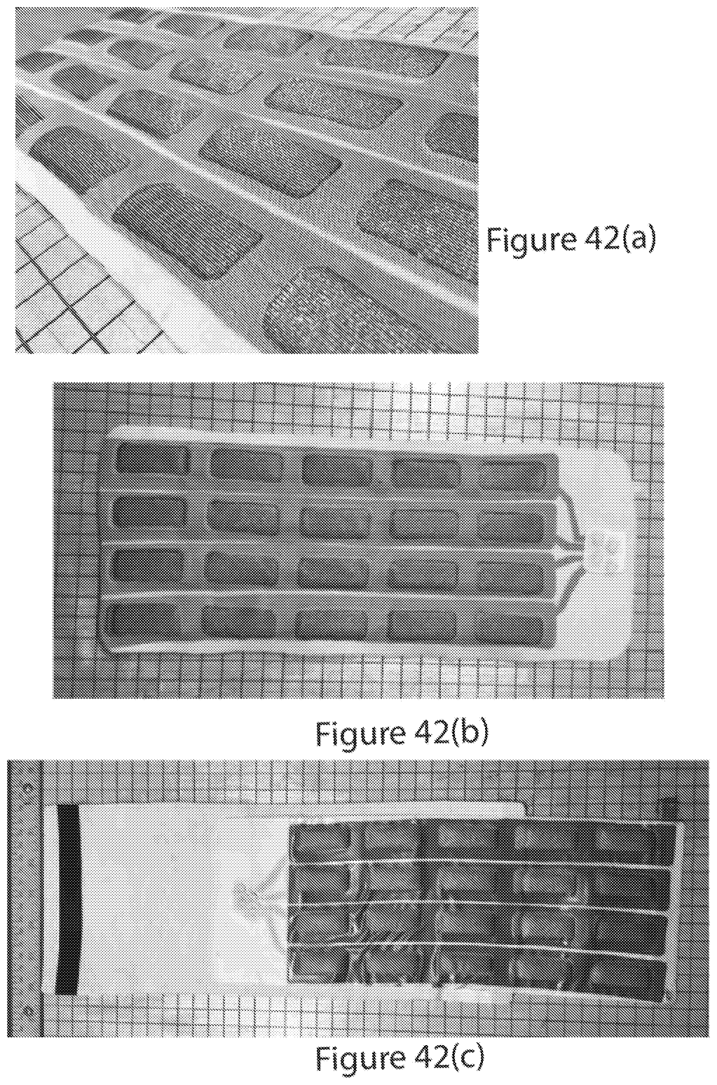

FIG. 42(a) shows a close-up of individually addressable dry electrode strips ganged on a stretchable fabric substrate constructed with fluid permeable dry electrode fabric, conductivity enhancing fluid charged foam and fluid absorbent holding material. FIG. 42(b) shows a top view of the individually addressable dry electrode strips ganged on a stretchable fabric substrate. FIG. 42(c) shows a top view of an assembled elastic wrap and the individually addressable dry electrode strips ganged on a stretchable fabric substrate. The inventive elastic bandage and dry electrode system includes a number of dry electrodes fixed on the elastic bandage. The dry electrode are supported by the elastic bandage and include at least one electrode. Typically, two or more electrodes are provided to apply an EMS signal to at least one of the muscles and nerves of the patient. The dry electrode(s) may further include the at least one urging member, such as the conductivity enhancing fluid charged foam, and the dry electrode and the elastic support interact to hold the at least one electrode in electrical contact with the skin of the user.

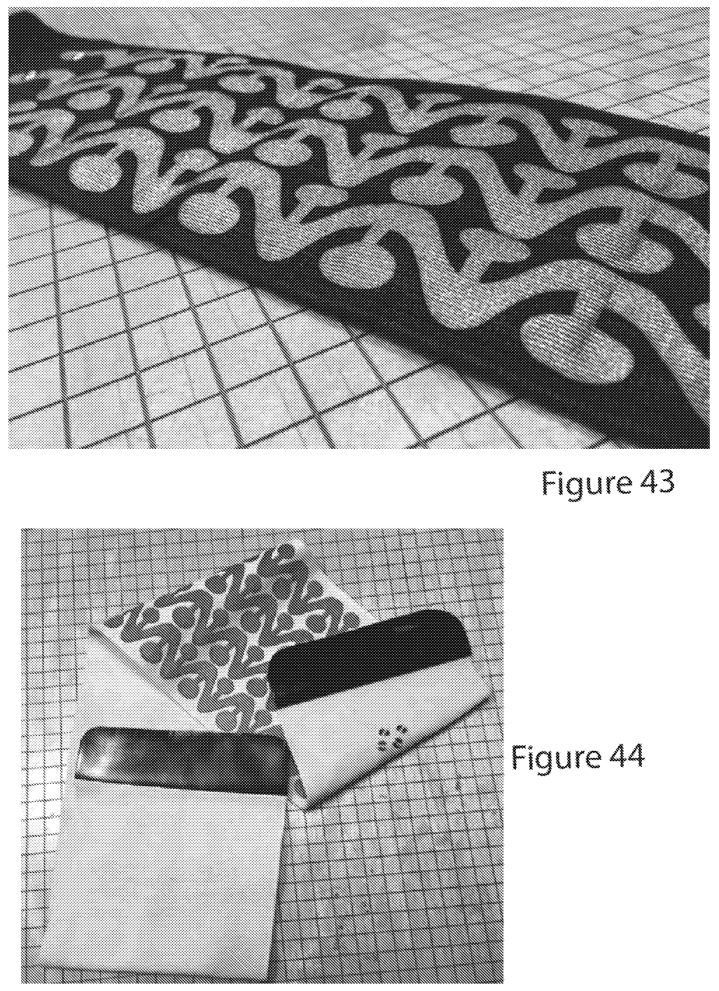

FIG. 43 is a close-up view showing individually addressable dry electrode strips having a stretchable serpentine pattern ganged on a stretchable fabric substrate. FIG. 44 shows an assembled elastic wrap having integrally fixed individually addressable dry electrode strips having a stretchable serpentine pattern. When wrapping the elastic bandage, the dry electrodes are placed at a suitable location to apply the EMS (or TENS, NMES, etc.) electrical signal through the skin to the underlying nerves and/or muscles. In accordance with an embodiment, a Velcro strip or other suitable closure retaining mechanism is provided at an end and elsewhere along the length of the bandage to facilitate wrapping the elastic bandage so that a conductive surface of the dry electrodes is urged into face to face electrical contact with the skin of the user. The elastic bandage may be suitably long enough so that the electrodes have an adequate urging to maintain suitable face to face electrical contact, and so that the remaining portion of the elastic bandage can be wrapped to provide support and compression to a joint or other body part of the user.

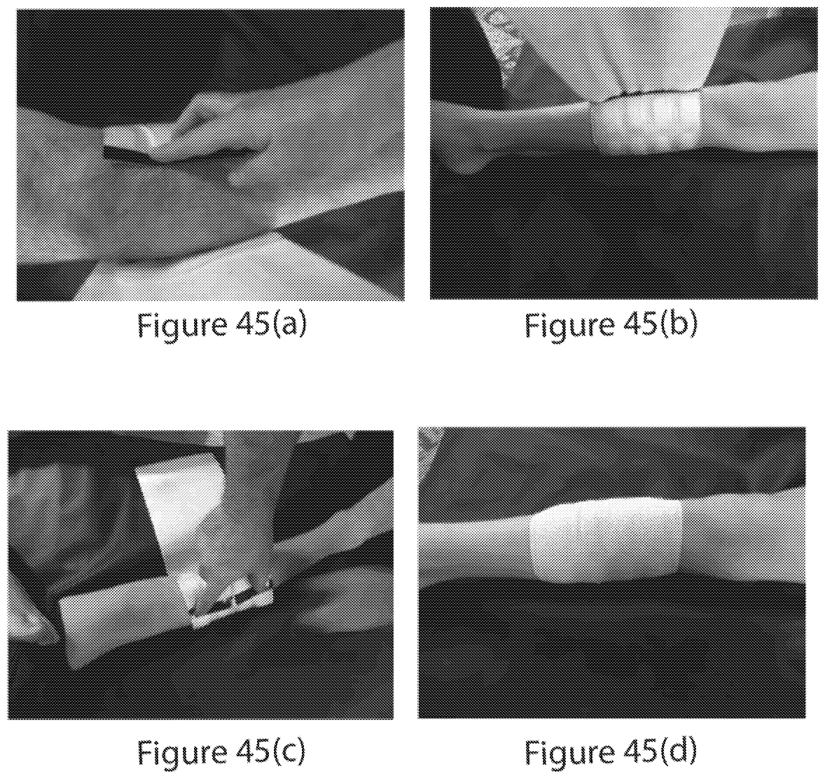

FIG. 45(a) shows a first step to wrapping an embodiment of the individually addressable dry electrode strips assembled on an elastic bandage wrap on the calf of a patient. FIG. 45(b) shows a second step to wrapping the embodiment of the individually addressable dry electrode strips assembled on an elastic bandage wrap. FIG. 45(c) shows a third step to wrapping the embodiment of the individually addressable dry electrode strips assembled on an elastic bandage wrap. FIG. 45(d) shows a fourth step to wrapping the embodiment of the individually addressable dry electrode strips assembled on an elastic bandage wrap.

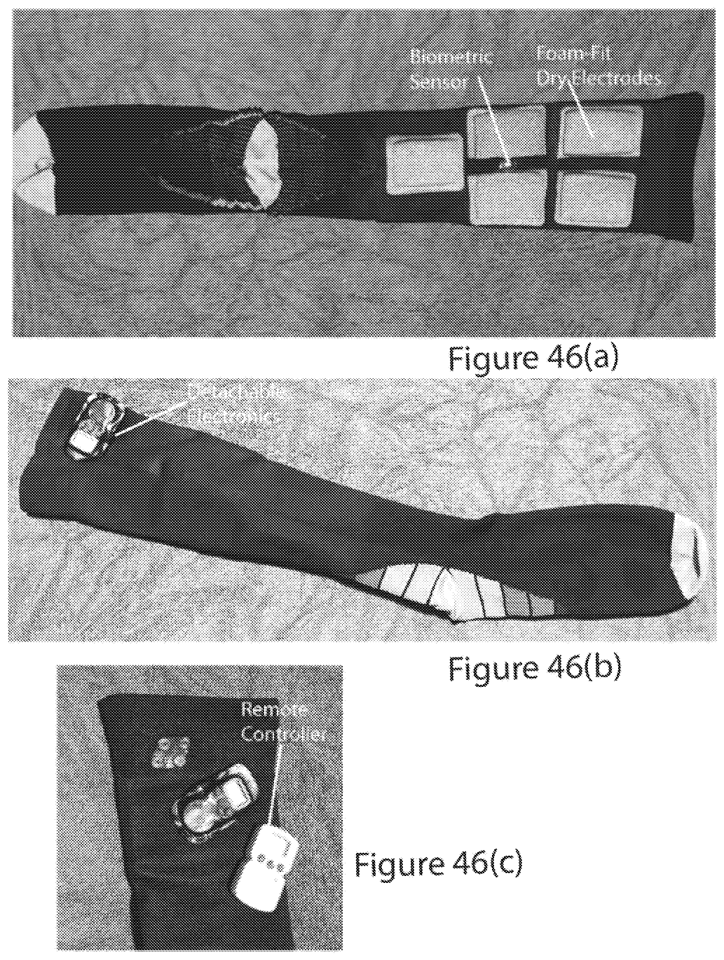

FIG. 46(a) shows the inner side of a compression stocking having dry electrodes with fluid permeable dry electrode fabric, conductivity enhancing fluid charged foam and fluid absorbent holding material for applying electrical muscle stimulation signals to at least one of nerves and muscles of the patient. FIG. 46(b) shows the outer side of the compression stocking with detectable electronics for generating the EMS signal. FIG. 46(c) shows the electronics detached from snap connectors on the compression stocking and a remote control for user control of the electronics.

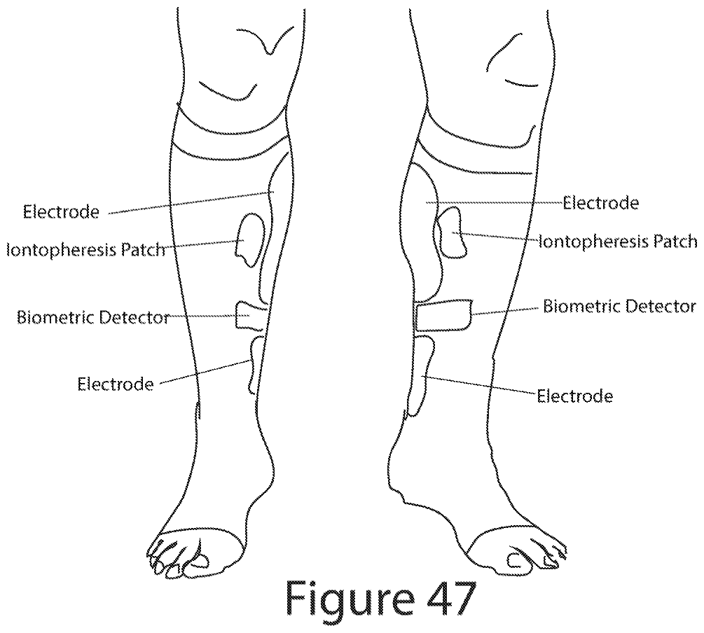

FIG. 47 illustrates a non-limiting embodiment of the inventive digital therapeutic device configured as a pair of leg stockings for applying an EMS therapy to the calf muscles of a patient. In this embodiment, the digital therapeutic device is a wearable electronic that uses electronic muscle contractions to pump blood through the blood vessels of the lower legs and prevent clots from forming. The wearable electronic includes a biometric parameter detector that generates a signal analyzed, for example, by an artificial intelligence agent embedded in a microprocessor and memory of the wearable electronic circuit to modify the applied electrical signal and optimize the involuntary muscle contractions

Each leg stocking includes at least one pair of electrodes for applying at least one of an electrical muscle stimulation (EMS) and a transcutaneous electrical nerve stimulation (TENS) signal to at least one muscle of a patient. In this case, the distinction intended by this description is to indicate for some of the utility of the leg stocking, an electrical signal that causes the release of, for example, endorphins, is applied (i.e., TENS) while other utility is obtained when the applied signal is directed at causing involuntary muscle contractions and/or nerve stimulation related to muscle functions (i.e., EMS). The electronic circuit that generates the applied signal is capable of selectively generating both, in this case, a TENS and an EMS signal. At least one biometric detector detects a biometric signal from the body of the patient. A drug delivery mechanism, such as an iontophoresis patch may be included. As described herein, the inventive digital therapeutic device may include all or some of these elements, in addition to other components, depending on the intended use.

FIG. 47 shows a simplified version of the inventive digital therapeutic device that includes a biometric detector and electrodes for applying EMS or TENS or other applied electrical signal treatment. Included with the electronics of the inventive digital therapeutic device may be a battery, microprocessor, signal generator, signal detector, communication system, memory and other associated components.

The inventive digital therapeutic device is shown as a legging or stocking worn on the lower legs, however, the configuration of the inventive digital therapeutic can also include a wrist band, arm band, sleeve, shirt, shorts, socks, ankle band, or any other suitable wearable electronic garment configuration. The features and components of the digital therapeutic device described herein can be incorporated into a brace, wrap, joint support, bracelet, belt, ring, glove, bandage, patch, or other configurations of a wearable electronics device.

An embodiment of the inventive digital therapeutic can be used for detecting blood drug level of a pharmaceutical medicinal compound and a biometric parameter, and sending data related to the detected level and parameter. In accordance with this embodiment, the reporting of the detected drug level and biometric parameter can be used to help treat the individual patient and/or be used in an aggregated form for data analysis, for example, for drug discovery or treatment determinations. The pharmaceutical medicinal compound may be, for example, for inhibiting an initiation of coagulation of blood.

An embodiment of the inventive digital therapeutic can be used for detecting a first biometric parameter and then determining if a drug should be delivered to the patient. A second detected biometric parameter can be used to determine how to apply a treatment that may be affected by the delivery of the drug. In accordance with this embodiment, the detection of the first biometric parameter can trigger the release of a drug, such as through iontophoresis, control the delivery through an intravenous drip, sending an alert to the patient, a family member, a nurse or caregiver, or through another drug delivery mechanism. The detection and analysis of the second biometric parameter can result in the adjustment of an applied treatment, where the second biometric parameter may be affected by the delivery of the drug and the action it takes on the patient's body. The first and second biometric parameters may be the same detected parameter, such as heartbeat or blood flow, or may be different parameters. Also, each biometric parameter may be determined from a combination of detected characteristics. For example, surface vein blood flow, body temperature and skin color change at the calf of a patient may be a combination biometric parameter indicative of a change in DVT, diabetes, circulatory, or thrombosis conditions.

As an example of a drug delivery mechanism that can be controlled for on-demand application (for example, by a remote caregiver or AI agent receiving an alert) iontophoresis can be utilized. In this case, iontophoresis uses as an example, a polarized electrical current to push same-charged medications into the blood stream through the skin. Liquid medications are either positively or negatively charged, then an electrical potential applied through the drug to the skin surface to drive the drug into the body. The drug can be forced subcutaneously according to a timer, or in response to a sensed condition, or biometric, such as blood sugar level, blood flow, blood pressure or heartbeat, or in response to a patient activation, or other triggering mechanism.

The digital therapeutic can be used so that ingestion can be positively determined of a target drug along with a biometric indicator incorporated in the same pill or capsule or otherwise imparted into the patient at the same time as the target drug. The ingestion is positively determined using detection of the biometric indicator as a positive indication that the target drug has been taken by the patient. A pill, or other drug delivery mechanism, containing a target drug may include a biometric indicator that is detectable and used to indicate, for example, patient adherence (the taking of a pill containing the target drug and the biometric indicator, the availability of the target drug into the blood stream, the timed release of the target drug, etc.). For example, in the case of the timed release of the target drug, the biometric indicator can be delivered with the same time-release mechanism as that incorporated with the target drug. The biometric indicator can be, for example, an additional component added to the chemistry of a new or pre-existing drug. In accordance with an embodiment, the biometric indicator is a benign water-soluble chemical compound that does not adversely alter normal body functionality, is detected from analysis of the sweat, and does not adversely affect the beneficial actions of the target drug.

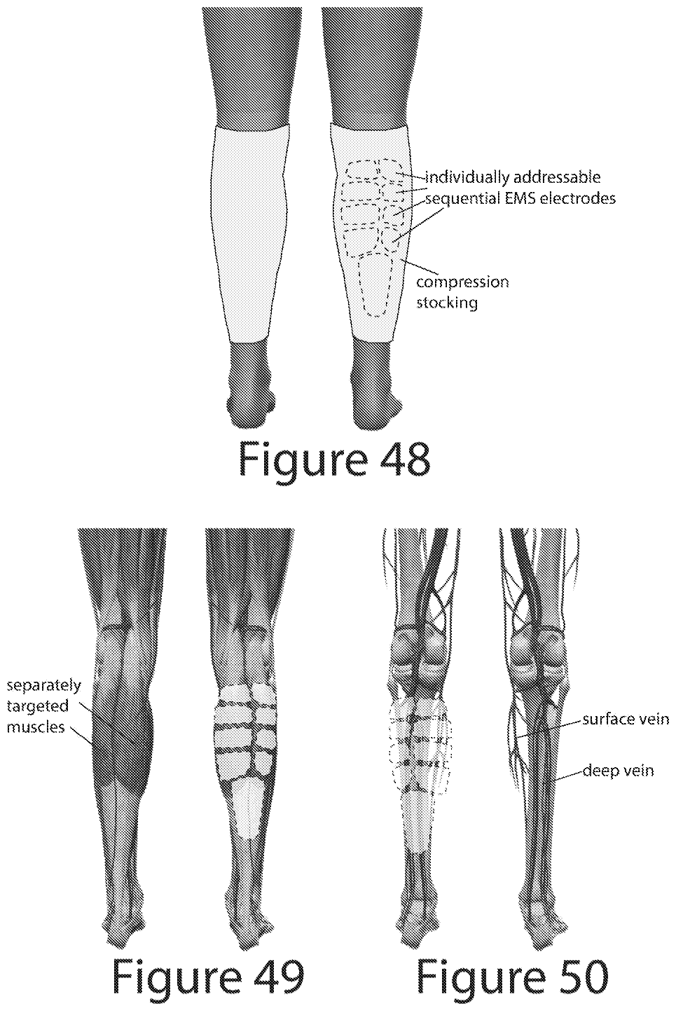

FIG. 48 illustrates an embodiment of the inventive digital therapeutic worn on the calf(s) of a patient. FIG. 49 illustrates the location of EMS applying electrodes relative to muscles of the calf of a patient.

As described in detail herein, and by way of example, the inventive digital therapeutic device may be effective alone or as an adjunct to anticoagulation medicines including, but not limited to, rivaroxaban (Xarelto), which may be also co-administered with acetylsalicylic acid (ASA) alone or with ASA plus clopidogrel or ticlopidine, for the prevention and treatment of atherothrombotic events in patients after an acute coronary syndrome (ACS) and/or atherothrombotic events in patients with coronary artery disease (CAD) or symptomatic peripheral artery disease (PAD) at high risk of ischemic events.

The use of the inventive digital therapeutic device may be effective alone or as an adjunct to conventional drug therapies, future drugs under development or to be developed, other medical devices and/or a combination of other physical, chemical and/or cognitive therapies. The inventive digital therapeutic device configured with electrodes for multiple sequentially applied EMS signals can be employed for the following uses, including but not limited to the prevention and treatment of venous thromboembolism (VTE), deep vein thrombosis (DVT), pulmonary embolism (PE), stroke and systemic embolism including patients with nonvalvular atrial fibrillation, atherothrombotic events (e.g., in patients after an ACS or in patients with CAD/PAD).

There is a potential for inadequate anticoagulation during the transition from one anticoagulation drug (e.g. Xarelto) to another (e.g. Vitamin K Antagonists (e.g., Warfarin)). The inventive digital therapeutic device may be an effective additional mechanism to help ensure atherothrombotic events or other complications do not occur during such transition. An anticoagulation medication, such as rivaroxaban, is a highly selective direct factor Xa inhibitor with oral bioavailability. Inhibition of factor Xa interrupts the intrinsic and extrinsic pathway of the blood coagulation cascade, inhibiting both thrombin formation and development of thrombi.

As an alternative or in addition to conventional drug therapies, the inventive digital therapeutic device can be effective in the prevention, for example, of venous thromboembolism (VTE) in patients undergoing elective hip or knee replacement surgery and in the treatment of deep vein thrombosis (DVT) and pulmonary embolism (PE), and prevention of recurrent DVT and PE.

An embodiment of the inventive digital therapeutic can be used for detecting a biometric parameter and using the analysis of the detected biometric parameter to automatically adjust an applied therapy. The biometric parameter can be, but is not limited to, heartbeat, blood flow, body temperature, skin color, blood and/or sweat chemistry, respiration, blood oxygen level, EMG or other electrical signal, a detectable chemical compound not normally present in the blood, or the like. The applied therapy can include, but is not limited to, EMS, TENS, compression, drug selection, delivery and/or dosage modification, heat, cold or the like. In accordance with this embodiment, the detection and analysis of the biometric parameter can be used to help treat the individual patient with an automatically optimized applied therapy.

In accordance with an embodiment, a method comprises applying a therapeutic treatment to a user. A biometric parameter indicative of a physiological change dependent on the applied therapeutic treatment is detected. The applied therapeutic treatment is modified dependent on the detected biometric signal.

In accordance with another embodiment, an apparatus is provided comprising at least one processor, and at least one memory including computer program code. The at least one memory and the computer program code are configured, with the at least one processor, to cause the apparatus to perform at least the following: apply a therapeutic treatment to a user, detect a biometric parameter indicative of a physiological change dependent on the applied therapeutic treatment, and modify the applied therapeutic treatment dependent on the detected biometric signal.

In accordance with another embodiment, a computer program product comprises a computer-readable medium bearing computer program code embodied therein for use with a computer. The computer program code comprising code for applying a therapeutic treatment to a user, code for detecting a biometric parameter indicative of a physiological change dependent on the applied therapeutic treatment, and code for modifying the applied therapeutic treatment dependent on the detected biometric signal.

In accordance with another embodiment of the inventive digital therapeutic, an electrical muscle stimulation signal is applied having at least one signal characteristic. The electrical muscle stimulation signal is applied to at least one muscle of a user. A biometric parameter indicative of a physiological change dependent on the applied electrical muscle stimulation signal is detected. The applied electrical muscle stimulation signal is modified dependent on the detected biometric signal.

An inventive digital therapeutic device includes a wearable electronic garment for applying an electrical muscle stimulation signal through the skin to induce involuntary contractions in one or more muscles of a user. A biometric signal detector detects a biometric parameter indicative of a physiological change dependent on the applied electrical muscle stimulation signal. A microprocessor controls the application of the electrical signal dependent on the detected biometric signal.

FIG. 50 illustrates the location of EMS applying electrodes relative to blood vessels of the calf(s) of a patient. An embodiment of the inventive digital therapeutic can be used for detecting blood flow, for example, at surface veins in the calf or at another body part. The blood flow at the surface veins in the calf will be particularly useful to indicating the flow of blood from the deep veins in the legs, and those particularly susceptible to DVT. The blood flow biometric parameter is analyzed to automatically adjust an applied EMS therapy. The biometric parameter can also include, but is not limited to, the detection of heartbeat, body temperature, skin color, blood and/or sweat chemistry, respiration, blood oxygen level, or the like, which may be done in conjunction simultaneously or intermittently with the blood flow detection. The aggregate data from multiple sensors and biometric parameters may improve the overall performance of the system for automatically optimizing the applied therapy. The applied therapy can include, but is not limited to, being done in combination along with EMS, TENS, compression, drug selection, delivery and/or dosage modification, heat, cold or the like. In accordance with this embodiment, the detection and analysis of the blood flow biometric parameter can be used to help treat the individual patient with an automatically optimized applied EMS therapy.

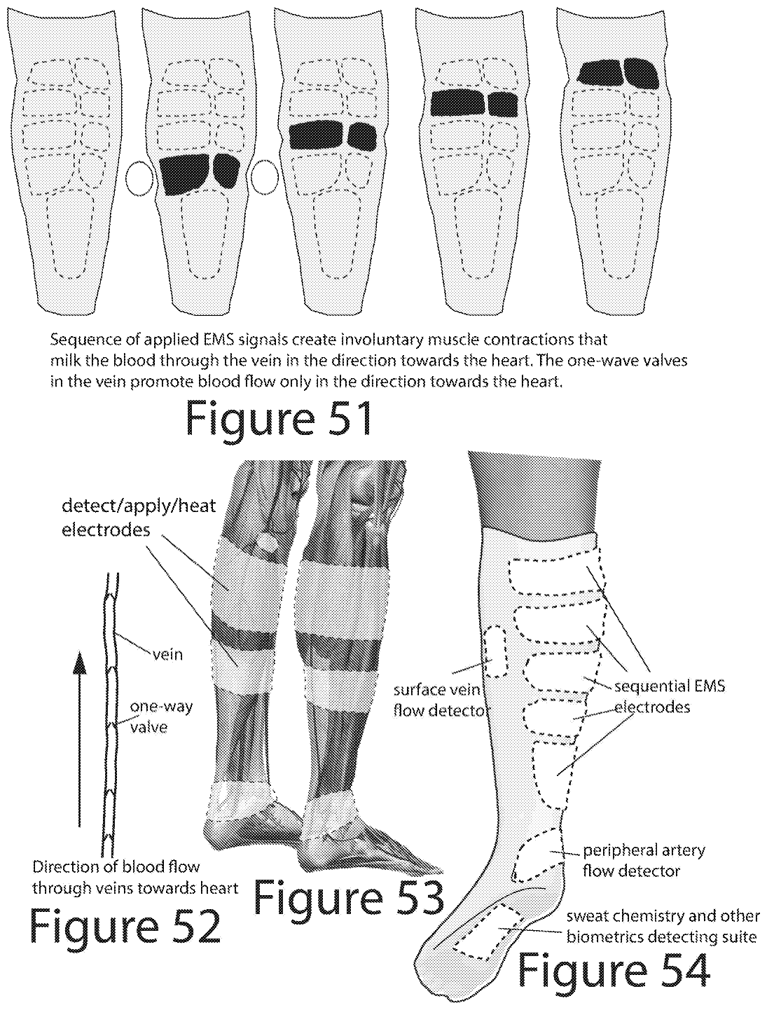

FIG. 51 illustrates sequentially applied EMS signals applied via the EMS applying electrodes of the inventive digital therapeutic device. In accordance with the inventive digital therapeutic, to treat and/or prevent an atherothrombotic event, an electrical muscle stimulation signal is applied having at least one signal characteristic. The electrical muscle stimulation signal is applied to at least one muscle adjacent to a blood vessel of a patient. The electrical muscle stimulation signals cause an involuntary contraction of the at least one muscle to impart a squeezing action on the blood vessel and promote a flow of blood through the blood vessel. A biometric parameter indicative of the flow of blood through the blood vessel is detected. The applied electrical muscle stimulation signal is modified dependent on the detected biometric signal.

As a non-limiting example, also shown, for example, in FIG. 26, the biometric can be determined using a strain gauge that measures a tightness in the calf muscles. When successive strain gauge readings are compared, the analyzed biometric may indicate that swelling is occurring in the region of the calf muscles indicating poor blood flow in the direction back to the heart in that region. In this example, it may be advantageous to adjust the applied therapy characteristic to increase the strength of the sequentially applied EMS signal or the duration of the sequentially applied EMS therapy (i.e., extend the treatment time), to help improve blood flow through the veins back to the heart and reduce the swelling. In this way, the adjusted applied therapy characteristic is used to optimize the applied therapy that is applied to the patient.

The biometric parameter may be dependent on a therapeutic action of a pharmaceutical medicinal compound. The applied electrical muscle stimulation signal is modified in response to the therapeutic action of the pharmaceutical medicinal compound. The biometric signal may be dependent on at least one detectable biometric parameter. The biometric parameter may be detectable dependent on at least one of skin temperature, skin color, blood flow, pulse, heartbeat, blood pressure, skin tightness, swelling, blood chemistry, sweat chemistry, an electronic biomarker, a chemical biomarker, and electromyography. The blood vessel may be a deep vein where the blood flow is promoted in the direction towards the heart.

FIG. 52 illustrates a deep vein showing the direction of blood flow towards the heart. The blood flow biometric parameter can also include, for example, a combined parameter that includes skin color and/or skin temperature measured in conjunction with blood flow (with the possibility for analysis, for example, to get an indication of a change in the effectiveness of a medication extrapolated or directly determined from the measured parameters, for example, blood flow, skin color, skin temperature, etc.). The applied electrical muscle stimulation signal may be applied as a sequence of electrical signals through two or more pairs of electrodes for sequentially squeezing the blood vessel along a longitudinal axis of the blood vessel to promote the flow of blood through the blood vessel in a direction determined by the sequential squeezing and one-way vascular valves within the blood vessel. For example, the therapy may be a relatively stronger sequentially applied EMS signal that causes muscles to contract and milk blood through a vein in the direction of the heart or through an artery in the direction away from the heart.

Blood circulation starts at a phase when the heart relaxes between two heartbeats and blood flows from both atria (the upper two chambers of the heart) into the ventricles (the lower two chambers). In this phase, the ventricles then expand and in a following phase, called the ejection period, both ventricles pump the blood into the large arteries. The left ventricle pumps oxygen-rich blood into the aorta and the blood travels ultimately to the capillary network where oxygen, nutrients and other substances are released and carbon dioxide and waste substances are taken on, and the blood is then collected in veins and travels to the right atrium and into the right ventricle. The blood flows more slowly in veins than in arteries, which is why thromboses occurs most often in the veins. The deep veins in the calf and thigh are most often affected by deep vein thrombosis or phlebothrombosis.

Blood returning from the legs occurs mainly through the deep veins. Venous valves are bicuspid flaps made of elastic tissue that keep blood moving in one direction. The low pressure of the blood flow in the venous system varies from relatively high flow (during muscle contraction) to low flow, for example, when sedentary. Blood flow through the deep veins is also affected by gravity, the collapsible nature of the venous wall, the presence of valves, and the large volume of blood carried in the deep veins.

Once the blood has passed from the arteries through the capillaries, it is flowing at a slower rate because little pressure remains to move the blood along. Blood flow in the veins below the heart is helped back up to the heart by the muscle pump. Many veins are located in the muscles and the squeezing action of the muscles promotes the flow of blood through the veins. For example, movement of the leg squeezes the veins, which pushes the blood toward the heart. When the muscles contract the blood within the veins is squeezed up the vein and the valves open. When the muscle is at rest, the valves close helping to prevent the backward flow of blood. This is referred to as the muscle pump.

In a healthy leg, veins have smooth and elastic walls designed to adapt to the changes in pressure within a vein. The venous valves keep blood moving in one direction back toward the heart, and as the leg muscles contract, the venous valves open to allow one-way flow in the direction of the heart. When the muscles relax, the valves close to prevent back-flow.

But if the walls of a vein are damaged by varicosis or thrombosis, the vein can dilate, and the valves fail to adequately function to promote one-way blood flow. The blood can then pool in the veins, which may cause even more valves to fail over time. In this case, for example, when the patient stands upright, the blood that should be transported back to the heart can stagnate in the legs. The pressure in the surface veins directly under the skin then rises and the veins become swollen. Fluid may collect, especially in the feet and ankles, causing swelling, and the skin above the ankles may become thin and discolored or even break to form a venous stasis ulcer.

In accordance with a non-limiting embodiment of the inventive digital therapeutic device, these biometric parameters related to the flow of blood in the deep veins are detected and used to modify the application of EMS to cause sequential involuntary muscle contractions. The sequential involuntary muscle contractions are timed to most efficiently promote blood flow through the deep veins in the direction towards the heart.

FIG. 53 illustrates biometric detecting, EMS/TENS applying, heat apply electrodes in accordance with an embodiment of the inventive digital therapeutic device. As a non-limiting example, an embodiment of the inventive digital therapeutic device may be used alone or as an adjunct to anticoagulation medicines for the prevention and treatment of atherothrombotic events in patients. An embodiment of the inventive digital therapeutic can be used for detecting multiple biometrics including, but not limited to surface vein blood flow, skin color, heartbeat, arterial pulse, swelling, tightness, the presence of an administered radioisotope, the detection of a biomarker such as, but not limited to, D-dimer protein, blood oxygen, CO2, lactose, glucose, urea (obtained directly from the blood or through the sweat) or other biometric indicator useful for adjusting an applied therapy. The applied therapy can include, but is not limited to, and be done alone in combination along with, EMS, TENS, compression, drugs (with modification to selection, delivery and/or dosage), heat, cold, etc.

FIG. 54 illustrates the inventive digital therapeutic device configured as a leg stocking and having multiple biometric sensors and multiple electrodes. As an exemplary biometric detector, a sweat chemistry sensor can be disposed in the location of the bottom of the foot. The human body has approximately 2-4 million sweat glands found all over the body, except on the nails, ears and lips. The most concentrated area of sweat glands is on the bottom of the foot while the least concentrated area of sweat glands is on the back. The inventive digital therapeutic device can include a sweat chemistry and other biometrics detecting patch located in proximity to the bottom of the patient's foot. As described in more detail herein, the biometrics detecting patch can be constructed to promote adequate flow of sweat, even from a patient with little physical exertion, by providing a moisture barrier that captures slight amounts of sweat always present from the numerous sweat glands at the bottom of the foot and or by including a sweat inducing chemical or electrically stimulating sweating. A sweat promoting shoe, slipper, waterproof adhesive sheet, or other mechanism may be used to facilitate the adequate capture of sweat.

Another example of a biometric detector is a peripheral artery blood flow or pulse detector. An optical and/or electrical circuit can be used to detect the blood flow or pulse from the peripheral artery located at the heel of the foot. An optical surface vein blood flow detector can be located near the calf muscles. The surface veins drain into the deep vein, enabling deep vein blood flow to be extrapolated from the detection of blood flow at the surface veins.

An embodiment of the inventive digital therapeutic can be used for detecting heartbeat, for example, at a peripheral artery on the ankle or at another body part. The heartbeat biometric parameter is analyzed to automatically adjust an applied therapy, such as EMS therapy. The biometric parameter can also include, but is not limited to, the detection of blood flow, body temperature, skin color, blood and/or sweat chemistry, respiration, blood oxygen level, other blood gas, dissolved or solid level, or the like, which may be done in conjunction simultaneously or intermittently with the heartbeat detection. The aggregate data from multiple sensors and biometric parameters may improve the overall performance of the system for automatically optimizing the applied therapy. The applied therapy can include but is not limited to being used in combination along with EMS, TENS, compression, drug selection, delivery and/or dosage modification, heat, cold or the like. In accordance with this embodiment, the detection and analysis of the heartbeat biometric parameter can be used to help treat the individual patient with an automatically optimized applied EMS therapy.

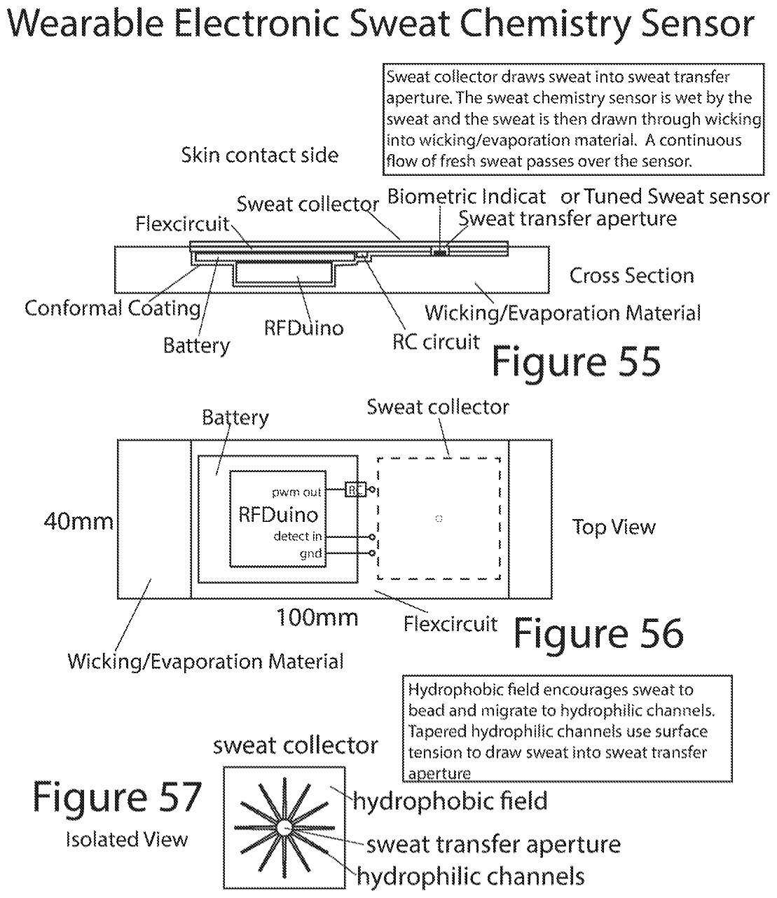

FIG. 55 shows a cross section of an inventive digital therapeutic device sweat chemistry sensor tuned to detect at least one biometric indicator associated with the presence of a therapeutic drug in the blood stream of a patient. FIG. 56 shows a top view of the inventive digital therapeutic device sweat chemistry sensor tuned to detect at least one biometric indicator. FIG. 57 is an isolated view showing a sweat collector of the inventive sweat chemistry sensor.

A sweat collector draws sweat into a sweat transfer aperture. The sweat chemistry sensor is wet by the sweat and then the sweat is drawn through wicking into wicking/evaporation materials. A continuous flow of fresh sweat passes over the sensor. For many applications, including fitness and military uses, a sweat sensor patch constructed as described herein can be fixed to the skin at a variety of convenient locations, such as the waistband of underwear or running shorts. For the application described herein for DVT or other lower leg conditions, a moisture barrier can be fixed in place to enclose the sweat chemistry sensor within a vapor-resistant barrier in order to capture an adequate quantity of sweat from the numerous sweat glands at the bottom of the foot.

A hydrophobic field encourages sweat to bead and migrate to hydrophilic channels. Tapered hydrophilic channels use surface tension to draw sweat into the sweat transfer aperture. Hydrophobic and hydrophilic screen printable inks are available from companies such as Cytonix and Wacker.

Many water-soluble components in the blood can be detected through sweat chemistry analysis. Lactate, Glucose and Urea are three important blood chemistry measurements. Lactate is the output of the anaerobic system; after that it performs its most important function. It is the main fuel for the aerobic system during competition and much of training. Lactate is a major fuel source for the heart and the brain as well as skeletal muscles during strenuous efforts. Measuring lactate is a way of assessing how strong each energy system is, or essentially how well-conditioned an athlete is or the general health of a patient at a specific point in time. In accordance with the inventive digital therapeutic device, different drug and blood chemistry components can be used as biometric indicators and detected biometrics. For example, if the blood chemistry of a patient indicates dehydration, an alert can be generated that is sent to a caregiver, nurse, family member, the patient, a doctor, etc., and received via cellphone, digital assistant, computers, etc. to indicate that the patient needs attention.

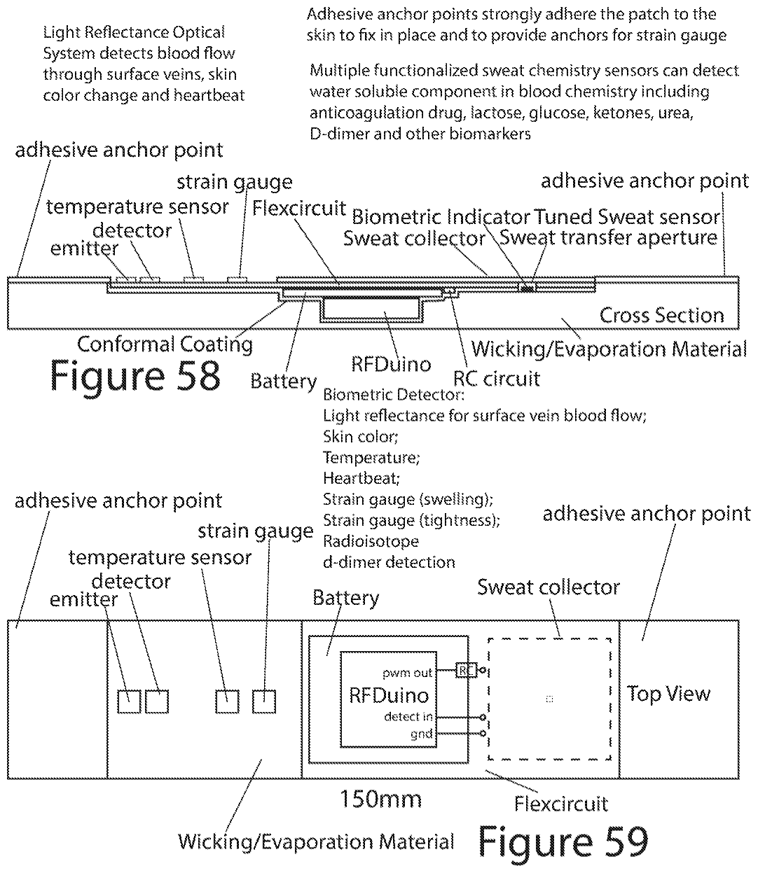

FIG. 58 shows a cross section of an inventive digital therapeutic device sensor patch with a suite of biometric detectors. FIG. 59 shows a top view of the inventive digital therapeutic device sensor patch with a suite of biometric detectors. The inventive digital therapeutic device sensor patch includes adhesive anchor points to strongly adhere the patch to the skin to fix in place and provide anchors for a strain gauge biometric detector. A light reflectance optical system detects blood flow though surface veins, skin color, blood oxygen, heartbeat and other optically obtained biometric parameters. One or more functionalized sweat chemistry sensors can detect water soluble components in blood chemistry that are present in sweat, including water soluble components of anticoagulation drugs, lactose, glucose, ketones, urea, D-dimer and other biomarkers. The biometrics detectable by the inventive digital therapeutic can be distributed at various portions of a wearable electronic, such as a stocking, and/or one or more of the biometric detectors can be incorporated into a stand-alone patch.

The biometric parameters that are detected can include, but are not limited to, light reflectance, surface vein blood flow, skin color, temperature, heartbeat, strain gauge to detect swelling and/or skin tightness, chemical or other biometric indicator. D-dimer or other body-produced biomarker, sweat chemistry indicative of blood chemistry, and other biometric parameters.

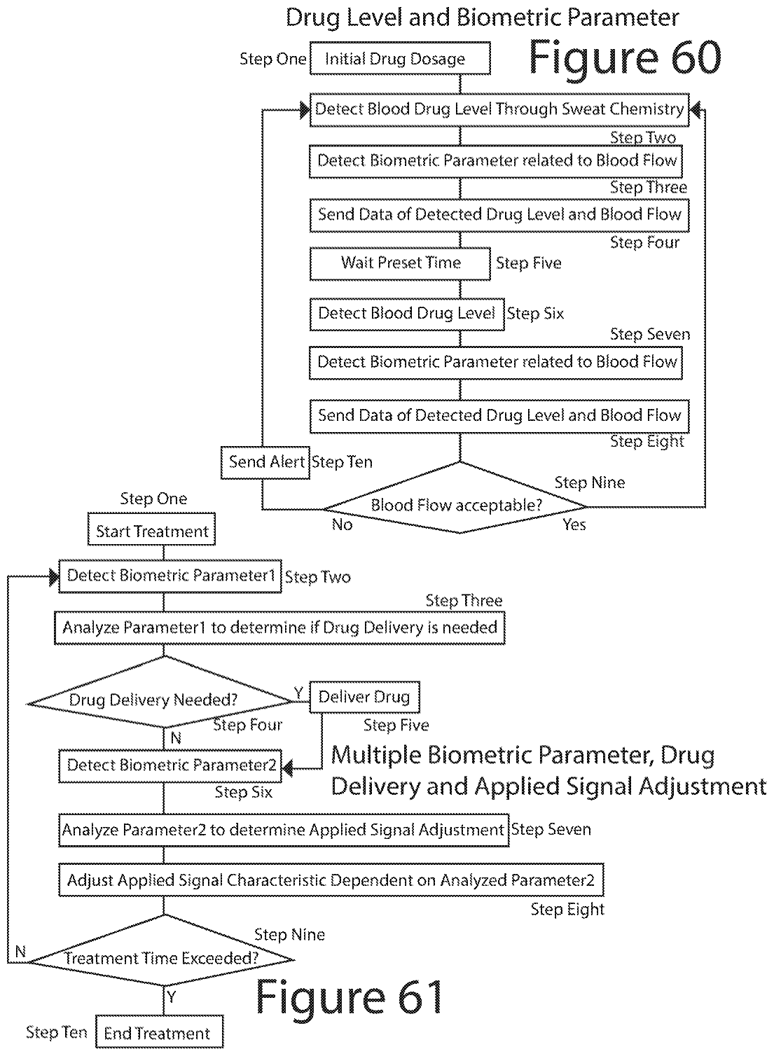

FIG. 60 is a flow chart showing an algorithm for drug level and biometric parameter detection. An embodiment of the inventive digital therapeutic can be used for detecting blood drug level and a biometric parameter, and sending data related to the detected level and parameter. In accordance with this embodiment, the reporting of the detected drug level and biometric parameter can be used to help treat the individual patient and/or be used in an aggregated form for data analysis, for example, for drug discovery or treatment determinations.

Referring to FIG. 14, an initial drug dosage is taken (Step One), and after a period of time the blood drug level is detected, for example, through sweat chemistry analysis (Step Two). A biometric parameter, such as related to blood flow, is detected (Step Three). As a non-limiting example, the blood flow can be detected using light reflectance from the skin of the patient to determine blood flow through surface veins. Data of the detected drug level and blood flow are sent (Step Four). For example, the detected data can be sent using a wireless connection to a network gateway and on through to a network server. The network server can be located remotely, for example, a cloud server, for the aggregation of data collected from a population of patients of the inventive digital therapeutic. After waiting a preset time (Step Five), the blood drug level is again detected (Step Six) and then the biometric parameter related to blood flow is again detected (Step Seven). The data of the detected drug level and blood flow is again sent to a collecting database, such as a networked cloud server (Step Eight). If the detected data indicates that the blood flow is acceptable (Step Nine) the blood drug level is again detected through sweat chemistry analysis (Step Two). However, if the detected blood flow is not acceptable, for example, indicating that too little blood is flowing through the veins of the patient's legs and there is danger of a clot forming, an alert can be sent (Step Ten). The alert can be sent, for example, using a wireless link from the inventive digital therapeutic to a wireless gateway device and onto a network such as the Internet. Using known Internet of Things protocols, the detection of unacceptable blood flow (Step Nine) can trigger the sending of an alert (Step Ten) that includes an email, pager, text, SMS, phone call, or other communication transmission that can alert the patient, a caregiver, provider, payor or other entity involved in the patient's care or the health care system in general. After the alert is sent, the blood drug level is again detected through sweat chemistry analysis (Step Two).

As search of the patent, product and scientific literature shows a number of device constructions and techniques that may be modified for use with the inventive digital therapeutic device for detecting blood flow as a biometric parameter. An example of an ultrathin, soft, skin-conforming sensor technology for continuous and precise blood flow mapping is described in Epidermal devices for noninvasive, precise, and continuous mapping of macrovascular and microvascular blood flow, Science Advances 30 Oct. 2015: Vol. 1, no. 9, e1500701 DOI: 10.1126/sciadv.1500701. In this example, an array of thin (100 nm) metallic thermal actuators and sensors designed for monitoring blood flow beneath a targeted area. U.S. Pat. No. 4,494,550, issued 22 Jan. 1985 to Blazek et al, shows an example of blood flow detection using light reflection rheology. A plurality of sources of radiation are directed on to the skin of and a radiation receiver measures the amount of radiation reflected back by the cutaneous vascular plexus. A temperature sensor simultaneously measures the skin temperature and an electronic circuit detects the progress of the reflected or dispersed amount of radiation and the skin temperature as a function of time. As another example of a biometric detector for measuring blood flow, a photopletismograph (PPG) is an optical device that can detect and measure a blood flow within body tissue.

Kyocera Corporation has developed one of the smallest known optical blood-flow sensors, which measures the volume of blood flow in subcutaneous tissue. When light is reflected on blood within a blood vessel, the frequency of light varies—called a frequency or Doppler shift—according to the blood-flow, velocity. The new sensor utilizes the relative shift in frequency (which increases as blood flow accelerates) and the strength of the reflected light (which grows stronger when reflected off a greater volume of red blood cells) to measure blood-flow volume. The sensor is only 1 mm high, 1.6 mm long and 3.2 mm wide and is designed for use in small devices such as mobile phones and wearable devices.

As a device that is less invasive and inconvenient as drawing blood samples, a biometric parameter and/or biometric indicator detector suitable for the inventive digital therapeutic device may employ the concept of using a microneedle system. For example, researchers at the University of British Columbia and the Paul Scherrer Institute (PSI) in Switzerland have reported a microneedle drug monitoring system designed to puncture the outer layer of skin but not the next layers of epidermis and the dermis, which house nerves, blood vessels and active immune cells. The use of a biometric detector based on a microneedle for the inventive digital therapeutic may have the advantages of both convenience, minimal invasiveness and rapid detection, after the ingestion or other delivery of a drug or biometric indicator. Instead of blood, the fluid found just below the outer layer of skin is used to detect and monitor chemicals in the bloodstream. The microneedle may collect just a tiny bit of this fluid, less than a millionth of a milliliter, and a reaction occurs on the inside of the microneedle that can detect blood chemistry using an optical sensor.

FIG. 61 is a flow chart showing an algorithm for detecting multiple biometric parameters used to determine when and in what quantity to deliver a drug and how to adjust an applied treatment signal. An embodiment of the inventive digital therapeutic can be used for detecting a first biometric parameter that then determines if a drug should be delivered to the patient. A second detected biometric parameter can be used to determine how to apply a treatment that may be affected by the delivery of the drug. In accordance with this embodiment, the detection of the first biometric parameter can trigger the release of a drug, such as through iontophoresis, control the delivery through an intravenous drip, send an alert to the patient, a family member, a nurse or caregiver, or through another drug delivery mechanism. The detection and analysis of the second biometric parameter can result in the adjustment of an applied treatment, where the second biometric parameter may be affected by the delivery of the drug and the action it takes on the patient's body. The first and second biometric parameters may be the same detected parameter, such as heartbeat or blood flow, or may be different parameters. Also, each biometric parameter may be determined from a combination of detected characteristics. For example, surface vein blood flow, body temperature and skin color change at the calf of a patient may be a combination biometric parameters indicative of a change in DVT conditions.

Skin color can be detected using optical systems. Full-color skin imaging using RGB LED and floating lens in optical coherence tomography, Yang B-W, Chen X-C. Full-color skin imaging using RGB LED and floating lens in optical coherence tomography. Biomedical Optics Express. 2010; 1(5):1341-1346. doi:10.1364/BOE.1.001341 shows an example of an LED based skin color sensor system that can be modified in accordance with the inventive digital therapeutic to detect skin color as a biometric parameter. It is noted that many of the various biometric detector can share common components, reducing costs and enabling high speed sampling of different biometric parameters for the different exemplary embodiments described herein.

The Kardia Mobile ECG by AliveCor is an example of an ECG device with well-known electronics that can be modified in accordance with the inventive digital therapeutic to detect heartbeat and other heart related measurements. There are many small and inexpensive examples of blood pulse oximeters, automatic blood pressure readers, and skin temperature sensors that can be modified in accordance with the inventive digital therapeutic to detect temperature, blood pressure, pulse, Hood oxygen and other related biometric parameters.

In addition to the well-known strain sensors, a stretchable strain has been recently reported by the University of Houston, Highly Sensitive and Very Stretchable Strain Sensor Based on a Rubbery Semiconductor, ACS Appl. Mater. Interfaces, 2018, 10 (5), pp 5000-5006, 2018. A stretchable strain sensor with printable components has recently been reported by the University of Florida, Highly Stretchable and Wearable Strain Sensor Based on Printable Carbon Nanotube Layers/Polydimethylsiloxane Composites with Adjustable Sensitivity, ACS Appl. Mater. Interfaces, 2018, 10 (8), pp 7371-7380.

Referring to FIG. 15, treatment is started, which may include the ingestion of a pill or a transcutaneous injection of a drug, an applied electrical signal, or the like (Step One) and biometric parameter1 is detected (Step Two). Parameter1 is analyzed to determine if drug delivery is needed (Step Three). For example, the level of a drug in the body can be determined by blood chemistry analyzed directly through transdermal microcapillary or other blood drawing, or through sweat chemistry analysis. The detection of the level of the drug can be extrapolated by a biometric indicator ingested or delivered along with the drug. Alternatively, or additionally, parameter 1 may be a biometric change in the patient's body caused by the drug or lack of sufficient drug levels in the body of the patient. The analysis of parameter 1 is used to determine if drug delivery is needed (Step Four), and if so, the correct dosage and timing (e.g., fast acting or time released) of the drug is delivered to the patient (Step Five). The deliver may include the administration of a pill or capsule, an injection, a manually or automatically controlled intravenous drip, a remotely controlled iontophoresis patch, or other manual or automatic drug delivery mechanism.

As an example of a drug delivery mechanism that can be controlled for on-demand application (for example, by a remote caregiver or AI agent receiving an alert) iontophoresis can be utilized. In this case, iontophoresis uses as an example, a polarized electrical current to push same-charged medications into the blood stream through the skin. Liquid medications are either positively or negatively charged, then an electrical potential applied through the drug to the skin surface to drive the drug into the body. The drug can also be forced subcutaneously according to a timer, or in response to a sensed condition, such as blood sugar level, blood pressure, skin temperature or heartbeat, or in response to a patient activation, or other triggering mechanism.

After deciding whether or not the drug delivery is needed, parameter2 is then detected (Step Six). Parameter2 is analyzed to determine if and how to adjust an applied treatment (Step Seven), such as a change in the characteristic of an applied electrical signal. As a non-limiting example, the applied treatment may be the sequential inducement of involuntary muscle contractions in the calf of the patient to cause a milking action to occur in the veins of the leg in the direction of blood flow returning to the heart. The applied signal or treatment is adjusted depending on the analyzed parameter2 (Step Eight). It is then determined if the treatment time has been exceeded (Step Nine). If it has not, then parameter 1 is again detected (Step Two). For example, if the patient is to have the sequential inducement of involuntary muscle contractions for two hours per treatment cycle, then after the expiration of two hours from the start of treatment (Step One), the treatment cycle ends (Step Ten). Note, that the end of the treatment of the applied signal may end while the drug delivery portions of the flow chart continue (for example, Steps Two through Five) and then the remaining steps related to the detection of parameter2 may be restarted after a predetermined time period.

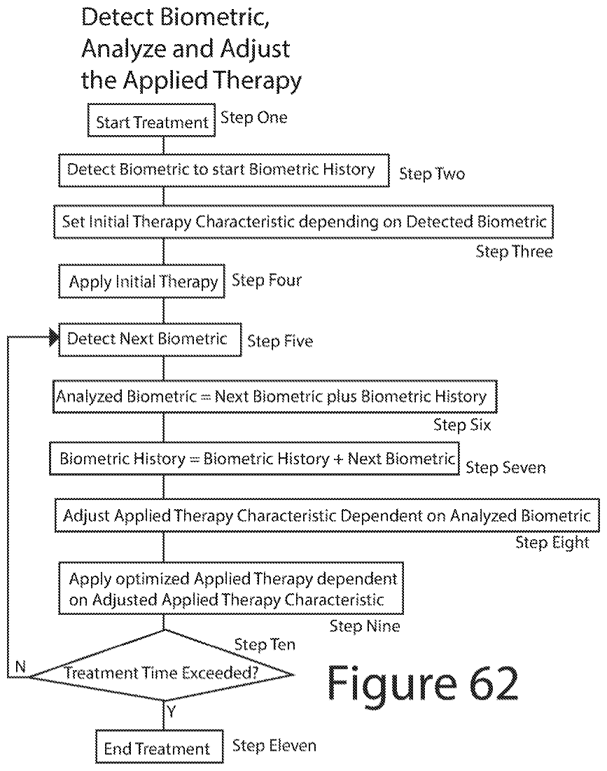

FIG. 62 is a flow chart showing an algorithm for biometric parameter detection and analysis, and then the adjustment of an applied therapy dependent on that analysis. An embodiment of the inventive digital therapeutic can be used for detecting a biometric parameter and using the analysis of the detected biometric parameter to automatically adjust an applied therapy. The biometric parameter can be, but is not limited to, heartbeat, blood flow, body temperature, skin color, blood and/or sweat chemistry, respiration, blood oxygen level, or the like. The applied therapy can include, but is not limited to, EMS, TENS, compression, drug selection, delivery and/or dosage modification, heat, cold or the like. In accordance with this embodiment, the detection and analysis of the biometric parameter can be used to help treat the individual patient with an automatically optimized applied therapy.

Referring to FIG. 16, treatment is started, which may include the previous or concurrent ingestion of a pill or a transcutaneous injection of a drug, an applied electrical signal, or the like (Step One) and a biometric parameter(s) is detected to start a biometric history (Step Two). The detected biometric or biometric parameter can be, for example, blood flow. An initial therapy characteristic is set depending on the detected biometric (Step Three). For example, the therapy may be a sequentially applied EMS signal that causes muscles to contract and milk blood through a vein in the direction of the heart or through an artery in the direction away from the heart. The therapy could be another form of physical, pharmacological, electroceutical or physiological therapy, such as drug, sound, heat, cold, pressure, acupuncture, or the like. Alternatively or in addition, cognitive therapy may be used and include the immersion of the patient into a virtual or augmented reality scene in conjunction with the detection of biometric parameters and the administration of EMS, physical, pharmacological, electroceutical or physiological therapy or other therapies.

The initial therapy is applied having the characteristic set depending on the detected biometric (Step Four). A next biometric is then detected (Step Five). The next biometric may be the same type (e.g., blood flow) or a different type (e.g., sweat chemistry). The next biometric plus the biometric history (which will typically depend on previously detected biometrics) is analyzed to create an analyzed biometric (Step Six). The biometric history is then updated to include the next biometric (Step Seven). As a non-limiting example, the biometric can be determined using a strain gauge that measures a tightness in the calf muscles. When successive strain gauge readings (that is, the next biometric and the biometric history) are compared, the analyzed biometric may indicate that swelling is occurring in the region of the calf muscles indicating poor blood flow in the direction back to the heart in that region. In this example, it may be advantageous to adjust the applied therapy characteristic to increase the strength of the sequentially applied EMS signal or the duration of the sequentially applied EMS therapy (i.e., extend the treatment time), to help improve blood flow through the veins back to the heart and reduce the swelling. In this way, the adjusted applied therapy characteristic is used to optimize the applied therapy that is applied to the patient (Step Nine). If the treatment time is exceeded (Step Ten) the treatment is ended (Step Eleven), otherwise the next biometric is detected (Step Five) and the flowchart steps continue.

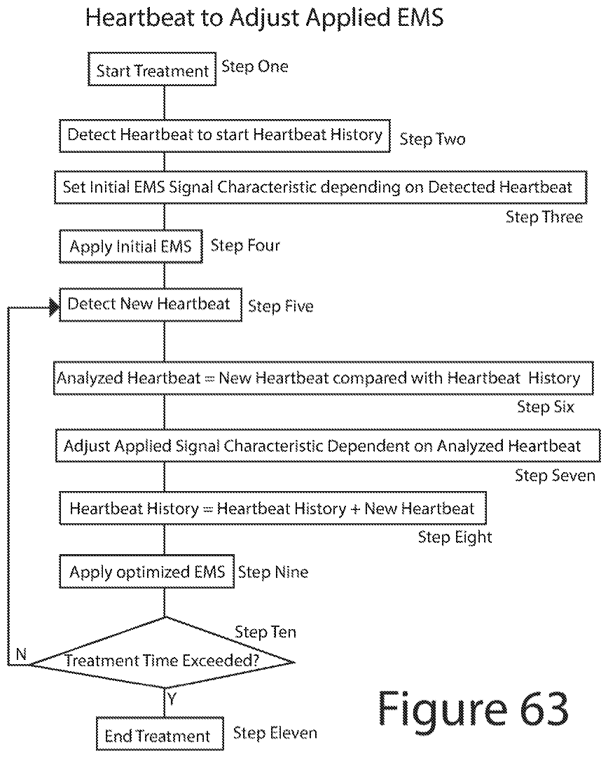

FIG. 63 is a flow chart showing an algorithm for using a detected heartbeat biometric parameter to adjust an applied EMS therapy. The biometric signal may be dependent on heartbeat and the applied electrical muscle stimulation signal is modified to impart the squeezing action dependent on the heartbeat. The heartbeat biometric signal may be detected as at least one of a biometric optical signal and an biometric electrical signal from at least one biometric detector in contact with a skin surface of the patient. The electrical muscle stimulation signal may be applied to the at least one muscle through the skin surface from at least one electrode in contact with the skin surface, and the biometric detector may include the at least one electrode that applies the electrical muscle stimulation signal also used for detecting the biometric electrical signal. The biometric signal may be dependent on surface vein blood flow and the applied electrical muscle stimulation signal may be modified to impart the squeezing action dependent on the surface vein blood flow.

An embodiment of the inventive digital therapeutic can be used for detecting heartbeat, for example, at a peripheral artery on the ankle or at another body part. The heartbeat biometric parameter is analyzed to automatically adjust an applied EMS therapy. The biometric parameter can also include, but is not limited to, the detection of blood flow, body temperature, skin color, blood and/or sweat chemistry, respiration, blood oxygen level, blood pressure, or the like, which may be done in conjunction simultaneously or intermittently with the heartbeat detection. The aggregate data from multiple sensors and biometric parameters may improve the overall performance of the system for automatically optimizing the applied therapy. The applied therapy can include but is not limited to being used in combination along with EMS, TENS, compression, drug selection, delivery and/or dosage modification, heat, cold or the like. In accordance with this embodiment, the detection and analysis of the heartbeat biometric parameter can be used to help treat the individual patient with an automatically optimized applied EMS therapy.

Referring to FIG. 17, treatment is started, which may include the previous or concurrent ingestion of a pill or a transcutaneous injection of a drug, an applied electrical signal, or the like (Step One) and a heartbeat biometric parameter is detected to start a heartbeat biometric history (Step Two). The heartbeat biometric parameter can also include, for example, combined parameters that include blood flow measured in conjunction with heartbeat (with the possibility for analysis, for example, to get an indication of a change in blood pressure extrapolated from a baseline low blood pressure data point with a measured heartbeat and blood flow reading and a baseline raised blood pressure data point also with a measured heartbeat and blood flow reading).

An initial EMS therapy characteristic is set depending on the detected heartbeat biometric (Step Three). For example, the therapy may be a relatively weaker sequentially applied EMS signal that causes muscles to contract and milk blood through a vein in the direction of the heart or through an artery in the direction away from the heart. The therapy could be another form or combination of therapies, such as drug, sound, heat, cold, pressure, acupuncture, or the like. The initial EMS therapy is applied having the characteristic set depending on the detected heartbeat biometric (Step Four). A next heartbeat biometric is then detected (Step Five). The next heartbeat biometric may be detected alone or in conjunction with one or more other types of biometrics (e.g., blood flow, sweat chemistry). The next heartbeat biometric plus the heartbeat biometric history (which will typically depend on previously detected heartbeat biometrics) is analyzed to create an analyzed heartbeat biometric (Step Six). The heartbeat biometric history is then updated to include the next heartbeat biometric (Step Seven).

As a non-limiting example, other biometrics can be determined for example, by detecting light reflectance to indicate a change in skin color. When successive skin color readings (for example, taken along with the next heart biometric and the heartbeat biometric history) are compared, the analyzed skin color biometric may indicate that skin color in the region of the calf muscles indicates poor blood flow in the direction back to the heart. In this example, it may be advantageous to adjust the applied therapy characteristic to increase the strength of the sequentially applied EMS signal or the duration of the sequentially applied EMS therapy (i.e., extend the treatment time), to help improve blood flow through the veins back to the heart and reduce the swelling. In this way, the adjusted applied EMS signal characteristics are used to optimize the applied EMS therapy that is applied to the patient (Step Nine).

An alert may also be sent out to indicate to the patient, the caregiver, the healthcare coverage provider, researchers, or other interested parties that during the course of treatment under the conditions determined by the biometric detections and applied treatment, specific beneficial, neutral or negative results occurred for this particular patient. An aggregate of such data from a population could be effective to assist in optimizing and improving the healthcare treatment, reduce costs and in general improve the outcomes of similarly situated patients. If the treatment time is exceeded (Step Ten) the treatment is ended (Step Eleven), otherwise the next biometric is detected (Step Five) and the flowchart steps continue.

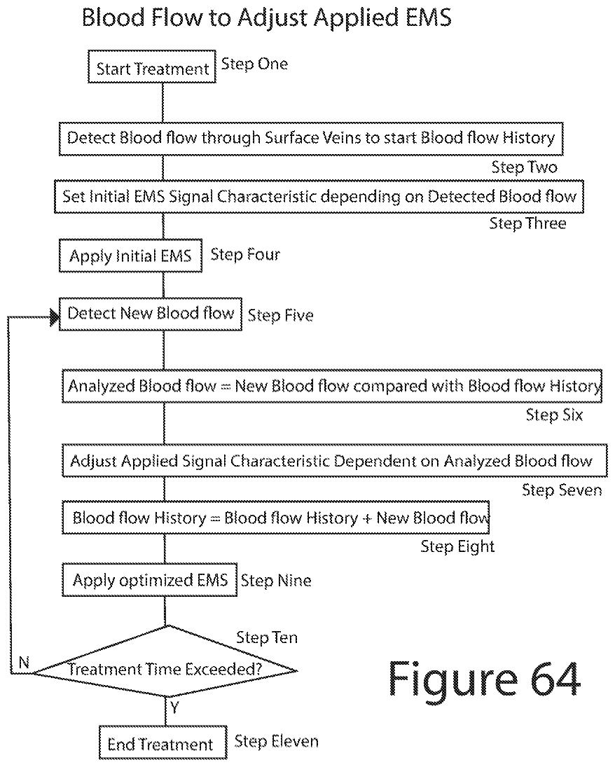

FIG. 64 is a flow chart showing an algorithm for using a detected blood flow biometric parameter to adjust an applied EMS therapy. An embodiment of the inventive digital therapeutic can be used for detecting blood flow, for example, at surface veins in the calf or at another body part. The blood flow at the surface veins in the calf will be particularly useful to indicating the flow of blood from the deep veins in the legs, and those particularly susceptible to DVT. The blood flow biometric parameter is analyzed to automatically adjust an applied EMS therapy. The biometric parameter can also include, but is not limited to, the detection of heartbeat, body temperature, skin color, blood and/or sweat chemistry, respiration, blood oxygen level, blood flow, or the like, which may be done in conjunction simultaneously or intermittently with the blood flow detection. The aggregate data from multiple sensors and biometric parameters may improve the overall performance of the system for automatically optimizing the applied therapy. The applied therapy can include, but is not limited to, being done in combination along with EMS, TENS, compression, drug selection, delivery and/or dosage modification, heat, cold or the like. As with any of the embodiments described herein, the selection of biometric, environmental, or other measured condition is not limited to specific metrics but will depend on the particular application and treatment, data collection, and/or other use of the detected metric. Also, the treatment employed in any of the embodiments described herein is not limited to a specific treatment or action but will depend on the intended use and desired outcome of the combined detected metrics and applied treatments. As an example, in accordance with this embodiment, the detection and analysis of the blood flow biometric parameter can be used to help treat the individual patient with an automatically optimized applied EMS therapy.

Referring to FIG. 18, treatment is started, which may include the previous or concurrent ingestion of a pill or a transcutaneous injection of a drug, an applied electrical signal, or other therapeutic treatment (Step One) and a blood flow biometric parameter is detected to start a blood flow biometric history (Step Two). The blood flow biometric parameter can also include, for example, a combined parameter that includes skin color and/or skin temperature measured in conjunction with blood flow (with the possibility for analysis, for example, to get an indication of a change in the effectiveness of a medication extrapolated from the measured parameters, for example, blood flow, skin color, skin temperature, etc.). An initial EMS therapy characteristic is set depending on the detected blood flow biometric (Step Three). For example, the therapy may be a relatively stronger sequentially applied EMS signal that causes muscles to contract and milk blood through a vein in the direction of the heart or through an artery in the direction away from the heart. The therapy could be another form or combination of therapies, such as drug, sound, heat, cold, pressure, acupuncture, or the like. The initial EMS therapy is applied having the characteristic set depending on the detected blood flow biometric (Step Four). A next blood flow biometric is then detected (Step Five). The next blood flow biometric may be detected alone or in conjunction with one or more other types of biometrics (e.g., heartbeat, skin tightness, swelling, temperature, color, respiration, blood chemistry, sweat chemistry, etc.). The next blood flow biometric plus the blood flow biometric history (which will typically depend on previously detected blood flow biometrics) is analyzed to create an analyzed blood flow biometric (Step Six). The blood flow biometric history is then updated to include the next blood flow biometric (Step Seven).

As a non-limiting example, other biometrics can be determined for example, by detecting a change in skin temperature. When successive skin temperature readings (for example, taken along with the next blood flow biometric and the blood flow biometric history) are compared, the analyzed skin temperature biometric may indicate that skin temperature in the region of the calf muscles indicates improved blood flow in the direction back to the heart. In this example, it may be advantageous to adjust the applied therapy characteristic to decrease the strength of the sequentially applied EMS signal or the duration of the sequentially applied EMS therapy (i.e., reduce the treatment time), to help improve the comfort and convenience for treating the patient while optimizing the total treatment considerations that include patient comfort and treatment adherence, convenience, battery life, and any other detectable, determinable or assumed condition that will promote the total treatment to promote the health of the patient by assisting in the blood flow through the veins back to the heart. In this way, the adjusted applied EMS therapy characteristic is used to optimize the applied EMS therapy that is applied to the patient (Step Nine). An alert may also be sent out to indicate to the patient, the caregiver, the healthcare coverage provider, researchers, or other interested parties indicating the patient's adherence to prescribed treatment and/or under what conditions the prescribed treatment can be modified for improved patient outcome. If the treatment time is exceeded (Step Ten) the treatment is ended (Step Eleven), otherwise the next biometric is detected (Step Five) and the flowchart steps continue.

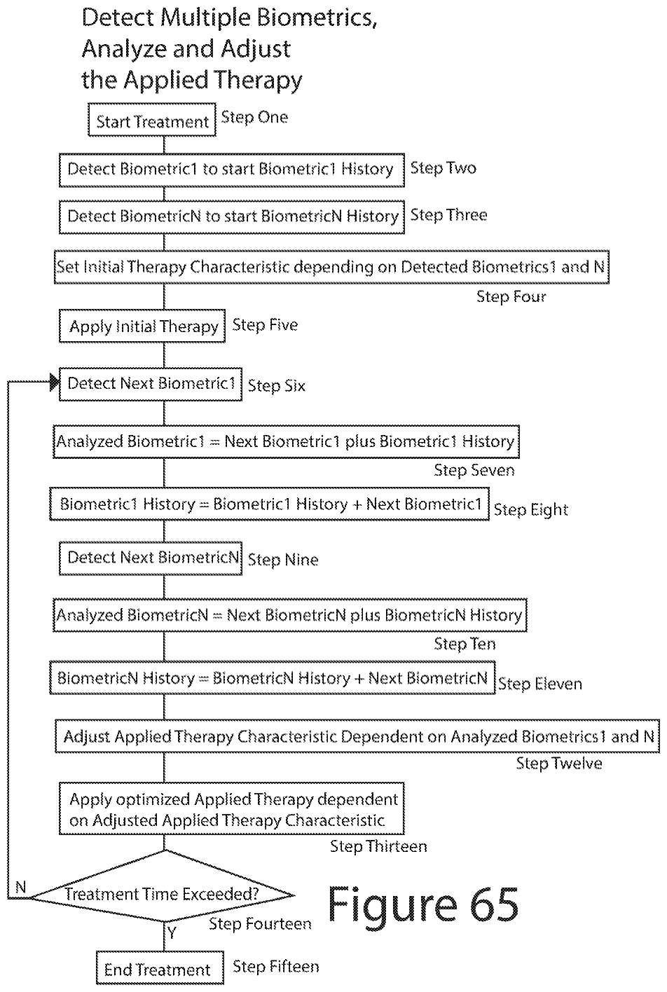

FIG. 65 is a flow chart showing an algorithm for using multiple detected biometric parameters to adjust an applied therapy. As a specific non-limiting example, an embodiment of the inventive digital therapeutic device may be used alone, as a primary treatment, as complementary treatment, and/or as an adjunct to anticoagulation medicines for the prevention and treatment of atherothrombotic events in patients.

An embodiment of the inventive digital therapeutic can be used for detecting multiple biometrics including, but not limited to surface vein blood flow, skin color, heartbeat, arterial pulse, swelling, tightness, the detection of a biomarker such as, but not limited to, D-dimer protein and other thrombosis biomarkers, blood oxygen, CO2, lactose, glucose, urea (obtained directly from the blood or through the sweat) or other biometric parameter useful for adjusting an applied therapy. The applied therapy can include, but is not limited to, and be done alone or in combination with, EMS, TENS, pneumatic compression, mechanical squeezing (for example, using band actuated by a shape memory metal or other mechanical actuator to apply a squeezing force to the muscles), drugs (with modification to selection, delivery and/or dosage), heat, cold, etc.

Referring to FIG. 19, treatment is started, which may include the previous or concurrent ingestion of a pill or a transcutaneous injection of a drug, an applied electrical signal, or the like (Step One) and a first biometric parameter (biometric1) is detected to start a first biometric history (biometric1 history) (Step Two). A subsequent biometric parameter (biometricN) is detected (Step Three) to start a subsequent biometric history (biometricN history). The number of multiple biometric parameters can include several concurrent or intermittently acquired biometrics obtained from a variety of sensors.

A feature of the inventive digital therapeutic device may be to utilize two or more different sensors to determine two or more different biometric parameters that are affected by the applied therapy and/or progression of treatment and/or change in conditions of the patient and/or external factors such as ambient temperature and/or air pressure. The utilization of two or more biometric parameters may enable a higher signal to noise ratio of the obtained useful data used to adjust one or more applied therapy characteristics and/or as data that is transmitted for aggregate population studies and/or used in the ongoing treatment of the particular patient.

After the detection of the biometric parameters, an initial therapy characteristic is set depending on those detected biometric parameters (Step Four) and the initial therapy is applied (Step Five). For example, if the applied therapy is sequential EMS signals applied to the calf muscles to create a milking action of blood in deep veins in the direction towards the heart, biometric parameters of pulse and blood flow can determine the timing and intensity of the applied EMS signals to optimize the blood flow while minimizing any discomfort to the patient.

After the application of the initial therapy, the steps of an iterative loop are performed with measured results of the patient's reaction to the applied therapy taken into consideration to automatically adjust the characteristics of the applied therapy. During this feedback loop, a next biometric1 is detected (Step Six) and analyzed (Step Seven) by comparing, contrasting or otherwise determining a change, if any, of the most recent detection of biometric1 (that is, next biometeric1) with the biometric1 history (which may include all or some of the cumulative previously taken biometric1 detections). The biometric1 history is then updated to include the just pervious bioinetric1 detection (next biometric1). For each of the subsequent biometrics (biometricN), a next biometricN is detected (Step Nine) and analyzed (Step Ten) by comparing, contrasting or otherwise determining a change, if any, of the most recent detection of biometricN with the biometricN history. The biometricN history is then updated to include the just pervious biometricN detection (next biometricN) (Step Eleven).

The analyzed biometrics 1 through N are then used to adjust an applied therapy characteristic (Step Twelve) and an optimized applied therapy that is dependent on the adjusted applied therapy characteristic is applied to the patient (Step Thirteen). If the treatment time (which could be one of the characteristics of the applied therapy that is adjusted) has not been exceeded (Step Fourteen), the feedback loop begins again the next biometric1 is detected (Step Six). If the treatment time has expired (Step Fourteen), the treatment ends (Step Fifteen).

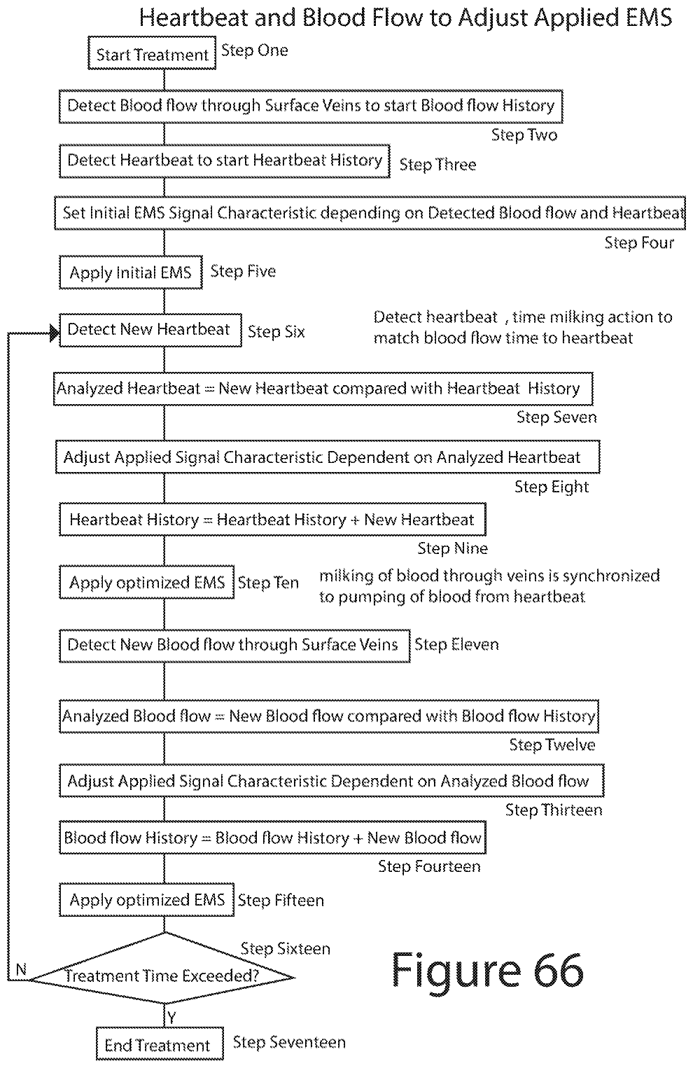

FIG. 66 is a flow chart showing an algorithm for using multiple detected biometric parameters of heartbeat and blood flow to adjust an EMS applied therapy. Treatment is started, which may include the previous or concurrent ingestion of a pill or a transcutaneous injection of a drug, an applied electrical signal, or the like (Step One) and a first biometric parameter (blood flow) is detected to start a heartbeat history (blood flow history) (Step Two). A subsequent biometric parameter (heartbeat) is detected (Step Three) to start a subsequent biometric history (heartbeat history). The multiple biometric parameters of blood flow and heartbeat can be obtained concurrently (simultaneous) or intermittently from different sensors (e.g., an optical sensor used for blood flow and an electrical sensor used for heartbeat) located at different locations on the body or can be obtained from data collected from the same sensor (e.g., an optical sensor that can detect both blood flow and heartbeat) but analyzed to determine the different parameters.

The different biometric parameters of blood flow and heartbeat may be affected by the applied EMS therapy and/or the progression of treatment and/or change in conditions of the patient and/or external factors such as ambient temperature or air pressure. For example, when in the pressurized cabin of an airplane, the inventive digital therapeutic device can sense the prolonged increase pressure and automatically adjust the EMS signal intensity, treatment duration, length of involuntary contractions, etc.

After the detection of the biometric parameters, an initial EMS signal characteristic is set depending on those detected heartbeat and blood flow biometric parameters (Step Four) and the initial EMS therapy is applied (Step Five). After the application of the initial EMS therapy, the steps of an iterative loop are performed with measured results of the patient's reaction to the applied therapy taken into consideration to automatically adjust the characteristics of the applied therapy. During this feedback loop, a next or new heartbeat is detected (Step Six) and analyzed (Step Seven) by comparing, contrasting or otherwise determining a change if any, of the most recent detection of heartbeat (that is, next heartbeat) with the heartbeat history (which may include all or some of the cumulative previously taken heartbeat detections). The analyzed heartbeat biometric is used to adjust the applied signal characteristic of an EMS signal (Step Eight). The heartbeat history is then updated (Step Nine) to include the just pervious heartbeat detection (next heartbeat). The optimized EMS signal is applied, for example, causing a sequence of involuntary muscle contractions in the calf muscles to cause a milking action of blood in a deep vein in a direction back towards the heart and synchronized to the detected heartbeat. A next or new blood flow through surface veins reading is detected (Step Eleven) and analyzed (Step Twelve) by comparing, contrasting or otherwise determining a change, if any, of the most recent detection of blood flow with the blood flow history (or only the just previous blood flow detection). The analyzed blood flow biometric is used to adjust the applied signal characteristic of an EMS signal (Step Thirteen). The blood flow history is then updated to include the just pervious blood flow detection (next blood flow) (Step Fourteen). The newly optimized EMS signal is applied to the patient via the inventive digital therapeutic (Step Fifteen).

In accordance with this non-limiting, exemplary embodiment, the analyzed biometrics of heartbeat and blood flow are used to adjust an applied EMS signal characteristic and an optimized applied EMS therapy that is dependent on the adjusted applied EMS signal characteristic is applied to the patient. Heartbeat and blood flow are just example biometric parameters, and one or more biometric parameter may by employed to automatically adjust, or alert that an adjustment is desired, of an applied treatment. Also, the detected biometric(s) can be used to trigger data logging and/or transmission, if, for example, a threshold is exceeded indicating it is desirable to log or transmit data related to the detected biometric. If the treatment time has not been exceeded (Step Sixteen), the feedback loop begins again and the next heartbeat is detected (Step Six). If the treatment time has expired (Step Sixteen), the treatment ends (Step Seventeen).

An embodiment of the inventive digital therapeutic device comprises a wearable electronic garment having at least one pair of electrodes for applying an electrical muscle stimulation signal through the skin to induce involuntary contractions in one or more muscles adjacent to a blood vessel. The involuntary muscle contractions induce a squeezing action on the blood vessel and promote a flow of blood through the blood vessel. A biometric signal detector detects a biometric parameter indicative of the flow of blood through the blood vessel. A microprocessor controls the application of the electrical signal dependent on the detected biometric signal. The biometric parameter may be dependent on a therapeutic action of a pharmaceutical medicinal compound. The applied electrical muscle stimulation signal may be modified in response to the therapeutic action of the pharmaceutical medicinal compound. The biometric signal may be dependent on at least one detectable biometric parameter. The biometric parameter may be detectable dependent on at least one of skin temperature, skin color, blood flow, pulse, heartbeat, blood pressure, skin tightness, swelling, blood chemistry, sweat chemistry, an electronic biomarker, a chemical biomarker, and electromyography.

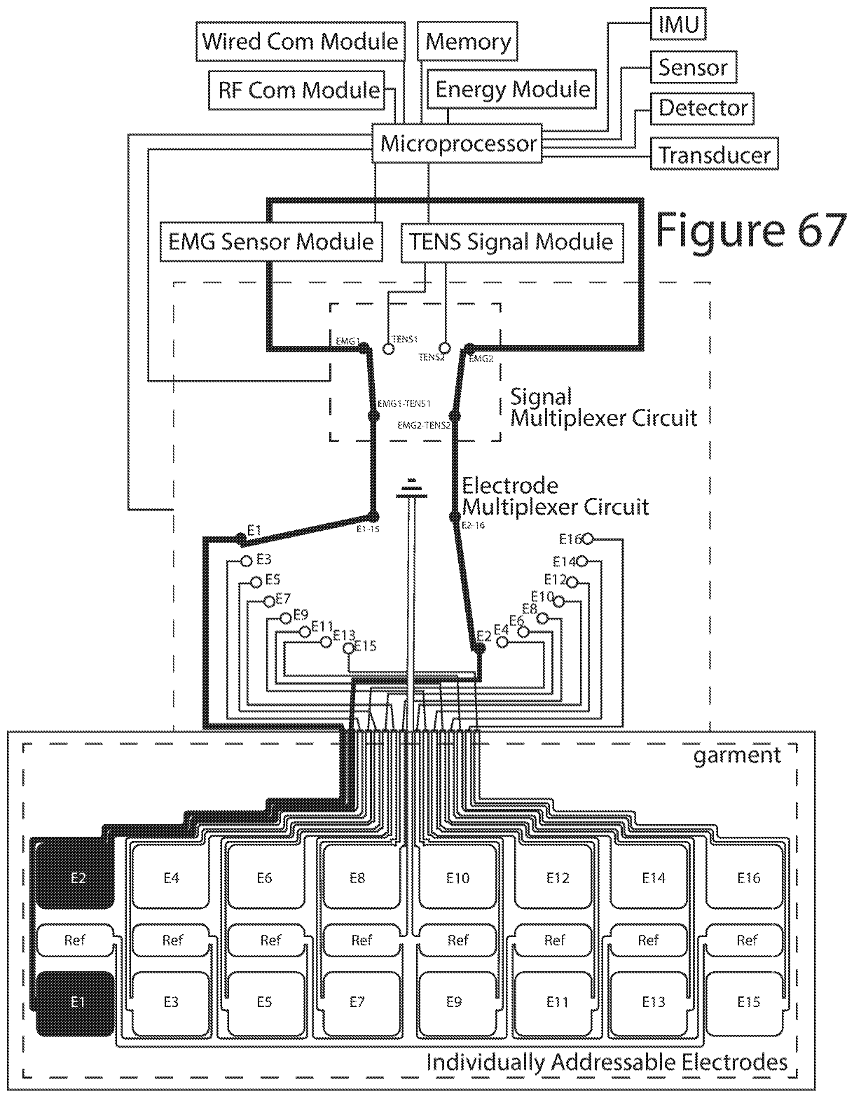

FIG. 67 illustrates an exemplary embodiment showing bi-directional electrical signals applied through a plurality of individually addressable electrodes routed through an electrode multiplex circuit and a signal multiplex circuit for applying a sequential EMS signal and detecting biometric feedback from the calf, or other body part, of a patient. In accordance with an aspect of the invention, a digital therapeutic device garment is provided with a plurality of individually addressable electrodes supported by the garment for applying a sequential EMS signal and detecting biometric feedback from the calf of a patient. The individually addressable electrodes are for at least one of applying stimulation electrical signals to the skin of a patient and detecting biometric electrical signals from the skin of the patient.

At least one of a signal detector for detecting the biometric electrical signals and a signal generator for generating the stimulation electrical signals are provided. An electrode multiplex circuit addresses the plurality of individually addressable electrodes by at least one of routing the biometric electrical signals from the skin of the patient through more than one of the plurality of individually addressable electrodes to the signal detector and routing the stimulation electrical signals from the signal generator through more than one of the plurality of individually addressable electrode to the skin of the patient. A microprocessor controls the signal detector, the signal generator, the electrode multiplex circuit and other circuit components.

The microprocessor can control the electrode multiplex circuit to route the biometric electrical signals from the skin of the patient sequentially through more than one of the plurality of individually addressable electrodes to the signal detector. In accordance with this embodiment, a single EMS signal source can service multiple individually addressable electrodes with the EMS signal routed as desired for an intended therapy, such as for the sequential squeezing of the deep veins in the legs to promote blood flow in the direction back to the heart. One or more EMS signal channels can be multiplexed and signals routed so that even a large array of individually addressable electrodes can be serviced by one or a few signal generators, for example, to provide a finer spatial resolution of the applied EMS signal than indicated by number of electrodes shown in the drawings.

The microprocessor can control the electrode multiplex circuit to route the biometric electrical signals (indicating, for example, muscle activity, heartbeat, etc.) from the skin of the patient simultaneously through more than one of the plurality of individually addressable electrodes to the signal detector. The microprocessor can control the electrode multiplex circuit to route the stimulation electrical signals from the signal generator simultaneously through more than one of the plurality of individually addressable electrodes to the skin of the patient. The microprocessor can control the electrode multiplex circuit to route the stimulation electrical signals from the signal generator sequentially through more than one of the plurality of individually addressable electrodes to the skin of the patient. The microprocessor can control the electrode multiplex circuit to route the stimulation electrical signals from the signal generator simultaneously through more than one of the plurality of individually addressable electrodes to the skin of the patient.

That is, in the case of DVT, the electrodes can be sequentially targeted to apply EMS signals to the muscles in the legs of the patient, causing contractions synchronized with the heartbeat and create a milking action through the sequential muscle contractions to promote blood flow through the veins and in the direction towards the heart. Of course, if desired, the contractions can be timed and sequenced to promote blood flow in the direction away from the heart and through the arteries in the legs (or other body part with a different wearable electronic configuration and/or applied electrical signals timing, characteristics, etc.) as well.

FIG. 67 illustrates an embodiment of an inventive wearable electronic that can be used as an interface for selectively applying transcutaneous electrical nerve stimulation and selectively detecting electromyography through the same electrodes and/or circuit components.

A wide range of biometric and ambient conditions can be detected and made available for therapeutic and research purposes. An important use of the inventive digital therapeutic platform is to contribute enabling technologies and products to ultimately create a cradle-to-grave system that obtains biometrics from a patient, forever secures the data integrity, privacy, and access, and makes this secured data available to feed big data pattern recognition to improve global human health.

There are uses of an inventive biometric/Blockchain/AI model described herein that apply to other emerging sensor-based IoT systems for uses including, but not limited to, monitoring crop growth, livestock, sanitation, and mitigating infant mortality in developing countries. While not specifically illustrated herein, many of the techniques, hardware and software could be employed for these other uses.

With regards to the biometric/BC/AI platform described herein, the general premise is that soon biometrics will be collected from a large segment of the population nearly all day/every day. Over time (eventually, a lifetime) this data may be far more valuable at predicting and preventing disease and other health issues than yearly physical examinations. This health advantage will be very compelling and cause more sales and use of digital therapeutic devices for collecting more biometrics more of the time.

For population studies, this data may be invaluable to medical researchers, NGOs and government organizations. A distributed ledger technology can be adapted to make this data available, in bulk, as anonymous, accurate information (e.g., only demographics, nothing to identify the individual) while also making it securely and permanently stored to be analyzed by a trusted receiver (e.g., a health care provider) over the lifetime of the individual. The content and quantity of the individual's data made available can be adjusted depending on the data recipient.

The inventive digital therapeutic can be used to provide secure and accurate open-source or privately controlled access to a vast amount of collected biometric data to researchers around the world. However, when many people are wearing some form of biometric detection technology every day (for example, a “smart” T-shirt or underwear), it may not be efficient to put online, for example, every heartbeat, so some filtering and compression of collected data is needed. The biometric data can be filtered to detect anomalies or potential anomalies in the collected biometric data. There also needs to be a data security layer as close to the source (the user or patient) as possible. The inventive digital therapeutic platform uses wearables, blockchain and AI to collects biometric data, such as heartbeat and sweat chemistry from a living organism, like a human or a pet or livestock, anonymously and securely store that data using block chain technology and use AI to look for patterns in the data to determine health aspects of the population such as heart disease and diabetes.

The biometric data can be used for authentication, for example, a wearable electronic configured as socks or underwear is a great target garment for the digital therapeutic device. A detected heartbeat signature that is unique to the individual can be used for secure identification, with near field communication used to send the heartbeat signature from the digital therapeutic device from a smartphone through a WiFi or cellular connection to an online server to securely authenticate the user.

Blockchain distributed ledger is highly valued technology that could store copious biometric data and make it available forever, transparently and securely. AI agents connected to the cloud could look for patterns in this biometric data that is generated by every human body with a statistically relevant database made available through widespread use of the inventive digital therapeutic for a particular condition or class of patient, or from a general population, demographic, geographic, cultural, ethnic or other grouping. These patterns may unlock opportunities for vast improvements in personal and global population health.

Although shown as a legging or stocking, the inventive digital therapeutic can be formed in a number of scalably manufacturable clothing configurations with a large array of many individually addressable electrodes connected to a few or even a single detection and application electronic unit. The architecture of the digital therapeutic is adapted to mass production as a roll-to-roll manufactured printed electronic garment with embedded sensors and transducers. Applications beyond those described herein include biometric sensing for health and fitness, stroke rehabilitation, tremor mitigation and pain relief. The Blockchain distributed ledger technology is adapted to make this data available, in bulk, as anonymous, accurate information (only demographics, nothing to identify the individual) while also making it securely and permanently stored to be analyzed by a trusted receiver (e.g., health care provider) over the lifetime of the individual.

The inventive digital therapeutic device can be used to provide secure and accurate privately controlled open-source access to a vast amount of collected biometric data to researchers around the world. Garments that incorporate the aspects of the digital therapeutic device described herein will provide bi-directional capabilities to detect/analyze/apply signals to/from the human body and distribute a large array of individually addressable electrodes and sensors, enabling a single sensor or signal generator to service many electrodes, with multiple small electrodes forming physiology matching patterns.

Artificial Intelligence Agents are becoming adept at finding hidden patterns in copious datasets. These hidden patterns can be used to assist researchers in drug discovery, medical device R&D, etc., looking for biomarkers in electrical and chemical activities of the human body. There are a host of other biometric and environmental data applications that could be useful for AI for R&D, some of which have not been conceived yet. The inventive digital therapeutic device can be used by these future applications to easily capture and make available a full range of biometrics from large population samples.

The digital therapeutic device product architecture, manufacturing methods, and applications, can be used for capturing high fidelity biometric data from the human body. The inventive digital therapeutic has advantages that include a low cost, easy to use form factor, for wearable products that have a wide range of sensors and embedded AI software. The bi-directional detect/analyze/apply capability enables proactive responses such as active transdermal drug delivery and transcutaneous electrical nerve stimulation automatically applied or remotely triggered by a monitoring physician. Biometric data can be captured and transmitted continuously or at selected times with data access provided directly to a care-provider, enabling early diagnosis and ongoing monitoring, and to a researcher to gain valuable insights and assistance through AI analysis. This data detection is direct from the human body and can be provided through a wireless connection for the wearable electronic digital therapeutic device for Blockchain and AI database collection, access and analysis. The inventive digital therapeutic device for biometric capture is adapted to mass production as a roll-to-roll manufactured printed electronic garment with embedded sensors and transducers. In addition to AI assisted R&D, applications also include biometric sensing for health and fitness, stroke rehabilitation, tremor mitigation and pain relief.

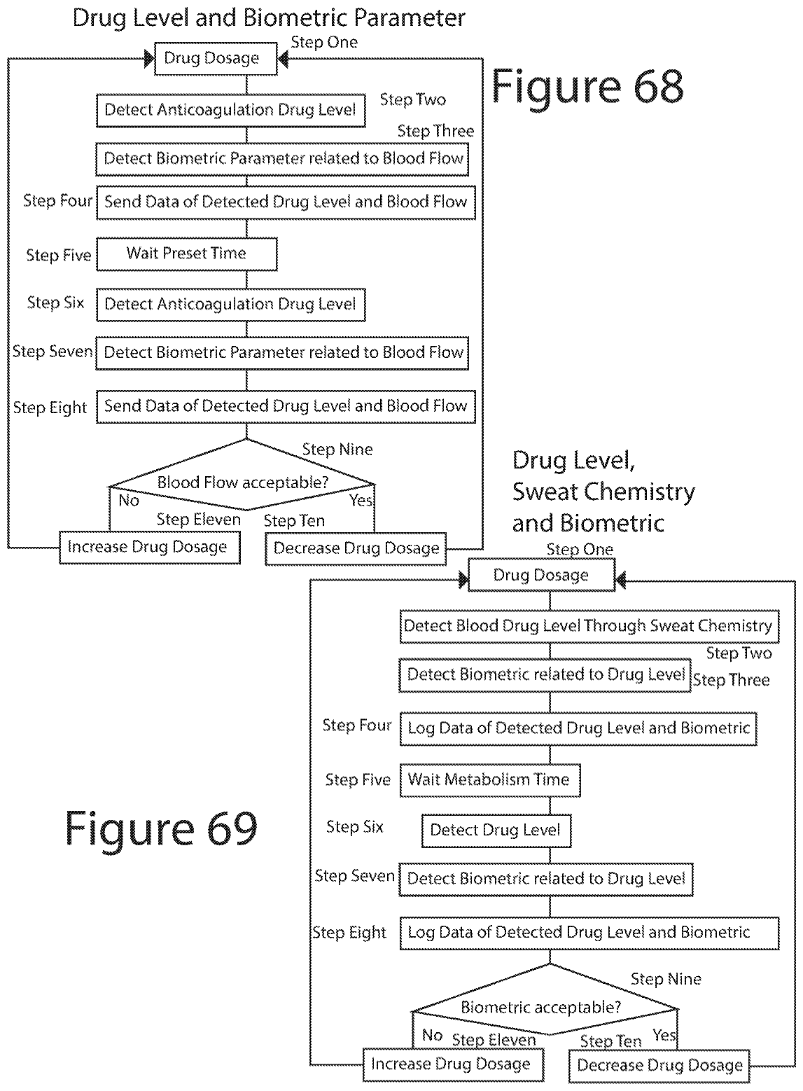

FIG. 68 is a flow chart showing an algorithm for detecting the blood level of a drug, detecting a biometric parameter related to a physiological effect of the drug, sending data related to the detected drug level and biometric parameter and using the detected blood level and biometric parameter to indicate or automatically adjust a dosage of the drug. This exemplary algorithm, like all the examples and embodiments described herein, is intended to be a non-limiting example of computer code, microprocessors, electronic circuits and digital therapeutic device devices employed in a desired treatment and/or monitoring of a patient.

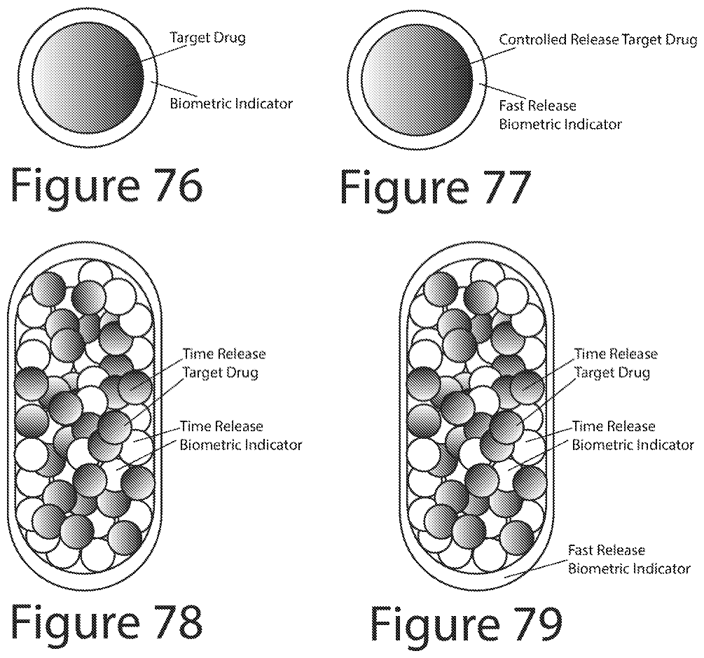

Referring to FIG. 68, an administered drug dosage is taken (Step One). For example, an anticoagulation drug may be orally administered, although the drug may be administered through other delivery mechanisms including but not limited to a transdermal patch, an intravenous drip, inhalation, eye drops, nasal spray, a transdermal injection, an implanted drug release mechanism, or other drug delivery vehicle. In this example, an anticoagulation drug level is detected (Step Two) and a biometric parameter, for example, related to blood flow is detected (Step Three). The anticoagulation drug level can be detected directly, such as through blood or sweat chemistry detection, or may be inferred, for example, through the biometric indicator mechanism described herein, for example, in conjunction with FIG. 76 through 34. The biometric parameter may be, for example, a single biometric parameter including blood flow, blood pressure, skin color, skin temperature, skin/muscle tightness, skin/muscle swelling, pulse, air temperature, humidity, air pressure, time of day, or other environmental and/or biometric that is dependent on a treatment, disease, health or ambient condition around a patient.

The inventive digital therapeutic device can include onboard data collection, reception and transmission which may be for example, wireless WiFi, cellular, Bluetooth, or other wired or wireless data transfer mechanism. The detected drug level and blood flow are transmitted (Step Four), for example, wirelessly from the digital therapeutic device to an internet gateway device and then over the Internet or other network to a caregiver, datacenter, or the like. A preset time is allowed to elapse (Step Five) between detections of the anticoagulation drug level for the effectiveness of the ingested drug to modify the detected biometric parameter, and/or for other reasons including the metabolism of the anticoagulation drug by the body, or other factors that will change either or both the detected drug level or the detected biometric parameter.

After waiting the preset time (Step Five), the drug level of the anticoagulation drug is again detected (Step Six) and the biometric parameter related to blood flow is again detected (Step Seven). The data of the detected drug level and the detected blood flow are again transmitted (Step Eight). It is noted that the inventive digital therapeutic device may be configured to detect and/or send either or both of the detected data. Based on the detected data, a decision may be made to log and/or transmit data, modify treatment, send an alert, and/or other action taken in response to the detected data.

In this exemplary algorithm, the decision is based on whether or not the blood flow is acceptable (Step Nine). If the detected blood flow is acceptable, the drug dosage may be decreased (Step Ten) (or kept at the same level) at the next drug dosage administration to the patient. If the detected blood flow is not acceptable, the drug dosage may be increased (Step Eleven) at the next drug dosage administration.

In this example, the algorithm may be used as a mechanism to use the feedback of the detected biometric to assist in the dosage of the drug. As noted, in this example, the drug is an anticoagulation drug that will affect the blood flow by preventing the formation of blood clots or blood vessel restrictions. The detection may span multiple days, weeks months or even years of treatment, with the effect of the anticoagulation drug as it relates to blood flow being gathered over many administrations of the drug. Although the description of this embodiment pertains to anticoagulation drugs and blood flow, other example uses of the inventive digital therapeutic controlled by a microprocessor may include the detection of pain signals, such as EMG detected muscle spasms, instead of detected blood flow, and other drugs, such as pain medications, muscle relaxers, etc. may be administered and their effect on the detected biometric measured over time.

FIG. 69 is a flow chart showing an algorithm for detecting the blood level of a drug through sweat chemistry detection; detecting a biometric parameter related to a physiological effect of the drug; sending data related to the detected drug level and biometric parameter; and using the detect blood level and biometric parameter to indicate or automatically adjust a dosage of the drug.

An administered drug dosage is taken (Step One). For example, the drug may be an orally administered pill or capsule and its level in the body detected through sweat chemistry detection, using a surrogate biometric detector such as described herein, or another biometric more directly related to the drug (Step Two). A biometric parameter related to the drug level is detected (Step Three). For example, the blood pressure of the patient may change depending on the effect of the drug level.

The detected dug level and biometric are logged, for example, as stored data with a time stamp registered along with the data onto a server, computer storage device, or onboard memory of the inventive digital therapeutic device (Step Four). A preset time is allowed to elapse, for example to allow for the metabolism of the administered drug to either become effective or to begin to be excreted from the body (Step Five). In this example, a predetermined expected metabolism or activation or deactivation time is allowed to elapse between detections of the drug level which may modify the detected biometric parameter. The metabolism, activation and/or deactivation times may relate to the effect the administered drug has on the treatment and/or on the detected biometric.

After waiting the preset time (Step Five), the drug level of the drug is again detected (Step Six) and the biometric parameter related to the drug is again detected (Step Seven). The data of the detected drug level and the detected biometric are again logged (and/or transmitted) (Step Eight). Based on the detected data, a decision is made. In this exemplary algorithm, the decision is based on whether or not the biometric data indicates acceptable conditions (Step Nine). For example, is there adequate drug levels remaining in the blood, or is there a detected improvement or expected treatment results that can be determined from the detected biometric. If the detected biometric is acceptable, the drug dosage may be decreased (Step Ten) (or kept at the same level) at the next drug dosage administration to the patient. If the detected blood flow is not acceptable, the drug dosage may be increased (Step Eleven) at the next drug dosage administration. The increase or decrease in the drug dosage may be done automatically, for example, using an iontophoretic patch as described herein, which can be controlled by a microprocessor in response to a received wireless signal, or automatically where an algorithm or AI agent is employed to analyze the biometric data, including the drug level, and adjust the drug dosage, or an alert may be transmitted for manual drug dosage change, for example, under the guidance of the patient's physician. The automatic control of the drug dosage, or an alert sent to a caregiver to begin the process of modifying the patient drug dosage, may employ the feedback of the detected biometric and the projected or actually measured drug level.

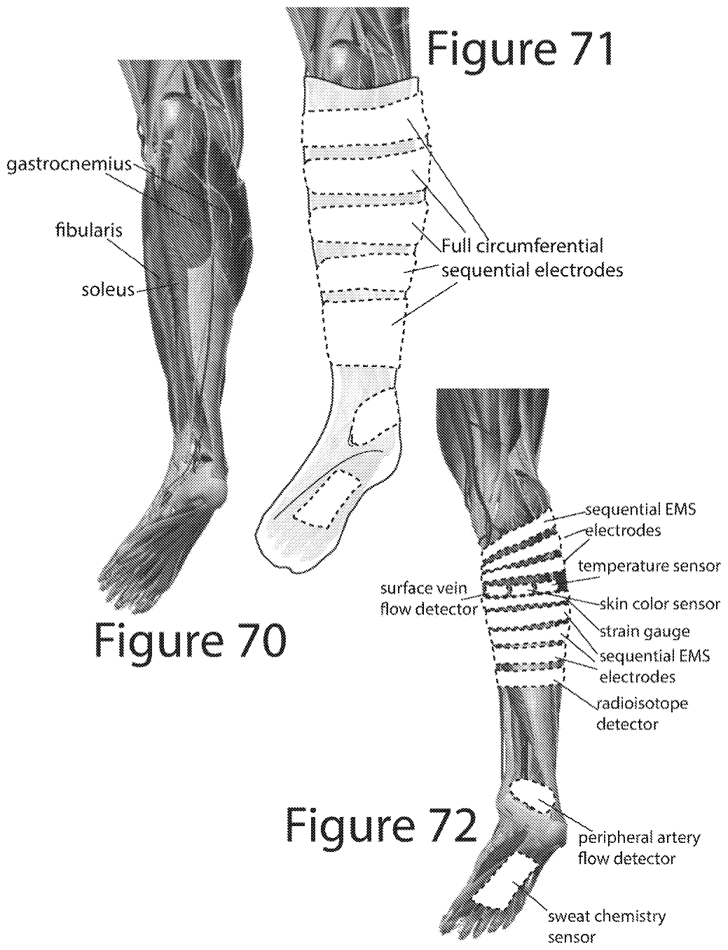

FIG. 70 illustrates the location of muscles of the calf of a patient targeted for sequential involuntary contractions in accordance with an embodiment of the inventive digital therapeutic. Calf and lower leg muscles including the gastrocnemius, fibularis and soleus muscles can be controlled individually or simultaneously to involuntarily contract and milk the blood in the deep veins of the legs in the direction back towards the heart.

FIG. 71 illustrates an embodiment of the inventive digital therapeutic having circumferential electrodes for applying a sequential EMS signal effective for causing simultaneous contractions in multiple targeted muscles synchronized with an expected and/or detected biometric parameter. In this embodiment, the circumferential electrodes are formed as rings encompassing the muscles of the calf and lower leg. The electrodes may have full closed circular ring shape or open semi-circular geometry, they may be small in size to focus the applied signal to a specific nerve or muscle, or they may be large to provide a wider area of signal applied to the skin. The size, geometry and location will depend on factors including the patient's physiology, intended treatment, patient tolerance, and other individual and generic factors. When the electrodes are energized sequentially, two or more of the lower leg muscles are caused to simultaneously contract and squeeze blood through the vein towards the heart, with the strongest local contraction of each muscle possibly in the vicinity of the energized electrodes or a recruitment of motor units caused by the applied signal may cause a cascading muscle contracting event.

FIG. 72 illustrates an embodiment of the inventive digital therapeutic having multiple biometric detectors and multiple individually addressable electrodes to enable the use of multiple detected biometric parameters to adjust an applied therapy. The biometric parameters may include a strain gauge formed from an elastic resistance strip that reversibly changes a detectable resistance value based on being stretched. The sweat chemistry sensor may comprise a stretchable electrochemical sweat sensor made, for example, by the deposition of carbon nanotubes (CNTs) on top of patterned Au nanosheets (AuNS) as reported by the graduate school of converging science and technology, Korea University, Seoul (see, for example, Skin-Attachable, Stretchable Electrochemical Sweat Sensor for Glucose and pH Detection, ACS Applied Materials & Interfaces 2018 10 (16), 13729-13740 DOI: 10.1021/acsami.8b03342). This is one example of a sweat chemistry sensor that could be employed as part of the inventive digital therapeutic, for the detection of glucose and pH. In this case, CoWO4/CNT and polyaniline/CNT nanocomposites are coated onto the CNT-AuNS electrodes, respectively. A reference electrode is prepared via chlorination of silver nanowires. A change in electrical signal characteristics among the electrodes is indicative of the detected glucose and pH. By modifying the functionalized components, other chemicals present in the sweat can be targeted for detection. By providing multiple sweat chemistry sensor or by creating a patchwork of differently functionalized areas of a multiplexed array of sweat chemistry sensors, a variety of chemicals present in sweat (or the absence thereof) can be determined. To induce adequate sweat even from a sedentary patient, a company called Eccrine Systems has demonstrated a gel containing carbachol, a chemical used in eyedrops is effective to induce sweat. The gel was tested on sensors alone and in combination with memory foam padding (to provide better contact between the sensor and the skin) and iontophoresis, where an electrical current at 0.2 milliamps drives carbachol into the upper layer of the skin and locally stimulates sweat glands but causes no physical sensation or discomfort.

In accordance with the inventive digital therapeutic, a material, such as cabachol can be used to help stimulated sweat, for example, from the bottom of the foot where there are a high number of sweat glands. The cabachol or other sweat inducing chemical may be included at the skin contact surface of the same electrodes that apply an EMS signal and/or detect an EMG signal, and a mechanism, such as that described herein with reference to FIGS. 9 through 13 used for detecting the presence or absence of chemicals in sweat. Using other functionalized chemistry, a sweat chemistry sensor can be designed to target a specific chemical signature present in the sweat of the patient. In general, any water-soluble blood component will be present in the sweat of the patient, enabling a non-invasive biometric detector to be employed in accordance with the inventive wearable electronic digital therapeutic device.

As a non-limiting example, other biometrics can be determined for example, by detecting light reflectance to indicate a change in skin color. When successive skin color readings (for example, taken along with the next heart biometric and the heartbeat biometric history) are compared, the analyzed skin color biometric may indicate that skin color in the region of the calf muscles indicates poor blood flow in the direction back to the heart. In this example, it may be advantageous to adjust the applied therapy characteristic to increase the strength of the sequentially applied EMS signal or the duration of the sequentially applied EMS therapy (i.e., extend the treatment time), to help improve blood flow through the veins back to the heart and reduce the swelling. In this way, the adjusted applied EMS therapy characteristic is used to optimize the applied EMS therapy that is applied to the patient. An alert may also be sent out to indicate to the patient, the caregiver, the healthcare coverage provider, researchers, or other interested parties that during the course of treatment under the conditions determined by the biometric detections and applied treatment, specific beneficial, neutral or negative results occurred for this particular patient. An aggregate of such data from a population could be effective to assist in optimizing and improving the healthcare treatment, reduce costs and in general improve the outcomes of similarly situated patients.

The blood flow biometric parameter can also include, for example, a combined parameter that includes skin color and/or skin temperature measured in conjunction with blood flow (with the possibility for analysis, for example, to get an indication of a change in the effectiveness of a medication extrapolated from the measured parameters, for example, blood flow, skin color, skin temperature, etc.). For example, the therapy may be a relatively stronger sequentially applied EMS signal that causes muscles to contract and milk blood through a vein in the direction of the heart or through an artery in the direction away from the heart.

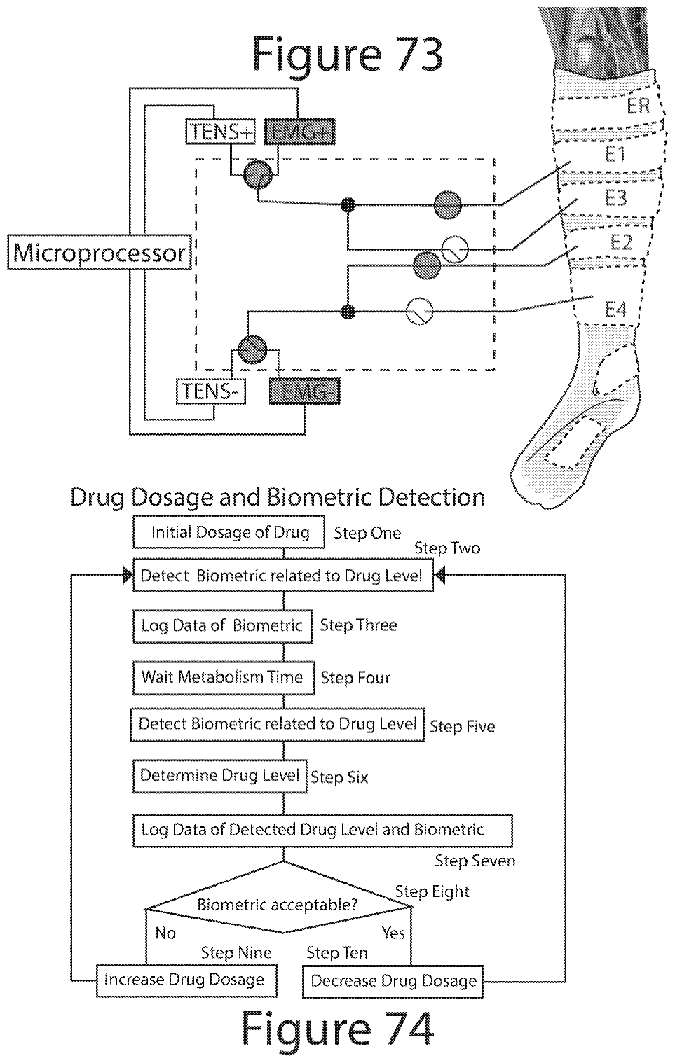

FIG. 73 illustrates the inventive digital therapeutic for selectively applying transcutaneous electrical muscle and/or nerve stimulation as an applied therapy and selectively detecting electromyography as a biometric parameter through the same electrodes and/or circuit components.

In accordance with an exemplary embodiment, a wearable electronics device architecture and fabrication method are provided for generating a wearable electronic digital therapeutic that may include a high-speed multiplexing electronic circuit connecting an array of many individually addressable electrodes to a single or a few detection and application electronic units. The architecture of the digital therapeutic is adapted to mass production as a single, batch or roll-to-roll manufactured printed electronic garment with embedded sensors, electrodes and transducers.

Exemplary embodiments of the digital therapeutic utilize existing stretch fabrics (such as Lycra and Spandex), printing techniques (including screen printing, stamping and inject printing), and mature roll-to-roll lamination processing technology that has previously been used, for example, in the sign making industry. In accordance with an exemplary manufacturing process, these previously known manufacturing techniques are modified to create a new high yield batch and roll-to-roll manufacturing process for fabricating wearable electronics digital therapeutic products.

The inventive digital therapeutic device is capable of detecting the data from EMG electrodes, accelerometers and inertia sensors fixed to the body appendages. For signal generation and application, the digital therapeutic multiplex electronic circuit works with a microprocessor to create a selectable array of individually addressable electrodes. To keep costs and complexity low, the detection and application of electrical signals from/to the body is achieved using a multiplex circuit with numerous addressable electrodes allowing for a high resolution and large skin surface area coverage. For the application of the generated signal to the body, the generated signal is directed through the multiplex circuitry so that the signal will be selectively applied to the precisely targeted muscles and nerves. The inventive digital therapeutic enables easy patient-customization, calibration and change in the use of the garment. For example, the foot stocking show in FIG. 73 can be reconfigured for the thighs, torso, arms and other body parts. In addition to the uses of the inventive digital therapeutic wearable electronics, other uses include the secure aggregation of biometric data, nonopioid pain relief, accelerated learning, sports augmentation and training, and military applications such as remote unmanned vehicle sensing and control.

FIG. 74 is a flow chart showing an algorithm for extrapolating or detecting the taking of a target drug from with a biometric parameter affected by the action of the target drug on the body. An initial dosage of a target drug is taken (Step One). A biometric parameter or indicator is detected, for example, through sweat chemistry analysis, EMG, skin color, blood flow, or other detectable biometric (Step Two). The biometric may be detected via detection mechanisms including but not limited to direct blood chemistry analysis, a measured biometric such as EMG, skin temperature, skin color or other change in a detectable parameter. The biometric may be optionally logged or transmitted (Step Three). The detected data can be stored locally in a memory associated with the inventive wearable electronic digital therapeutic device, or a remote memory located in a smart phone, a network server, a computer or other external device. The detected data can be filtered, compressed or otherwise conditioned prior to storage or transmission. A predetermined or calculated period of time is allowed to elapse to enable, for example, the metabolism, activation, deactivation, therapeutic action, or other change to occur in the blood levels of at least one of the target drug and the biometric (Step Four). After the time has elapsed, the biometric related to the drug level is detected (Step Five) and the target drug level may optionally be detected directly or indirectly by extrapolation from the detected biometric indicator (Step Six). The biometric and/or the drug level may be optionally logged or transmitted (Step Seven). As a further option, the detected biometric and/or the detected or extrapolated drug level may be used to inform a healthcare provider, a family member, the patient, an AI agent, a healthcare payor, a researcher, or other party, about data that can be used to adjust the dosage of the drug taken by the patient or indicated for a population that takes the drug.

The detected biometric and/or drug level can be used to determine if a biometric is acceptable (Step Eight). If it is not acceptable, an alert and/or other indication of the biometric not being acceptable can be used to increase or decrease the drug dosage. For example, if a larger dosage would increase the effectiveness of the drug, the drug dosage may be increased (Step Nine), if the detected biometric indicates the drug dosage is too great, the drug dosage may be decreased (Step Ten). The feedback loop iterates from the detection of the biometric (Step Two).

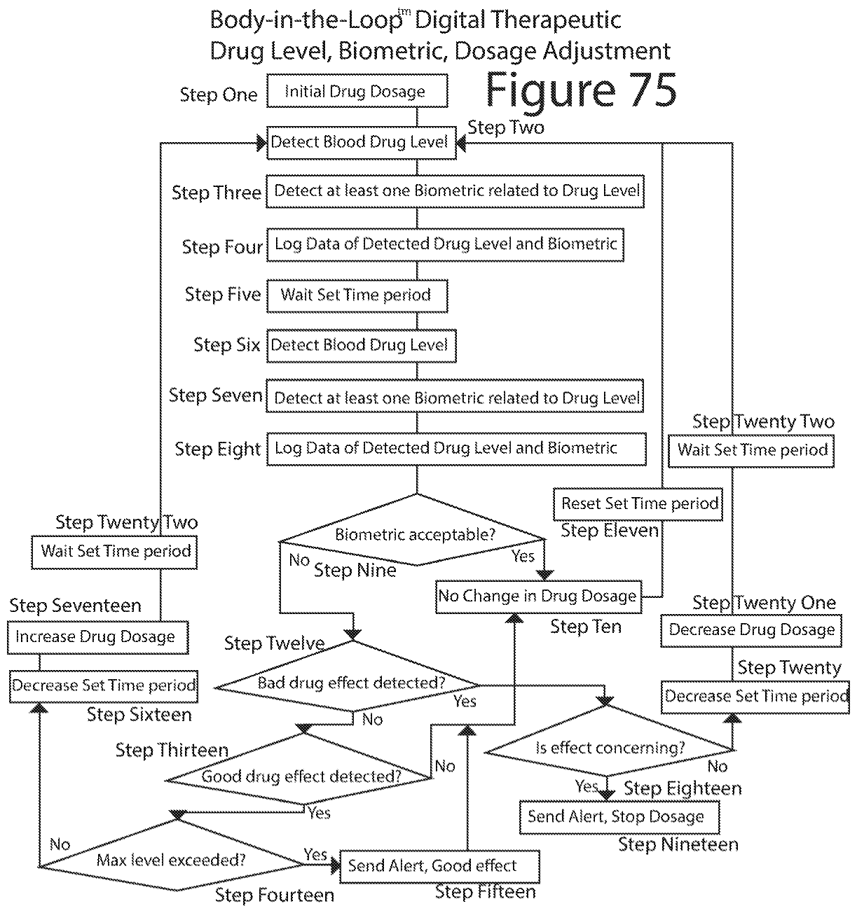

FIG. 75 is a flow chart showing a Body-on-the-Loop™ algorithm for detecting the blood level of a drug, detecting a biometric parameter related to at least one of a biometric indicator taken along with the drug and/or a physiological effect of the drug, logging data related to the detected drug level and biometric parameter and using the detect blood level and biometric parameter to indicate or automatically adjust a dosage of the drug.

An initial drug dosage is administered to the patient (Step One). After at least the expected time for the drug to enter the blood stream, the blood drug level is detected (Step Two). At least one biometric is detected related to the drug level (Step Three). The biometric can be one or more biometric parameters related to a physiological effect caused by the drug or one or more biometric indicators that are added to or taken along with the drug. For example, in the case of DVT, the physiological effect of taking an anticoagulant that inhibits Factor Xa, Factor XI, or inhibits other steps in the coagulation cascade, may be a detectable through improved blood flow through the surface veins of the patient. To collect the most meaningful data, the time period and number of drug administrations may be long and numerous.

The detected data of the drug level and/or the biometric are logged and/or transmitted (Step Four). A set time period is allowed to elapse (Step Five) and then the blood drug level is detected again (Step Six) and the at least one biometric related to the drug level is detected again (Step Seven). The newly detected data of the drug level and/or the biometric are logged and/or transmitted (Step Eight). The detected biometric is analyzed to determine if it is acceptable (Step Nine). That is, for example, in the case of a blood thinner or anticoagulant, if the blood flow through the surface veins indicates improvement or an acceptable level, the biometric of blood flow through the surface veins will be acceptable. If the biometric is acceptable, no change in drug dosage is warranted (Step Ten) and the set time period is reset (Step Eleven) and the blood drug level is again detected (Step Two). Note, if two or more biometric parameters are detected, then a determination of acceptability of both can be made (Step Nine), if both indicate that the biometrics are acceptable, then no change in drug dosage is warranted (Step Ten).

If, on the other hand, one or both of the biometrics is not acceptable (Step Nine), then it is determined if a bad drug effect has been detected (Step Twelve). If, for example, the detected biometric is an extrapolated or directly measured blood pressure, and the blood pressure rises or falls in a beneficial direction due to the drug dosage, a bad drug effect is not detected (Step Twelve) and a good drug effect may have been detected (Step Thirteen). If the good drug effect is detected it is determined if the maximum dosage for the target drug has been taken (Step Fourteen). If it has not, then the set time period is decreased (Step Sixteen) so that new biometric detections will occur sooner, and the drug dosage is increased (Step Seventeen). The step to increase may be a communication sent to a caregiver, insurance provider, researcher, patient, etc., alerting to the benefit of the change in blood pressure or an automatic increase in the dosage applied, for example, through an iontophoresis patch.

If the maximum level of the drug has been reached and would be exceeded by increasing the dosage, an alert is sent indicating the good effect indicated by the detected biometric (Step Fifteen) and no change in the drug dosage is taken (Step Ten), the time period is reset (Step Eleven) and the next blood drug level is detected (Step Two).

If a bad drug effect is detected (Step Twelve) for example, if the detected biometric is an extrapolated or directly measured blood pressure, and the blood pressure rises or falls beyond a predetermined threshold due to the drug dosage, a determination is made whether or not the effect is concerning (Step Eighteen). If the effect is concerning, an elevated alert is sent and the dosage is stopped (Step Nineteen).

If the bad drug effect is not immediately concerning and the patient should be administered the next dosage of drugs, the time set time period may be decreased (Step Twenty) and the drug dosage decreased (Step Twenty One). After determining the increase in the drug dosage (Step Seventeen) or decrease in the drug dosage (Step Twenty One), the set time period is allowed to elapse (Step Twenty Two) after the new increased or decreased dosage of the drug, and the blood drug level is again detected (Step Two).

The flowcharts described herein indicate a number of options and decisions based on one or more detected biometrics and how a particular patient's body responds to a drug dosage. The decisions and options triggered by the decisions or other process flow steps may be modified depending on a particular circumstance without limiting the scope of the inventive aspects disclosed.

FIG. 76 is a cross section of a pharmaceutical pill having a target drug and a biometric indicator, where the biometric indicator is a chemical analyte detectable by the inventive digital therapeutic for positively indicating patient adherence to the ingestion of the target drug. The inventive therapeutic chemistry (target drug) with detectable biometric indicator is used in combination with the inventive digital therapeutic device to enable a reliable determination of a patient's adherence to a prescribed drug therapy. The result is a highly useful, positive indication that can be used to alert a caregiver, hospital, insurance provider, manufacturer, researcher and other interested parties when a patient has taken an intended dosage, or an unscheduled dosage, or a prescribed or over the counter drug. Along with the positive indication of the presence or absence of a drug dosage, biometric indicators may also be determined by the same digital therapeutic device that detail the physiological effects of the administered drug, or the effects of the lack of the drug in the case where the drug dosage is missed or otherwise does not occur.

In accordance with an embodiment, a novel pharmaceutical medicinal compound includes a first compound having a determined therapeutic action on a patient and a second compound acting as a biometric indicator and having a chemical analyte detectable by a wearable electronic therapeutic device. The detection of the chemical analyte by the wearable electronic digital therapeutic device indicates the presence of the pharmaceutical medicinal compound in the patient. The chemical analyte may be detectable by the wearable electronic therapeutic device for positively indicating patient adherence to ingestion of the pharmaceutical medicinal compound. The first compound may comprise a core of a medicinal pill; and wherein the second compound comprises a coating on the core of the medicinal pill. The first compound may be formulated as a controlled release pharmaceutical having at least a portion of the first compound having a delayed release as a bioactive chemical available to perform a therapeutic action, meaning that before the first component is released as a bioactive chemical it does not perform the therapeutic action. The second compound may be formulated as a fast-available biometric indicator with the chemical analyte detectable prior to the delayed release of the first compound as a bioactive chemical. The chemical analyte may be detectable faster than the controlled release pharmaceutical becomes a bioactive chemical.

A third compound may be included acting as another biometric indicator. The third component comprises the chemical analyte and is formulated as a fast-available biometric indicator. The chemical analyte of the third compound may be detectable faster than said at least a portion of the first compound becomes a bioactive chemical. The first compound may comprise component, such as a granular, liquid or solid component, contained within a capsule. The capsule has a shell structure for containing the first compound and includes at least a portion of the second compound as a component of the shell structure. The first compound may be formulated as a controlled release pharmaceutical having at least a portion of the first compound having a delayed release as a bioactive chemical available to perform a therapeutic action. The second compound is formulated as a controlled release biometric indicator having the chemical analyte detectable at a rate corresponding to the rate that the portion of the first compound becomes a bioactive chemical.

A third compound may be included acting as another biometric indicator. The third component may comprise the chemical analyte and is formulated as a fast-available biometric indicator. The chemical analyte of the third compound is detectable faster than the portion of the first compound becomes a bioactive chemical. A third compound acting as another biometric indicator may comprise another chemical analyte and is formulated as a fast-available biometric indicator. The chemical analyte of the third compound may be detectable faster than the portion of the first compound becomes a bioactive chemical.

The first compound may comprise a liquid, solid, or granular component contained within a capsule. The second compound may comprise another liquid, solid or granular component contained with the capsule. The capsule may have a shell structure for containing the first compound and the second compound and includes at least a portion of the third compound as a component of the shell structure. The first compound and the second compound may comprise a core of a medicinal pill, and the third compound may comprise a coating on the core of the medicinal pill.

A device for detecting the ingestion of a pharmaceutical medicinal compound may comprise a wearable electronic digital therapeutic device including a biometric indicator detector for detecting a biometric indicator having a chemical analyte for positively indicating patient adherence to ingestion of the pharmaceutical medicinal compound. The pharmaceutical medicinal compound includes a first compound having a determined therapeutic action on the patient. A second compound acts as the biometric indicator and has the chemical analyte detectable by the wearable electronic digital therapeutic device. The detection of the chemical analyte by the wearable electronic digital therapeutic device indicates at least one of the absence and presence of the pharmaceutical medicinal compound ingested by the patient. The wearable electronic digital therapeutic device may further include a data transmitter for transmitting data indicating at least one of the absence and presence of the pharmaceutical medicinal compound ingested by the patient. The pharmaceutical medicinal compound may be, for example, for inhibiting an initiation of coagulation of blood.

FIG. 77 is a cross section of a pharmaceutical pill having a controlled release target drug and a fast release biometric indicator, where the fast release biometric indicator provides a relatively quicker detectable signal compared to the controlled release target drug for positively indicating patient adherence to the ingestion of the target drug.

FIG. 78 is a cross section of a capsule containing a time released target drug and a time released biometric indicator, where the biometric indicator remains detectable for a duration that relates to the time release of the target drug to provide an indication of the activity of the target drug from ingestion through to full or partial metabolism (or other activation or deactivation mechanism).

FIG. 79 is a cross section of a capsule containing a time released target drug and a time released biometric indicator, where the biometric indicator remains detectable for a duration that relates to the time release of the target drug, the capsule shell contains a fast release biometric indicator to provide a relatively quicker detectable signal compared to the slow release biometric indicator for positively indicating patient adherence to the ingestion of the target drug.

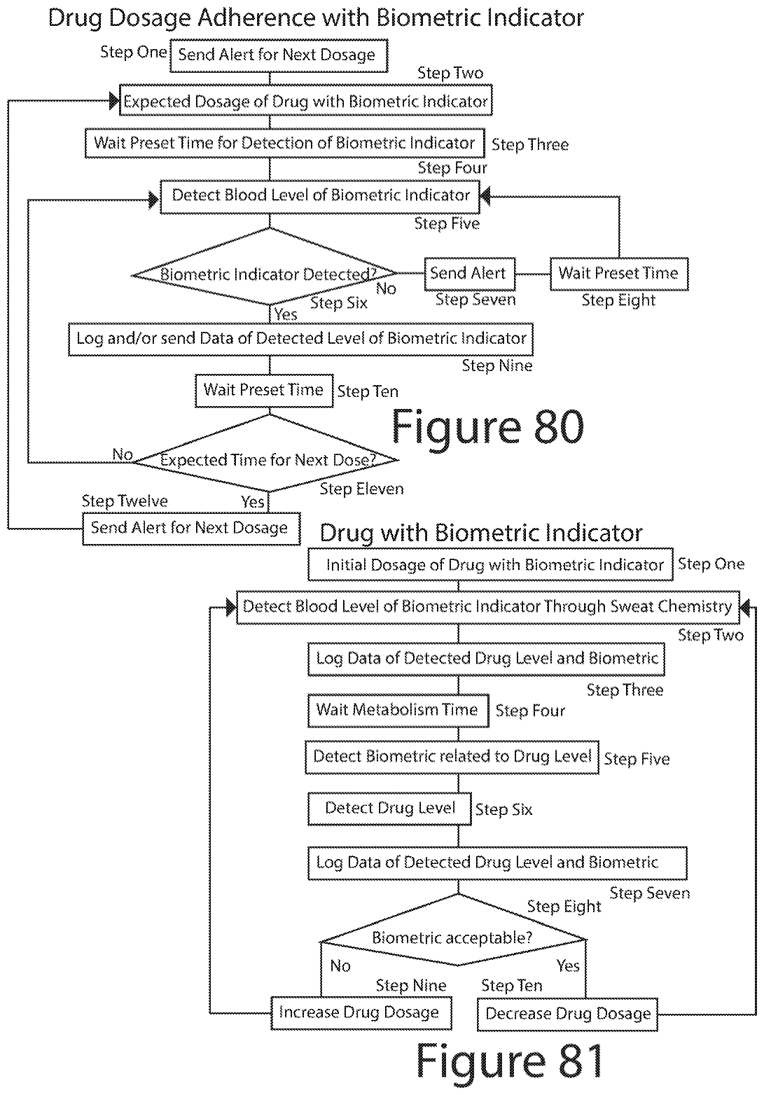

FIG. 80 is a flow chart showing an algorithm for detecting a patient's adherence to a scheduled drug ingestion of a target drug through the detection of the presence of a biometric indicator. The biometric indicator is detectable by the inventive digital therapeutic for positively indicating patient adherence to the ingestion of the target drug. The target drug and biometric indicator may be taken as a pill, capsule, other delivery mechanisms including but not limited to a transdermal patch, an intravenous drip, inhalation, eye drops, nasal spray, a transdermal injection, an implanted drug release mechanism, or other drug delivery vehicle. A fast release biometric indicator may provide a relatively quicker detectable signal compared to the controlled release target drug for positively indicating patient adherence to the ingestion of the target drug. The biometric indicator may remain detectable for a duration the relates to the time release of the target drug to provide an indication of the activity of the target drug from ingestion through to full or partial metabolism (or other drug activation/deactivation mechanism). The biometric indicator may remain detectable for a duration that relates to the time release of the target drug, where a capsule shell contains a fast release biometric indicator to provide a relatively quicker detectable signal compared to the slow release biometric indicator for positively indicating patient adherence to the ingestion of the target drug.

The fast release and time-release biometric indicators can include microencapsulation within a shell material that can be controlled so that a release of the biometric is timed as desired. For example, the shell material may have a rate of dissolving in the stomach that releases the biometric indicator so that a concentration of the biometric indicator is detectable over time that matches the expected therapeutic action of the target drug (for example, with a calculable concentration of the target drug dependent on the detected concentration of the biometric indicator and/or an expected therapeutic effect of the target drug dependent on the detected concentration of the biometric indicator). A biometric parameter, such as blood flow, extrapolated or directly measured blood pressure, skin temperature, EMG measurement, etc., can be used to confirm or adjust the calculated expectations of the target drug effect/concentration.

An alert can be sent to a smartphone, through email, text, phone call, or an alarm timer to remind a patient that it is time for the next dosage of a target drug (Step One). It is then expected that the patient will have taken the target drug with the biometric indicator, where the biometric indicator is a chemical analyte detectable by the inventive wearable electronic digital therapeutic device (Step Two). Depending on the target drug, the drug itself may be detectable as the chemical analyte for drug dosage adherence and so the drug itself can be the biometric indicator detected by the inventive digital therapeutic device. Otherwise, ideally a physiologically inert or non-troubling chemical or other biometric indicator is included along with the target drug, where the biometric indicator is readily detectable by a sensor or detector included with or in addition to the inventive wearable electronic digital therapeutic device. As one example, an organosulfur compound such as asparagusie acid (C4H6O2S2) may be employed as the chemical analyte or as a metabolic precursor to the detected chemical analyte used as a biometric indicator, By way of example only, asparagusic acid is a harmless compound that is readily excreted through normal kidney function, other compounds are also usable. A test can be performed on the patient to ensure that the asparagusic acid or whatever chemical analyte is chosen, is detectable for that particular patient through sweat chemistry analysis and that no false positives or negatives are likely, Asparagusic acid is known to only be naturally found in asparagus, so the patient merely has to avoid eating the plant.

A preset wait time elapses for the detection of the biometric indicator (Step Three). For example, in the case of an inhaled fast release biometric indicator, the wait time can be just a few seconds after inhalation. In the case of a pill, even a fast release biometric indicator may take longer, and up to several minutes to be detectable in the blood and even longer to be detected through the sweat using one or more of the biometric detectors, such as a sweat chemistry sensor, described herein, or other suitable detector/sensor mechanism.

After the preset time (Step Three), an attempt is made to detect the biometric indicator (Step Three). If the biometric indicator is detected (Step Four), then the detected the presence and/or level of the biometric indicator can be logged and/or data transmitted indicating the detected level (or simply a go/no go presence/absence detection) as an indication of the patient's adherence to the prescribed course of drug therapy.

If after the preset time has elapsed (Step Two) and the biometric indicator is not detected (Step Three) then an assumption may be made that the patient has not adhered to the prescribed course of drug therapy, and an alert is sent (Step Seven) by email, text, phone call, pager, SMS, or other communication method to let a caregiver, service provider, family member, the patient, the insurer, and/or the healthcare provider know that the patient may not have taken the prescribed drug dosage. After sending the alert, especially in the case where a reminder is sent directly to the patient or on-site caregiver present with the patient, an expectation is assumed that the patient has taken the target drug and biometric indicator, and a preset wait time can then again be allowed to elapse (Step Eight) before attempting to detect the biometric indicator (Step Four).

If the biometric indicator is detected (Step Six), after the data indicating the level or presence of the biometric indicator is logged and/or transmitted, a preset time may be allowed to elapse (Step Ten). If the expected time for the next dosage has not elapsed (Step Eleven), then the blood level of the biometric indicator can be detected again (Step Four) and if detected (Step Six) an indication of the detection logged or transmitted (Step Nine) to obtain a chronological history of the blood level of the biometric indicator as an indication of the blood level of the target drug (which may be particularly useful in the case where the biometric indicator has a blood level that correlates with the expected blood level over time of the target drug). When it is expected that the patient should be taking the drug dosage (Step Eleven) an alert for the next dosage can be sent out to remind the patient, caregiver, etc., that it is time for the next dosage to be taken (Step Twelve) and there the expectation that the patient will have taken the drug dosage with the biometric indicator is tested (Step Two).

This process can continue for as long as the target drug is prescribed for the patient, for days, weeks, months or even years, providing a detailed history of the patient's adherence to the prescribed course of drug therapy. If biometric parameters such as those described herein with regards to the other embodiments are also detected, logged and/or transmitted, a detailed history of the patient's therapy, course of treatment, measured results of treatment, etc., will be made available to improve the care given to the particular patient, and in the aggregate, provide significant data along with that of other patients, to assist in new drug discovery, treatment modifications, and a number of other advantages of the beneficial cycle created by detection, transmission, storage and analysis of biometric data taken directly from the patient during the course of drug therapy and/or other treatments.

FIG. 81 is a flow chart showing an algorithm for detecting the taking of a target drug along with a biometric indicator incorporate in the same pill or capsule or otherwise imparted into the patient at the same time or at a known time relative to the taking of the target drug. The detection of the biometric indicator is used as a positive indication that the target drug has been taken by the patient. A pill, capsule, or other drug delivery mechanism, containing a target drug may include a biometric indicator that is detectable and used to indicate, for example, patient adherence (e.g., the taking of a pill containing the target drug and the biometric indicator), the availability of the target drug into the blood stream, the timed release of the target drug, the metabolism and/or excretion of the target drug, etc. For example, in the case of the timed release of the target drug, the biometric indicator can be delivered with the same time-release mechanism as that incorporated with the target drug. The biometric indicator can be, for example, an additional component added to the chemistry of a new or pre-existing drug. In accordance with an embodiment, the ideal biometric indicator is a non-troubling water-soluble chemical compound that does not adversely alter normal body functionality, is detected from analysis of the sweat, and does not adversely affect the beneficial actions of the target drug. As an example, a compound containing polyhydroxyalkanoates (PHAs) which has biodegradable and biocompatible properties may be used as the biodetector.

An initial dosage of a target drug is taken along with a biometric indicator (Step One). A blood level of the biometric indicator is detected, for example, through sweat chemistry analysis (Step Two). The biometric indicator may be detected via other detection mechanisms including but not limited to direct blood chemistry analysis, a measured biometric such as EMG, skin temperature, skin color or other change in a detectable parameter. The detected drug level and/or the biometric indicator are logged (Step Three). The detected data can be stored locally in a memory associated with the inventive wearable electronic digital therapeutic device, or a remote memory located in a smart phone, a network server, a computer or other external device. The detected data can be filtered, compressed or otherwise conditioned prior to storage or transmission. A predetermined or calculated period of time is allowed to elapse to enable, for example, the metabolism, activation, deactivation, therapeutic action, or other change to occur in the blood levels of at least one of the target drug and the biometric indicator (Step Four). After the time has elapsed, the biometric related to the drug level is detected (Step Five) and the target drug level may optionally be detected directly or indirectly by extrapolation from the detected biometric indicator (Step Six). The data of the detected drug level and/or biometric is logged and/or transmitted to obtain, for example, a chronological history of the blood level of the biometric indicator as an indication of the blood level of the target drug (particularly in the case where the biometric indicator has a blood level that correlates with the expected blood level over time of the target drug).

As a further option, the detected biometric and/or the detected or extrapolated drug level may be used to inform a healthcare provider, a family member, the patient, an AI agent, a healthcare payor, a researcher, or other party that data has been provided that can be used to adjust the dosage of the drug taken by the patient or indicated for a population that takes the drug. The detected biometric and/or drug level can be used to determine if a biometric is acceptable (Step Eight). If it is not acceptable, an alert, automatic action, and/or other indication of the biometric not being acceptable can be used to increase or decrease the drug dosage. For example, if a larger dosage would increase the effectiveness of the drug as indicated by the detected biometric, the drug dosage may be increased (Step Nine), if the detected biometric indicates the drug dosage is too great or indicates the drug dosage is working correctly, the drug dosage may be decreased (Step Ten) to determine the minimum dosage required for an optimized treatment. The feedback loop iterates from the detection of the biometric (Step Two).

The inventive digital therapeutic device and these example processes implemented as a software/hardware solution creates a drug/device combination therapy that puts the patient's own body into a real-time feedback loop. The embodiments described herein can be used for many types of diseases and conditions, and work with a large number of prescribed or over the counter drugs, herbal remedies, or other applications where an ingested chemical modifies a detectable biometric. These therapies available through the inventive digital therapeutic device can be used as complementary or alternatives to drugs and surgery, and typically can typically continue for as long as the target drug is prescribed for the patient, and/or be employed before or after the prescribed drug is taken as treatment by the patient.

The data detection, transmission, and storage described herein provide a detailed history of the patient's adherence to the prescribed course of drug therapy. The biometric parameters such as those described herein with regards to the embodiments can also be detected, logged and/or transmitted, enabling a detailed history of the patient's therapy, course of treatment, measured results of treatment, etc., and can be made available to improve the care given to the particular patient, and in the aggregate, provide significant data along with that of other patients, to assist in new drug discovery, treatment modifications, and a number of other advantages of the beneficial cycle created by detection, transmission, storage and analysis of biometric data taken directly from the patient during the course of drug therapy and/or other treatments.

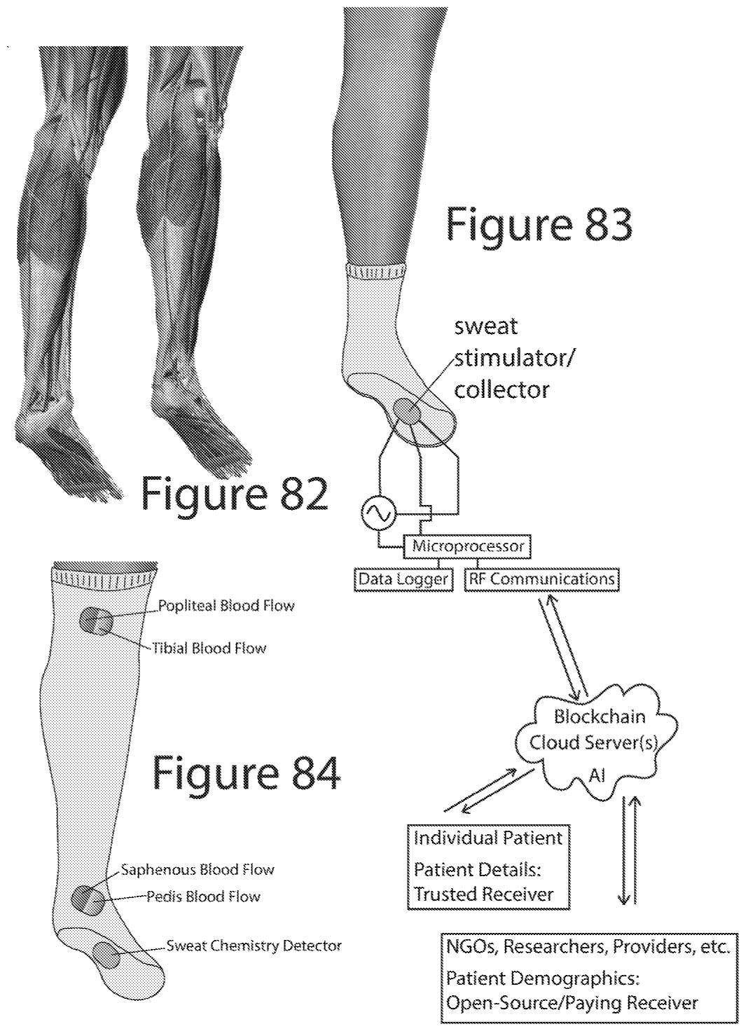

FIG. 82 shows the legs of a patient showing the location of popiliteal and tibial blood vessels at the back of the knee and saphenous and pedis blood vessels at the ankle. The location of muscles of the calf of a patient can be targeted for applying an electroceutical therapy, such as through the EMS induced ripple sequential involuntary contractions described herein. As an example of an applied electroceutical therapy, calf and lower leg muscles including the gastrocnemius, fibularis and soleus muscles can be controlled individually or simultaneously to involuntarily contract and milk the blood in the deep veins of the legs in the direction back towards the heart. Edema in the area of these deep veins can be determined, for example, by detecting a change in the circumference encompassing the calf muscles, ankles, foot, knee, or other body part. In accordance with the inventive wearable electronic digital therapeutic device, popliteal, tibial, saphenous and pedis blood flow can be determined through optical, mechanical or electrical transducers and sensors, and through probabilistic algorithmic analysis of the detected signals to determine blood flow through the various blood vessels.

FIG. 83 illustrates a sock showing a sweat stimulator/collector and block diagram of electronics. This example of the inventive wearable electronic digital therapeutic garment shows a convenient configuration that is comfortable, washable and is the same general construction of a garment that is typically worn nearly every day by patients and healthy members of the human population. A sweat chemistry sensor is shown. However, as with all the described embodiments, this sensor can be any one or more of the various biometric parameter detectors described herein or available currently or through advancements in enabling technologies.

FIG. 84 illustrates an embodiment of the inventive wearable electronic digital therapeutic device having blood vessel detectors and sweat chemistry sensors. The wearable electronic stocking includes a biometric parameter detector, such as blood flow and/or sweat chemistry detectors. The detectors generate signals analyzed, for example, by an artificial intelligence agent embedded in a microprocessor and memory of a wearable electronic circuit. As an example, in the case of an applied EMS signal used to activate the muscle pump for thrombotic conditions or other blood flow concerns, the analyzed signal can be used to modify the applied electrical signal and optimize the involuntary muscle contractions that initiate the muscle pump that forces blood through the deep veins back towards the heart. The detected biometric signals can be useful for a variety of healthcare and fitness related applications, described herein and for other uses that are apparent from these descriptions and drawings.

As an example, each leg stocking may include at least one pair of electrodes for applying at least one of an electrical muscle stimulation (EMS) and/or a transcutaneous electrical nerve stimulation (TENS) signal to at least one muscle of a patient (see, for example, FIG. 8). At least one biometric detector detects a biometric signal from the body of the patient. A drug delivery mechanism, such as an iontophoresis patch may be included. As described herein, the inventive digital therapeutic device may include all or some of these elements, in addition to other components, depending on the intended use.

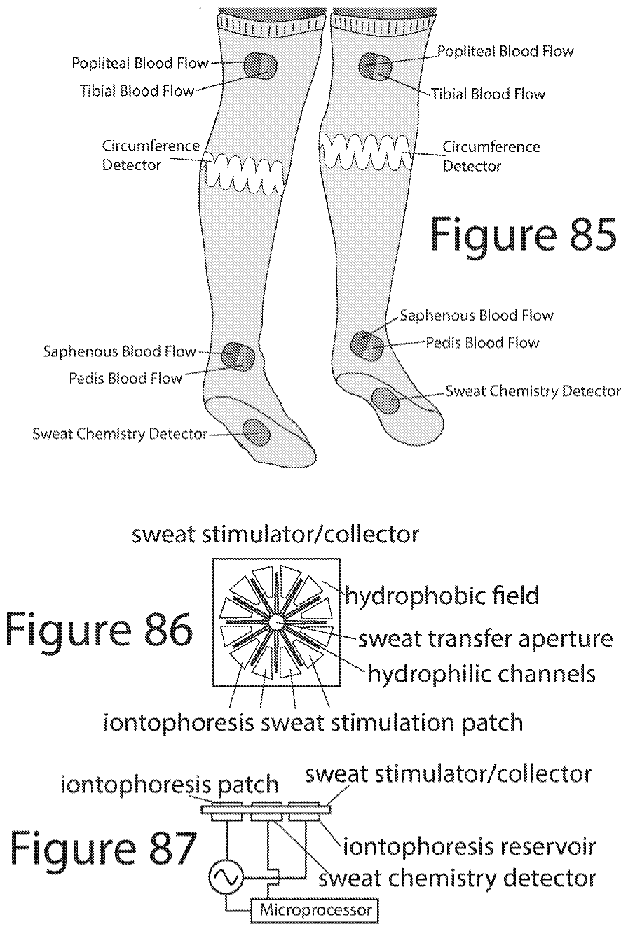

FIG. 85 illustrates an embodiment of the inventive wearable electronic digital therapeutic for the analysis of a therapeutic effect based on an activated physiological change and one or more detected biometric parameters.

The retention of fluid in the legs indicates a number of potentially serious medical conditions. For example, DVT or thrombophlebitis is often occurring in one swollen kg (especially the calf), where swelling is caused as blood pools in the area of the deep veins. Congestive heart failure, if the heart is too weak to pump blood with enough pressure the blood does not travel back towards the heart. Varicose veins and chronic versus insufficiency, such as when the valves inside the leg veins do not keep the blood flowing up toward the heart, Renal issues, where the kidneys are not filtering sufficient water and waste materials. Certain medications can also cause legs to swell, such as calcium channel blockers, anti-inflammatory, hormone treatments and even some antidepressants.

A drug, such as an anticoagulant, is often prescribed for the treatment and prevention of thrombosis. Typically, the anticoagulant works by inhibiting one or more of the factors of the blood coagulation cascade.

As shown in FIG. 4, in accordance with an embodiment of the inventive wearable electronic digital therapeutic device, the analysis can be made of a therapeutic effect based on an activated physiological change and one or more detected biometric parameters. For example, the activated physiological change can be the on-demand stimulation of sweat. One detected biometric parameter can be the detection of a coagulation cascade factor present in the sweat. Another detected biometric parameter may be the detection of blood flow through blood vessels in a body part, such as the legs. Another detected biometric parameter may be a change in the circumference of the leg caused by edema. Other biometric parameters are listed and described elsewhere. As shown in FIG. 4, a sweat chemistry detector can be included for determining a concentration of a coagulation cascade factor in the blood, a blood gas component, or other soluble molecule or small particulate. In accordance with an exemplary embodiment, the therapeutic effect may be the result of an administered therapeutic, such as a pharmaceutical therapy, e.g., an anticoagulant and/or an electroceutical therapy, e.g., EMS applied to activate the muscle pump. In addition or as an alternative to analyzing a therapeutic effect, disease progression or modification can be determined by monitoring the one or more detected biometric parameters.

The choice of the one or more detected biometric parameters may depend on the physiological condition, disease, fitness level, treatment being monitored, or other use case for the analysis of the therapeutic effect. Biometric parameters can be detected as an alternative or in addition to the ones described herein. For example, the biometric detection of biomarkers, such as thrombin and/or d-dimer, may be used for treatment and monitoring of conditions related to the contact system for coagulation and inflammation.

Skin color can be detected using optical systems. Full-color skin imaging using RGB LED and floating lens in optical coherence tomography, Yang B-W, Chen X-C. Full-color skin imaging using RGB LED and floating lens in optical coherence tomography. Biomedical Optics Express. 2010; 1(5):1341-1346. doi:10.1364/BOE.1.001341 shows an example of an LED based skin color sensor system that can be modified in accordance with the inventive digital therapeutic to detect skin color as a biometric parameter. It is noted that many of the various biometric detector can share common components, reducing costs and enabling high speed sampling of different biometric parameters for the different exemplary embodiments described herein.

The Kardia Mobile ECG by AliveCor is an example of an ECG device with well-known electronics that can be modified in accordance with the inventive digital therapeutic to detect heartbeat and other heart related measurements. There are many small and inexpensive examples of blood pulse oximeters, automatic blood pressure readers, and skin temperature sensors that can be modified in accordance with the inventive digital therapeutic to detect temperature, blood pressure, pulse, blood oxygen and other related biometric parameters.

In addition to the well-known strain sensors, a stretchable strain has been recently reported by the University of Houston, Highly Sensitive and Very Stretchable Strain Sensor Based on a Rubbery Semiconductor, ACS Appl. Mater. Interfaces, 2018, 10 (5), pp 5000-5006, 2018. A stretchable strain sensor with printable components has recently been reported by the University of Florida, Highly Stretchable and Wearable Strain Sensor Based on Printable Carbon Nanotube Layers/Polydimethylsiloxane Composites with Adjustable Sensitivity, ACS Appl. Mater. Interfaces, 2018, 10 (8), pp 7371-7380.

The biometric parameters may include a strain gauge formed from an elastic resistance strip that reversibly changes a detectable resistance value based on being stretched. The sweat chemistry sensor may comprise a stretchable electrochemical sweat sensor made, for example, by the deposition of carbon nanotubes (CNTs) on top of patterned Au nanosheets (AuNS) as reported by the graduate school of converging science and technology, Korea University, Seoul (see, for example, Skin-Attachable, Stretchable Electrochemical Sweat Sensor for Glucose and pH Detection, ACS Applied Materials & Interfaces 2018 10 (16), 13729-13740 DOI: 10.1021/acsami.8b03342). This is one example of a sweat chemistry sensor that could be employed as part of the inventive digital therapeutic, for the detection of glucose and pH. In this case, CoWO4/CNT and polyaniline/CNT nanocomposites are coated onto the CNT-AuNS electrodes, respectively. A reference electrode is prepared via chlorination of silver nanowires. A change in electrical signal characteristics among the electrodes is indicative of the detected glucose and pH. By modifying the functionalized components, other chemicals present in the sweat can be targeted for detection. By providing multiple sweat chemistry sensor or by creating a patchwork of differently functionalized areas of a multiplexed array of sweat chemistry sensors, a variety of chemicals present in sweat (or the absence thereof) can be determined.

FIG. 86 is a top view of components of a sweat chemistry sensor that includes an activatable physiological change in form of induced sweat stimulation. FIG. 87 is a cross sectional view of an iontophoresis patch sweat chemistry sensor.

Others have reported on prolonged and localized sweat stimulation by iontophoretic delivery of the slowly-metabolized nicotinic cholinergic agonist carbachol. Iontophoresis was performed with either carbachol or pilocarpine in order to stimulate sweat in subjects at rest. (see, for example, Prolonged and localized sweat stimulation by iontophoretic delivery of the slowly-metabolized cholinergic agent carbachol, Simmers, Li, Kasting, Heikenfeld, J Dermatol Sci. 2018 January; 89(1):40-51. doi: 10.1016/j.jdermsci.2017.10.013, which is incorporated by reference herein).

In the case of a sweat chemistry sensor, sweat can be stimulated on demand using, for example, iontophoresis of sweat from the glands on the bottom of the foot. To induce adequate sweat even from a sedentary patient, an electrical signal is applied to cause a sweat simulating chemical to pass into the skin and stimulate the sweat glands to produce sweat. Then, when the electrical signal is not applied, the stimulated sweat product stops.

The sensor element is wet by the sweat and then the sweat is drawn through wicking into wicking/evaporation materials. A continuous flow of fresh sweat passes over the sensor so that a continuous logging of data can be achieved, and an alert triggered based on analysis of the chemicals in the sweat. A hydrophobic field encourages sweat to bead and migrate to hydrophilic channels. Tapered hydrophilic channels use surface tension to draw sweat into the sweat transfer aperture. Hydrophobic and hydrophilic screen printable inks are available from companies such as Cytonix and Wacker and can be used to mass produce the collector skin facing portions using simple screen-printing techniques for forming a sweat collector having a flow through structure.

Any water-soluble component in the blood can be detected through sweat chemistry analysis. For example, lactate, glucose and urea are three important blood chemistry measurements. Chemical analytes can be detected to indicate patient adherence to a drug taking regime, to determine the metabolism of an ingested drug and/or to detect biomarkers generated by the body in response to stress, disease or health related conditions. As described herein, the on-demand activation of sweat for analysis of detected biomarkers, such as soluble proteins, can be used to for the analysis of a therapeutic effect based on an activated physiological change and one or more detected biometric parameters.

As shown in FIG. 6, an iontophoresis reservoir holds the sweat inducing material. An iontophoresis patch is disposed in face-to-face contact with the skin surface containing sweat glands (e.g., at the bottom of the foot). A microprocessor controls the application of an electrical signal to cause the sweat inducing material to be driven into the skin and activate the sweat glands to produce sweat on demand. A sweat chemistry detector element is functionalized for detecting one or more target molecules. Chemicals present in the sweat are detected by a functionalized sweat chemistry detector element, which generates a signal dependent on the presence of the detected sweat chemistry, this signal is received by the microprocessor indicting the presence or absence of the target molecules.

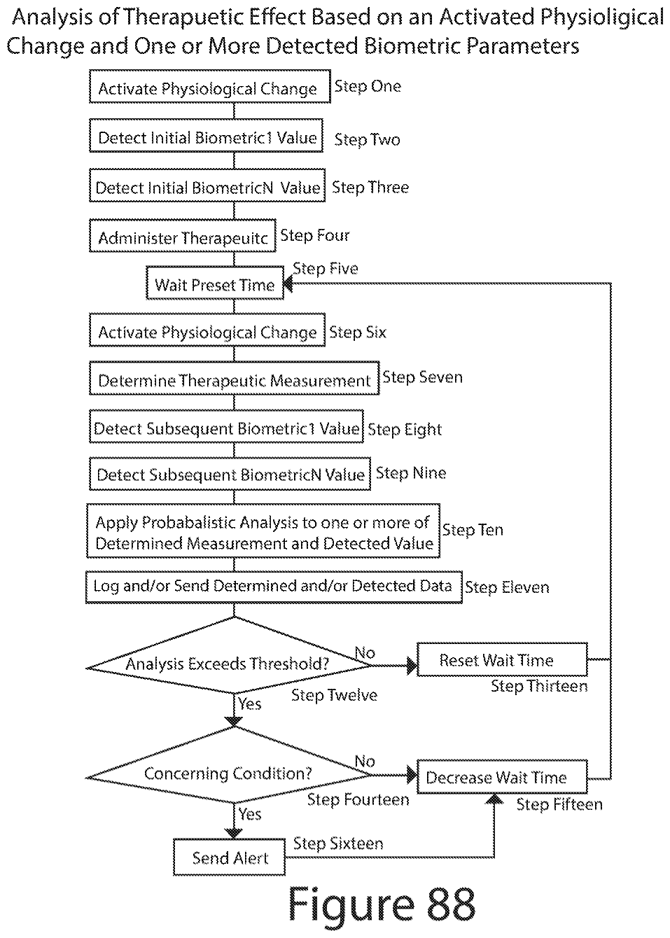

FIG. 88 is a flow chart illustrating an algorithm for analysis of a therapeutic effect based on an activated physiological change and one or more detected biometric parameters.

A physiological change is activated (Step One). For example, the activated physiological change can be on-demand sweat stimulation, applied EMS to activate the muscle pump or other involuntary muscle contraction or the like. An initial biometric1 value is detected (Step Two) and any number of additional biometric values (e.g., biometric2-biometricN) may also be detected (Step Three). A therapeutic is then administered (Step Four). After waiting a preset time (Step Five), the physiological change is again activated (Step Six) and therapeutic measurement is determined (Step Seven). Subsequent biometeric1 through biometricN values are detected (Step Eight and Step Nine), and a probabilistic analysis is applied to one or more of the determined measurement (e.g., the therapeutic measurement) and the biometric values (e.g., a comparison of the initial biometric values with the subsequent biometric values) (Step Ten).

The determined and/or detected data is logged and/or sent (Step Eleven). For example, the inventive wearable electronic digital therapeutic device can include a microprocessor controlling a memory for locally logging data pertaining to the activation of the physiological change, the detection of biometric values, the administering of the therapeutic(s), the determined therapeutic measurement and other details regarding the patient, disease progression or treatment, local environment (e.g., temperature, humidity, air quality), etc. The memory can also be located on a smartphone, local computer or networked device, or any wired or wirelessly connected memory mechanism. Data can be sent raw or filtered and/or compressed wirelessly or over a wired connection to an internet cloud server, local machine, smartphone, tablet, etc. The microprocessor onboard the wearable electronic device and/or the smartphone, cloud server, local machine, etc., can receive the data and determine that an alert should be transmitted via email, SMS, text, pager, or other communication mechanism. The data can be stripped of any identifying information of the patient as necessary to comply with governmental, healthcare provider, insurance and/or patient required privacy issues, or otherwise composed as needed to be effective for use in maintaining and monitoring the health of the individual patient and/or made useful in an aggregate form with data received from a population of patients and/or user.

It is determined if the probabilistic analysis indicates that the measurement or detected data exceeds a threshold (Step Twelve). For example, if the activated physiological change is the stimulation of sweat and the detected biometric values include a biomarker indicative of factor in the coagulation cascade, the concentration or presence of that factor in the sweat of the patient when compared with another biometric value, such as a change in blood flow, may indicate the exceeding of a threshold. If the threshold was not exceeded in Step Twelve, then the wait time can be reset (adjusted up or down or remain the same) (Step Thirteen) and this preset time is allowed to elapse (Step Five) before continuing to monitor the patient using an on-demand activation of the physiological change.

If the threshold is exceeded (Step Twelve), then it is determined if there is a concerning condition indicated by the probabilistic analysis exceeding the threshold (Step Fourteen). For example, if the exceeded threshold indicates that concentration of a coagulation factor and the change in blood flow shows there may be conditions present in the blood stream of the patient that promotes clot formation, such a condition may be deemed to be concerning. If there is not a concerning condition present, then the wait time may be decreased (Step Fifteen) to avoid, for example, the activation of sweat stimulation preserving a reservoir of a sweat stimulation chemical and/or avoiding exposing the patient to more of the sweat stimulation chemical than is necessary. The preset wait time is then allowed to elapse (Step Five) before continuing to monitor the patient using an on-demand activation of the physiological change.

If the exceeded threshold indicates that there may be conditions present where such a condition may be deemed to be concerning, an alert may be sent to a caregiver, family member, insurance company, the patient, a cloud-based data collection system, or other entity and/or actor to facilitate improved care of the patient and/or to improve the healthcare system.

In accordance with an aspect of the invention, a wearable electronic therapeutic device has one or more biometric detectors each for detecting one or more biometric parameters. The biometric parameters are dependent on at least one physiological change to a patient in response to a therapeutic treatment. A microprocessor receives the one or more biometric parameters and applies probabilistic analysis to determine if at least one physiological change threshold has been exceeded dependent on the probabilistic analysis of the two or more biometric parameters. An activation circuit activates an action depending on the determined exceeded physiological change. The action that is activated can be applying an electroceutical treatment in addition or as an alternative to a pharmaceutical treatment.

In accordance with an aspect of the invention, a method, comprises: detecting one or more biometric parameters, where the biometric parameters are dependent on at least one physiological change to a patient in response to a therapeutic treatment; receiving the one or more biometric parameters and applying probabilistic analysis to determine if at least one physiological change threshold has been exceeded dependent on the probabilistic analysis of the two or more biometric parameters; and activating an action depending on the determined exceeded said at least one physiological change.

In accordance with as aspect of the invention, an apparatus, comprises: at least one processor; and at least one memory including computer program code, the at least one memory and the computer program code configured, with the at least one processor, to cause the apparatus to perform at least the following: detect one or more biometric parameters, where the biometric parameters are dependent on at least one physiological change to a patient in response to a therapeutic treatment; receive the one or more biometric parameters by the at least one processor and apply probabilistic analysis to determine if at least one physiological change threshold has been exceeded dependent on the probabilistic analysis of the two or more biometric parameters; and activate an action depending on the determined exceeded said at least one physiological change.

In accordance with an aspect of the invention, a computer program product comprises a computer-readable medium bearing computer program code embodied therein for use with a computer, the computer program code comprising: code for detecting one or more biometric parameters, where the biometric parameters are dependent on at least one physiological change to a patient in response to a therapeutic treatment; code for receiving the one or more biometric parameters and applying probabilistic analysis to determine if at least one physiological change threshold has been exceeded dependent on the probabilistic analysis of the two ore more biometric parameters; and code for activating an action depending on the determined exceeded said at least one physiological change.

The therapeutic treatment may include an anticoagulant for treating a cardiovascular condition, and the physiological changes can include an indication changes in the cardiovascular condition. The action can include transmitting an alert, modifying the therapeutic treatment, and transmitting data dependent on at least one of the at least one physiological change, the one or more biometric parameters, the therapeutic treatment and the probabilistic analysis.

The probabilistic analysis can comprise determining from a data set of the one or more biometric parameters whether the data set is acceptable for deciding that the at least one physiological change threshold has been exceeded. The probabilistic analysis may further comprise applying a statistical weighting to each of the one or more biometric parameters, where the statistical weighting is dependent on a predetermined value of a ranking of importance in detecting each of the at least one physiological change for said each of the one or more biometric parameters relative to others of the one or more biometric parameters. At least one of the biometric values is determined from one or more water soluble molecules; and further comprising an on-demand sweat stimulator for stimulating the production of sweat by the patient and a sweat chemistry sensor for sensing the one or more water soluble molecules.

There are a number of ways to apply probabilistic analysis to the measurement and/or detected values to gain insights into the analysis of a therapeutic effect.

As an example, US Patent Application US20080104006A1, entitled Multimodal Fusion Decision Logic System Using Copula Model, invented by Fred Kiefer and now assigned to Qualcom, Inc., which is incorporated herein in its entirety, describes a method of deciding whether a data set is acceptable for making a decision.

This reference describes a number of possible probabilistic analysis tools that can be utilized in accordance with the inventive wearable electronic digital therapeutic device and is used by way of illustration of statistical methodologies that can be employed to determine a physiological condition from one or more detected biometric parameters.

As an exemplary multimodal fusion decision logic system, a first probability partition array and a second probability partition array may be provided, where one or both of the probability partition arrays may be a Copula model. A no-match zone can be established and used to calculate a false-acceptance-rate (“FAR”) and/or a false-rejection-rate (“FRR”) for the data set. The FAR and/or the FAR may be compared to desired rates. Based on the comparison, the data set may be either accepted or rejected.

Similar to the Multimodal Fusion Decision Logic System and other techniques disclosed in the '006 application, the algorithms described herein may be used to combine information from two or more biometric modalities, based on the same or two different detected parameters. The combined information may allow for more reliable and more accurate identification of a potentially concerning physiological change than is possible with systems based, for example, on a single biometric modality. The combination of information from more than one biometric modality is sometimes referred to herein as “biometric fusion”.

The '006 application describes techniques for confirm an individual's identity based on biometric modalities that may include (but are not limited to) fingerprint identification, iris recognition, voice recognition, facial recognition, hand geometry, signature recognition, signature gait recognition, vascular patterns, lip shape, ear shape and palm print recognition. In accordance with the present invention, instead of using biometric modalities like fingerprints to identify an individual, detected physiologically changing parameters are used to make a decision on when to send out an alert indicating, for example, that an at-risk patient is undergoing a concerning physiological change. As an example described herein, two or more physiologically change indicating biometric parameters, such as leg circumference, skin color and/or skin temperature, can be statistically evaluated using techniques described in the '006 application as applied to non-changing biometrics such as fingerprints, and other similar disclosures, to obtain a novel and useful mechanism for providing an early warning of stroke conditions. This is just an example application of the inventive system.

In accordance with the present invention, probability partition arrays may be used, such as those described in the '006 application, and the methods employed for deciding whether a data set is acceptable can be used to determine the potential for a concerning physiological change to be occurring. For example, the inventive wearable electronic digital therapeutic device can be utilized along with a method of deciding whether a detected physiological change obtained from a data set is acceptable for making a decision. For example, before deciding to send an alert indicting that an at-risk patient may be experience a concerning physiological change, it is first determined whether a set of biometrics is acceptable for making a decision about whether a detected change in one or more detected biometric parameters is concerning. The data set may be comprised of information pieces about detected biometric parameters or external parameters detected from one or more body parts or external source, using a variety of detectors, including, but not limited to, light reflectance/absorption, skin color, temperature, heartbeat, strain gauge (swelling), strain gauge (tightness), radioisotope (in-hospital, short duration), sweat chemistry, blood chemistry, transdermal interstitial fluid, arterial blood flow, venous blood flow, chemical biomarker, electrical biomarker, environmental parameter, protein precursors, cytokines, and the like.

The detected biometrics can be taken from the patient's body, for example, the legs of the patient, and may have at least two types of information pieces, i.e., the data set may have at least two modalities. For example, each parameter set represented in a database of collected biometric samples may by represented by two or more biometric samples, for example, a blood flow sample and a pulse sample. A first probability partition array (“Pm(i,j)”) may be provided. The Pm(i,j) may be comprised of probability values for information pieces in the data set, each probability value in the Pm(i,j) corresponding to the probability of an of the detected biometric parameters indicating a concerning physiological change. As mentioned in the '006 application, Pm(i,j) may be similar to a Neyman-Pearson Lemma probability partition array. A second probability partition array (“Pfm(i,j)”) may be provided, the Pfm(i,j) being comprised of probability values for information pieces in the data set, each probability value in the Pfm(i,j) corresponding to the probability of a false match. Pfm(i,j) may be similar to a Neyman-Pearson Lemma probability partition.

The use of two or more biometric samples can also be used to identify a no-match zone. For example, the no-match zone may be identified by identifying a first index set (“A”), the indices in set A being the (i,j) indices that have values in both Pfm(i,j) and Pm(i,j). A second index set (“Z∞”) may be identified, the indices of Zo being the (i,j) indices in set A where both Pfm(i,j) is larger than zero and Pm(i,j) is equal to zero. FARZ∞ may be determined, where FARZ∞=1−Σ(i,j)εZ∞ Pfm(i, j). FARZ∞ may be compared to a desired false-acceptance-rate (“FAR”), and if FARZ∞ is greater than the desired false-acceptance-rate, than the data set may be rejected for failing to provide an acceptable false-acceptance-rate. If FARZ∞ is less than or equal to the desired false-acceptance-rate, then the data set may be accepted, if false-rejection-rate is not important.

If false-rejection-rate is important, further steps may be executed to determine whether the data set should be rejected. The method may further include identifying a third index set ZM∞, the indices of ZM∞ being the (i,j) indices in Z∞ plus those indices where both Pfm(i,j) and Pm(i,j) are equal to zero. A fourth index set (“C”) may be identified, the indices of C being the (i,j) indices that are in A but not ZM∞. The indices of C may be arranged such

that P fm [symbol] (i,j)kPm [symbol] (i,j)k>=Pfm [symbol] (i,j)k+1 Pm [symbol] (i,j)k+1 to provide an arranged C index. A fifth index set (“Cn”) may be identified. The indices of Cn may be the first N (i,j) indices of the arranged C index, where N is a number for which the following is true: FARZ∞∪CN=1−Σ(i,j)εZ∞Pfm(i,j)−Σ(i,j)εCNPfm(i,j)≤FAR. The FRR may be determined, where FRR=Σ(i,j)εCNPm(i,j), and compared to a desired false-rejection-rate. If FRR is greater than the desired false-rejection-rate, then the data set may be rejected, even though FARZ∞ is less than or equal to the desired false-acceptance-rate. Otherwise, the data set may be accepted.

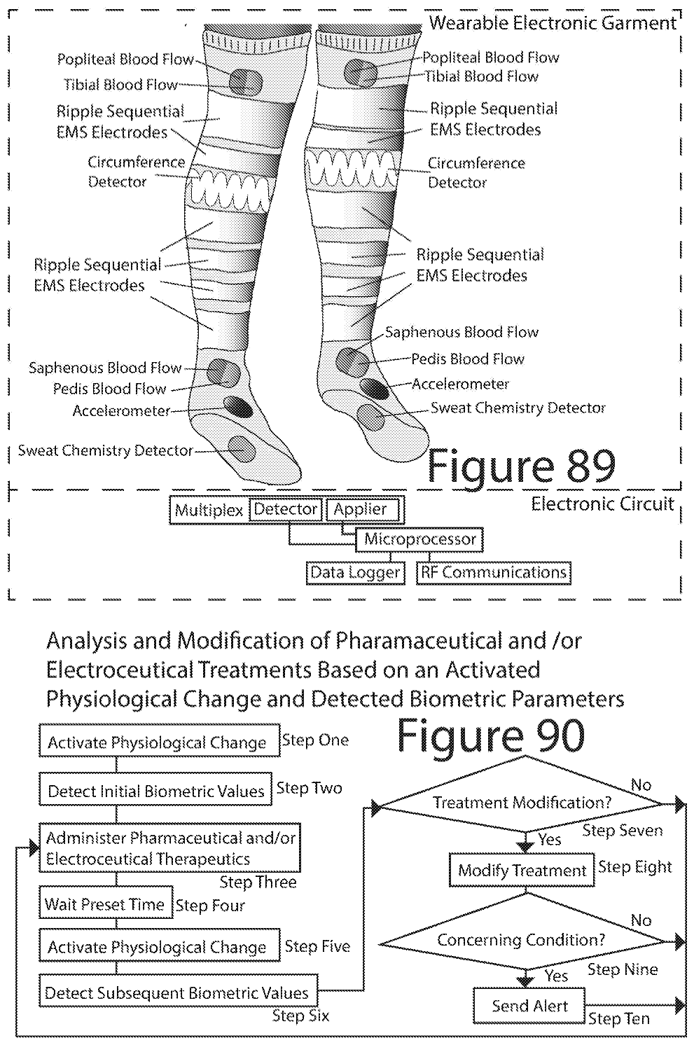

FIG. 89 illustrates an embodiment of the inventive wearable electronic digital therapeutic for the analysis and modification of pharmaceutical and/or electroceutical treatments based on an activated physiological change and detected biometric parameters. As a non-limiting embodiment of the inventive digital therapeutic device a pair of leg stockings are configured for applying an EMS therapy to the calf muscles of a patient. In this embodiment, the digital therapeutic device is a wearable electronic that uses electronic muscle contractions to pump blood through the blood vessels of the lower legs and prevent clots from forming. A series of EMS electrodes are circumferentially positioned in face-to-face electrical communication with the skin of the patient. For clarity, FIG. 89 and other drawings show at least some of the electrical components on the outside of the wearable electronic garment. In actual usage, electrical components, such as the EMS electrodes, will be underneath the wearable electronic garment. The EMS electrodes are also capable of detecting electrical signals from the patient. For example, EMG, pulse, heart rhythm, and other electrical signals can be detected by the same electrodes from the patient that apply electrical signals to the patient.

Biometric parameters are detected using electrical, optical and chemical systems that detect biometric parameters including but not limited to popliteal blood flow, tibial blood flow, saphenous blood flow, pedis blood flow, circumference, sweat chemistry, interstitial fluid chemistry, blood chemistry, temperature, color, EMG, motion or lack thereof, heart rhythm, ambient temperature and humidity, and the like. When configured for other body parts, additional biometric parameters are detectable including respiration, saliva, and other physiological change deterministic detectable signals from the patient's body or from the local environment.

An Electronic Circuit is connected with the Wearable Electronic Garment. Depending on the intended use, the EMS electrodes can be used for EMG or other signal detection so that bi-directional electrical signals are applied through a plurality of individually addressable electrodes routed through an electrode multiplex circuit and a signal multiplex circuit for applying a sequential EMS signal and detecting biometric feedback, for example, from the calf of a patient. In accordance with an aspect of the invention, a digital therapeutic device garment is provided with a plurality of individually addressable electrodes supported by the garment for applying a sequential EMS signal and detecting biometric feedback from the calf of a patient. The individually addressable electrodes are for at least one of applying stimulation electrical signals to the skin of a patient and detecting biometric electrical signals from the skin of the patient. At least one of a signal detector for detecting the biometric electrical signals and a signal generator for generating the stimulation electrical signals are provided. An electrode multiplex circuit addresses the plurality of individually addressable electrodes by at least one of routing the biometric electrical signals from the skin of the patient through more than one of the plurality of individually addressable electrodes to the signal detector and routing the stimulation electrical signals from the signal generator through more than one of the plurality of individually addressable electrode to the skin of the patient. A microprocessor controls the signal detector, the signal generator, the electrode multiplex circuit and other circuit components.

The microprocessor can control the electrode multiplex circuit to route the biometric electrical signals from the skin of the patient sequentially through more than one of the plurality of individually addressable electrodes to the signal detector. In accordance with this embodiment, a single EMS signal source can service multiple individually addressable electrodes with the EMS signal routed as desired for an intended therapy, such as for the sequential squeezing of the deep veins in the legs to promote blood flow in the direction back to the heart. One or more EMS signal channels can be multiplexed and signals routed so that even a large array of individually addressable electrodes can be serviced by one or a few signal generators, for example, to provide a finer spatial resolution of the applied EMS signal than indicated by number of electrodes shown in the drawings.

The microprocessor can control the electrode multiplex circuit to route the biometric electrical signals (indicating, for example, muscle activity, heartbeat, etc.) from the skin of the patient simultaneously through more than one of the plurality of individually addressable electrodes to the signal detector. The microprocessor can control the electrode multiplex circuit to route the stimulation electrical signals from the signal generator simultaneously through more than one of the plurality of individually addressable electrodes to the skin of the patient. The microprocessor can control the electrode multiplex circuit to route the stimulation electrical signals from the signal generator sequentially through more than one of the plurality of individually addressable electrodes to the skin of the patient. The microprocessor can control the electrode multiplex circuit to route the stimulation electrical signals from the signal generator simultaneously through more than one of the plurality of individually addressable electrodes to the skin of the patient.

FIG. 90 is a flow chart illustrating an algorithm for the analysis and modification of pharmaceutical and/or electroceutical treatments based on an activated physiological change and detected biometric parameters.

A physiological change is activated (Step One). Initial biometric values are detected (Step Two). A treatment of pharmaceutical and/or electroceutical therapeutics are administered (Step Three), and then a preset time elapses (Step Four). After the preset wait time, the physiological change is again activated (Step Five) and subsequent biometric values are detected (Step Six). It is determined based on the detected biometric values (for example, by comparing previous to subsequent biometric values) if any modification is necessary for the treatment of the patient (Step Seven), and if modification is necessary, the treatment is modified (Step Eight). If it is determined that treatment medication is not necessary, then the unmodified treatment is again administered (Step Three). After modifying the treatment in step eight, it is then determined if the detected biometric values indicates that a concerning condition exists (Step Nine). If there is no concerning condition, then the modified treatment from step eight is again administered (Step Three). If there is a concerning condition, then an alert is sent (Step Ten) and the modified treatment is again administered. It is noted that the biometric values could indicate that the modification of the treatment should be halting the treatment, changing the time, duration, dosage, intensity, or any other modification to the administered pharmaceutical and/or electroceutical treatment.

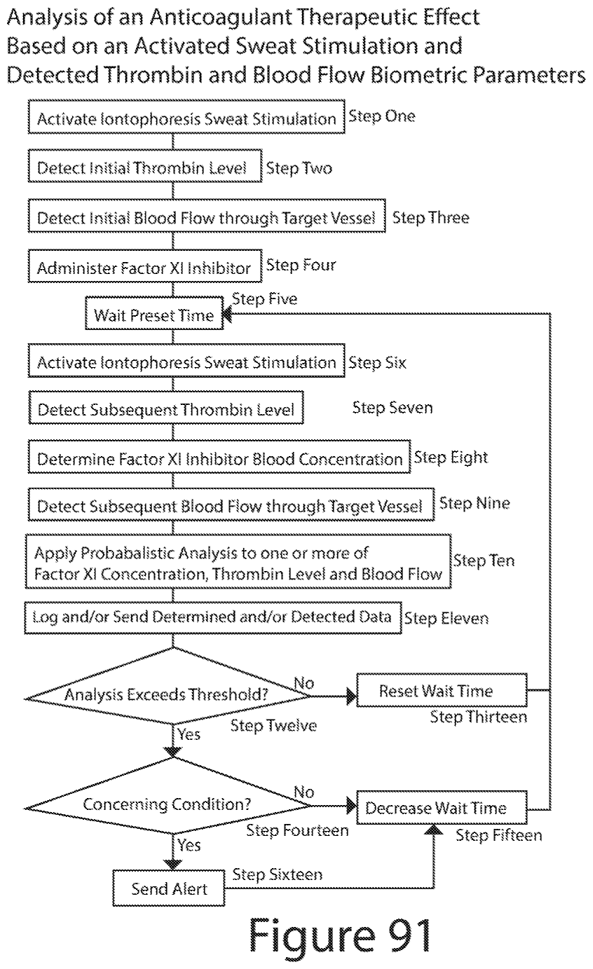

FIG. 91 is a flow chart illustrating an algorithm for analysis of an anticoagulant therapeutic effect based on an activated sweat stimulation detected biomarkers, such as thrombin and/or D-Dimer, and blood flow biometric parameters. Sweat stimulation is activated by iontophoresis where a sweat stimulating chemical, such as carbachol, is electrically driven on-demand into the skin (Step One). An initial level of a blood coagulation factor, such as biomarkers, such as thrombin and/or D-Dimer or other suitable biomarker, is detected (Step Two). As an example, a sweat chemistry sensor can be functionalized for detecting the biomarker thrombin, or D-Dimer, or other such detectable biomarker, to indicate the status of a thrombotic condition.

Initial blood flow through a target blood vessel is detected (Step Three). For example, the popliteal artery and/or vein or other blood vessel on the leg of a patient can be targeted for blood flow detection by the placement of an optical blood flow sensor against the skin in the location of the popliteal artery/vein, etc. The optical blood flow sensor may be disposed to detected capillary blood flow that receives and drains blood from the popliteal artery/vein and the blood flow through those vessels extrapolated from the capillary blood flow. The choice of the blood vessel to directly measure or extrapolate blood flow detection of depends on the goals of analysis. For example, if the analysis is to determine efficacy of an anticoagulant for the prevention or treatment of deep vein thrombosis, the target blood vessel can be the deep veins in the lower legs, and the flow rate can be approximated through the measurement of blood vessels located closer to the skin surface.

A therapeutic drug, for example, an anticoagulant such as a Factor XI inhibitor, is administered to the patient (Step Four). The administration may be through an oral dosage and the therapeutic drug may include a time release formulation to keep a desired concentration of the bioactive Factor XI inhibitor in the blood stream for a desired duration of time (for example, a single daily dosage) to provide a desired therapeutic effect (for example, prophylaxis against thrombosis).

A preset time is allowed to elapse (Step Five), for example, to allow for the therapeutic drug to achieve a degree of therapeutic effect and/or for a concentration of the bioactive therapeutic drug to become available in the blood stream.

Sweat stimulation is again activated through iontophoresis of a sweat stimulation chemical into the skin (Step Six). In accordance with the inventive wearable electronic digital therapeutic device, on-demand sweat stimulation is utilized so that there is an adequate quantity of sweat available at the time when the detection of a particular biomarker is desired even if the patient is sedentary. The sweat contains soluble biomarkers that are detectable through sweat chemistry sensors based on electrical property changes of functional structures that are connected to electrical traces in electrical communication with appropriate amplifying, signal conditioning and signal detecting circuits under the control of a microprocessor.

As examples of techniques and devices that can be utilized for the formation of sweat chemistry sensors, very small, inexpensive, sweat chemistry sensors can be manufactured using technologies where all or portions of the traces and/or functional structures can be nano-printed, for example, using the technique shown in PCT patent application of Busnaina, et al., PCT/US2008/012977, filed 2008 Nov. 21US patent, or US patent of John J. Daniels, U.S. Pat. No. 7,799,369 B2, issued 2010 Sep. 21.

As a non-limiting example, the sweat chemistry sensor may be functionalized to detect the presence and/or concentration of biomarkers, such as thrombin and/or D-Dimer. The presence of these biomarkers can be indicative of a physiological change that may be the result of a concerning condition. By combining the automatic detection of these biomarkers along with other biometric parameters, probabilistic analysis can be employed to improve the accuracy of determining the effectiveness of a drug and/or electroceutical therapy, enable automatic adjustments of the administered therapy, send alerts to caregivers, patients, family members, etc., provide useful continuously monitor data for drug discovery and other uses of large population data sets, enable adjustments of dosage, timing and other factors determined during pilot studies and clinical trials, and a number of other advantageous applications of the inventive wearable electronic digital therapeutic device.

As an example, in the case of thrombin, in the coagulation cascade, coagulation factor II is proteolytically cleaved to form thrombin in the first step of the coagulation cascade which ultimately results in the stemming of blood loss. F2 also plays a role in maintaining vascular integrity during development and postnatal life. Mutations in F2 leads to various forms of thrombosis and dysprothrombinemia. Therefore, the detection of the thrombin biomarker along with other biometric parameters such as blood flow through a target blood vessel (and other biomarkers and biometrics described herein and/or currently or in the future employed for human or animal healthcare) is utilized in accordance with the inventive wearable electronic digital therapeutic as an enhanced tool for health and wellness of the individual and for the global population. After the on-demand sweat stimulation results in sweat availability, a subsequent level of the biomarker (e.g., thrombin) is again detected (Step Seven).

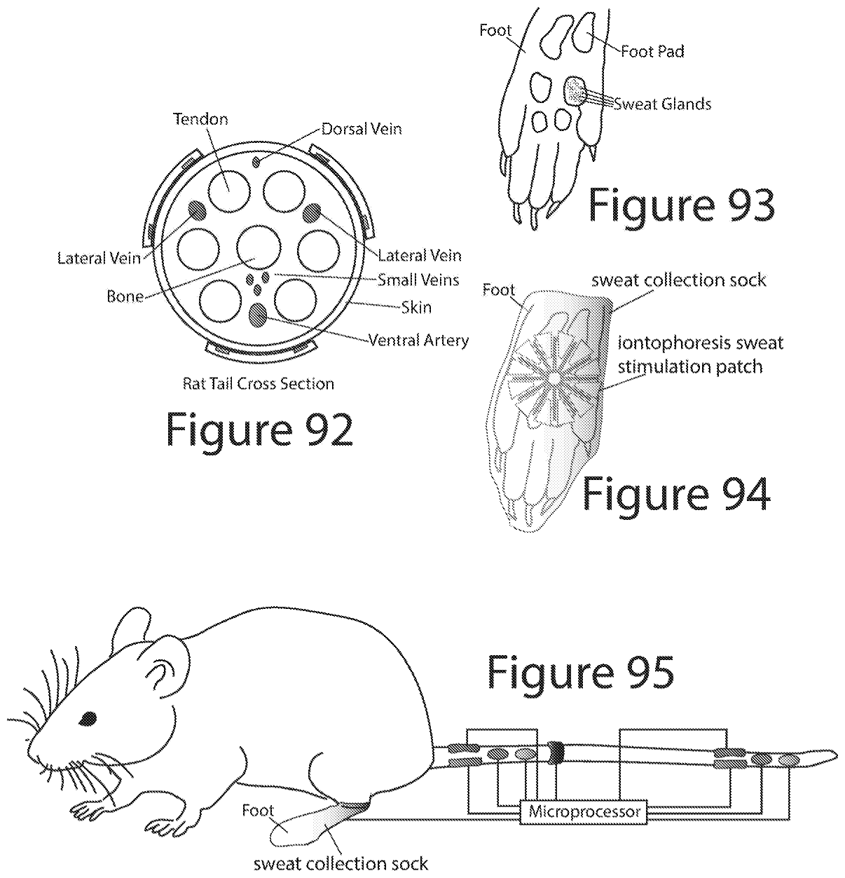

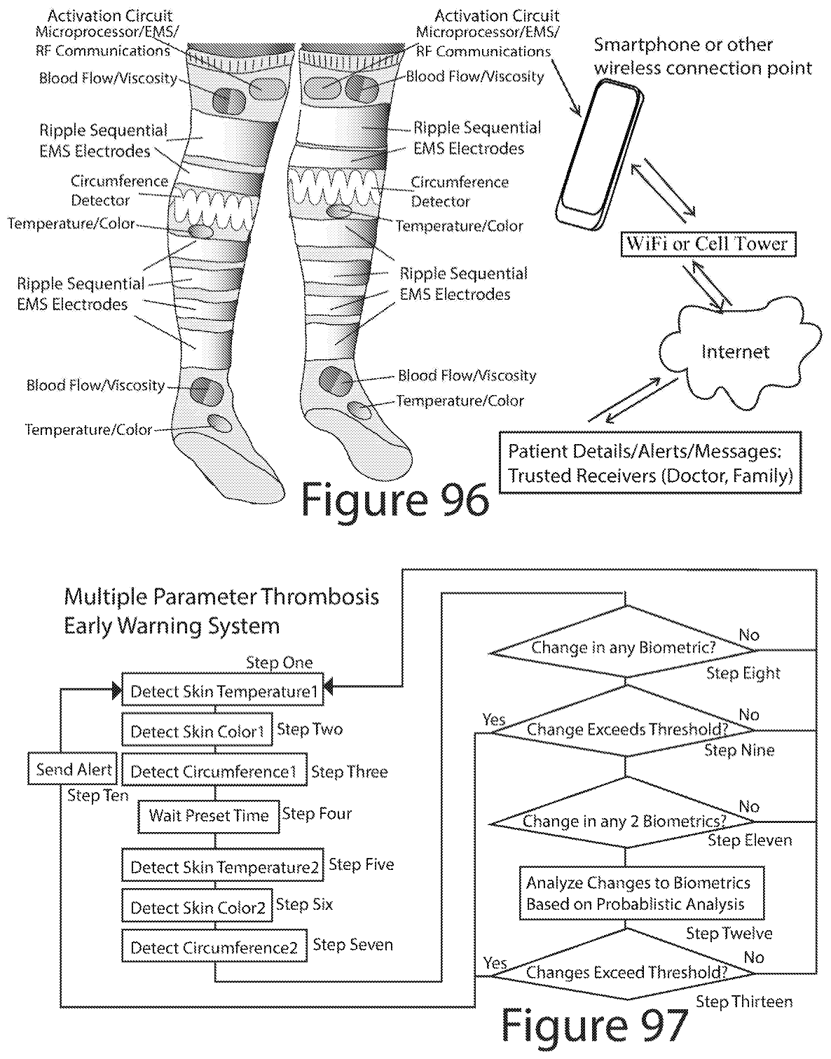

FIG. 92 is a cross section of a rodent tail showing the location of blood vessels and an optical detection system scaled for detecting biometric parameters on the rodent tail. FIG. 93 is an isolated view of a foot of a rodent showing foot pad and sweat glands. FIG. 94 is an isolated view of a foot of a rodent showing a sweat collection sock and iontophoresis sweat stimulation/chemistry detection patch. FIG. 95 illustrates a rodent with a biometric detection system installed on the tail the rodent.

Animal models can be used to effectively predict the efficacy of the inventive drug/device combination treatments described herein. A rodent can be utilized with biometric detectors applied to the body of the rodent to detect, for example, changes in the vascular system, changing in skin color, temperature and swelling. Blood flow and other biometric parameters can also be detected from the rodent, with sweat chemistry being determined through an on-demand sweat stimulation/sweat detection system applied to the foot of the rodent.

The embodiments of the inventive wearable electronic digital therapeutic device are electroceuticals that treat ailments with electrical impulses. Such devices have a long history in medicine, such as pacemakers for the heart, cochlear implants for the ears and deep-brain stimulation for Parkinson's disease.

Anticoagulants are used to treat cardiovascular conditions, such as for the prevention of stroke and systemic embolism in adult patients with non-valvular atrial fibrillation (AF) with one or more risk factors, the treatment of deep vein thrombosis (DVT), the treatment of pulmonary embolism (PE), the prevention of recurrent DVT and PE in adults, the prevention of venous thromboembolism (VTE) in adult patients undergoing elective hip replacement surgery, the prevention of VTE in patients undergoing knee replacement surgery, the prevention of atherothrombotic events (cardiovascular death, myocardial infarction or stroke) after an Acute Coronary Syndrome and other conditions related to the vascular system, etc.

FIG. 96 illustrates an embodiment of the inventive wearable electronic digital therapeutic device configured as a pair of stockings for a combination thrombosis/PAD detection with muscle pump EMS therapy, which may be applied as an alternative to, or complementary to, the use of an anticoagulant or other pharmaceutical treatment.

The inventive wearable electronic digital therapeutic device can be configured to worn on any part of the body and be used to detect biometric signals, collect and transmit biometric data, alerts or otherwise monitor a patient. The digital therapeutic device can be used to improve the treatment of existing pharmaceutical medicines through the complementary use of electroceutical therapy. As a non-limiting example, a digital therapeutic device is provided for activating the muscle pump through an applied electrical muscle stimulation signal modified depending on a detected therapeutic action of an anticoagulant drug.

A wearable electronic garment has at least one pair of electrodes for applying an electrical muscle stimulation signal through the skin of a patient to induce involuntary contractions in one or more muscles adjacent to a deep vein blood vessel. The involuntary muscle contractions induce a squeezing action on the blood vessel and promote a flow of blood through the blood vessel in a direction towards a heart of the patient. A biometric signal detector detects a biometric parameter indicative of the flow of blood through the blood vessel. The biometric parameter is dependent on a therapeutic action of a pharmaceutical medicinal compound for inhibiting an initiation of coagulation of blood. A microprocessor modifies the application of the electrical signal dependent on the detected biometric signal. The applied electrical muscle stimulation signal is modified in response to the therapeutic action of the pharmaceutical medicinal compound.

The biometric parameter may be detectable dependent on at least one of skin temperature, skin color, blood flow, pulse, heartbeat, blood pressure, blood viscosity, skin tightness, swelling, blood chemistry, sweat chemistry, an electronic biomarker, a chemical biomarker, and electromyography, or other suitable biometric or ambient condition. The applied electrical muscle stimulation signal can be applied as a sequence of electrical signals through two or more pairs of electrodes for sequentially squeezing the blood vessel along a longitudinal axis of the blood vessel to promote the flow of blood through the blood vessel in the direction towards the heart determined by the sequential squeezing and one-way vascular valves within the blood vessel.

The biometric signal may be dependent on heartbeat and the applied electrical muscle stimulation signal is modified to impart the squeezing action dependent on the heartbeat. The heartbeat biometric signal can be detected as at least one of a biometric optical signal and an biometric electrical signal from at least one biometric detector in contact with a skin surface of the patient.

The electrical muscle stimulation signal may be applied to the at least one muscle through the skin surface from at least one electrode in contact with the skin surface. The biometric detector includes the at least one electrode that applies the electrical muscle stimulation signal used also for detecting the heartbeat from the biometric electrical signal. The biometric signal can be dependent on surface vein blood flow and the applied electrical muscle stimulation signal is modified to impart the squeezing action dependent on the surface vein blood flow.

FIG. 97 is a flowchart of an algorithm for a combination thrombosis/PAD detector with muscle pump EMS activation system.

A wearable electronic therapeutic device has one or more biometric detectors each for detecting one or more biometric parameters. The skin temperature1 value is detected (Step One), skin color1 value is detected (Step Two) and Circumference1 is detected (Step Three). These initial parametric values are compared with later obtained values where the biometric parameters are dependent on at least one physiological change to a patient in response to a therapeutic treatment. After waiting a preset time (Step Four), parametric values are again detected (Steps Five-Seven). A microprocessor receives the one or more biometric parameters. If there hasn't been a change in any of the biometric parameters (Step Eight) then the loop repeats and the skin temperature1 value is again detected (Step One). If there has been a change in any of the biometric parameters (Step Eight), the microprocessor applies probabilistic analysis to determine if at least one physiological change threshold has been exceeded (Step Nine). The probabilistic analysis and exceeding the threshold can be based on only one of the biometric parameters, for example, by noting a fast-changing condition caused by the swelling of the leg. If the threshold has not been exceeded, then the loop repeats and the skin temperature1 value is again detected (Step One). In addition, or alternatively, if there is a change in two or more biometrics (Step Eleven), the biometrics are analyzed based on probabilistic analysis of the two or more biometrics (Step Twelve) and the exceeding of the threshold may be dependent on the probabilistic analysis of the two or more biometric parameters (Step Thirteen). If the threshold has not been exceeded, then the loop repeats and the skin temperature1 value is again detected (Step One). However, in steps nine or thirteen, if the threshold is exceeded an alert can be sent (Step Ten) before beginning the loop again at step one. The alert can be sent by an activation circuit that activates an action depending on the determined exceeded physiological change. In addition to, or as an alternative, to sending an alert the action that is activated can be dependent on an applied pharmaceutical treatment, for example, activating the applying of an electroceutical treatment in addition or as an alternative to a pharmaceutical treatment. As a non-limiting example, the pharmaceutical treatment may be an anticoagulant drug taken by an at-risk patient. In this case, the detected biometric parameters may indicate an alert should be sent, and/or an electroceutical treatment should be applied.

Other biometric parameters can be detected as an alternative or in addition to the ones described above. For example, the biometric detection of biomarkers, such as thrombin and/or d-dimer, may be used for treatment and monitoring of conditions related to the contact system for coagulation and inflammation.

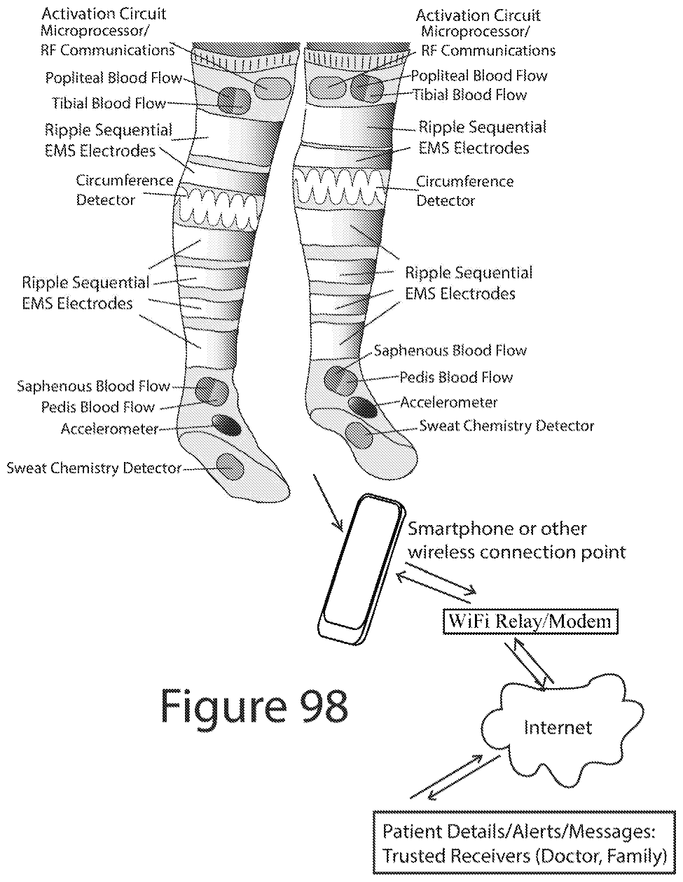

FIG. 98 illustrates an embodiment of the inventive wearable electronic digital therapeutic device configured as a pair of stockings for a combination thrombosis/PAD detection with muscle pump EMS therapy. The embodiment shown is an APP Controlled stocking for at-risk patients of peripheral vascular disease. Other configurations include an arm sleeve, leggings, neck brace, torso shirt, and other wearable electronic garments.

In accordance with the inventive wearable electronic, a pair of comfortable, washable, stockings detect early physiological changes indicating thrombotic or Peripheral Artery Disease (PAD) conditions; and apply electrical stimulation to automatically activate the muscle pump to help return blood flow towards the heart. Early signs of Peripheral Vascular Disease (PVD) may be detected days or even weeks before a patient would normally be prompted to seek medical advice. PVD affects both the veins and the arteries and can often be detected through changes occurring in the lower legs. A wearable electronic stocking includes two or more biometric sensors that constantly monitor the legs checking for signs of swelling, changes in skin temperature and color, along with changes over time of blood flow. The market for this product is large and growing, for example, 8.5 million people in the United States have PAD, including 12-20% of individuals older than age 60.

The stockings may be controlled by a smartphone APP with a user-friendly interface, with large text size and limited but meaningful information. The patient's muscle pump EMS treatment can be applied automatically, with automatic modification to intensity, duration and other characteristics of the applied EMS signal modified depending on the detected biometric parameters (for example, so that the duration and intensity is only as much as necessary). It is expected that in most cases the EMS signal will feel like a gentle massage. The patient can also manually control the EMS treatment and monitor the actuation of the muscles from an onscreen image that shows where the EMS signal is being sequentially applied. A small list of other optional APP features is shown in the drawings. Other user-interface configurations, such as a wrist watch, are also possible.

An example of the medical indication for the stockings include lower extremities peripheral vascular disease used, for example, by an intended patient group that are thrombosis and PAD patients at-risk of a secondary PVD event. Sensors are used for biometric detection of physiological changes with computer algorithm analysis to determine threshold exceeding change(s). A doctor/patient automatic alert can be provided in addition to an EMS muscle pump actuation. The target biometric parameters include changes to circumference (edema), skin temperature/color, and blood flow.

The inventive wearable electronic digital therapeutic device has electrodes for applying an electrical muscle stimulation signal through the skin of a patient to induce involuntary contractions in muscles adjacent to a deep vein blood vessel, activating the muscle pump. The involuntary muscle contractions induce a squeezing action on the blood vessel and promote a flow of blood through the blood vessel in a direction towards the heart of the patient.

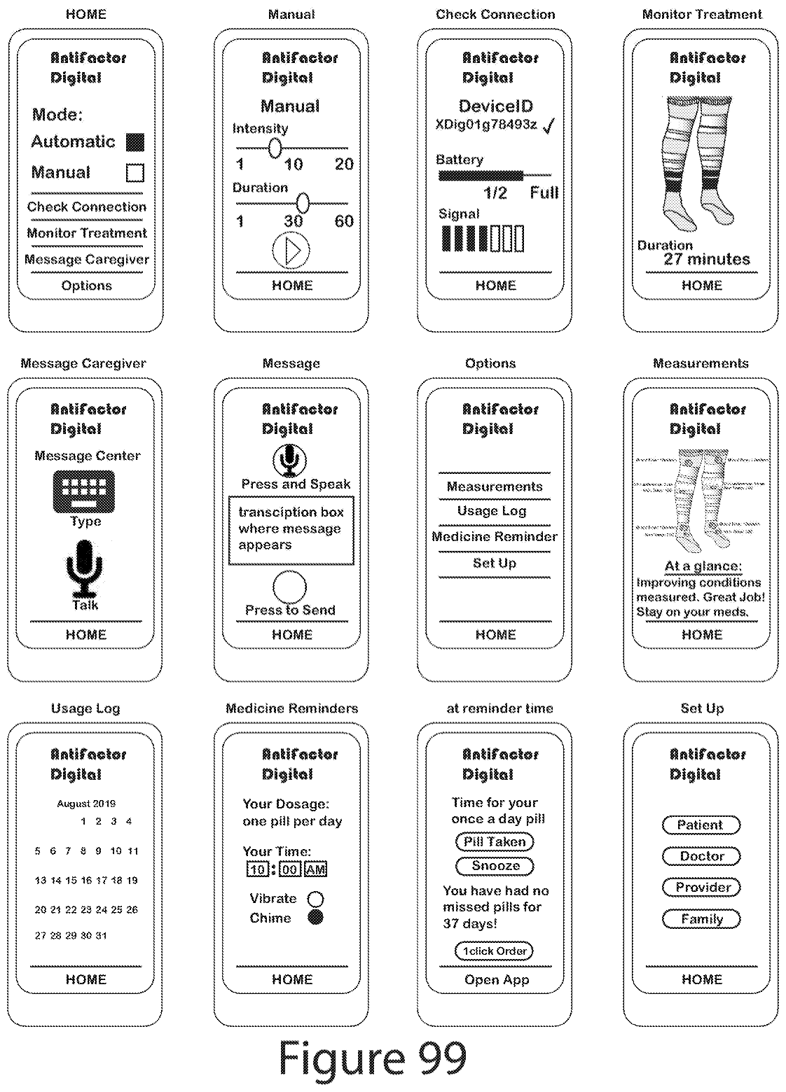

FIG. 99 illustrates a series of user interface screens for an inventive thrombosis/PAD detection stockings with muscle pump EMS therapy. Biometric parameters indicative of peripheral vascular disease (thrombosis and/or PAD) are detected and provide early warning of changes that may put an at-risk patient back in the hospital. The biometric parameters are also dependent on a therapeutic action of Xarelto and the application of the electrical signal is automatically modified dependent on the detected biometric signal in response to the therapeutic action of, for example, an anticoagulant medicinal compound.

A smartphone APP graphical user interface is used to select automatic or manual control of the muscle pump feature. The user interface has large icons and text, with simple actions to check the battery and wireless connection status, and even monitor the treatment as the EMS signals are sequentially applied to the legs.

These user interface smartphone screenshots are for illustration only. The specific flow, user selections and options will be explored through a rapid MVP process coinciding with the development of the wearable electronic stockings, but they show a great use of a powerful computing/communications smartphone device for transforming digital health. The smartphone interface provides a convenient Dr./Patient engagement opportunity, allowing the patient to either type or simply speak (with automatic voice-to-text recognition) quick messages that are sent to the doctor. Other options can be provided through the power of the ubiquitous smartphone, including an enhancement to the system to add a corresponding smartwatch connection. The user-interface options can include viewing current biometric measurements, with an “at a glance” assurance that all is well with the user interface providing encouragement to “stay on your meds.”

A usage log gives the patient a quick indication of how long and when the muscle pump feature was used. A simple medicine reminder screen lets the patient select the time for a gentle reminder to take their pill, and a simple confirmation of adherence. As with all the biometric and user interactions, this adherence data can be provided to the caregiver, the drug company and/or the insurance provider to help ensure constant treatment improvements. Again, the user interface offers an opportunity to encourage the patient to continue adherence noting the days of uninterrupted pill taking and can also include a 1-click Order option that pops up at the correct lime to ensure the patient always has their monthly drug supply. A simple set up screen can also be provided with access limited for sensible patient use and monitoring. A short survey can be automatically displayed, and the patient answers transmitted to assist in remote monitoring.

A major mechanism promoting venous return during normal locomotory activity (e.g., walking, running) is the muscle pump system. Peripheral veins, particularly in the legs and arms, have one-way valves that direct flow away from the limb and toward the heart. Veins physically located within large muscle groups undergo compression as the muscles surrounding them contract, and they become decompressed as the muscles relax. Therefore, with normal cycles of contraction and relaxation, the veins are alternately compressed and decompressed (i.e., “pumped”). Researchers have found dial EMS can be used to increase the venous flow of the lower limbs and concluded that EMS could be a potential method for venous thromboprophylaxis.

Peripheral artery disease (PAD) is estimated to affect more than 20% of people older than 65 years. Peripheral artery disease signs and symptoms include coldness in the lower leg or foot especially when compared with the other leg, a change in the color of the legs, shiny skin on the legs and no pulse or a weak pulse in legs or feet. DVT usually (although not always) affects one leg.

Symptoms of DVT include pain, swelling and tenderness in one leg (usually the calf), a heavy ache in the affected area, warm skin in the area of clot formation and red skin, particularly at the back of the leg below the knee.

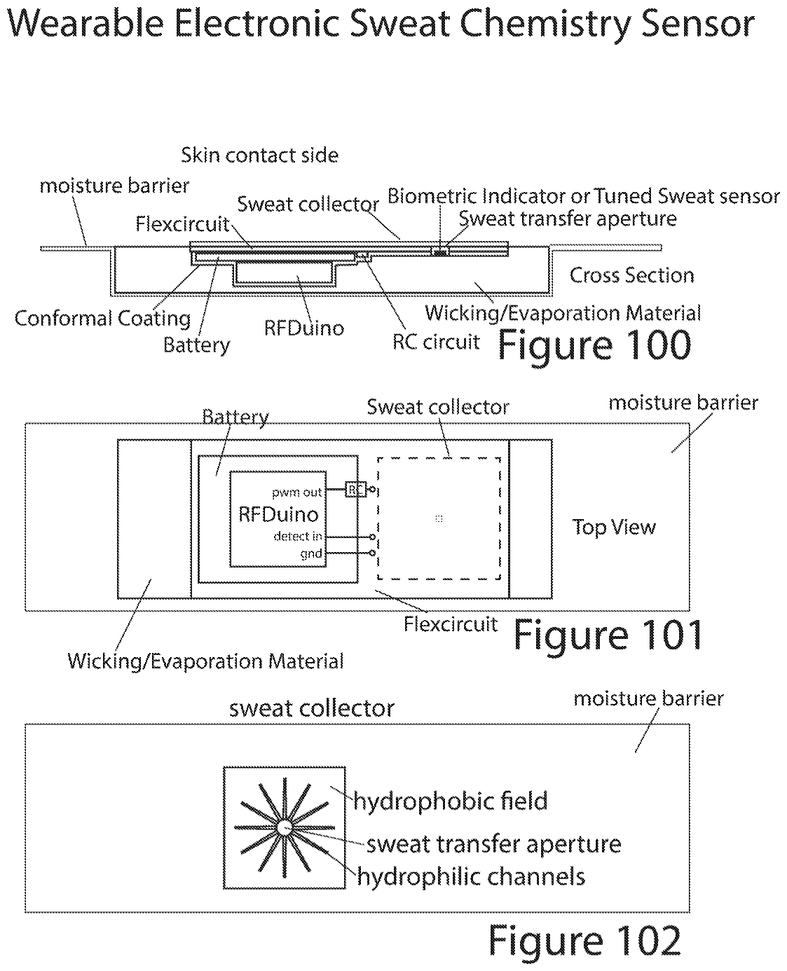

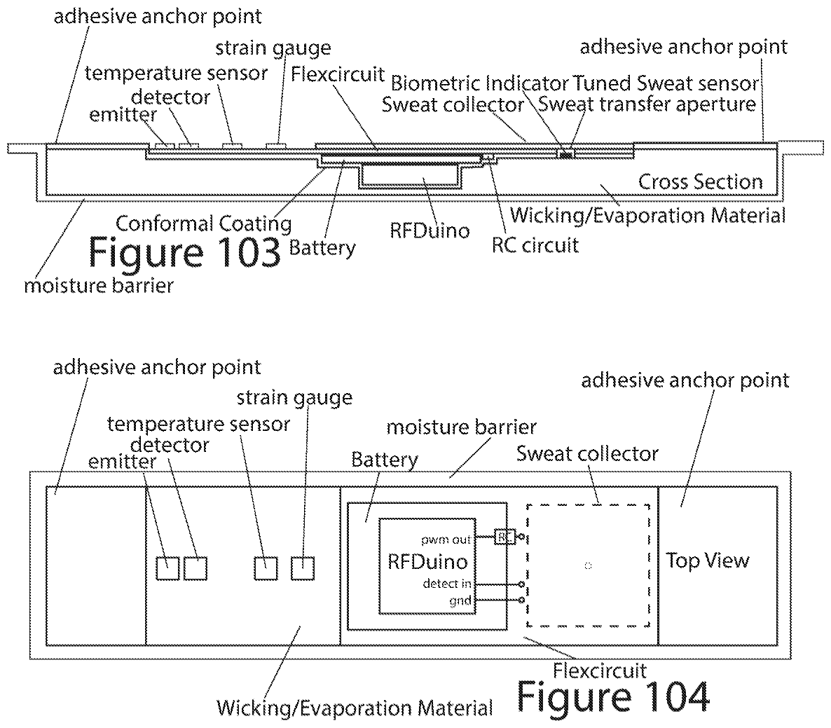

FIG. 100 shows a cross section of an inventive digital therapeutic device sweat chemistry sensor tuned to detect at least one biometric indicator associated with the presence of a therapeutic drug in the blood stream of a patient FIG. 101 shows a top view of the inventive digital therapeutic device sweat chemistry sensor tuned to detect at least one biometric indicator FIG. 102 is an isolated view showing a sweat collector of the inventive sweat chemistry sensor.

A sweat collector craws sweat into a sweat transfer aperture. The sweat chemistry sensor is wet by the sweat and then the sweat is drawn through wicking into wicking/evaporation materials During detection, a flow of fresh sweat passes over the sensor. For many applications, including fitness and military uses, a sweat sensor patch constructed as described herein can be fixed to the skin at a variety of convenient locations, such as the at the waistband of underwear or running shorts. For the application described herein for DVT or other lower leg conditions, a moisture barrier can be fixed in place to enclose the sweat chemistry sensor within a vapor-resistant barrier in order to capture an adequate quantity of sweat, for example, from tire numerous sweat glands at the bottom of the foot.

A hydrophobic field encourages sweat to bead and migrate to hydrophilic channels. Tapered hydrophilic channels use surface tension to draw sweat into the sweat transfer aperture. Hydrophobic and hydrophilic semen printable inks are available from companies such as Cytonix and Wacker.

Many water-soluble components in the blood can be detected through sweat chemistry analysis. Lactate, Glucose and Urea are three important blood chemistry measurements. Lactate is the output of the anaerobic system after that it performs the function as the main fuel for the aerobic system during physical rehabilitation of a patient or competition and much of training of an athlete. Lactate is a major fuel source for the heart and the brain as well as skeletal muscles during strenuous efforts Measuring lactate is a w ay of assessing how strong each energy system is, or essentially how well-conditioned an athlete is or the general health of a patient at a specific point in time. In accordance with the inventive digital therapeutic device, different drug and blood chemistry components can be used as biometric indicators and detected biometrics. For example, if the blood chemistry of a patient indicates dehydration, an alert can be generated that is sent to a caregiver, nurse, family member, the patient, a doctor, etc, and received via cellphone, digital assistant, computers, etc. to indicate that the patient needs attention.

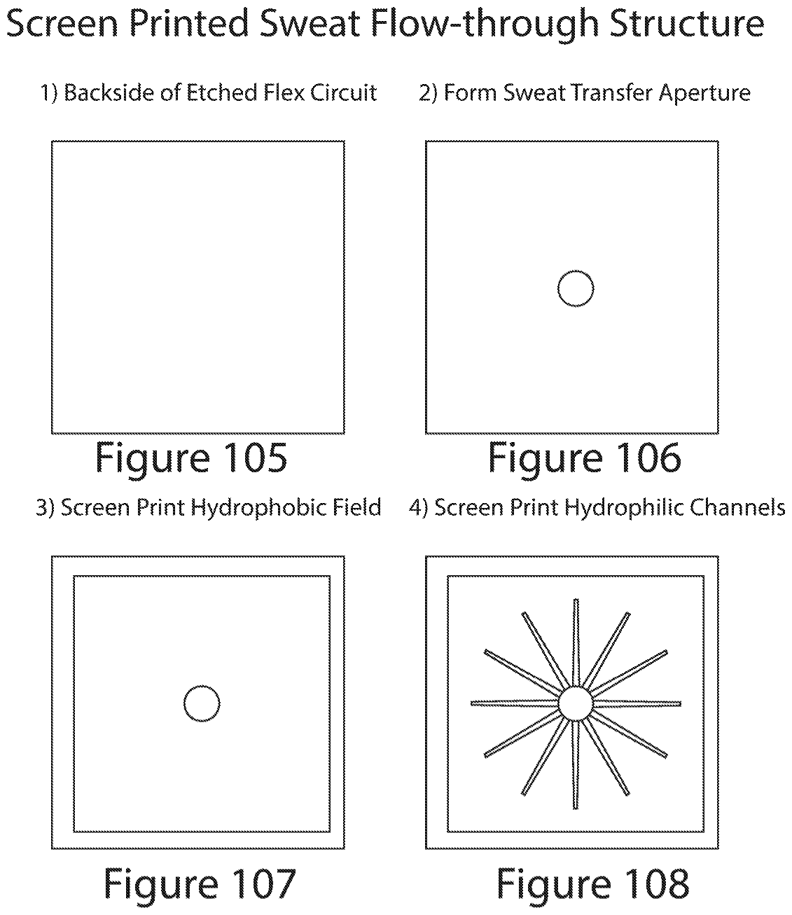

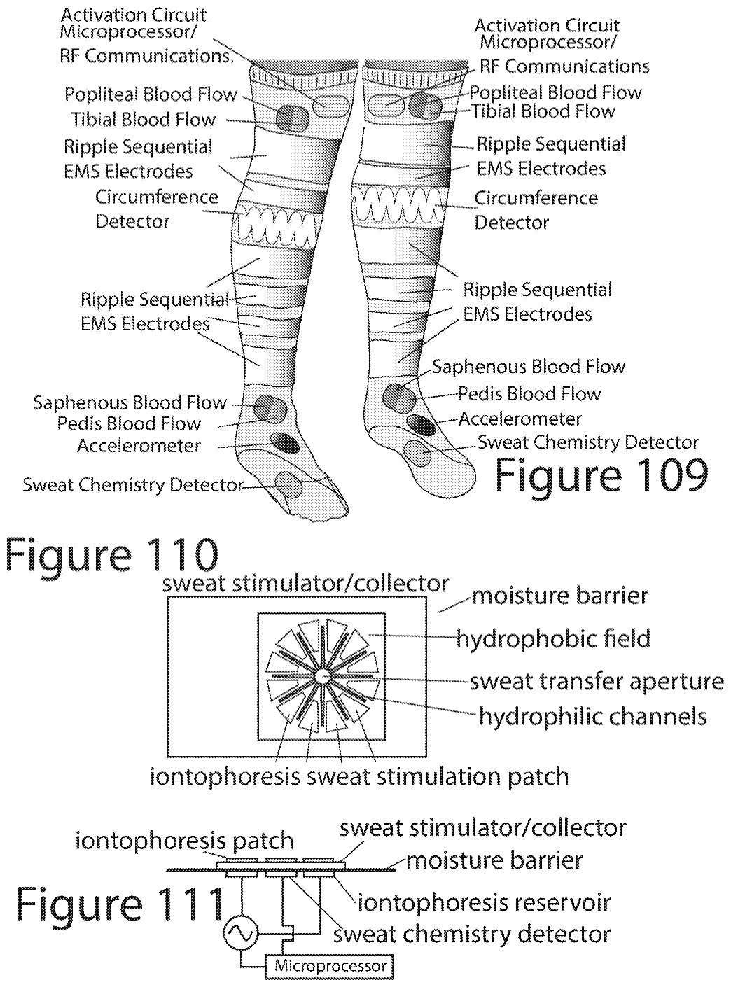

FIG. 105 shows a first step in forming a sweat collector having a flow through structure. FIG. 106 shows a second step in forming a sweat collector having a flow through structure FIG. 107 shows a third step in forming a sweat collector having a flow through structure. FIG. 108 shows a fourth step in forming a sweat collector having a flow through structure.

FIG. 109 shows a cross section of an inventive digital therapeutic device sensor patch with a suite of biometric detectors FIG. 104 shows a top view of the inventive digital therapeutic device sensor patch with a suite of biometric detectors. The inventive digital therapeutic device sensor patch includes adhesive anchor points to strongly adhere the patch to the skin to fix in place and provide anchors tor a strain gauge biometric detector. A light reflectance optical system detects blood flow though surface wins, skin color, blood oxygen, heartbeat and other optically obtained biometric parameters. One or more functionalized sweat chemistry sensors can detect water-soluble components in blood chemistry that are present in sweat, including water-soluble components of anticoagulation drugs, lactose, glucose, ketones, urea, D-dimer and other biomarkers. The biometrics detectable by the inventive digital therapeutic can be distributed at various portions of a wearable electronic, such as a stocking, and/or one or more of the biometric detectors can be incorporated into a stand-alone patch.

The biometric parameters that are detected can include, but are not limited to, light reflectance, surface vein blood flow, skin color, temperature, heartbeat, strain gauge to detect swelling and/or skin tightness, chemical or other biometric indicator, D-dimer or other body-produced biomarker, sweat chemistry indicative of blood chemistry, and other biometric parameters.

FIG. 109 illustrates an embodiment of the inventive wearable electronic digital therapeutic for the analysis of a therapeutic effect based on an activated physiological change and multiple detected biometric parameters:

The retention of fluid in (be legs indicates a number of potentially serious medical conditions. For example DVT or thrombophlebitis is often occurring in one swollen leg (especially the calf), where swelling is caused as blood pools in the area of the deep veins Congestive heart failure can occur if the heart is too weak to pump blood with enough pressure and the blood docs not travel back towards the heart. Varicose veins and chronic venous insufficiency, such as when the valves inside the leg veins do not keep the blood flowing up toward the heart. Renal issues, where the kidneys are not filtering sufficient water and waste materials. Certain medications can also cause legs to swell, such as calcium channel blockers, anti-inflammatory, hormone treatments and even some antidepressants.

A drug, such as an anticoagulant, is often prescribed for the treatment and prevention of thrombosis Typically, the anticoagulant works by inhibiting one or more of the factors of the blood coagulation cascade.

As shown in FIG. 10, in accordance with an embodiment of the inventive wearable electronic digital therapeutic device, the analysis can be made of a therapeutic effect based on an activated physiological change and one or more detected biometric parameters. For example, the activated physiological change can be the on-demand stimulation of sweat. One detected biometric parameter can be the detection of a coagulation cascade factor present in the sweat. Another detected biometric parameter may be the detection of blood flow through blood vessels m a body part, such as the legs Another detected biometric parameter may be a change in the circumference of the leg caused by edema. Other biometric parameters are listed and described elsewhere herein. As shown, a sweat chemistry detector can be included for determining a concentration of a coagulation cascade factor in the blood, a blood gas component, or other soluble molecule or small particulate, in accordance with an exemplars embodiment, the therapeutic effect may be the result of an administered therapeutic, such as a pharmaceutical therapy, e.g., an anticoagulant and/or an electroceutical therapy, e.g., EMS applied to activate the muscle pump. In addition, or as an alternative, to analyzing a therapeutic effect, other physiological responses such as disease progression or modification can be determined by monitoring the one or more detected biometric parameters.

The choice of the one or more detected biometric parameters may depend on the physiological condition, disease, fitness level, treatment being monitored, or other use ease for the analysis of the therapeutic effect. Biometric parameters can be detected as an alternative or in addition to the ones described herein. For example, the biometric detection of biomarkers, such as thrombin and/or d-dimer, may be used for treatment and monitoring of conditions related to the contact system for coagulation and inflammation.

In accordance with the inventive wearable electronic therapeutic device, a temperature sensor simultaneously measures the skin temperature and an electronic circuit detects the progress of the reflected or dispersed amount of radiation and the skin temperature as a function of time. Other biometrics, such the chemistry of the blood can also be included in the detected biometries used to determine and treat concerning conditions of a patient.

As a device that is less invasive and inconvenient as compared to drawing blood samples, a biometric parameter and/or biometric indicator detector suitable for the inventive digital therapeutic device may employ the concept of using a microneedle system. For example, researchers at the University of British Columbia and the Paul Scherrer Institute (PSI) in Switzerland have reported a microneedle drug monitoring system designed to puncture the outer layer of skin but not the next layers of epidermis and the dermis, which house nerves, blood vessels and active immune cells.

The use of a biometric detector based on a microneedle for the inventive digital therapeutic may have the advantages of both convenience, minimal invasiveness and rapid detection, after the ingestion or other delivery of a drug or biometric indicator. Instead of blood, the fluid found just below the outer layer of skin is used to detect and monitor chemicals in the bloodstream. The microneedle may collect just a tiny bit of this fluid, less than a millionth of a milliliter, and a reaction occurs on the inside of the microneedle that can detect blood chemistry using an optical sensor.

Skin color can be detected using optical systems, full-color skin imaging using RGB LED and floating lens in optical coherence tomography, disclosed by Yang B-W, Chen X-C. Full-color skin imaging using RGB LED and floating lens in optical coherence tomography. Biomedical Optics Express. 2010; 1(5): 1341-1346. doi: 10.1364/BOE.1.001341 shows an example of an LED based skin color sensor system that can be modified in accordance with the inventive digital therapeutic to detect skin color as a biometric parameter. It is noted that many of (be various biometric detector can share common components, reducing costs and enabling high speed sampling of different biometric parameters for the different exemplary embodiments described herein.

The Kardia Mobile ECG by AliveCor is an example of an ECG device with well-known electronics that can be modified in accordance with the inventive digital therapeutic to detect heartbeat and other heart related measurements. There are many small and inexpensive examples of blood pulse oximeters, automatic blood pressure readers, and skin temperature sensors Unit can be modified in accordance with the inventive digital therapeutic to detect temperature, blood pressure, pulse, blood oxygen and other related biometric parameters.

In addition to the well-known strain sensors, a stretchable strain has been recently repotted by the University of Houston, Highly Sensitive and Very Stretchable Strain Sensor Based on a Rubbery Semiconductor, ACS Appl. Mater Interfaces. 2018, 10(5), pp 5000-5006, 2018. A stretchable strain sensor with printable components has recently been reported by the University of Florida. Highly Stretchable and Wearable Strain Sensor Based on Printable Carbon Nanotube Layers/Polydimethylsiloxane Composites with Adjustable Sensitivity, ACS Appl Mater interfaces, 2018, 10 (8), pp 7371-7380.

The biometric parameters may include a strain gauge formed from an elastic resistance strip that reversibly changes a detectable resistance value based on being stretched. This can be made simply by the printing of a carbon ink on a stretchable substrate, which could also be, or can be supported by, the fabric of a wearable electronic device such as a stocking or sleeve.

The sweat chemistry sensor max comprise a stretchable electrochemical sweat sensor made, for example, by the deposition of carbon nanotubes (CNTs) on top of patterned Au nanosheets (AuNS) as reported by the graduate school of converging science and technology, Korea University, Seoul (see, for example, Skin-Attachable, Stretchable Electrochemical Sweat Sensor for Glucose and pH Detection, ACS Applied Materials & Interlaces 2018 10 (16). 13729-13740 DOI: 10.1021/acsami.8b03342). This is one example of a sweat chemistry sensor that could be employed as part of the inventive digital therapeutic, for the detection of glucose and pH In this ease. CoWO4/CNT and polyaniline/CNT nauocomposites are coated onto the CNT AuNS electrodes, respectively. A reference electrode is prepared via chlorination of silver nanowires. A change in electrical signal characteristics among the electrodes is indicative of the detected glucose and pH. By modifying the functionalized components, other chemicals present in the sweat can be targeted for detection. By providing multiple sweat chemistry sensor or by creating a patchwork of differently functionalized areas of a multiplexed array of sweat chemistry sensors, a variety of chemicals present in sweat (or the absence thereof) can be determined.

FIG. 110 is a top view of components of a sweat chemistry sensor that includes an activatable physiological change in form of induced sweat stimulation and a moisture barrier to retain the sweat induced from the sweat stimulation. FIG. 111 is a cross sectional view of an iontophoresis patch swear chemistry sensor with a moisture barrier.

In accordance with an embodiment of the inventive digital therapeutic device, patient adherence to the ingestion of an anticoagulant medicinal compound is determined and an alert sent to a trusted receiver. A wearable electronic digital therapeutic device includes a sweat chemistry sensor for sensing one or more water-soluble metabolites present in blood of a patient for positively indicating patient adherence to ingestion of the anticoagulant medicinal compound. The anticoagulant medicinal compound includes tin initially ingested molecular structure that is insoluble in water that is metabolized after ingestion into the one or more water-soluble metabolites. The detection of the one or more water-soluble metabolites by the wearable-electronic digital therapeutic device indicates an adherence by the patient to the ingestion of the anticoagulant medicinal compound. An on-demand sweat stimulator stimulates the production of sweat by the patient. The sweat is received by the sweat chemistry sensor for sensing the one or more water-soluble metabolites. A data transmitter is provided for transmuting data indicating the patient adherence to the ingestion of the pharmaceutical medicinal compound. The anticoagulant medicinal compound can include the water-insoluble molecule:

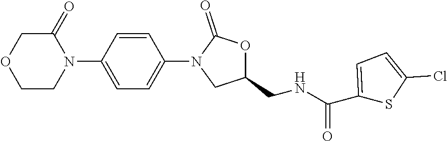 [SMILES: O=C(NC[C@H]1CN(c2ccc(N3CCOCC3=O)cc2)C(=O)O1)c1ccc(Cl)s1]

and the one or more water-soluble metabolites comprises at least one of the molecules:

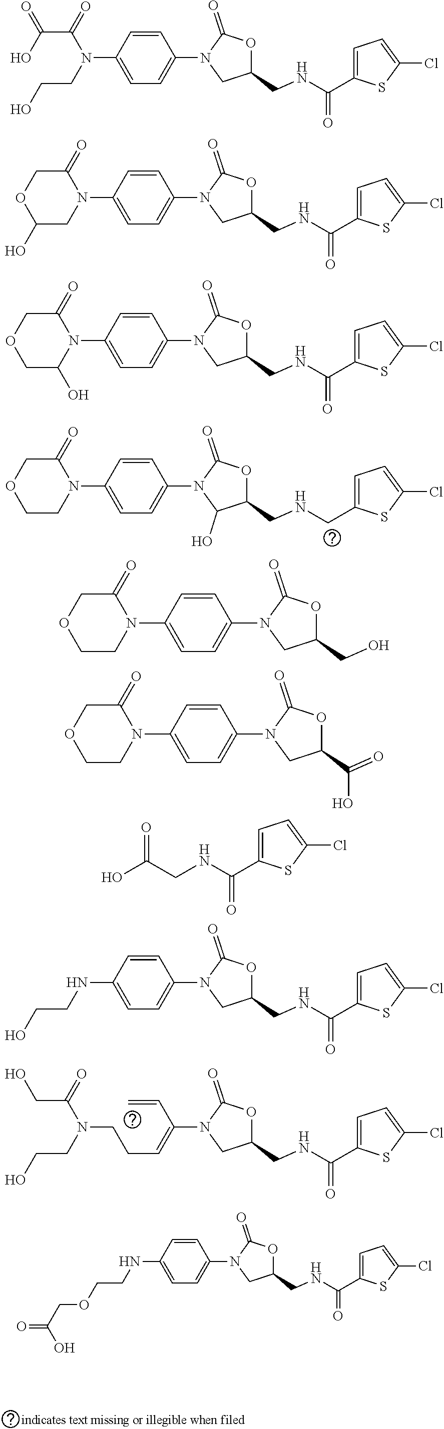

The wearable electronic digital therapeutic device con be configured as at least one of a sock, stocking, ankle bracelet, wrist watch display, wrist bracelet, finger ring, caning, sleeve, shirt, shorts, shirt, leggings, cap, article of clothing and body part accessory.

As shown in FIG. 12, an iontophoresis reservoir holds the sweat inducing material. An iontophoresis patch is disposed in face-to-face contact with the skin surface containing sweat glands (e.g., at the wrist, arm, leg, bottom of the foot, etc.). A microprocessor controls the application of an electrical signal to cause the sweat inducing material to be driven into the skin and activate the sweat glands to produce sweat on demand. A sweat chemistry detector element is functionalized for detecting one or more target molecules. Chemicals present in the sweat are detected by a functionalized sweat chemistry detector element, which generates a signal dependent on the presence of the detected sweat chemistry, this signal is received by the microprocessor indicting the presence or absence of the target molecules.

In accordance with an embodiment, a digital therapeutic device is provided for detecting a patient adherence to the ingestion of a medicinal compound. A wearable electronic digital therapeutic device includes a chemistry sensor for sensing one or more water-soluble metabolites present in blood of a patient for positively indicating patient adherence to ingestion of the medicinal compound. The medicinal compound includes an initially ingested molecular structure that is insoluble in water that is metabolized after ingestion into the one or more water-soluble metabolites. The detection of the one or more water-soluble metabolites by the wearable electronic digital therapeutic device indicates an adherence by the patient to the ingestion of the medicinal compound.

The chemistry sensor can alternatively or additionally be at least one of a sweat, blood, urine, fecal, interstitial fluid, and blood chemistry sensor. An on-demand sweat stimulator can be provided for stimulating the production of sweat by the patient, where the sweat is received by the chemistry sensor for sensing the one or more water-soluble metabolites. A data transmitter can be provided for transmitting data indicating the patient adherence to the ingestion of the pharmaceutical medicinal compound.

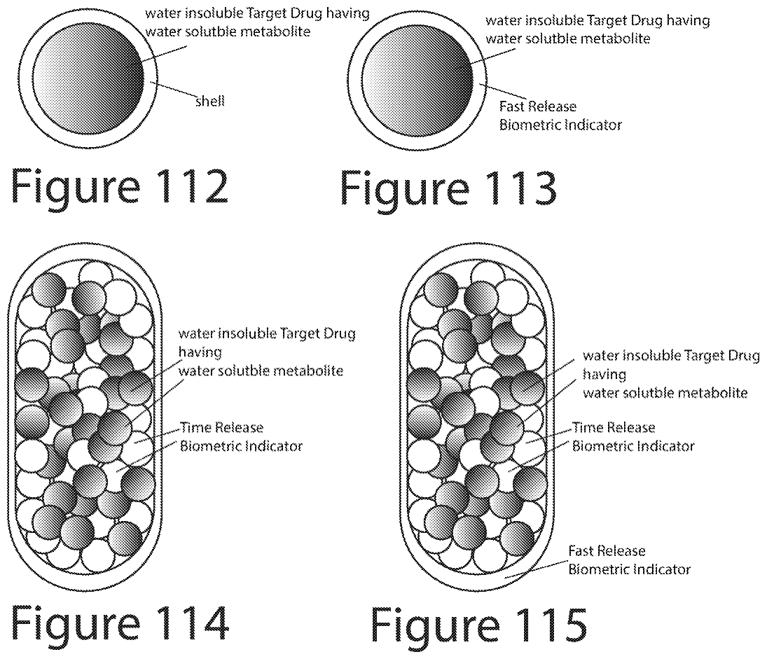

FIG. 112 is a cross section of a pharmaceutical pill including a water insoluble target drug having a water-soluble metabolite and a shell, where the water soluble metabolite is detectable by the inventive digital therapeutic for positively indicating patient adherence to the ingestion of the target drug.

The inventive therapeutic chemistry (target drug) with detectable biometric indicator is used in combination with the inventive digital therapeutic device to enable a reliable determination of a patient's adherence to a prescribed drug therapy. The result is a highly useful, positive indication that can be used to alert a caregiver, hospital, insurance provider, manufacturer, researcher and other interested patties when a patient has taken an intended dosage, or an unscheduled dosage, or a prescribed or over the counter drug. Along with the positive indication of the presence or absence of a drug dosage, water-soluble metabolites of the ingested or otherwise delivered target drug can be useful as biometric indicators and may also be determined by the same digital therapeutic device that detail the physiological effects of the administered drug, or the effects of the hick of the drug in the ease where the drug dosage is missed or otherwise docs not occur.

FIG. 113 is a cross section of a pharmaceutical pill having a water insoluble target drug having a water soluble metabolite and a fast release biometric indicator, where the fast release biometric indicator provides a relatively quicker detectable signal compared to the metabolism of the target drug for positively indicating patient adherence to the ingestion of the target drug through detection of the biometric indicator and for determining therapeutic conditions of the target drug from the detection of the metabolite:

FIG. 114 is a cross section of a capsule containing a water insoluble target drug having a water soluble metabolite and a tune released biometric indicator, where the biometric indicator remains detectable for a duration that relates to the time release of the target drug to provide an indication of the activity of the target drug from ingestion through to full or partial metabolism (or other activation/deactivation mechanism) for comparison with the detection of the water soluble metabolite;

FIG. 115 is a cross section of a capsule containing a time released water insoluble target drug having a water soluble metabolite and a time released biometric indicator, where the biometric indicator remains detectable for a duration that relates to the time release of the target drug, the capsule shell contains a fast release biometric indicator to provide a relatively quicker detectable signal compared to the slow release biometric indicator for positively indicating patient adherence to the ingestion of the target drug;

An inventive pharmaceutical medicinal compound comprises a first compound having a determined therapeutic action on a patient, and a second compound acting as a biometric indicator and having a metabolite acting as a chemical analyte detectable by a wearable electronic therapeutic device. The detection of the chemical analyte by the wearable electronic digital therapeutic device indicates the presence of the pharmaceutical medicinal compound in the body of the patient. The chemical analyte can be detectable by the wearable electronic therapeutic device for positively indicating patient adherence to ingestion of the pharmaceutical medicinal compound. The first compound may comprise a core of a medicinal pill. The second compound may comprise a coating on the core of the medicinal pill.

The compound can be formulated as a controlled release pharmaceutical having at least a portion of the first compound having a delayed release of a bioactive chemical available to perform a therapeutic action. The second compound can be formulated as a last-available biometric indicator with the chemical analyte detectable prior to the delayed release of the first compound as a bioactive chemical, wherein the chemical analyte is detectable faster than the controlled release pharmaceutical becomes a bioactive chemical.

A third compound can be provided acting as another biometric indicator, wherein the third component comprises another chemical analyte and is formulated as a fast-available biometric indicator and wherein the chemical analyte of the third compound is detectable faster than said at least a portion of the first compound becomes a bioactive chemical. The first compound can comprise a granular component contained within a capsule; and wherein the capsule has a shell structure for containing the first compound and includes at least a portion of the second compound as a component of the shell structure. The first compound can be formulated as a controlled release pharmaceutical having at least a portion of the first compound having a delayed release as a bioactive chemical available to perform a therapeutic action, and the second compound can be formulated as a controlled release biometric indicator having the chemical analyte detectable at a rate corresponding to the rate said at least a portion of the first compound become a bioactive chemical. A third compound acting as another biometric indicator can be provided, wherein the third component comprises the chemical analyte and is formulated as a fast-available biometric indicator, wherein the chemical analyte of the third compound is detectable faster than said at least a portion of the first compound becomes a bioactive chemical.

A third compound can be provided acting as another biometric indicator, wherein the third component comprises another chemical analyte and is formulated as a fast-available biometric indicator, wherein the chemical analyte of the third compound is detectable foster than said at least a portion of the first compound becomes a bioactive chemical. The first compound may comprise a granular component contained within a capsule and the second compound comprises another granular component contained with the capsule. The capsule can have a shell structure for containing the first compound and the second compound and include at least a portion of the third compound as a component of the shell structure. The first compound and the second compound can comprise a core of a medicinal pill, and wherein the third compound comprises a coating on tire core of the medicinal pill. The chemical analyte and another chemical analyte can each comprise cither the same or a different chemical not normally present in the blood of the patient. The therapeutic action of the first compound can be inhibiting an initiation of coagulation of blood and the chemical analyte comprises a chemical not normally present in the blood of the patient.

A device for detecting the ingestion of a pharmaceutical medicinal compound may comprise a wearable electronic digital therapeutic device including a biometric indicator detector for detecting a biometric indicator having a chemical analyte for positively indicating patient adherence to ingestion of the pharmaceutical medicinal compound. The pharmaceutical medicinal compound includes a first compound having a determined therapeutic action on the patient. A second compound acts as the biometric indicator and has the chemical analyte detectable by the wearable electronic digital therapeutic device. The detection of the chemical analyte by the wearable electronic digital therapeutic device indicates at least one of an absence and presence of the pharmaceutical medicinal compound ingested by the patient. The wearable electronic digital therapeutic device max further include a data transmitter for transmitting data indicating at least one of an absence and presence of the pharmaceutical medicinal compound ingested by the patient. The pharmaceutical medicinal compound may be, for example, tor inhibiting an initiation of coagulation of blood.

An embodiment includes a device for detecting the ingestion of a pharmaceutical medicinal compound comprising a wearable electronic digital therapeutic device. The wearable electronic digital therapeutic device includes a biometric indicator detector for detecting a biometric indicator having a chemical analyte detectable after ingestion of the pharmaceutical medicinal compound. The pharmaceutical medicinal compound includes a first compound having a determined therapeutic action on the patient as the first compound becomes bioactive within the body of a patient, and a second compound acting as the biometric indicator and having the chemical analyte detectable by the wearable electronic digital therapeutic device. The detection of the chemical analyte indicates the ingestion of the pharmaceutical medicinal compound. The chemical analyte can be detectable at a rate indicative of the bioactivity of the first compound. The biometric indicator can comprise at least one water-soluble metabolite having the chemical analyte wherein the pharmaceutical medicinal compound includes the first compound having a determined therapeutic action on the patient and having at least one therapeutic action metabolite formed as the first compound becomes bioactive within the body of a patient. The second compound can act as the biometric indicator and have at least one chemical analyte of the at least one-water-soluble metabolite detectable by the wearable electronic digital therapeutic device, wherein the at least one-water-soluble metabolite is formed by the body of the patient at a rate indicative of the bioactivity of the first compound A data transmitter can be provided for transmitting data indicating at least one of an absence and presence of the pharmaceutical medicinal compound ingested by the patient.

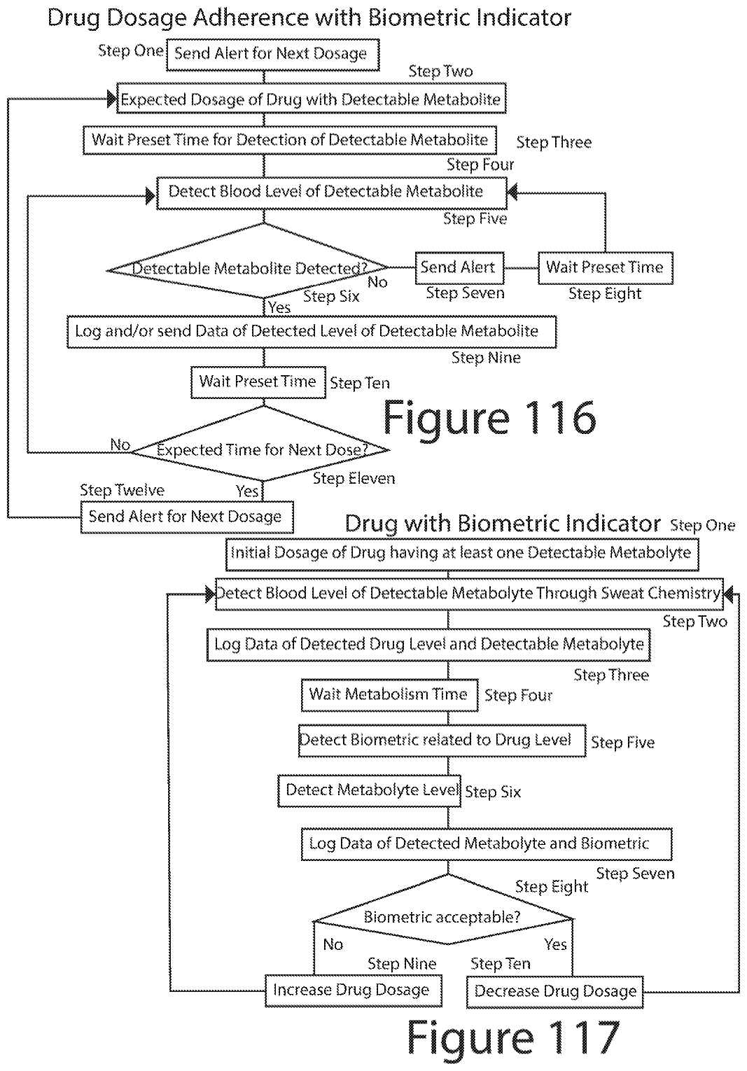

FIG. 116 is a flow chart showing an algorithm for detecting a patient s adherence to a scheduled drug ingestion of a target drug through the detection of the presence of a biometric indicator, where the biometric indicator is a detectable metabolite. The detectable metabolite is detectable by the inventive digital therapeutic for positively indicating patient adherence to the ingestion of the target drug. The target drug may be token as a pill, capsule, other delivery mechanisms including but not limited to a transdermal patch, an intravenous drip, inhalation, eye drops, nasal spray, a transdermal injection, an implanted drug release mechanism, or other drug delivery vehicle.

A biometric parameter, such as blood flow, extrapolated or directly measured blood pressure, skin temperature. EMG measurement, etc., can be used to confirm or adjust the calculated expectations of the target drug effect/concentration.

An alert can be sent to a smartphone, through email, text, phone call, or an alarm timer to remind a patient dial it is lime for the next dosage of a target drug (Step One). It is then expected that the patient will have taken the target drug (Step Two).

A preset wait time elapses for the detection of the detectable metabolite (Step Three). For example, in the case of an inhaled fast release biometric indicator, the wait time can be just a few seconds after inhalation. In the case of a pill, even a fast release detectable metabolite may take longer, and up to several minutes to be detectable in the blood and even longer to be detected through the sweat using one or more of the biometric detectors, such as a sweat chemistry sensor, described herein, or other suitable detector/sensor mechanism.

After the preset time (Step Three), an attempt is made to detect the detectable metabolite (Step Three). If the detectable metabolite is detected (Step Four), then the detected presence and/or level of the detectable metabolite can be logged and/or data transmitted indicating the detected level (or simply a go/no-go presence/absence detection) its an indication of the patients adherence to the prescribed course of drug therapy.

If after the preset time has elapsed (Step Two) and the detectable metabolite is not detected (Step Three) then an assumption may be made that the patient has not adhered to the prescribed course of drug therapy, and an alert is sent (Step Seven) by email, text, phone call, pager, SMS, or other communication method to let a caregiver, service provider, family member, the patient, the insurer, and/or the healthcare provider know that the patient may not have taken the prescribed drug dosage. After sending the alert, especially in the case where a reminder is sent directly to the patient or on-site caregiver present with the patient, an expectation is assumed that the patient has taken the target drug and biometric indicator, and a preset wait time can then again be allowed to elapse (Step Eight) before attempting to detect the delectable metabolite (Step Four).

If the detectable metabolite is detected (Step Six), after the data indicating the level or presence of (ire detectable metabolite is logged and/or transmitted, a preset time may be allowed to elapse (Step Ten). If the expected time for the next dosage has not elapsed (Step Eleven), then the blood level of the detectable metabolite can be detected again (Step Four) and if detected (Step Six) an indication of the detection logged or transmitted (Step Nine) to obtain a chronological history of the blood level of the detectable metabolite as an indication of the blood level of the target drug (which may be particularly useful in the case where the detectable metabolite has a blood level that correlates with the expected blood level over lime of the target drug). When it is expected that the patient should be taking the drug dosage (Step Eleven) an alert for the next dosage can be sent out to remind the patient, caregiver, etc., that it is time for the next dosage to be taken (Step Twelve) and there the expectation that the patient will have taker the drug dosage with the detectable metabolite is tested (Step Two).

This process can continue for as long as the target drug is prescribed for the patient, for days, weeks, months or even years, providing a detailed history of the patient's adherence to the proscribed course of drug therapy. If biometric parameters such as those described heroin with regards to the other embodiments are also detected, logged and/or transmitted, a detailed history of the patient's therapy, course of treatment, measured results of treatment, etc., will be made available to improve the care given to the particular patient, and in the aggregate, provide significant data along with that of other patients, to assist in new drug discovery, treatment modifications, and a number of other advantages of the beneficial cycle created by detection, transmission, storage and analysis of biometric data taken directly from the patient during the course of drug therapy and/or other treatments.

FIG. 117 is a flow chart showing an algorithm for detecting the taking of a target drug along with a biometric indicator incorporate in the same pill or capsule or otherwise imparted into the patient at the same time or at a known time relative to the taking of the target drug. The detection of the biometric indicator is used as a positive indication that the target drug has been taken by the patient A pill, capsule, or other drug delivery mechanism, containing a target drug may include a biometric indicator that is detectable and used to indicate, for example, patient adherence (e.g., (be taking of a pill containing the target drug and the biometric indicator), the availability of the target drug into the blood stream, the timed release of the target drug, the metabolism and/or excretion of the target drug, etc. For example, in the ease of the timed release of the target drug, the biometric indicator can be delivered with the same time-release mechanism as that incorporated with the target drug. The biometric indicator can be, for example, an additional component added to the chemistry of a new or pre-existing drug. In accordance with an embodiment, the ideal biometric indicator is a non-troubling water-soluble chemical compound that docs not adversely alter normal body functionality, is detected from analysis of the sweat, and docs not adversely affect the beneficial actions of the target drug. As an example, a compound containing polyhydroxyalkanoates (PHAs) which has biodegradable and biocompatible properties may be used as the detected chemical analyte.

An initial dosage of a target drug is taken along with a biometric indicator (Step One). A blood level of the biometric indicator is detected, for example, through sweat chemistry analysis (Step Two). The biometric indicator may be detected via other detection mechanisms including but not limited to direct blood chemistry analysis, a measured biometric such as EMG, skin temperature, skin color or other change in a detectable parameter. The detected drug level and/or the biometric indicator are logged (Step Three).

The detected data can be stored locally in a memory associated with the inventive wearable electronic digital therapeutic device, or a remote memory located in a smart phone, a network server, a computer or other external device. The detected data can be filtered, compressed or otherwise conditioned prior to storage or transmission. A predetermined or calculated period of time is allowed to elapse to enable, for example, the metabolism, activation, deactivation, therapeutic action, or other change to occur in the blood levels of at least one of the target drug and the biometric indicator (Step Four). After the time has elapsed, the biometric related to the drug level is detected (Step Five) and the target drug level may optionally be detected directly or indirectly by extrapolation from the detected biometric indicator (Step Six). The data of the detected drug level and/or biometric is logged and/or transmitted to obtain, for example, a chronological history of the blood level of the biometric indicator as an indication of the blood level of the target drug (particularly in the case where the biometric indicator has a blood level that correlates with the expected blood level over time of the target drug).

As a further option, the detected biometric and/or the detected or extrapolated drug level may be used to inform a healthcare provider, a family member, the patient, an AI agent, a healthcare payor, a researcher, or other party that data has been provided that can be used to adjust the dosage of the drug taken by the patient or indicated for a population that takes the drug. The detected biometric and/or drug level can be used to determine if a biometric is acceptable (Step Eight) If it is not acceptable, an alert, automatic action, and/or other indication of the biometric not being acceptable can be used to increase or decrease the drug dosage. For example, if a larger dosage would increase the effectiveness of the drug as indicated by the detected biometric, the drug dosage may be increased (Step Nine), if the detected biometric indicates the drug dosage is too great or indicates the drug dosage is working correctly, the drug dosage may be decreased (Step Ten) to determine the minimum dosage required for an optimized treatment. The feedback loop iterates from the detection of the biometric (Step Two).

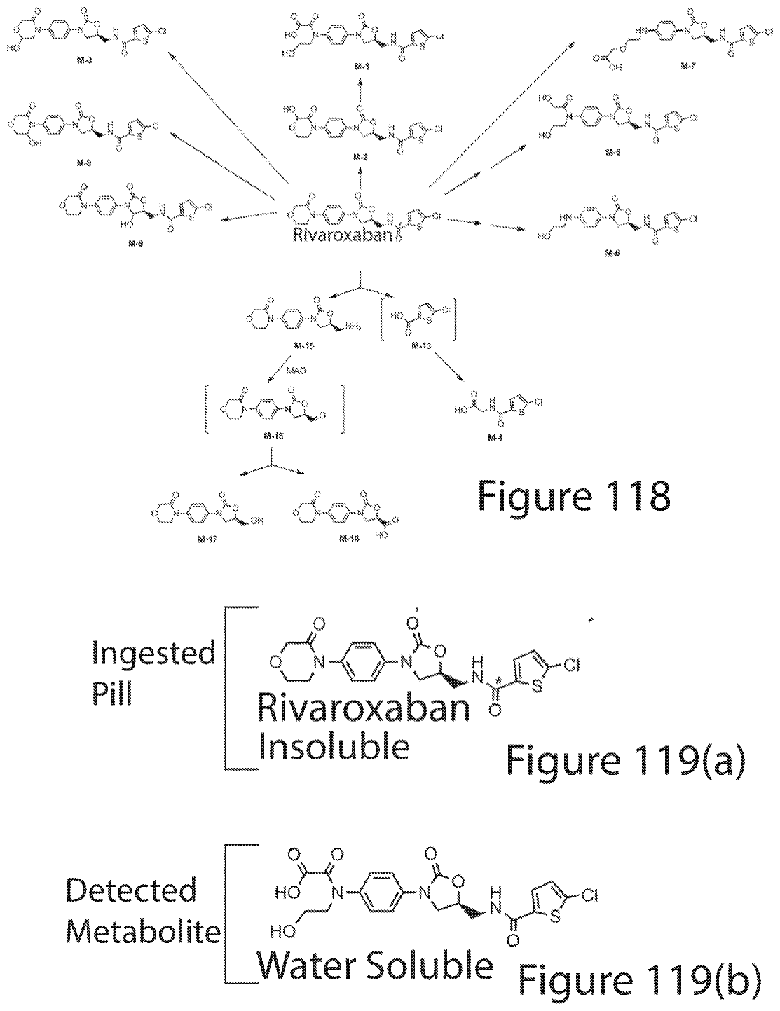

FIG. 118 shows a water-insoluble anticoagulant drug and molecular pathways to water-soluble metabolites. In this example, the water-insoluble anticoagulant drug is rivaroxaban which has known pathways to water-soluble metabolites (Metabolism and excretion of rivaroxaban—an oral, direct Factor Xa inhibitor—in rats, dogs and humans, Weinz et al., dmd.aspetjournals.org at ASPET Journals on Nov. 14, 2018). Pharmacokinetic studies of rivaroxaban in animal models demonstrate that rivaroxaban is absorbed rapidly after oral dosing (absolute bioavailability 57-66% and 60-86% in rats and dogs, respectively). Rivaroxaban has an advantageous pharmacokinetic profile with dose proportional increase in area under the concentration-time curve (AUC), and is shown to be rapidly excreted via renal and faecal/biliary routes (Weinz C, Buetchorn U, Daehler H P, Kohlsdorfer C, Pleiss U, Sandmann S. Schlemmer K H. Schwarz T, and Steinke W (2005) Pharmacokinetics of BAY 59-7939 an oral, direct Factor Xa inhibitor in rats and dogs. Xenobiotica 35:891-910). Rivaroxaban has also been shown to denonstrate dose-proportional pharmacokinetics and predictable pharmacodynamics in single- (up to 80 mg) and multiple-dose studies in healthy subjects and patients with no evidence of accumulation. In addition, rivaroxaban showed high oral bioavailability with a rapid absorption, and was safe and well tolerated Kubitza D. Becka M. Voith B. Zuehlsdorf M, and Wensing G (2005a) Safety, pharmacodynamics, and pharmacokinetics of single doses of BAY 59-7939, an oral, direct factor Xa inhibitor. Clin Pharmacol Ther 78:412-421).

FIG. 119(a) shows a water-insoluble molecule of a therapeutic medicinal compound. FIG. 119(b) shows a water-soluble molecule that is a metabolite of the water-insoluble molecule. In accordance with an embodiment, a digital therapeutic device is provided for detecting patient adherence to taking an anticoagulant medicinal compound. The digital therapeutic detects for example, a patient adherence to the ingestion of an anticoagulant medicinal compound. A wearable electronic digital therapeutic device includes a sweat chemistry sensor for sensing one or more water-soluble metabolites present in blood of a patient for positively indicating patient adherence to ingestion of the anticoagulant medicinal compound. The anticoagulant medicinal compound includes an initially ingested molecular structure that is insoluble in water that is metabolized after ingestion into the one or more water-soluble metabolites. The detection of the one or more water-soluble metabolites by the wearable electronic digital therapeutic device indicates an adherence by the patient to the ingestion of the anticoagulant medicinal compound. An on-demand sweat stimulator stimulates the production of sweat by the patient, w here the sweat is received by the sweat chemistry sensor for sensing the one or more water-soluble metabolites A data transmitter transmits data indicating the patient adherence to the ingestion of the pharmaceutical medicinal compound. The anticoagulant medicinal compound may comprise the water-insoluble molecule:

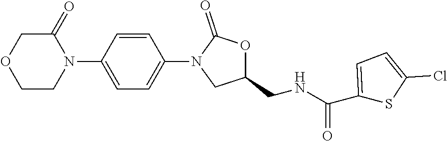 [SMILES: O=C(NC[C@H]1CN(c2ccc(N3CCOCC3=O)cc2)C(=O)O1)c1ccc(Cl)s1]

and the one or more water-soluble metabolites comprises at least one of the molecules:

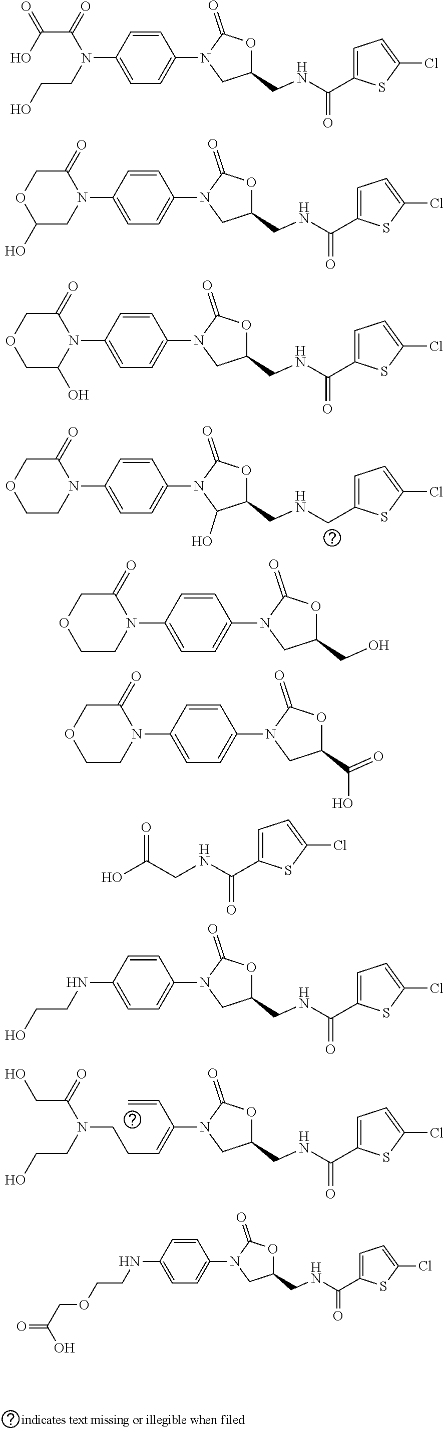

The wearable electronic digital therapeutic device can be configured as a sock, stocking, ankle bracelet, wrist watch display, wrist bracelet, finger ring, earring, sleeve, shirt, shorts, shirt, leggings, cap, or other article of clothing or body part accessory.

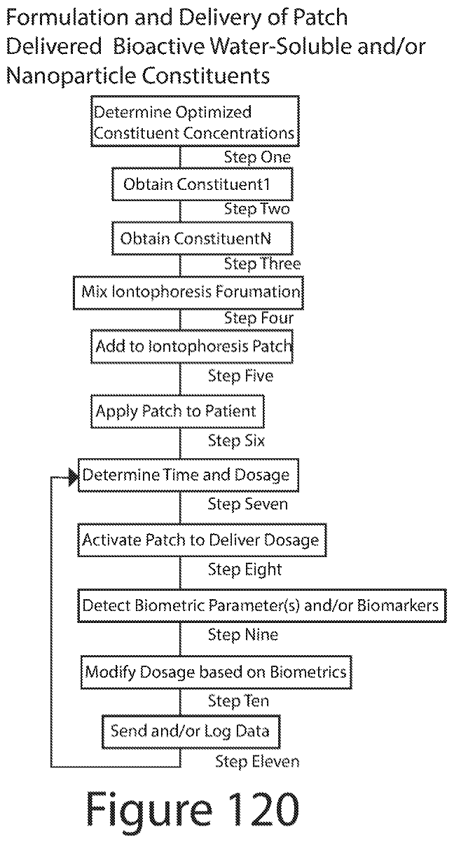

FIG. 120 is a flow chart illustrating an algorithm for the formulation and delivery of patch delivered bioactive water-soluble and/or nanoparticle constituents of a therapeutic medicinal compound(s). Optimized concentrations of bioactive water-soluble and/or nanoparticle constituents are determined. The constituents are, for example, the bioactive compounds of an ingested water-insoluble drug. For example, constituents of a coagulation factor inhibitor can be formed into nanoparticulated and/or water-soluble constituents that can be delivered transdermal from a medicinal patch, iontophoresis patch, microneedle patch or other wearable configuration.

Constituent 1 though constituent N are obtained (Steps Two and Three). The constituents can be obtained through nanoparticulation of chemical compounds that can be delivered transdermal using time release formulas, or on-demand iontophoresis through medicinal, iontophoresis and/or microneedle patch constructions. In the ease of an iontophoresis patch construction, an iontophoresis formulation can be mixed (Step Four) of the constituents so that the optimized constituent concentrations are delivered to the patient for a desired therapeutic effect. The formulation is added to an iontophoresis patch (Step Five).

When ready to use, the patch is applied to the patient Onboard microprocessor, activation and communication circuitry can be used to determine a time and dosage (Step Seven). The patch is activated to deliver the dosage (Step Eight), For example, the application of an electrical signal to the iontophoresis patch con be used to drive the bioactive constituents through the skin to be taken up by blood of the patient. Alter a preset period of time, biometric parameters and/or biomarkers are detected and analyzed to determine physiological changes indicative of a therapeutic effect caused by and/or metabolism of the constituents (Step Nine). The timing and dosage of the next activation of the patch is modified depending on the determined physiological and/or projected physiological changes (Step Ten). For example, depending on predetermined and analyzed data, the duration of activation of the patch to deliver the dosage can be modified and/or in the ease of multiple patches each with for administering different constituents, the mix of delivered constituents can be modified based on the detected biometries. The detected biometric data, ambient information, and other relevant detected or extrapolated data, is sent and/or logged (Step Eleven) and then an onboard or remote microprocessor again determines the time and dosage for the next patch delivered bioactive constituent(s) (Step Seven).

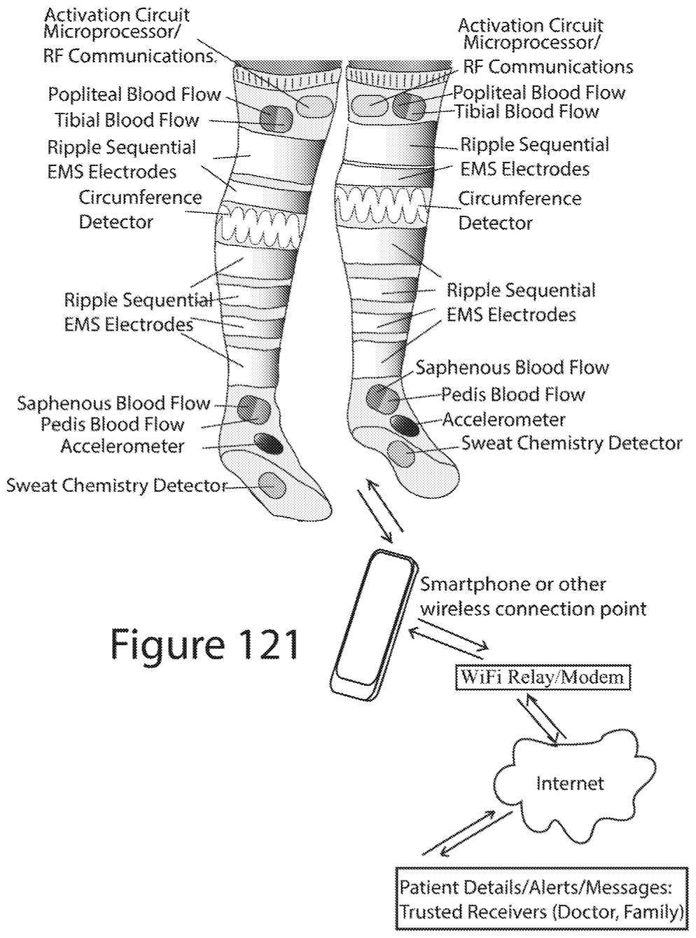

FIG. 121 illustrates an embodiment of the inventive wearable electronic digital therapeutic device for the analysis and/or modification of pharmaceutical and/or electroceutical treatments based on an activated physiological change and detected biometric parameters. A wearable electronic therapeutic device has one or more biometric detectors each for detecting one or more biometric parameters. The biometric parameters are dependent on at least one physiological change to a patient in response to a therapeutic treatment. A microprocessor receives the one or more biometric parameters and applies probabilistic analysis to determine if at least one physiological change threshold has been exceeded dependent on the probabilistic analysis of the two or more biometric parameters. An activation circuit activates an action depending on the determined exceeded said at least one physiological change.

The therapeutic treatment can include an anticoagulant for treating a cardiovascular condition, where the at least one physiological change includes an indication of a change in the cardiovascular condition. The activated action can include transmitting an alert, modifying the therapeutic treatment, and transmitting data dependent on at least one of the at least one physiological change, the one or more biometric parameters, the therapeutic treatment and the probabilistic analysis. The probabilistic analysis may comprise determining from a data set of the one or more biometric parameters whether the data set is acceptable for deciding that the at least one physiological change threshold has been exceeded.

The probabilistic analysis may further comprise applying a statistical weighting to each of the one or more biometric parameters. The statistical weighting can be dependent on a predetermined value of a ranking of importance in detecting each of the at least one physiological change for said each of the one or more biometric parameters relative to others of the one or mom biometric parameters. The at least one of the biometric values is determined from one or more water-soluble molecules; and further comprising an on-demand sweat stimulator for stimulating the production of sweat by the patient and a sweat chemistry sensor for sensing the one or more water-soluble molecules.

In accordance with an embodiment, a digital therapeutic device is provided comprising a wearable electronic therapeutic device having one or more biometric detectors each for detecting one or more biometric parameters. The biometric parameters are dependent on at least one physiological change to a patient. A microprocessor receives the one or more biometric parameters and determines if at least one physiological change threshold has been exceeded dependent on the one or more biometric parameters. An activation circuit activates an action depending on the determined physiological change. The action includes at least one of transmitting an alert, modifying a therapeutic treatment, and transmuting data dependent on at least one physiological change, the one or more biometric parameters, and the therapeutic treatment.

The at least one physiological change can be in response to an applied therapeutic treatment that is at least one of a pharmaceutical treatment and an electroceutical treatment. An additional treatment can also be applied, such as a pneumatic and/or mechanical compression sleeve treatment. The therapeutic treatment can include an anticoagulant for treating a cardiovascular condition, and the physiological change include an indication of a change in the cardiovascular condition.

The action can include transmitting an alert, modifying a therapeutic treatment, and transmitting data dependent on at least one of the at least one physiological change, the one or more biometric parameters, and the therapeutic treatment. The microprocessor can analyze the one or more biometric parameters using probabilistic analysis comprising determining from a data set of the one or more biometric parameters whether the data set is acceptable for deciding that the at least one physiological change threshold has been exceeded. The probabilistic analysis can further comprise applying a statistical weighting to each of the one or more biometric parameters, where the statistical weighting is dependent on a predetermined value of a ranking of importance in detecting each of the at least one physiological change for said each of the one or more biometric parameters relative to others of the one or more biometric parameters. At least one of the one or more biometric values can be determined from one or more water-soluble molecules. An on-demand sweat stimulator for stimulating the production of sweat by the patient and a sweat chemistry sensor provided for sensing the one or more water-soluble molecules.

The inventive wearable electronic digital therapeutic device can be configured to be worn on any part of the body and be used to detect biometric signals, collect and transmit biometric data, alerts or otherwise monitor a patient. The digital therapeutic device can be used to improve the treatment of existing pharmaceutical medicines through the complementary use of electroceutical therapy. As a non-limiting example, a digital therapeutic device is provided for activating the muscle pump through an applied electrical muscle stimulation signal modified depending on a detected therapeutic action of an anticoagulant drug.

A wearable electronic garment has at least one pair of electrodes for applying an electrical muscle stimulation signal through the skin of a patient to induce involuntary contractions in one or more muscles adjacent to a deep vein blood vessel. The involuntary muscle contractions induce a squeezing action on the blood vessel and promote a flow of blood through the blood vessel in a direction towards a heart of the patient. A biometric signal detector detects a biometric parameter indicative of the flow of blood through the blood vessel. The biometric parameter is dependent on a therapeutic action of a pharmaceutical medicinal compound for inhibiting an initiation of coagulation of blood A microprocessor modifies the application of the electrical signal dependent on the detected biometric signal, the applied electrical muscle stimulation signal is modified in response to the therapeutic action of the pharmaceutical medicinal compound.

The biometric parameter may be detectable dependent on at least one of skin temperature, skin color, blood flow, pulse, heartbeat, blood pressure, blood viscosity, skin tightness, swelling, blood chemistry, sweat chemistry, an electronic biomarker, a chemical biomarker, and electromyography, or other suitable biometric or ambient condition. The applied electrical muscle stimulation signal can be applied as a sequence of electrical signals through two or more pairs of electrodes for sequentially squeezing the blood vessel along a longitudinal axis of the blood vessel to promote the flow of blood through the blood vessel in the direction towards the heart determined by the sequential squeezing and one-way vascular valves within the blood vessel.

The biometric signal may be dependent on heartbeat and the applied electrical muscle stimulation signal is modified to impart the squeezing action dependent on the heartbeat. The heartbeat biometric signal can be detected as at least one of a biometric optical signal and an biometric electrical signal from at least one biometric detector in contact with a skin surface of the patient.

The electrical muscle stimulation signal may be applied to the at least one muscle through (lie skin surface from at least one electrode in contact with the skin surface. The biometric detector includes the at least one electrode that applies the electrical muscle stimulation signal used also for detecting the heartbeat from the biometric electrical signal. The biometric signal can be dependent on surface vein blood flow and the applied electrical muscle stimulation signal is modified to impart the squeezing action dependent on the surface and/or deep vein blood flow.

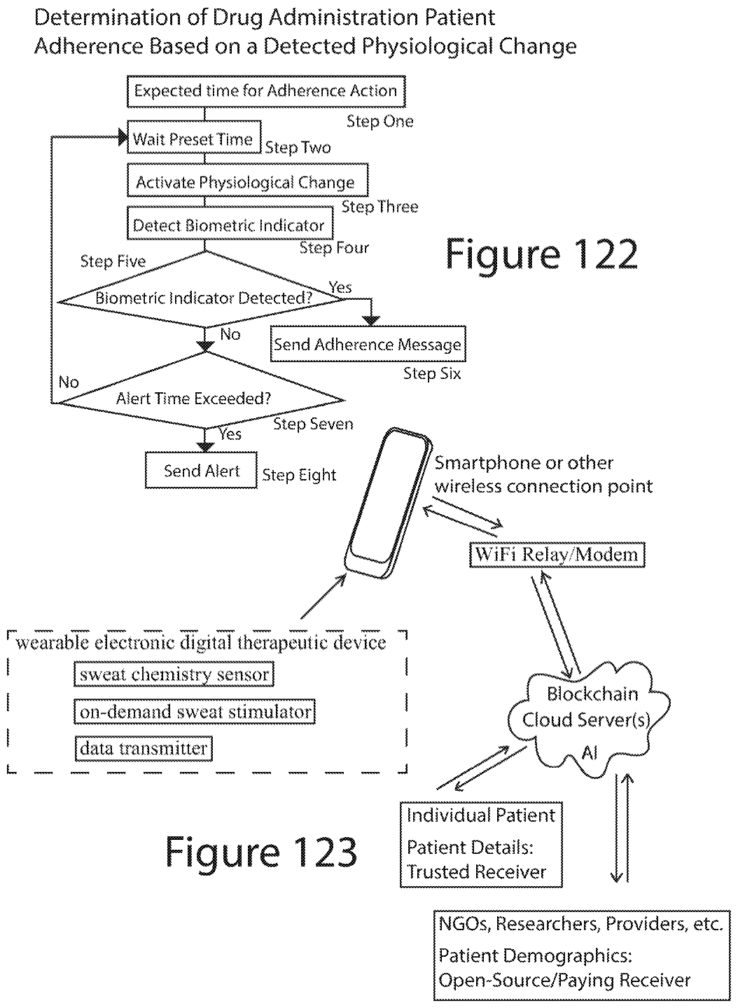

FIG. 122 is a flow chart illustrating an algorithm for determining drug administration patient adherence. As an example, determining the adherence may be done though an analysis based on an activated sweat stimulation detected biometrics or biomarkers. The process starts at an expected lime tor an adherence action (Step One) and a preset time is allowed to elapse (e.g., after a preset time from when a patient is expected to have ingested a medicinal compound pill to allow for the production of metabolites) (Step Two). A physiological chance is activated, such as sweat stimulation activated by iontophoresis where a sweat stimulating chemical, such as carbachol, is electrically driven on-demand into the skin (Step Three). The presence of a biometric indicator is detected to indicate drug administration patient adherence (Step Four). If the biometric indicator indicates drug administration patient adherence (Step Five), then an Adherence Message is sent (Step Six). If the biometric indicator is not present (Step Five), then it is determined if an Alert Time has been exceeded (Step Seven). If the Alert Time has been exceeded, then it is assumed that the patient has not taken or received a scheduled drug administration and an Alert is sent (Step Eight). If the Alert Time has not been exceeded (Step Seven), then a preset time is allowed to elapse (Step Two) be foie activating the physiological change and looking for the presence of the biometric indicator (Step Four).

As an example use, an initial blood flow through a target blood vessel is detected. For example, the popliteal artery and/or vein or other blood vessel on the leg of a patient can be targeted for blood flow detection by the placement of an optical blood flow sensor against the skin in the location of the popliteal artery/vein, etc. The optical blood flow sensor may be disposed to detected capillary blood flow that receives and drains blood from the popliteal artery/vein and the blood flow through those vessels extrapolated from the capillary blood flow. The choice of the blood vessel to directly measure or extrapolate blood flow detection of depends on the goals of analysis. For example, if the analysis is to determine efficacy of an anticoagulant for the prevention or treatment of deep vein thrombosis, the target blood vessel can be the deep veins in the lower legs, and the flow rate can be approximated through the measurement of blood vessels located closer to the skin surface.

A therapeutic drug, for example an anticoagulant such as a Factor XI inhibitor, is administered to the patient. The administration may be through an oral dosage and the therapeutic drug may include a time release formulation to keep a desired concentration of the bioactive Factor XI inhibitor in the blood stream for a desired duration of time (for example, a single daily dosage) to provide a desired therapeutic effect (for example, prophylaxis against thrombosis).

The preset time is allowed to elapse, for example, to allow for the therapeutic drug to achieve a degree of therapeutic effect and/or for a concentration of the bioactive therapeutic drug to become available in the blood stream.

Sweat stimulation can be activated through iontophoresis of a sweat stimulation chemical into the skin, for accordance with the inventive wearable electronic digital therapeutic device, on-demand sweat stimulation is utilized so that there is an adequate quantity of sweat available at the time when the detection of a particular biomarker is desired even if the patient is sedentary. The sweat contains soluble biomarkers that are detectable through sweat chemistry sensors based on electrical property changes of functional structures that are connected to electrical traces in electrical communication with appropriate amplifying, signal conditioning and signal detecting circuits wider the control of a microprocessor.

As examples of techniques and devices that can be utilized for the formation of sweat chemistry sensors, very small, inexpensive, sweat chemistry sensors can be manufactured using technologies w here all or portions of the traces and/or Functional structures can be nano-printed, for example, using the technique shown in PCT patent application of Busnaina, et al. PCT/US2008/012977, filed 2008 Nov. 21 US patent, or US patent of John J. Daniels, U.S. Pat. No. 7,799,369 B2, issued 2010 Sep. 21.

As a non-limiting example, the sweat chemistry sensor may be functionalized to detect the presence and/or concentration of biomarkers, such as thrombin and/or D-Dimer. The presence of these biomarkers can be indicative of a physiological change that may be the result of a concerning condition. By combining the automatic detection of these biomarkers along with other biometric parameters, probabilistic analysis can be employed to improve the accuracy of determining the effectiveness of a drug and/or electroceutical therapy, enable automatic adjustments of the administered therapy, send alerts to caregivers, patients, family members, etc., provide useful continuously monitor data for drug discovery and other uses of large population data sets, enable adjustments of dosage, timing and other factors determined during pilot studies and clinical trials, and a number of other advantageous applications of the inventive wearable electronic digital therapeutic device.

As an example, in the case of thrombin, in the coagulation cascade, coagulation factor II is proteolytically cleaved to form thrombin in the first step of the coagulation cascade which ultimately results in the stemming of blood loss. F2 also plays a role in maintaining vascular integrity during development and postnatal life. Mutations in F2 leads to various forms of thrombosis and dysprothrombinemia. Therefore, the detection of the thrombin biomarker along with other biometric parameters such as blood flow through a target blood vessel (and other biomarkers and biometrics described herein and/or currently or in the future employed tor human or animal healthcare) is utilized in accordance with the inventive wearable electronic digital therapeutic as an enhanced tool for health and wellness of the individual and for the global population After the on-demand sweat stimulation results in sweat availability, a subsequent level of the biomarker (e.g., thrombin) is again detected.

FIG. 123 illustrates constituent parts of a system for remotely monitoring and controlling a wearable electronic digital therapeutic device and illustrates a wearable electronic having a sweat stimulator/collector and block diagram of electronics. This example of the inventive wearable electronic digital therapeutic garment can be constructed as a convenient configuration that is comfortable, washable and is the same general construction of a garment that is typically worn nearly every day by patients and healthy members of the human population, A sweat chemistry sensor is shown. However, as with all the described embodiments, this sensor can be any one or more of the various biometric parameter detectors described herein or available currently or through advancements in enabling technologies.

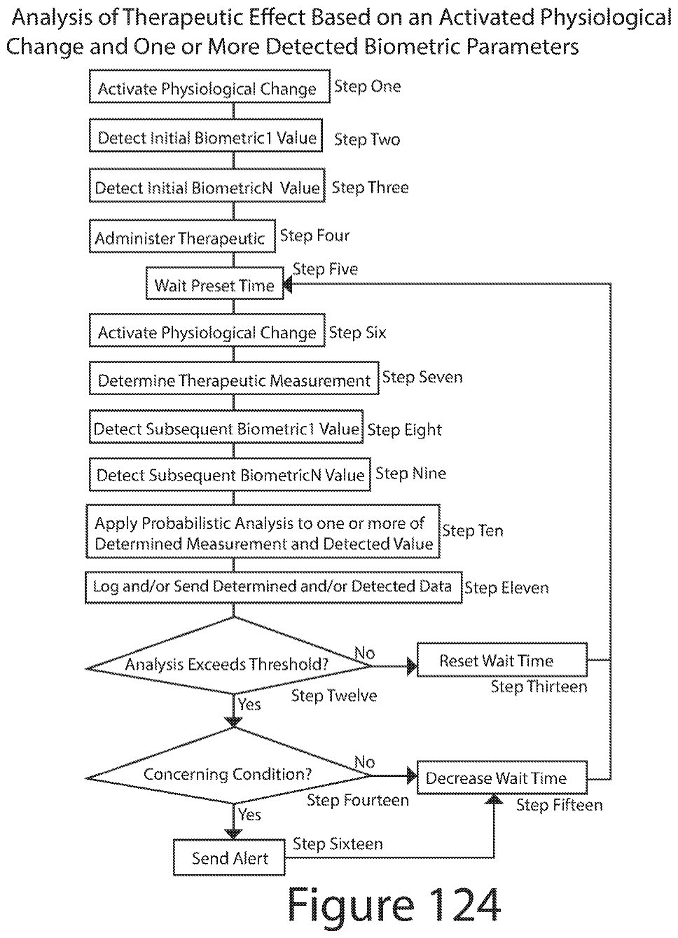

FIG. 124 is a flow chart illustrating an algorithm for analyzing a therapeutic effect based on an activated physiological change and detected biometric parameters A physiological change, such as simulating sweat, is activated (Step One). An initial biometric1 value is detected (Step Two) and additional biometric values up to initial biometricN value is detected (Step Three). A therapeutic is administered (Step Four). A preset wait time is allowed to elapse (Step Five) and then the physiological change is again activated (Step Six). A therapeutic measurement is determined (Step Seven), such as determining a concentration of a metabolite of an administered therapeutic medicinal compound or a change in blood flow through a vein in response to the administered therapeutic compound Subsequent Biometric1 through BiometricN values are detected (Step Eight. Step Nine). Probabilistic analysis is applied to one or more of the determined therapeutic measurement and the detected biometric values (Step Ten).

The determined and/or detected data is logged in onboard memory and/or on a remote server and/or sent to a trusted receiver (Step Eleven). It is determined the analysis indicates that a threshold has been exceeded (Step Twelve) For example, the threshold can be a change in blood flow indicating a poor response to the administered therapeutic. If the threshold is not exceeded, then the wait time is reset (Step Thirteen) and the preset wait time is allowed to elapse (Step Five) before continuing to activate the physiological change again (Step Six).

If the threshold is exceeded (Step Twelve) than it is determined if a concerning condition is present (Step Fourteen). For example, the biometric value detection and the determined therapeutic measurement may indicate that the dosage of the administered therapeutic is inadequate to maintain sufficient and/or improving blood flow. If the concerning condition is determined (Step Fourteen) an alert is sent and the wait time is decreased so that the physiological change is activated sooner (Steps Five and Six). Even if the concerning condition is not determined (Step Fourteen), since the threshold has been exceeded (Step Twelve), the wait time can still be decreased to that the activation, detection and analysis cycle is done sooner.

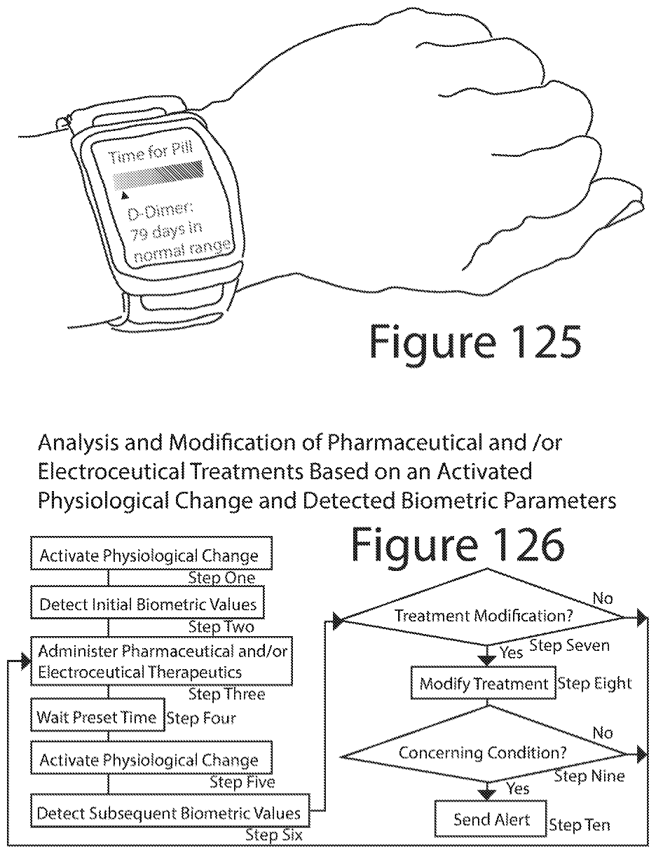

FIG. 125 illustrates an embodiment configured as a wristwatch, bracelet, sleeve or armband. A wearable electronic detects patient adherence to drug taking schedule and sends w ireless confirmation or alert to trusted receiver. A chemical analyte, which is preferably a metabolite of an administered drug, is detected by a wearable electronic to confirm patient adherence. The wearable electronic includes an on-demand sweat stimulator enabling the production of sweat only when needed for the detection of the chemical analyte. If the metabolite is the chemical analyte, no changes to the drug as presently packaged or new drug regulatory issues may be present. A water-soluble metabolite may be effective for detection via the inventive on-demand sweat stimulator and sweat chemistry sensor.

For cardiovascular use, this same system can be used for detecting biomarkers, such as d-dimer and other protein/components present in the coagulation cascade. The form-factor could be a simple bracelet, anklet or even a smartwatch with activity tracking and display interface enabling additional important Dr./Patient engagement advantages. A wireless signal is sent over the Internet to alert caregiver, hospital, insurer, drug company, family member or other trusted receiver.

As an example, a metabolite of an administered drug is detected to confirm patient adherence. An on-demand sweat stimulator is used to stimulate sweat when needed for the detection of the metabolite. A w ireless signal is sent over the Internet to alert caregiver, hospital, insurer, drug company, family member or other trusted receiver. The inventive wearable electronic is a non-invasive, positive indication of ingestion of an administered drug with wireless signal sent to caregiver or other misted receiver confirming patient adherence.

FIG. 126 is a flow chart illustrating an algorithm for modifying a combination of pharmaceutical and electroceutical treatments based on an activated physiological change and detected biometric parameters. A physiological change is activated (Step One) and initial biometric values are detected (Step Two). A pharmaceutical and/or electroceutical therapeutic is administered (Step Throe) and a preset time is allowed to elapse (Step Five). The physiological change is activated again (Step Five) to enable the detection of subsequent biometric values (Step Six) and it is determined based on the detected biometric values (e.g., by comparison and/or exceeding a predetermined threshold) if the therapeutic treatment should be modified (Step Seven). If the treatment does not require modification, the pharmaceutical and/or electroceutical therapeutic is again administered (Step Three). If the treatment should be modified (Step Seven) then the treatment is modified (Step Fight) and it is determined if there is a concerning condition present (Step Nine). If there isn't a concerning condition (Step Nine) then the cycle begins again and a physiological change is activated (Step Five). If there is a concerning condition (Step Nine) then an alert is sent (Step Ten).

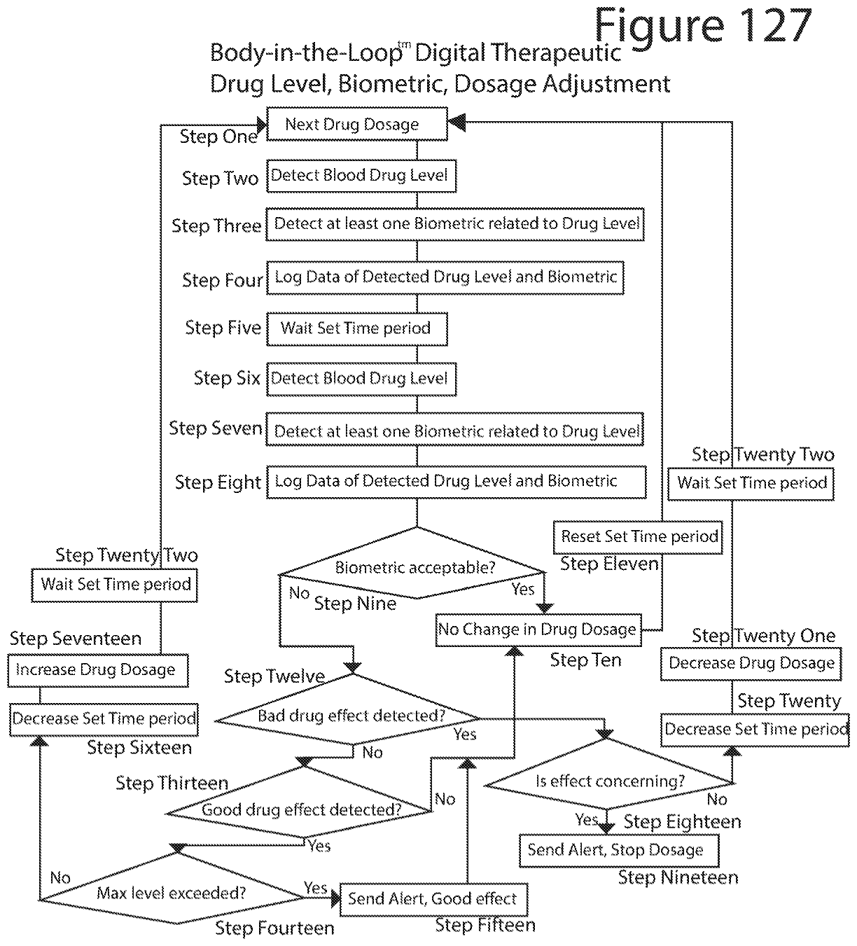

FIG. 127 is a flow chart illustrating an algorithm for using a Body-in-the-Loop™ digital therapeutic for dosage adjustment based on detected in-vivo drug levels and biometric parameter. A scheduled drug dosage is administered (Step One) and a blood drug level is determined (Step Two). At least one biometric related to the drug level is detected (Step Three) and the detected drug level and biometric is logged and/or transmitted (Step Four), A set time period is allowed to elapse (Step Five) and the blood drug level is detected again (Step Six). At least one biometric (elated to the blood drug level is detected (Step Eight) and the data is again logged and/or transmitted (Step Eight). If the biometric is determined to be acceptable (Step Nine) then the ding dosage is not changed (Step Ten), the set time period is reset (Step Eleven) and process flow continues back to another drug dosage without change from the last dosage (Step One), and then the detection of the blood drug level (Step Two) and of at least one biometric (Step Three).

If the biometric is not acceptable (Step Nine), then it is determined if a bad drug effect has been detected (Step Twelve), if it has then it is determined if the effect is concerning (Step Eighteen) and if it is, an alert is sent and the dosage is stopped (Step Nineteen). If the effect is not concerning (Step Eighteen) then since a bad drug effect has been detected (Step Twelve) the set time period is decreased (Step Twenty) and the next drug dosage is decreased (Step Twenty-one). The decreased set time period is allowed to elapse (Step Twenty-two) and process flow continues back to another drug dosage that has been decreased from the last dosage (Step One) and then the detection of the blood drug level (Step Two) and of at least one biometric (Step Three). Note that at Step One, the increase or decrease in the drug dosage can be an increase or decrease in the scheduled drug dosage frequency, and if the next drug dosage is not scheduled, then no drug is administered before process flow continues to detect the next blood drug level (Step Two).

If a bad drug effect is not detected (Step Twelve), it is determined if the biometric related to the blood drug level indicates that a good drug effect has been detected (Step Thirteen). If a good drug effect has been detected, that is it determined if a maximum level of the blood drug has been exceeded (Step Fourteen), if it has, and alert is sent indicating the good effect (Step Fifteen) and the process flow continues to Step Ten.

If the maximum level has not been exceeded (Step Fourteen), then the set time period is decreased (Step Sixteen) and the drug dosage is increased (Step Seventeen), then the set time period is allowed to elapse (Step Twenty-Two) before the process continues on to the next scheduled drug dosage (Step One). Note that if no drug dosage is scheduled, flow can continue right to Step Two so that periodic or continuous monitoring of the patient occurs depending on the changes to the set time period.

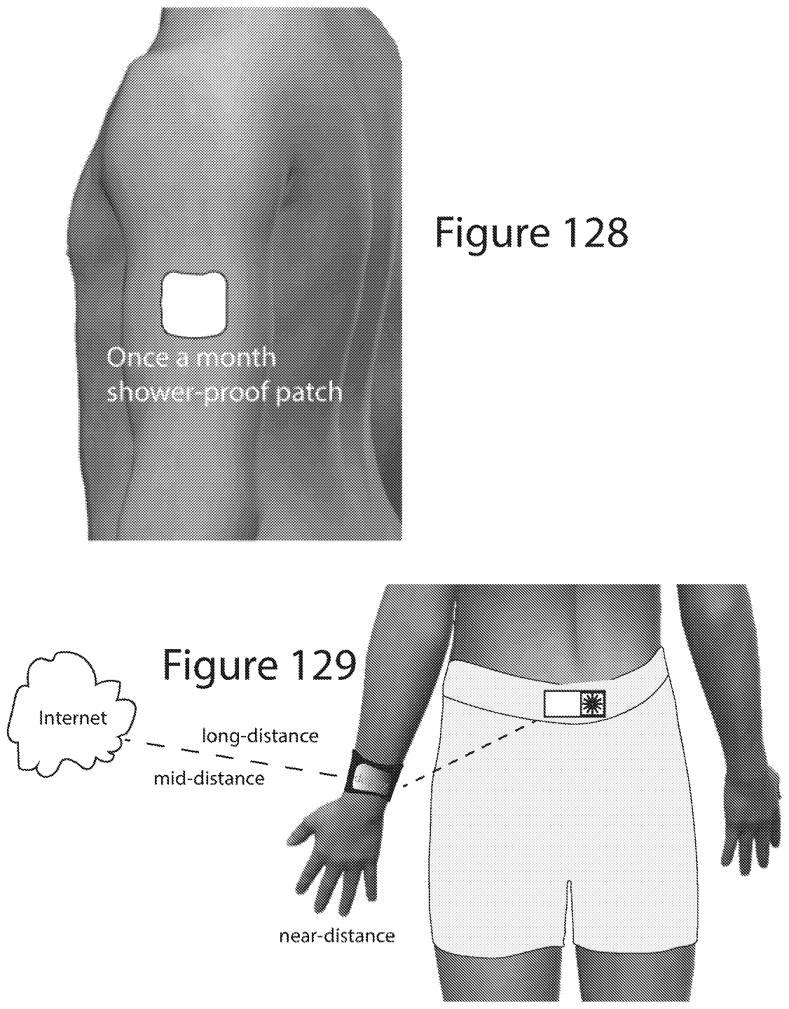

FIG. 128 illustrates a patch configuration of an embodiment. The patch, as with any of the wearable electronic configurations described herein, can include microprocessor(s), memory, battery, signal generation, activation, communication, signal detection and other components possibly configured as flexible and/or stretchable circuitry. The patch can disposable and/or have components such as batteries that are inductively rechargeable and reservoirs that are refillable.

FIG. 129 illustrates a multi-part configuration having a printed electronics flexible display with near-distance and mid/long-distance relayed wireless communications of an embodiment. A thin, lightweight, printed electronic circuit and display can be constructed, for example, using techniques described in US Patent Publication No. US20090176029A1, entitled Printer and Method for Manufacturing Electronic Circuits and Displays, inventor Daniels, filed 4 Sep. 2002, the disclosure of which is incorporated by reference herein in its entirety.

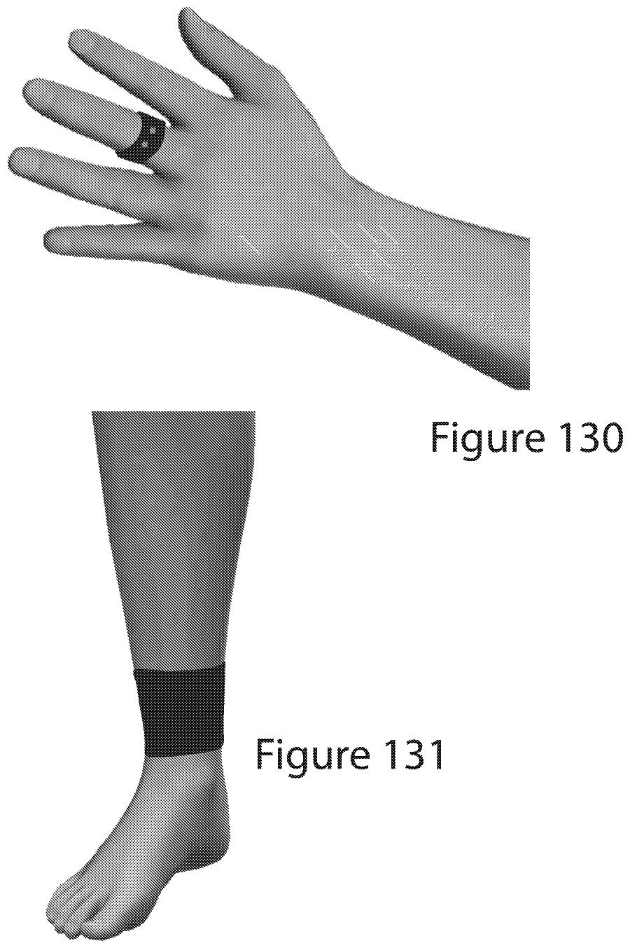

FIG. 130 illustrates a ring configuration of an embodiment. One or more RGB LEDS (or other illumination source) can be used to convey messages to the patient and/or for optically coupling to a wireless relay as a power savings feature. FIG. 131 illustrates an anklet configuration of an embodiment.

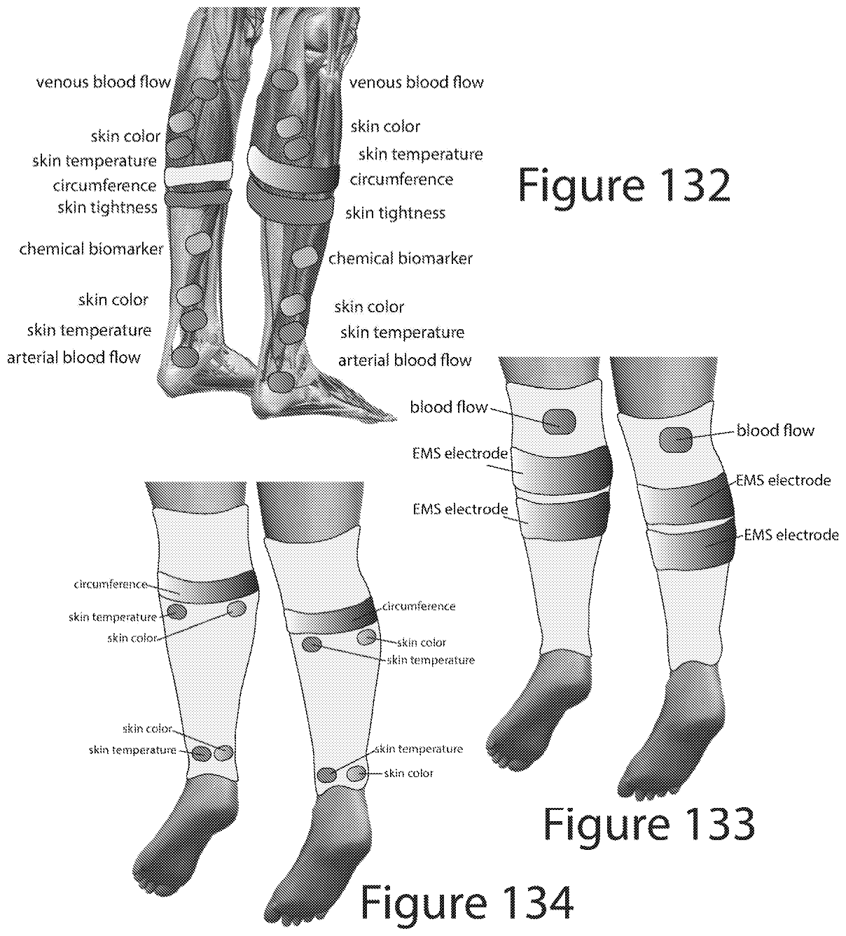

FIG. 132 illustrates the location of various biometric detectors/sensors/transmitters/processors/actuators. The analysis can be made of a therapeutic effect based on an activated physiological change and one or more detected biometric parameters. For example, the activated physiological change can be the on-demand stimulation of sweat. One detected biometric parameter can be the detection of a coagulation cascade factor present in the sweat. Another detected biometric parameter may be the detection of blood flow through blood vessels in a body part, such as the legs. Another detected biometric parameter may be a change in the circumference of the leg caused by edema. Other biometric parameters are listed and described elsewhere. A sweat chemistry detector can be included for determining a concentration of a coagulation cascade factor in the blood, a blood gas component, or other soluble molecule or small particulate. In accordance with an exemplary embodiment, the therapeutic effect may be the result of an administered therapeutic, such as a pharmaceutical therapy, e.g., an anticoagulant and/or an electroceutical therapy, e.g., EMS applied to activate the muscle pump. In addition, or as an alternative, to analyzing a therapeutic effect, disease progression or modification can be determined by monitoring the one or more detected biometric parameters.

The choice of the one or more detected biometric parameters may depend on the physiological condition, disease, fitness level, treatment being monitored, or other use case for the analysis of the therapeutic effect. Biometric parameters can be detected as an alternative or in addition to the ones described herein. For example, the biometric detection of biomarkers, such as thrombin and/or d-dimer, may be used for treatment and monitoring of conditions related to the contact system for coagulation and inflammation.

In accordance with an embodiment, the blood flow information obtained by a blood flow detector can be used to detect a change in blood viscosity that may indicate a concerning condition such as thickening of the blood due to thrombotic conditions. The volume of blood detected and blood flow measurements can be used, along with, for example, the relative concentration of red blood cells compared to other blood constituents to check for a change in viscosity due to a thrombotic event as opposed to a change in viscosity due to a change in hydration (e.g., if the patient consumes water and/or receives an intravenous saline drip). Since the viscosity will change depending on hydration, a detected change in blood viscosity might not indicate thickening or thinning of blood due to coagulation tendency changes. By detecting a value for the concentration of red blood cells (for example, by detecting a change in reflected red light from the blood vessel) and normalizing for a given volume, then it is possible to calculate a value compared to a threshold (which may be obtained from a baseline patient reading) to indicate that the blood is becoming more or less stickier or thicker not due to hydration changes but instead the presence of coagulation factors.

FIG. 133 illustrates an embodiment of a pharmaceutical/electroceutical combination treatment device for applying an electroceutical signal in combination with an administered pharmaceutical and detecting a biometric physiological response. EMS electrodes apply electrical signals to the body. The same electrodes can also be used as EMG electrodes to detect electrodes from the body. An Electronic Circuit is connected with the Wearable Electronic Garment. Depending on the intended use, the EMS electrodes can be used for EMG or other signal detection so that bi-directional electrical signals are applied through a plurality of individually addressable electrodes routed through an electrode multiplex circuit and a signal multiplex circuit for applying a sequential EMS signal and detecting biometric feedback, for example, from the calf of a patient. In accordance with an aspect of the invention, a digital therapeutic device garment is provided with a plurality of individually addressable electrodes supported by the garment for applying a sequential EMS signal and detecting biometric feedback from the calf of a patient. The individually addressable electrodes are for at least one of applying stimulation electrical signals to the skin of a patient and detecting biometric electrical signals from the skin of the patient. At least one of a signal detector for detecting the biometric electrical signals and a signal generator for generating the stimulation electrical signals are provided. An electrode multiplex circuit addresses the plurality of individually addressable electrodes by at least one of routing the biometric electrical signals from the skin of the patient through more than one of the plurality of individually addressable electrodes to the signal detector and routing the stimulation electrical signals from the signal generator through more than one of the plurality of individually addressable electrode to the skin of the patient. A microprocessor controls the signal detector, the signal generator, the electrode multiplex circuit and other circuit components.

The microprocessor can control the electrode multiplex circuit to route the biometric electrical signals from the skin of the patient sequentially through more than one of the plurality of individually addressable electrodes to the signal detector. In accordance with this embodiment, a single EMS signal source can service multiple individually addressable electrodes with the EMS signal routed as desired for an intended therapy, such as for the sequential squeezing of the deep veins in the legs to promote blood flow in the direction back to the heart. One or more EMS signal channels can be multiplexed and signals routed so that even a large array of individually addressable electrodes can be serviced by one or a few signal generators, for example, to provide a finer spatial resolution of the applied EMS signal than indicated by number of electrodes shown in the drawings.

The microprocessor can control the electrode multiplex circuit to mute the biometric electrical signals (indicating, for example, muscle activity, heartbeat, etc.) from the skin of the patient simultaneously through more than one of the plurality of individually addressable electrodes to the signal detector. The microprocessor can control the electrode multiplex circuit to route the stimulation electrical signals from the signal generator simultaneously through more than one of the plurality of individually addressable electrodes to the skin of the patient. The microprocessor can control the electrode multiplex circuit to route the stimulation electrical signals from the signal generator sequentially through more than one of the plurality of individually addressable electrodes to the skin of the patient. The microprocessor can control the electrode multiplex circuit to route the stimulation electrical signals from the signal generator simultaneously through more than one of the plurality of individually addressable electrodes to the skin of the patient.

FIG. 134 illustrates an embodiment of a pharmaceutical/electroceutical combination treatment device for monitoring physiological changes in response to an administered pharmaceutical and/or electroceutical treatment. The biometric parameters may include a stain gauge formed from an elastic resistance strip that reversibly changes a detectable resistance value based on being stretched.

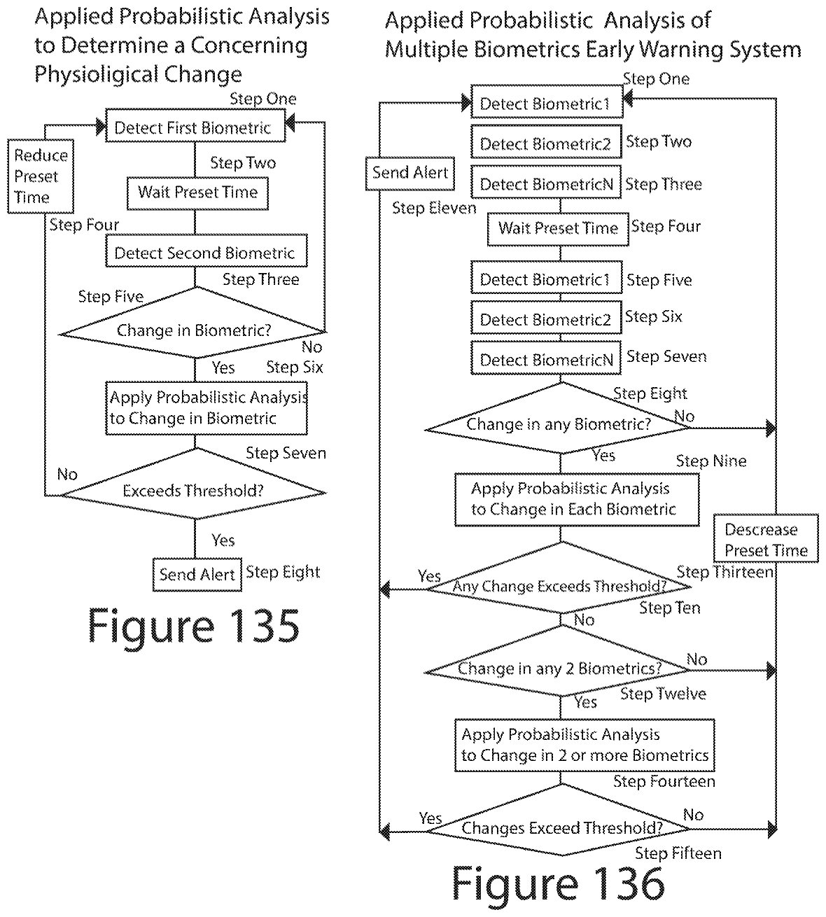

FIG. 135 is a flow chart illustrating an algorithm for applied probabilistic analysis to determine a concerning physiological change. As a simple, low cost early wearable electronic warning system, a first biometric value can be compared to a second biometric value to note a physiological change indicating that an at-risk patient may be undergoing a concerning condition, such as a thrombotic event. A patch, ring, bracelet, anklet, sock, belt, or other wearable electronic can be constructed to automatically detect one or more biometric parameters indicative of a physiological change indicating the start of a concerning condition detectable at a body part such as the legs, wrist, foot, torso, neck, etc.

A first biometric reading is taken (Step One). After a preset time (Step Two) a second biometric reading is taken (Step Three). The biometric readings are compared to see if there has been an change in the biometric reading occurring over time (Step Four). Probabilistic analysis is applied to determine if the change exceeds a threshold (Step Five) and if so, then an alert is sent (Step Six). If the change does not exceed a threshold (Step Six) then the present time can be reduced (optionally, Step Seven) so that the biometric readings are detected and compared sooner (Steps One, Two and Three). Any of the sensor, detectors, and/or biometric and biomarkers described herein or others available or detected but not specified can be utilized as the detected biometric described in this flow chart and other flowcharts discussed herein.

FIG. 136 is a flow chart illustrating an algorithm for an early warning system with applied probabilistic analysis of multiple biometric parameters. A first biometric, biometric2 through biometricN readings are taken (Step One, Two, Three). After a preset time (Step Four) a second biometric1, biometric2 through biometricN readings are taken (Step Five, Six, Seven). If there has been a change in any of the detected biometrics over the preset time (Step Fight), then probabilistic analysis is applied to the detected change in each biometric (Step Niue) If any analyzed change exceeds a threshold, which is predetermined or calculated for each type of biometric reading (Step Ten), then an alert is sent (Step Eleven) and process flow returns to detect the biometric1, biometric2 through biometricN readings (Step One, etc.). If the change threshold of none of the detected biometries has been exceeded (Step Ten), then it is determined if there was a change in two or biometrics over the preset time (Step Twelve). If there w as a change in two or more biometrics (Step Twelve) then probabilistic analysis is applied to the change in the the two or more biometries (Step Fourteen) and if that analysis indicates that the changes exceed a threshold (Step Fifteen), and if so, then an alert is sent (Step Eleven) and process flow continues to Step One. If there was not a change in any biometric over the preset time (Step Eight), then process flow continues to detect the biometric1, biometic2 through biometricN again (Steps One, Two, Three) If there wasn't a change in two or more biometries (Step Twelve) or the analyzed changes did not exceed the threshold (Step Fifteen), then to increase detection frequency or to conserve battery power, consumables such as sweat stimulation chemicals, and data collection memory and transmission, the preset time can be changed (Step Thirteen) depending on a desired increase or decrease in detection, etc., and process flow continues to Step One. Any of the sensor, detectors, and/or biometric and biomarkers described herein or others available or detected but not specified can be utilized as the detected biometric described in this flow chart and other flowcharts discussed herein.

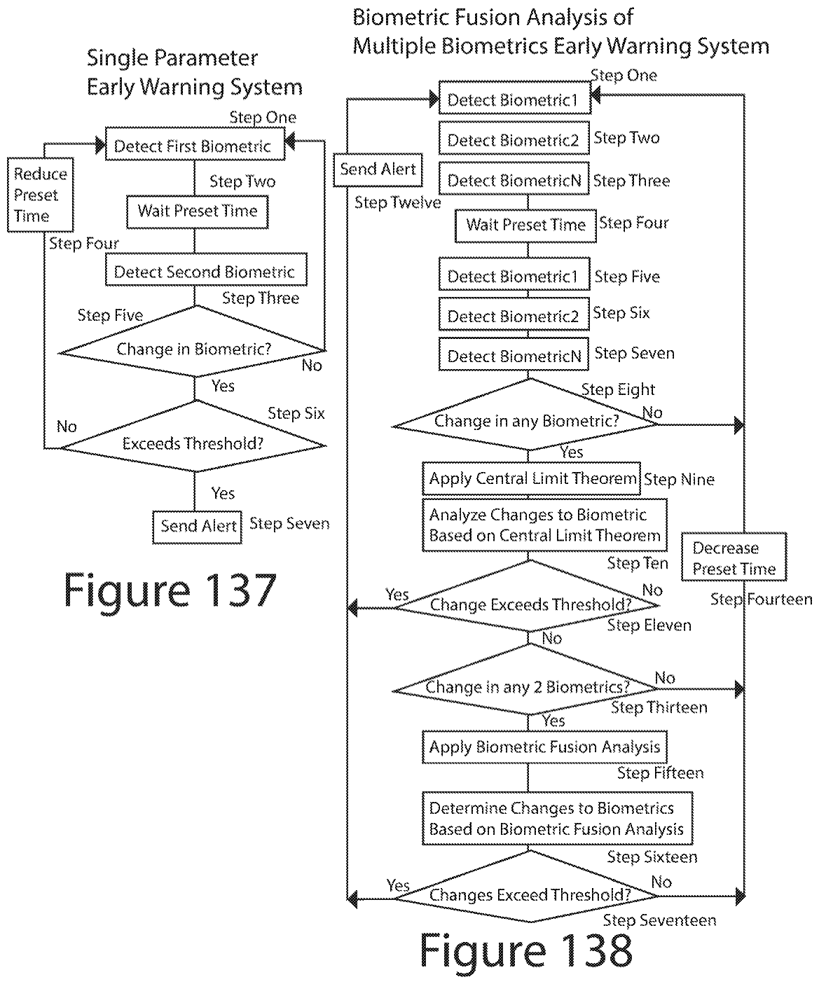

FIG. 137 is a flow chart illustrating an algorithm for a single parameter early warning system. As a simple, low cost wearable electronic early warning system, a first biometric value can be compared to a second biometric value to note a physiological change indicating that an at-risk patient may be undergoing a concerning condition, such as a thrombotic event. A patch, ring, bracelet, anklet, sock, belt, or other wearable electronic can be constructed to automatically detect one or more biometric parameters indicative of a physiological change indicating the start of a concerning condition detectable at a body part such as the legs, wrist, foot, torso, neck, etc.

A first biometric reading is taken (Step One). After a preset time (Step Two) a second biometric leading is taken (Step Three). The biometric readings are compared to sec if there has been an change in the biometric leading occurring over time (Step Four), if the change exceeds a threshold (Step Five) then an alert is sent (Step Six). If the change does not exceed a threshold (Step Six) then the present time can be reduced (optionally, Step Seven) so that the biometric readings are detected and compared sooner (Steps One. Two and Three). Any of the sensors, detectors, and/or biometric and biomarkers described herein or others available or detected but not specified can be utilized to obtain the detected biometric described in this flow chart and other flowcharts discussed herein.

FIG. 138 is a flow chart illustrating an algorithm for biometric fusion analysis of multiple biometries to determine a physiological change. Biometric fusion is the use of multiple types of biometric data, or methods of processing, to improve the performance of biometric systems. One type of fusion is score-level fusion, which is the combination of matcher scores to improve accuracy. The scores used in fusion can be obtained through the use of multiple types of data for each subject and is typically applied for identification (such as face and fingerprint, or fingerprints from different fingers), multiple samples from each subject, multiple matchers on a single type of data or combinations of these. In accordance with the inventive wearable electronic digital therapeutic device and applications described herein, biometric fusion analysis is used to improve the accuracy and effectiveness of using detected biometries for patient therapeutic, monitoring and diagnostic applications.

A first biometric1, biometric2 through biometricN readings are taken (Step One, Two, Three). After a preset time (Step Four) a second biometric1, biometric2 through biometricN readings are taken (Step Five, Six. Seven). If there has been a change in any of the detected biometries over the preset time (Step Eight), then probabilistic analysis, such as an application of the central limit theorem or other statistical analytic model, is applied (Step Nine) to analyze the detected change in each biometric (Step Ten). If the analyzed change exceeds a threshold, which is predetermined or calculated for each type of biometric reading (Step Eleven), then an alert is sent (Step Twelve) and process flow returns to detect the biometric1, biometic2 through biometricN readings (Step One, etc.) If the change threshold of none of the detected biometrics has been exceeded (Step Eleven), then it is determined if there was a change in two or biometries over the preset time (Step Thirteen). If there was a change in two or more biometries (Step Thirteen) then probabilistic analysis, such as biometric fusion analysis, is applied to the change in the two or more biometrics (Step Fifteen) and changes to biometries based on the biometric fusion analysis are determined (Step Sixteen).

If that analysis indicates that the changes exceed a threshold (Step Seven), and if so, then an alert is sent (Step Twelve) and process flow continues to Step One. If there was not a change in any biometric over the preset time (Step Fight), then process flow continues to detect the biometric1, biometric2 through biometricN again (Steps One, Two, Three). If there wasn't a change that exceeds the thresholds (Step Eleven) (Step Seventeen), then to conserve batter power, consumables such as sweat stimulation chemicals, and data collection memory and transmission, the preset time can be decreased (Step Fourteen) and process flow continues to Step One. Any of the sensor, detectors, and/or biometric and biomarkers described herein or others available or detected but not specified can be utilized as the detected biometric described in this flow chart and other flowcharts discussed herein.

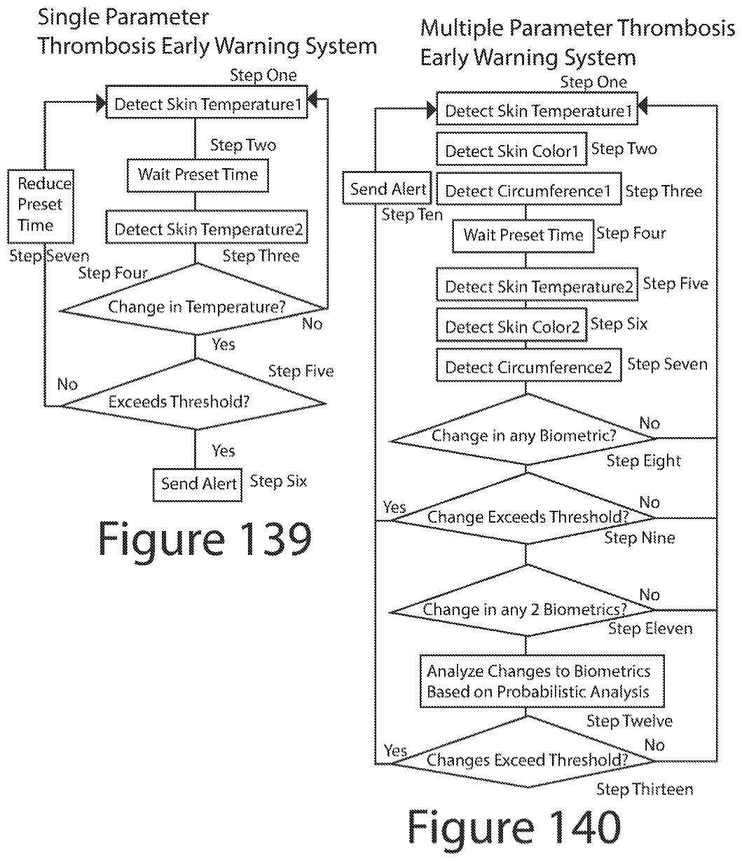

FIG. 139 is a flow chart illustrating an algorithm a single parameter thrombosis early warning system. As a simple, low cost early warning system, skin temperature at two or mom locations on the same leg, oi on both legs, can be detected and compared to note changes in skin temperatures indicating that an at-risk patient may be undergoing a thrombotic event. Wearable electronic stockings, for example, can be made comfortable, washable and easy to wear and automatically detect ox or more biometric parameters indicative of a physiological change indicating the start of a concerning cardiovascular condition detectable at the lower legs. (NOTE: can be configured tor other body pails as well).

A skin temperature1 reading is taken (which may be multiple readings at different locations on the same leg and/or both legs). After a preset time (Step Two) a skin temperature2 reading is taken (Step Three). The skin temperature readings are computed to see if there has been a change in temperature (Step Four), if the change exceeds a threshold (Step Five) then an alert is sent (Step Six). If the change docs not exceed a threshold (Step Six) then the present time can be reduced (Step Seven) so that the Skin Temperature1 is detected and compared sooner (Steps One. Two and Three).

FIG. 140 is a flow chart illustrating an algorithm a multiple parameter thrombosis early warning system A wearable electronic therapeutic device has one or more biometric detectors each for detecting one or more biometric parameters. The skin temperature1 value is detected (Step One), skin color1 value is detected (Step Two) and Circumference1 is detected (Step Throe). These initial parametric values are compared with later obtained values where the biometric parameters are dependent on at least one physiological change to a patient in response to a therapeutic treatment. After waiting a preset time (Step Four), parametric values are again detected (Steps Five-Seven). A microprocessor receives the one or more biometric parameters. If there hasn't been a change in any of the biometric parameters (Step Eight) then the loop repeats and the skin temperature1 value is again detected (Step One). If there has been a change in any of the biometric parameters (Step (right), the microprocessor applies probabilistic analysis to determine if at least one physiological change threshold has been exceeded (Step Nine). The probabilistic analysis and exceeding the threshold can be based on only one of the biometric parameters, for example, by noting a fast-changing condition caused by the swelling of the leg. If the threshold has not been exceeded, then the loop repeats and the skin temperature1 value is again detected (Step One), in addition, or alternatively, if there is a change in two or more biometries (Step Eleven), the biometrics are analyzed based on probabilistic analysis of the two or more biometrics (Step Twelve) and lire exceeding of the threshold may be dependent on the probabilistic analysis of the two or more biometric parameters (Step Thirteen). If the threshold has not been exceeded, then the loop repeats and the skin temperature1 value is again detected (Step One). However, in steps nine or thirteen, if the threshold is exceeded an alert can be sent (Step Ten) before beginning the loop again at step one. The alert can be sent by an activation circuit that activates an action depending on the determined exceeded physiological change. In addition to, or as an alternative, to sending an alert the action that is activated can be dependent on an applied pharmaceutical treatment, for example, activating the applying of an electroceutical treatment in addition or as an alternative to a pharmaceutical treatment. As a non-limiting example, the pharmaceutical treatment may be an anticoagulant drug taken by an at-risk patient. In this ease, the detected biometric parameters may indicate an alert should be sent, anchor an electroceutical treatment should be applied.

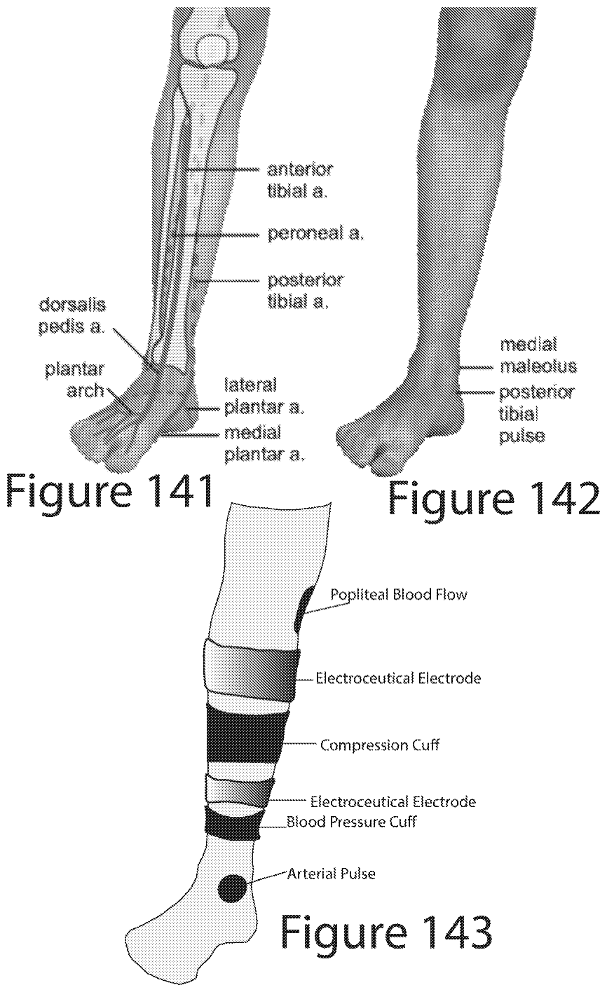

FIG. 141 illustrates lire location of blood vessels in the lower leg FIG. 142 illustrates the location of blood pulse/vessels under the skin surface of the lower leg. FIG. 143 illustrates a wearable electronic digital therapeutic device configured as a stocking for detecting biometric parameters including blood pressure using a blood pressure cuff and for using the pressure cuff for applying a compressive therapeutic action in combination with electroceutical and/or pharmaceutical therapies A stocking configuration of the inventive wearable electronics can be used for monitoring at-risk PAD patients using base-line measurements obtained from a doctor's visit or at the hospital of the ankle-brachial index (ABI) and blood flow at the ABI measurement points. The base-line ABI ratio and blood flow measurements are then compared to real-time measured blood flow to later determine if a change in PAD conditions is occurring.

Research literature supports the premise of this simple PAD monitoring as an early warning system for at-risk PAD patients. As an example biometric detector, a blood flow sensor from Kyocera can detect changes in blood flow A clinical ABI measurement is easy and routine and is likely already being done to determine if a patient has or is at-risk of PAD. The blood flow through a vessel is proportional to the blood pressure in that vessel, so blood flow measurements subsequent to ABI measurement are useful biometric parameters for determining a subsequent change in the ABI ratio. The use of low-cost blood flow sensors enables a low cost, easy to wear product to be made. Mathematical (Blood pressure and blood flow variation during postural change from sitting to standing: model development and validation, Mette S. Olufsen et al. J Appl Physiol. 2005 October, 99(4); 1523-1537) and software (Measurement and Interpretation of the Ankle-Brachial Index A Scientific Statement From the American Heart Association, Victor Aboyans et al., 16 Nov. 2012 Circulation. 2012; 126: 2890-2909) blood circulation models exist and are refined into algorithms for detection and alert of concerning changes in PAD conditions of an at-risk patient.

The ankle-brachial index (ABI) is the ratio of the systolic blood pressure (SBP) measured at the ankle to that measured at the brachial artery. ABI is an indicator of atherosclerosis at other vascular sites and can serve as a prognostic marker for cardiovascular events and functional impairment, even in the absence of symptoms of PAD. Because the ABI is a ratio, it's use in accordance with the inventive wearable electronic digital therapeutic device may not be affected by factors that raise or lower blood pressure. For example, changes in blood volume after hemodialysis do not alter the ABI, despite significant removal of fluid and reduction in blood pressure.

That is, once there is a base-line ABI measurement, tor the use described herein, there should be no problematic relative change in the blood pressure measurement at the ankle and brachial, the ABI should remain relatively the same if the condition of PAD is unchanged, liven if the patient has a different blood pressure (due to change in blood volume or exercise) from one ABI test to the next ABI test, the ABI ratio w ill remain the same for the described use if there has been no change in the condition of PAD.

Since the blood flow through a vessel is proportional to the blood pressure in that vessel, the blood flow measurements subsequent to the clinical ABI and blood flow baseline can be used as detected biometric parameters for extrapolating a change in the ABI ratio indicating a change in the PAD condition. Stated otherwise, the wearable electronic for monitoring at-risk PAD patients will not require the complexity, batten consumption, bulk and obtrusiveness of a mobile blood pressure cuff that replicates the clinical measurements taken for the ABI ratio, rather, the baseline ABI and blood pressure measurements are correlated to constantly monitored, changes in blood flow at the measurement points.

An embodiment of the wearable electronics and algorithm uses real-time biometries of PAD symptoms such as edema, skin temperature and skin color, along with the blood flow measurements compared with the base-line measurements to accurately determine changing PAD conditions have exceed a predetermined threshold, triggering a wireless alert sent to a caregiver, family member or other trusted receiver.

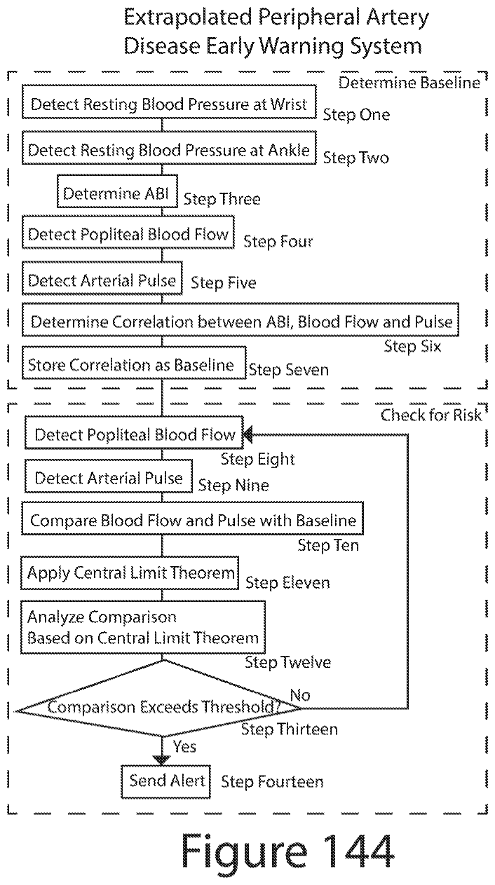

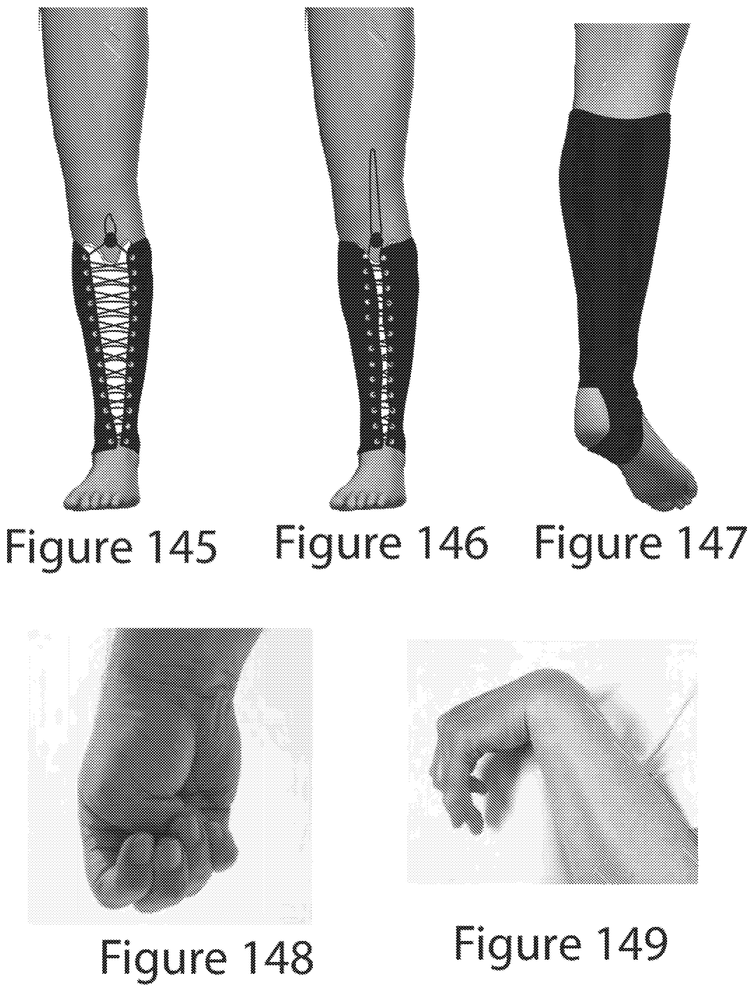

FIG. 144 is a flow chart illustrating an algorithm for extrapolating one or more biometric parameter for detecting a physiological change A baseline, resting blood pressure is detected at the wrist or arm (brachial) (Step One) and at the ankle (Step Two) to determine the baseline ABI (Step Three). Blood How (Step Four) and pulse (Step Five) are detected and a correlation is determined between ABI. Blood Flow and Pulse (Step Six). For example, since the blood flow through a vessel is proportional to the blood pressure in that vessel, the blood flow measurements and/or the pulse measurements should correlate with the ABI as baseline readings that can be used as detected biometric parameters for determine a change in the ABI ratio indicating a change in the PAD condition. Tins correlation may be stored as a baseline (Step Seven).

To check for PAD risk, the blood flow (Step Fight) and pulse (Step Nine) are subsequently detected and compared with the baseline (Step Ten). A statistical analysis, such as related to the central limit theorem can be applied (Step Eleven) to enable analysis of the comparison (Step Twelve) so that a statistically meaningful decision can be made to determine if the comparison exceeds a threshold (Step Thirteen) where the threshold indicates that an at-risk patient may have a change in physiological conditions indicating a change in peripheral artery disease risk causing an alert to be sent (Step Fourteen). If the comparison threshold is not exceeded, then the process flow returns to detect subsequent biometries to compare with the correlated baseline (Steps Fight through Fourteen).

An embodiment of the inventive wearable electronic digital therapeutic device utilizes probabilistic data quality improvement using statistical techniques such as the central limit theorem and sensor fusion, combined with cloud-based and/or local-embedded artificial intelligence.

As a non-limiting example of a probabilistic analysis methodology that is applicable to the inventive wearable electronic digital therapeutic device, the Central Limit Theorem is a statistical theory that states that the mean value of all samples taken from a particular population will be approximately equal to the mean of the population, provided a sufficiently large sample size is taken from a population with a finite level of variance. With increasing sample size, the resulting distribution of mean values (and related error bounds) begins to approach a normal distribution. As a related statistical analysis technique. Sensor Fusion uses sensors designed to measure disparate, but correlated data in order to deterministically capture relevant information from diverse data Embedded AI (Artificial Intelligence) is a method of employing computing capability local to the source of data collection, typically for the purposes of providing a closed loop actuation based on derived information.

When attempting to measure data using sensors, one is always concerned with data quality and ultimately with data utility. One key element associated with assuring data quality is the ability to capture and filter data outliers through outlier detection and sensor fusion. For example, in the case of the inventive wearable electronic digital therapeutic device, the data collected from various biometric detectors may include outliers that come into the data set because of sudden patient movement (getting up quickly from a sealed position), rapid environmental changes (stepping outside into the cold), temporary disruption in the connection between the sensor and body (repositioning of the wearable electronic), etc.

Various methods exist to assist with tightening up a band of data sets, including the employment of various statistical analysis methods and cross correlation techniques. In accordance with an exemplary embodiment, the Central Limit Theorem is coupled with data cross correlation relationships to improve data accuracy.

By combining the CLT with another methodology called “sensor fusion” this exemplary embodiment has the ability to produce higher data accuracy results while capturing raw data using lower quality (lower priced) sensors. (Salma. B., Malathi. P., Arokia, P., An Efficient Data Fusion Architecture for Location Estimation. International Journal of Engineering Research and Technology (IJERT)., ISSN No: 2278-0181, Vol. 3 Issue 1, January 2014) Sensor fusion is a process of incorporating a range of different sensors into a data capture process so as to more accurately bound the data output. To illustrate by way of example, a simple temperature sensor placed in a room has no way of “knowing” why a sudden temperature spike is measured. However, coupling this temperature sensor with motion sensors and/or light, vibration, sound sensors, might conclusively determine that a large crowd of people have entered the room, or a window has been opened and so on By combining these disparate detection devices, and aggregating data over time, and employing predictive algorithms, greater data accuracy can be obtained. The result is more useful and accurate information is generated.

In accordance with the exemplary embodiment, the CLT is coupled to data correlation relationships so that the mean values of error bands approach a normal distribution, provided the sample size is sufficiently large. As another illustrative example, test engineers routinely install instrumentation in the hot exhaust gas plume generated by gas turbine engines while operating in a test chamber. The most accurate measure of the hot exhaust gases is obtained by placing a thermocouple device directly in hot exhaust gas plume Unfortunately, no thermocouple device exists that can withstand long term exposure to this hot exhaust gases. Consequently, these devices begin to produce valuable, accurate gas temperature data, but then quickly burn up Using the CLT, and accessing data obtained from thermocouples that are placed at a safe operational distance from the peak gas plume region, the engineers accurately, and routinely reproduce an equivalent measurement for the temperatures produced at the location of the burnt thermocouple sensor. This is accomplished using mathematical correlation relationships based on the initial temperature trajectory elicited by the thermocouple in line with the hot plume correlated to the ongoing measurements obtained by the probes capturing data at safer locations. In essence, a “virtual sensor” is created using a combination of real-time captured data and mathematical correlation, in accordance, with the inventive wearable electronic digital therapeutic device, the data trajectory of multiple data sources (biometric detectors) is determined from baseline measurements (which may, for example, be taken at a doctor's office using mom accurate and expensive equipment and techniques) correlated with ongoing biometric measurements taken over a relatively much longer period of time using relatively lower cost biometric sensors and automatic data detection.

As an exemplary embodiment, a method includes obtaining initial baseline biometric data using a biometric sensor having relatively higher accuracy of a high accuracy detected biometric measurement. For example, the detected biometric measurement could ankle and brachial measurements obtained at a doctor's office to determine an ABI index. This higher accuracy detected biometric measure can then later be used as an initial baseline measurement to obtain extrapolated changes in the ABI index. One or more biometric parameters are detected using the inventive wearable electronic digital therapeutic device, where the biometric parameters are dependent on at least one physiological change to a patient in response to a therapeutic treatment and/or in a change in a concerning condition of the patient.

A microprocessor then receives the high accuracy detected biometric measurement (which may be stored in onboard memory) and the one or more biometric parameters. The microprocessor applies applying probabilistic analysis to determine if at least one physiological change threshold has been exceeded dependent on the probabilistic analysis of the high accuracy detected biometric measurement and the two or more biometric parameters. An action is then activated depending on the determined exceeded physiological change. The probabilistic analysis may include determining a trajectory (trend) of detected biometric data by correlating initial baseline biometric data obtained using a biometric sensor having relatively higher accuracy and a data set of detected biometric parameters obtained using sensors and detectors of the wearable electronic digital therapeutic device. By applying techniques of probabilistic analysis, such as the CLT and Sensor Fusion, baseline information can be compared with data that is ongoingly obtained as the patient goes about their daily lives.

An additional inherent value in the CLT is that by relying on the aggregation of mean values, we are able to more accurately predict and correlate the individual population values. For instance, in a system populated with a large quantity of sensors each of which produces data with a relatively high standard deviation, the CLT theorem tells us that through the aggregation of this large population sample size, we can more accurately predict the mean value of the measured phenomenon. Put another way, a large quantity of sensors, each of which generally produces data with a high degree of uncertainty, all exposed to a single data source, will produce a large volume of data which when aggregated, will fall into a more tightly correlated “normal” (bell curve) distribution, with a mean value and standard deviation that is within the bounds of the “actual” data value at the source.

In accordance with the inventive wearable electronic digital therapeutic device, the accurate prediction of unknown values is obtained by measuring related values across a large sample population and computing the mean and standard deviation of the correlated values relative to the desired measurement. Putting this into practice allows the wearable electronic devices to use a spectrum of relatively low accuracy sensors in order to generate data with a high probability of filling within a narrow mean value from a normal distribution of measured and correlated data.

Specific to installing low cost measurement devices into wearable garments, data of the highest practical accuracy is analyzed from the use of lower cost sensors with reduced size and power consumption. Additional features such as sensor form factor, flexibility, robustness and so on will also influence practical design decisions. By employing these statistical and mathematical methodologies to quantifying and tabulating our captured data, a practical method is developed for producing highly valuable information at a low relative infrastructure and financial cost. Specifically, using sensor fusion and Central Limit Theorem techniques, operating through embedded AI (artificial intelligence) via software running in real time, these inventive wearable garments can deliver highly accurate, clinical quality biometric data at a fraction of the cost as would be associated with utilizing laboratory grade sensors.

FIG. 148 shows a hand of a patient with upper limb contracture FIG. 149 shows a hand and forearm of a patient with upper limb contracture. Sine the natural resting position of the hand is curled and bent down at the wrist, when a patient loses adequate control over the muscles of the lower arm, the hand and arm will atrophy into the position shown in the photos. A contracture deformity is the result of stiffness or constriction in the connective tissues of your body. This can occur in muscles, tendons, ligaments, and skin. Contracture can also result as a deformity in joint capsules, the dense, fibrous connective tissue that stabilizes a joint Contracture deformity restricts normal movement and develops when usually pliable connective tissues become less flexible. A muscle contracture involves the shortening and lightening of the muscles.

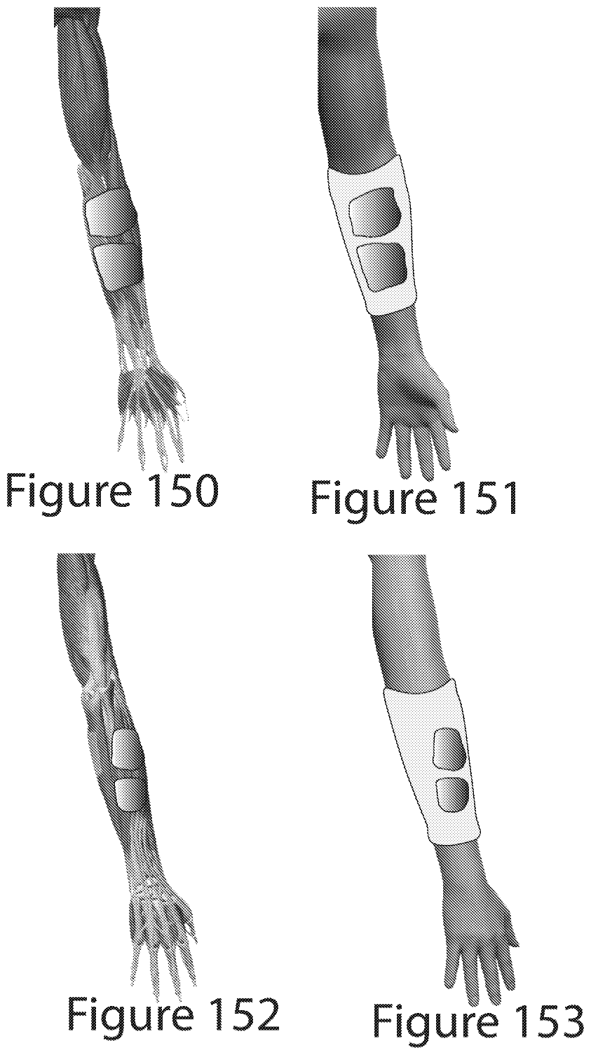

FIG. 150 shows the location of muscles and EMS electrodes for an embodiment of the inventive contracture sleeve. FIG. 151 illustrates an embodiment of the inventive contracture sleeve showing the location of electrodes for applying FMS to the lower forearm muscles. FIG. 152 shows the location of muscles mid FMS electrodes for an embodiment of the inventive contracture sleeve. FIG. 153 illustrates tin embodiment of the inventive contracture sleeve showing the location of electrodes for applying EMS to the upper forearm muscles.

In accordance with an embodiment of the inventive wearable electronic digital therapeutic device, two or more electrodes are in face-to-face electrical communication with the skin covering the muscles of the upper forearm, such as the extensor digitorum muscle and other muscles in the upper forearm. An applied EMS signal through the electrodes causes involuntary muscle contractions that cause the hand to pivot at the wrist. This involuntary movement causes the flexor digitorum muscle and other muscles of the lower forearm to stretch Electrodes in communication with the skin covering the muscles of the lower forearm can be provided to apply EMS, TENS and other treatment to improve tone and prevent antrophy, and to help stretch and strengthen the muscles of the lower arm of a stroke, MS, or other contracture prone patient.

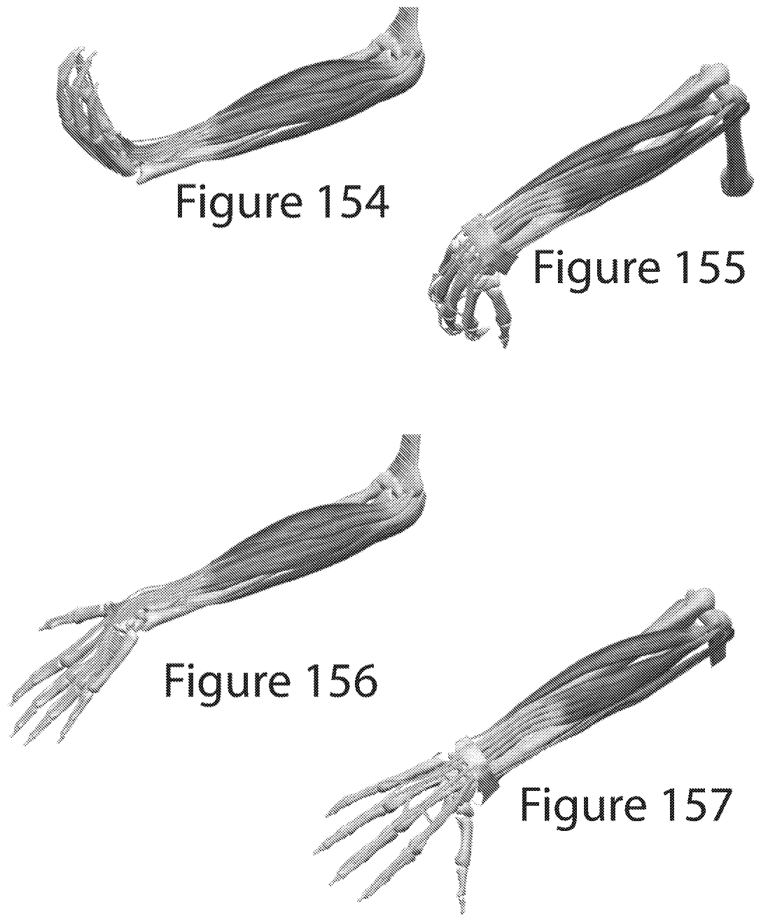

FIG. 154 shows the lower muscles of a forearm in a contracture position. FIG. 155 shows the upper muscles of a forearm in a contracture position. FIG. 156 shows the lower muscles of a forearm stretching a contracture FIG. 157 shows the upper muscles of a forearm stretching a contracture. Contracture is very common in people who have suffered a stroke and resulting paralysis. Other causes include diseases that are inherited or that develop in early childhood, such as muscular dystrophy, cerebral palsy, central nervous systems diseases such as multiple sclerosis or Parkinson's disease, and inflammatory diseases such as rheumatoid arthritis. Physical therapy and occupational therapy are two of the most common treatments for contractures, helping to increase a patient's range of motion and strengthen muscles. Physical therapy sessions require regular attendance for best results with hands-on therapy to improve mobility. Medication may be prescribed to reduce inflammation and pain. For example, in stroke or cerebral palsy patients, botulinum toxin (Botox) may be injected into muscles to reduce tension and minimize spasms.

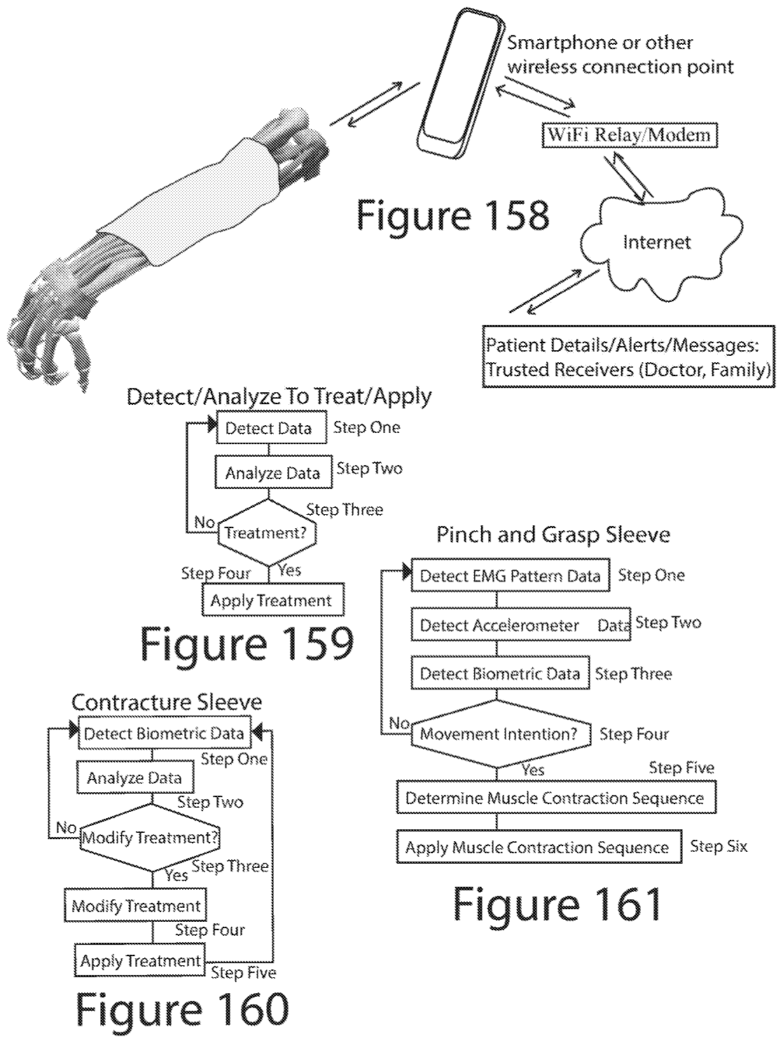

FIG. 158 illustrates constituent parts of a system for remotely monitoring and controlling a wearable electronic digital therapeutic device and illustrates a wearable electronic contracture sleeve and block diagram of electronics. This example of the inventive wearable electronic digital therapeutic garment can be constructed as a convenient configuration that is comfortable, washable and easy to wear by patients and healthy members of the human population. A contracture sleeve with a Smartphone interface is shown. However, as with all the described embodiments, this system can have algorithms, components and applications of any one or more of the various detect, analyze-to-treat and/or apply treatment configurations described herein.

FIG. 159 is a flow chart of an algorithm fora detect, analyze-to-treat, and apply wearable electronic digital therapeutic. The wearable electronic includes biometric and/or ambient sensors and detectors that detect data related to the patient. This detected data is analyzed by an onboard microprocessor and/or by a remote computer. The data is analyzed to determine the appropriate treatment tor the patient based on the detected data and/or a calculated determination based on the detected and an external condition, such as the passage of time. If treatment is not necessary,

FIG. 160 is a flow chart of an algorithm for a contracture sleeve. The contracture sleeve can be configured to automatically apply an EMS signal to the upper forearm muscles to cause involuntary muscle contractions to cause the hand to pivot at the wrist and stretch the contracture or muscle, ligament, etc., lightness in the patient's lower forearm muscles. The application of the EMS signal can be applied in response to detected data, such as detected biometric data. For example, if an accelerometer and/or EMG biometric data is detected (Step One), this data is analyzed (Step Two) to determine the treatment or modification to treatment (Step Three). If the analysis indicates that there is no treatment or modification to treatment needed, then the process flow continues to Step One. If the analysis indicates that there should be an applied treatment or applied modified treatment, then the treatment is modified (Step Four) and then applied (Step Five) For example, if a lack of stretching movement of the contracture hand is detected (Step One) than when the data is analyzed (Step Two) to indicate that there has been a lack of movement, it can be determined to modify an applied EMS treatment (Step Three) and the modification to the treatment determined (Step Four) and a modified EMS signal applied (Step Five). The modification may be, for example, an automatic series of involuntary muscle contractions that stretch the contracture hand when a duration (for example, an hour) has elapsed without adequate movement and stretching of the contracture hand.

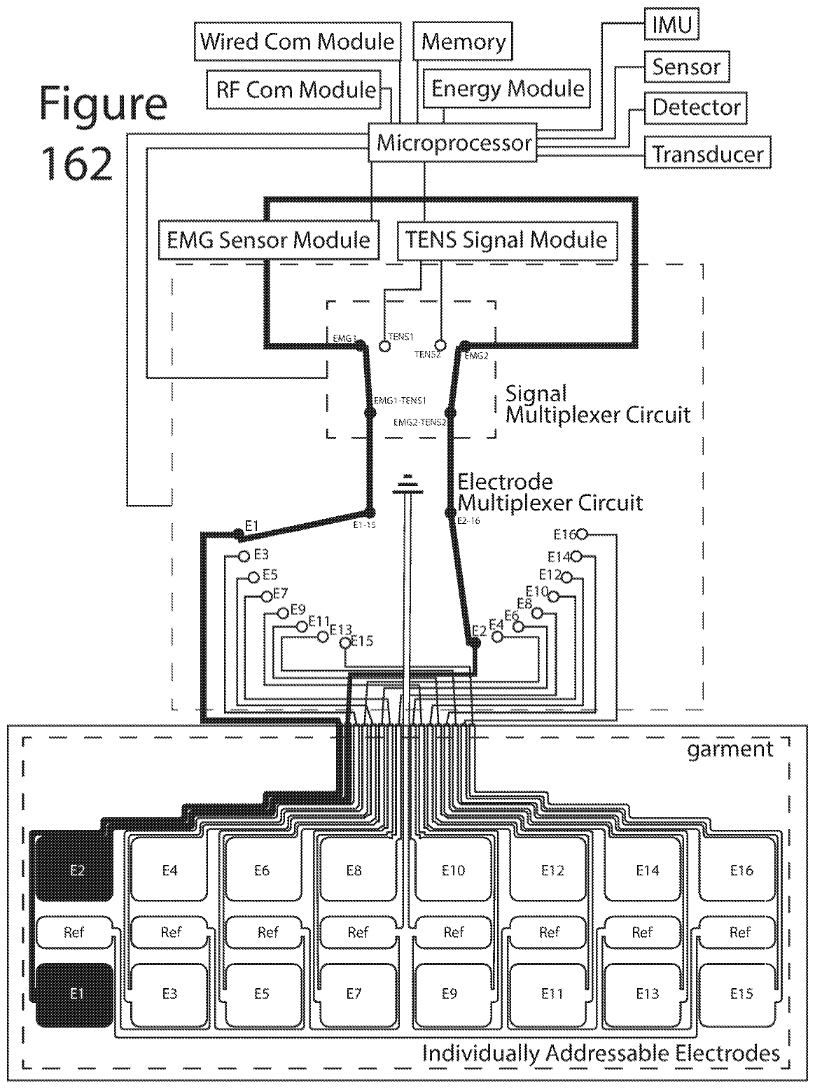

FIG. 161 is a flow chart of an algorithm for a pinch and grasp sleeve. Many daily life skills require the ability to pinch and grasp, for example, shin buttoning, pulling up pants, pulling on socks, opening a door, opening a closed bag, holding a pen, toothbrush, spoon, tying shoes, and many other skills needed for a normal every day life are often difficult or impossible for patients afflicted with stroke, trauma, MS, Parkinson's or oilier diseases or conditions. In accordance with an embodiment of the inventive wearable electronic digital therapeutic device, a forearm sleeve includes electrodes with EMS signals routed to the electrodes to cause involuntary muscle contractions to complete tin intended movement.

An EMG pattern data is detected (Step One) For example, the patient may have a small amount of control over the muscles in his forearm to show a movement intention to reach for an object but not enough control to extend the reach and grasp the object. To help determine the movement intention, accelerometer or other data (including a button pressed by the patient's other hand or voice command of the patient) can be detected (Step Two) Other biometric data, such as a gesture of the patient's other hand or other body part detected as EMG and/or accelerometer data) can also be detected to indicate that the movement intention (Step Three). This data is analyzed to determine if there is a movement intention (Step Four) and if not then process flow goes back to Step One to be reads to detect a movement intention. If there is a movement intention determined (Step Four) then the correct muscle contraction sequence is determined (Step Five) to complete the movement intention. A sequence of EMS signals are applied to the appropriate muscles at the appropriate timing to perform the movement intention (Step Six). Involuntary muscle contractions cause the completion of the movement intention, for example, causing the patient to roach, pinch and grasp an object.

FIG. 162 illustrates an exemplary embodiment showing bi-directional electrical signals applied through a plurality of individually addressable electrodes routed through an electrode multiplex circuit and a signal multiplex circuit for applying a sequential EMS signal and detecting biometric feedback from, for example, the arm muscles or calf of a patient. In accordance with an aspect of the invention, a digital therapeutic device garment is provided with a plurality of individually addressable electrodes supported by the garment for applying a sequential EMS signal and detecting biometric feedback from the muscles of a patient. The individually addressable electrodes are for at least one of applying stimulation electrical signals to the skin of a patient and detecting biometric electrical signals from the skin of the patient. At least one of a signal detector for detecting the biometric electrical signals and a signal generator for generating the stimulation electrical signals are provided. An electrode multiplex circuit addresses the plurality of individually addressable electrodes by at least one of routing the biometric electrical signals from the skin of the patient through more than one electrode or electrode pair of the plurality of individually addressable electrodes to the signal detector and routing the stimulation electrical signals from the signal generator through more than one electrode or electrode pair of the plurality of individually addressable electrode to the skin of the patient A microprocessor controls the signal detector, the signal generator, the electrode multiples circuit and other circuit components.

The microprocessor can control the electrode multiplex circuit to route the biometric electrical signals from the skin of the patient sequentially through more than one electrode or electrode pair of the plurality of individually addressable electrodes to the signal detector. In accordance with this embodiment, a single EMS signal source can service multiple individually addressable electrodes with the EMS signal routed as desired for an intended therapy, such as for the sequential squeezing of the deep veins in the legs to promote blood flow in the direction back to the heart or for sequentially applying involuntary muscle contractions to complete an intended movement One or more EMS signal channels can be multiplexed and signals routed so that even a large array of individually addressable electrodes can be serviced by one or a few signal generators, for example, to provide a finer spatial resolution of the applied EMS signal than indicated by number of electrodes shown in the drawings.

The microprocessor can control the electrode multiplex circuit to route the biometric electrical signals (indicating, for example, muscle activity, heartbeat, etc.) from the skin of the patient simultaneously through more than one electrode or electrode pair of the plurality of individually addressable electrodes to the signal detector. The microprocessor can control the electrode multiplex circuit to route the stimulation electrical signals from the signal generator simultaneously through more than one electrode or electrode pair of the plurality of individually addressable electrodes to the skin of the patient. The microprocessor can control the electrode multiplex circuit to route the stimulation electrical signals from the signal generator sequentially through more than one electrode or electrode pair of the plurality of individually addressable electrodes to the skin of the patient. The microprocessor can control the electrode multiplex circuit to route the stimulation electrical signals from the signal generator simultaneously through more than one electrode or electrode pair of the plurality of individually addressable electrodes to the skin of the patient.

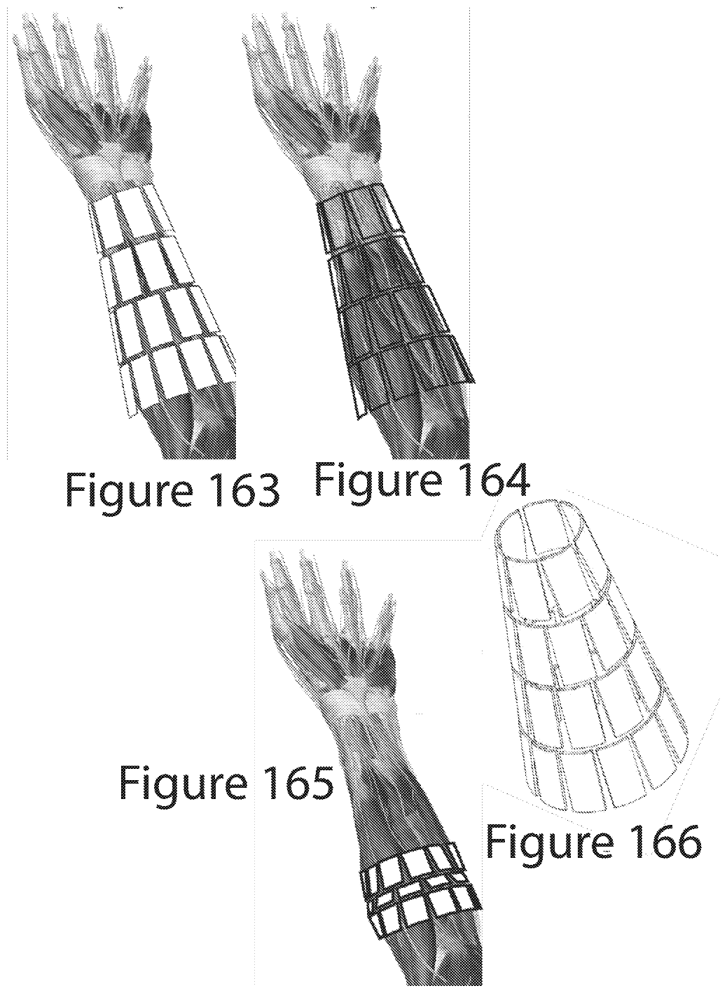

FIG. 162 illustrates an embodiment of an inventive wearable electronic that can be used as an interface for selectively applying transcutaneous electrical nerve stimulation and selectively detecting electromyography through the same electrodes and/or circuit components. FIG. 163 illustrates a plurality of individually addressable electrodes disposed tor receiving biometric electrical signals from and applying EMS signals to motor units underlying the skin of a patient. FIG. 164 illustrates the plurality of individually addressable electrodes showing the muscles and nerves underlying the skin of the patient. FIG. 165 shows a configuration of a plurality of individually addressable electrodes having a biometric signal detection/application electrodes disposed in pairs that approximately line up with the long axis of muscles in the forearm of a patient, along with reference electrodes disposed between the electrode pairs. FIG. 166 shows a three-dimensional representation of a pattern of individually addressable electrodes for a forearm sleeve.

The microprocessor can control the electrode multiplex circuit to route the biometric electrical signals from the skin of the patient sequentially through more than one electrode or electrode pair of the plurality of individually addressable electrodes to the signal detector. The microprocessor can control the electrode multiplex circuit to route the biometric electrical signals from the skin of the patient simultaneously through more than one electrode or electrode pair of the plurality of individually addressable electrodes to the signal detector. The microprocessor can control the electrode multiplex circuit to route the stimulation electrical signals from the signal generator simultaneously through more than one electrode or electrode pair of the plurality of individually addressable electrodes to the skin of the patient. The microprocessor can control the electrode multiplex circuit to route the stimulation electrical signals from the signal generator sequentially through more than one electrode or electrode pair of the plurality of individually addressable electrodes to the skin of the patient. The microprocessor can control the electrode multiplex circuit to route the stimulation electrical signals from the signal generator simultaneously through more than one electrode or electrode pair of the plurality of individually addressable electrodes to the skin of the patient.

FIG. 166 illustrates an electrode pattern for an HHMI forearm sleeve for detecting and applying electrical singles using a single signal detector and a single signal generator, with a multiplexor circuit system for routing the electrical signals. A signal multiplex circuit may be provided controlled by the microprocessor for routing the electrical signals from the signal generator to skin of the patient through the electrode multiplex circuit and to the signal detector from the skin of the patient through the electrode multiplex circuit. A memory may be provided controlled by the microprocessor for storing data dependent on the biometric electrical signals: and a communication module for transmitting the stored data for analysis by a remote network device. The housing may be comprised of an clastic fabric material, and the individually addressable electrodes are dry electrodes may be formed by printing elastic conductive ink.

A same individually addressable electrode of the plurality of individually addressable electrodes can both detects the biometric electrical signals from the skin and applies the stimulation electrical signals to the skin. The microprocessor can control the electrode multiplex circuit to address the plurality of electrodes for sampling the biometric electrical signals at a sampling rate effective for the detection by the signal detector of the biometric signals as electromyographic signals originating from subcutaneous motor units indicative of muscle contractions from two or more muscles of the patient.

The microprocessor can control the electrode multiplex circuit to address the plurality of electrode for applying the stimulation electrical signals as application pulses at a pulse rate effective to cause involuntary contractions of the muscles of the patient. The microprocessor can control the electrode multiplex circuit to address the plurality of individually addressable electrodes by at least one of sequentially and simultaneously routing both the biometric electrical signals from the skin of the patient through more than one electrode or electrode pair of the plurality of individually addressable electrodes to the signal detector and routing the stimulation electrical signals from the signal generator through more than one electrode or electrode pair of the plurality of individually addressable electrode to the skin of the patient. At least one of a inertial measurement unit, a sensor, a detector and a transducer may also be provided supported by the housing.

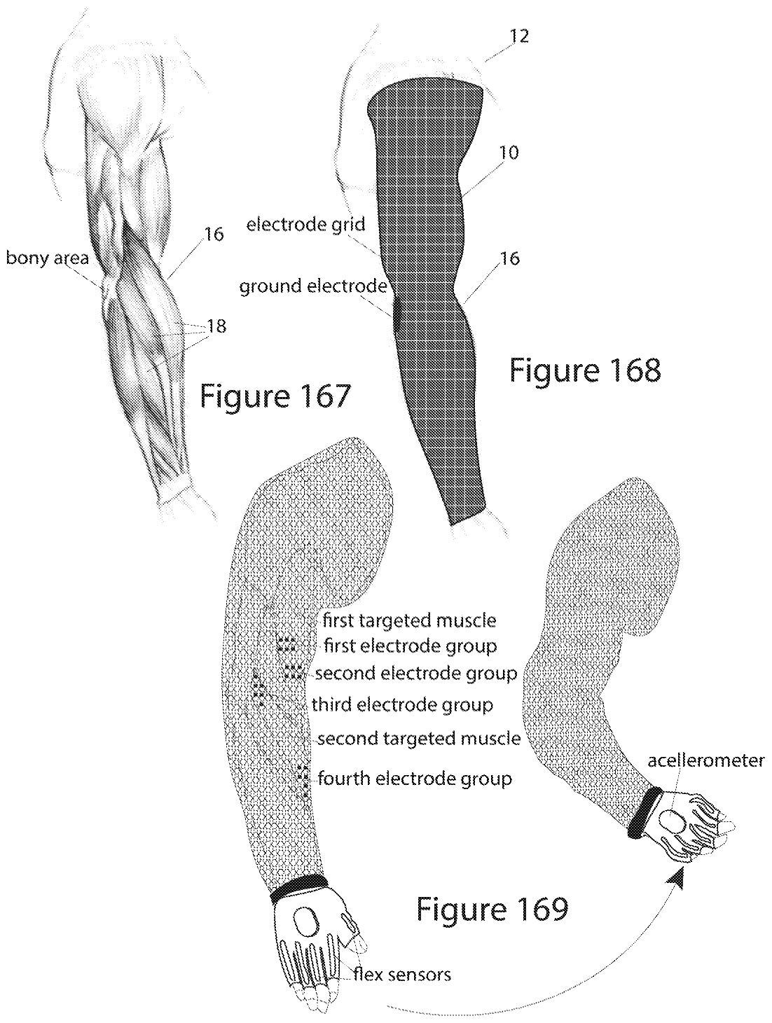

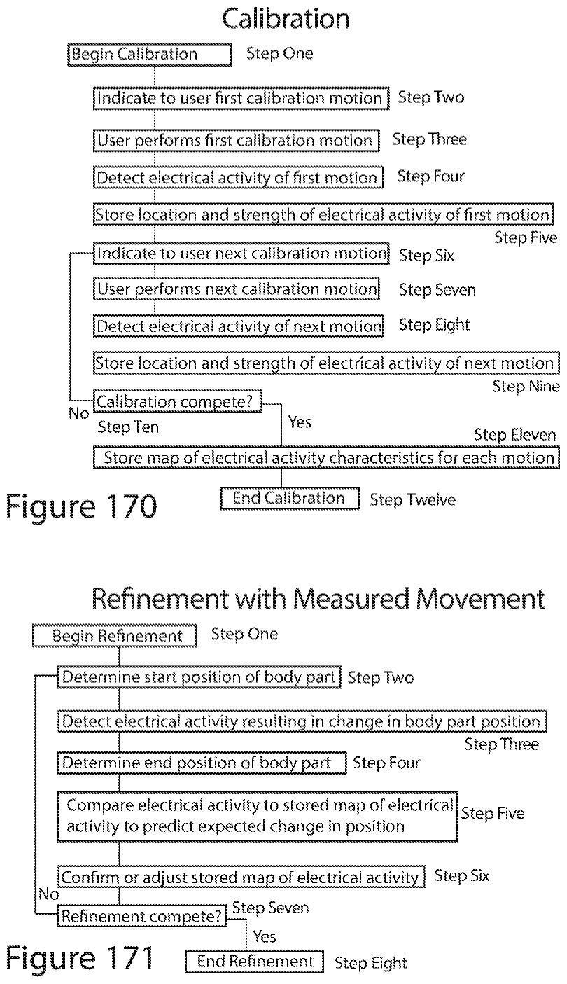

FIG. 167 illustrates a patient's arm 16 without skin showing the relative locations of the muscle groups 18 of the arm 16. FIG. 168 illustrates the arm 16 with the HHMI sleeve having electrodes 14 targeting individual muscles 18 or muscle groups 18. The HHMI sleeve may include tin x-y grid of relatively smaller signal receiving transducers or electrodes 14 and relatively larger signal applying electrodes 14 targeting individual muscles 18 or muscle groups 18, or as shown the electrodes 14 may be uniform in size and distribution. The HHMI may be in the form of a comfortable, easily worn garment that is worn with little restriction of movement.

Electrical signals are applied to the patient 12 via the plurality of electrodes 14. Each electrode is disposable in electrical communication with one or more biological components of the patient 12. At least one electrode is individually addressable to be selectively in an on-state or an off-stale. In the on-state the electrical signals flow through the electrode to at least one biological component of the patient 12. In the off-state the electrical signals do not flow through the electrode to the biological component Each electrode is individually addressable to detect electrical activity of the biological component during a signal detecting operation and apply the electrical signals to the biological component during a signal applying operation.

The HHMI may be configured as a sleeve that is part of a garment, or a self-contained wearable electronic that maps the sources of electrical activity (the subcutaneous muscles 18 and nerves) and hence determines the best locations to detect and to apply the electrical signals for a particular patient 12. For example, neuromuscular electrical stimulation is applied as a low frequency, relatively high intensity pulse.

In accordance with the inventive wearable electronic digital therapeutic device, an electronic circuit can include a TENS and EMS signal generator(s). For example, the activation of the muscle pump can be done with an EMS signal that might be of low enough intensity to be hardly noticable. In other words, for some patients, it may not take intense muscle contractions (and hence a strong EMS signal) to adequately pump blood through the deep veins. At the same time, conventional TENS usually has a quick onset of analgesia. But the effect wears off rapidly when the stimulation is turned off. However, the analgesic effect of low-frequency TENS takes longer to achieve but the pain relief produced by the endogenous opioids can last for a long time. Since the inventive wearable electronic stocking is comfortable and easily worn for extended periods, it can use used to apply a TENS signal that has rapid analgesic effect, an EMS signal thru effectively assists in activating the muscle pump, and a longer lasting low-frequency TENS signal so that the patient is always receiving electroceutical induced pain relief using the body's inherent electrical systems (without pain medications or as an adjunct) even while the muscle pump is being activated through EMS. Since the human body will tend to integrate an applied signal, it is possible to rapidly switch between TENS and EMS signals continuously (or intermittently) applied through the same electrodes. Alternatively applied EMS and TENS signals can maximize both the muscle pump effect to assist in blood flow through the deep veins with a variable TENS used that can be easily controlled by the patient through our smartphone app (or automatically controlled).

The pulse, which may be biphasic, triggers the alpha motor nerves that cause muscle movement. The higher the intensity of the electrical stimulus, cite more muscle fibers will be excited, resulting in a stronger contraction. The contraction can have different speeds and duration of the contraction dependent on the characteristics of the applied electrical signal. The characteristics of the applied electrical signal can be controlled to cause isometric and/or isotonic muscle contraction, where isometric muscle contraction leads to a tension in a muscle, without changing the length of the muscle, and isotonic muscle contraction, results in a shortening of the muscle. In accordance with the inventive haptic interface, a computer controls the characteristics of electrical signals applied to, for example, the motor neurons of the patient's nervous system to cause a desired sensation and/or muscle movement. Exciting the motor neurons via the body's nervous system produces a similar result as when the neurons are excited through the computer controlled electrical stimulation.

In accordance with the inventive haptic interface, computer controlled electrical signals can be applied with signed characteristics effective to stimulate one or more of the tactile receptors found in the skin. The signal characteristics can be controlled, for example, to selectively stimulate the receptors that have, for example, different receptive fields (1-1000 mm2) and frequency ranges (0.4-800 Hz). For example, broad receptive-field receptors like the Pacinian corpuscle produce vibration tickle sensations. Small field receptors Such us the Merkel's cells, produce pressure sensations.

The HHMI may also be used for applications including accelerated learning, brain damage rehabilitation, military and sports training and drone/robotic remote control and sensing. In some configurations, the HHMI includes a thin, flexible sleeve that is unobtrusively worn by a patient. The sleeve has many small electrodes 14 in contact with the skin surface, connected in a matrix and addressed, for example, using electronic techniques borrowed from active and passive matrix video displays. A lightweight, comfortable, haptic sleeve can be configured having electrode size and density enabling automatic calibration to the unique physiology of the patient. The haptic sleeve provides precise electrical activity detection (for example, to detect the muscles 18 and nerves involved in even subtle arm movement indicating the onset of a persistent Parkinsonian tremor) and nearly instantaneous electrical signal application (to cause involuntary and accurate muscle and nerve impulses that counteract and negate the undesirable arm trembling that would have otherwise occurred). The applied electrical signal and resultant muscle contraction is perceived as a massage sensation by the patient, in this case, the use of the HHMI provides a wearable electronic device used as a non-invasive, non-chemical means to effectively mitigate tremors caused, for example, by stroke, accident or by Parkinson's disease.

In accordance with another aspect of the invention, a plurality of haptic sensory cues am generated capable of being perceived by a patient 12. The haptic sensory cues are received by the patient 12 as computer controlled serially generated electrical signals. The electrical signals invoke an involuntary body pan movement causing at least an urging towards at least one of a predetermined motion. Alternatively, or additionally, the signals invoke a perception by the patient 12 having a predetermined somatosensory sensation dependent on the computer controlled serially generated electrical signals.

FIG. 169 shows an example where a specific muscle (bicep) is targeted for contraction by applying a transcutaneous electrical signal. The electrical signal is applied as a DC voltage between a first electrode group and a second electrode group. As shown in other circuits the circuit can be modified to include circuit elements so that the appropriate electrode group to invoke a desired muscle response can be determined for example, during a calibration mode. During a calibration mode (described in more detail below), these same first electrode group and second electrode group are used to detect the electrical activity generated when the patient 12 performs a known action, such as raising the hand to the chest (contracting the bicep muscle). Additionally, or alternatively, the appropriate electrode groups to invoke a desired muscle response can be extrapolated from the calibration data because live general physiology of a human arm 16 is well known. In this ease, the calibration mode and/or refinement inode provides fine tuning of a pre-determined electrode pattern, where the predetermined electrode pattern is based on human physiology and the fine tuning is based on the particular electrical activity detected for the patient 12 during the calibration mode. A strain gauge wire or the like can also be used to detect muscle movement, and/or a memory metal used to contract and apply a squeezing force, acting as conductive pathways to the electrodes 14 or provided as separate components.

The inventive haptic interface uses sensory feedback and algorithms to correctly control the characteristics of the electrical stimulation. Muscle contractions can be induced that result in the same movements of the body part of the patient 12 (e.g., fingers) as if performed voluntarily by the patient 12 using precisely controlled muscle movements.

Muscle contractions and changes in body part positions can be used as metrics during calibration and also to obtain feedback while the applied electrical stimulation causes an automatic and involuntary movement of the patient's body parts. The sleeve may include transducers used to measure changes in muscle shape or body part position, or to apply a pressure, such as a squeeze or vibration. For example, a shape memory alloy (which could be formed as a sheath around or otherwise in communication with the electrode lead conductors) or piezo-electric or mechanical vibrators, can be used under control of electrical signals from the computer or microprocessor, to apply haptic cues in the form of pressure or vibration.

The HHMI is constructed of layers of thin flexible materials, such as conductive stretchable fabrics, flexible insulators, flexible circuit boards, and tire like. The materials may be woven, spun, closed cell, open cell, thin film, or other suitable structure.

Layers, bonded layers, and constituent elements of the HHMI may be printed using a 3D printer or formed by a batch or roll-to-roll manufacturing process including lamination, screen printing, inkjet printing, self-assembly, vapor deposited, sprayed.

The HHMI can be fabricated as a sleeve, glove, legging, shirt, full body suit, etc., and has a flexible and comfortable snug fit that urges the electrodes 14 into face-to-face surface contact with the skin of the patient 12. Gel electrodes 14 can be used but have some drawbacks. The electrode construction described herein provides thin, flexible structures designed specifically for compression face-to-face contact Whatever the case, the transference of the electrical signal between the electrically conductive surface of the electrode and the skin of the patient 12 has to be effectively accommodated.

As shown, in FIG. 71, since every human body is different, at the beginning (step one) of a calibration mode a patient 12 is asked to perform a first calibration motion (step two). If the patient is too paralyzed to make a detectable movement, a caregiver can perform the calibration movement and sensors, such as an accelerometer and amplified EMG detected used to detect the calibration motion. The patient 12 performs a known motion (step three) that would cause nerve firings and muscle contractions, such as a motion that replicates a feeding motion. The electrical activity (amplified EMG) and/or physical movement of the first motion is detected (step four) and the characteristics of the body-generated electrical activity (e.g., electromyographic signals generated by the nerves and muscles 18 are sensed and stored (step five). In addition to the body-generated electrical activity, other physiological changes can be detected, such as a change in the shape of the patient's arm 16 caused by muscle contractions. These physiological changes are useful for calibrating the inventive Human Machine interface and also for determining patient's intended electrical signals Especially for patient's with weak EMG signal strength, other electrical and muscle activity that is detected and used tor calibration, control intentions, patient 12 conditions, etc., can include EKG, and EEG, as non-limiting examples.

A next calibration motion is indicated to the patient 12 (step six), the patient 12 performs the motion (step seven) the electrical activity is detected (step eight) and the characteristics of tire detected electrical activity is stored (step nine). If the calibration routine is not complete (step nine), then another next calibration motion is indicated (flow goes hack to step six). If the calibration routine is complete (step nine) than a mapping is made of the detected electrical activity characteristics for the calibration motions (step eleven). By this process, the electrical signals and the source of the electrical signals (muscles IX and nerves) associated with known motions am calibrated for the individual patient 12 and a map of the signal characteristics associated with corresponding muscles 18 and nerves for each respective calibration motion is stored for the patient 12.

In an auto-action mode, for example, for VR Stroke Rehabilitation, the calibration data is used to determine the characteristics of the computer-generated electrical activity causing a desired automatic and involuntary movement of the patient's body parts. The result is the patient perceives the involuntary movement as though caused by an externally applied force.

FIG. 171 is a flowchart showing an algorithm for refinement of the calibrated HHMI using measured movement of the patient 12. This exemplary algorithm provides for further customizing the HHMI to interface with a particular patient 12. Since every human body is different, in the calibration mode the patient performs a known task that causes nerve firings and muscle contractions, such as a motion that replicates a feeding motion. The characteristics of the body-generated electrical activity (e.g., electromyographic signals generated by the nerves and muscles 18 are sensed by the sensory transducers (i.e., the electrodes 14 shown herein throughout). The sensory transducers are used to calibrate the location, relative strength, etc. of each detected electrical signal. To refine the calibrated HHMI, a refinement process may be stalled (step one). A start position of a body part, such as the hand of tire patient 12, is determined using for example, a known position taken consciously by the patient 12, or the detection of the body part, such as a hand, using gyroscopes, accelerometers, IR detectors (e.g., Leap Motion), or others (step two) Tire electrical activity resulting in the change in body part position is detected (step three) as the body part moves from the start position to a determined end position (step four). For example, the hand of patient 12 can be voluntarily brought from a position where the arm 16 is relaxed and the hand is dropped down to where the hand is brought to touch the shoulder of the patient 12. This motion is made consistent by the patient 12, and allows for the determination of the start position (step two) with the hand dropped down at the patient 12 side, the detection of electrical activity that results in the change in body part position (step three) and the determination of the end position (step four) when the hand touches the shoulder. The detected electrical activity is then compared to a stored map of electrical activity obtained, for example, using the calibration algorithm. The detected electrical activity and the stored map are compared to predict the expected change in position. The stored map is then confirmed or adjusted if necessary, depending on the comparison (step six). If the refinement is complete (step seven), the algorithm ends (step eight). If it is not complete, the refinement continues again at step two.

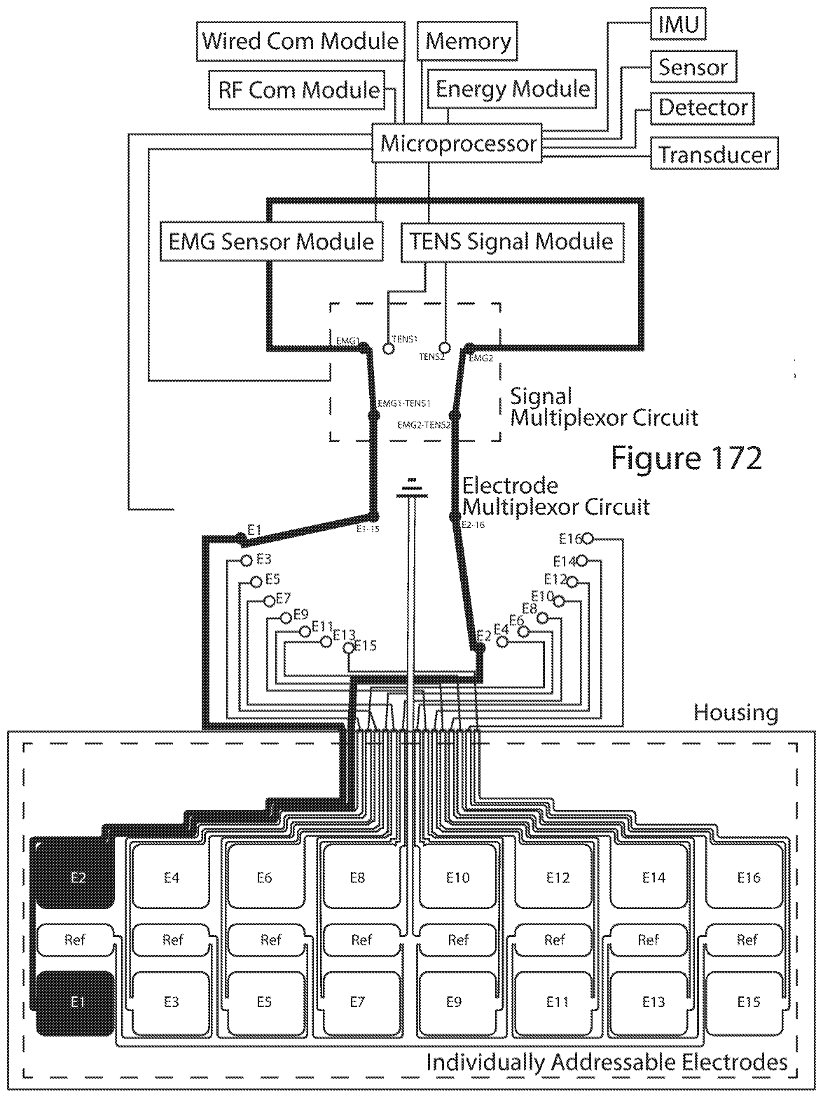

FIG. 172 illustrates an electrode pattern for an HHMI forearm sleeve for detecting and applying electrical singles using a single signal detector and a single signal generator, with a multiplexor circuit system for routing the electrical signals. FIG. 172 show s an HHMI sleeve having two sets of individually addressable electrodes, with each set having a multiplexor circuit system for routing electrical signals so that a small number of costly signal detection and signal generation electronics are usable with a large number of screen printed and laminated low cost individually addressable electrodes.

A signal multiplex circuit may be provided controlled by the microprocessor for routing the electrical signals from tire signal generator to skin of the patient through the electrode multiplex circuit and to the signal detector from the skin of the patient through the electrode multiplex circuit.

A memory may be provided controlled by the microprocessor for storing data dependent on the biometric electrical signals; and a communication module for transmitting the stored data for analysis by a remote network device.

The housing may be comprised of an elastic fabric material, and the individually addressable electrodes are dry electrodes may be formed by printing clastic conductive ink.

A same individually addressable electrode of the plurality of individually addressable electrodes can both detects the biometric electrical signals from the skin and applies the stimulation electrical signals to the skin, flic microprocessor can control the electrode multiplex circuit to address the plurality of electrodes for sampling the biometric electrical signals at a sampling rate effective for the detection by the signal detector of the biometric signals as electromyographic signals originating from subcutaneous motor units indicative of muscle contractions from two or more muscles of the patient.

The microprocessor can control the electrode multiplex circuit to address the plurality of electrode for applying the stimulation electrical signals as application pulses at a pulse rate effective to cause involuntary contractions of the muscles of the patient. The microprocessor can control the electrode multiplex circuit to address the plurality of individually addressable electrodes by at least one of sequentially and simultaneously routing both the biometric electrical signals from the skin of the patient, through more than one electrode or electrode pair of the plurality of individually addressable electrodes to the signal detector and routing the stimulation electrical signals from the signal generator through more than one electrode or electrode pair of the plurality of individually addressable electrode to the skin of the patient. At least one of a inertial measurement unit, a sensor, a detector and a transducer may also be provided supported by the housing.

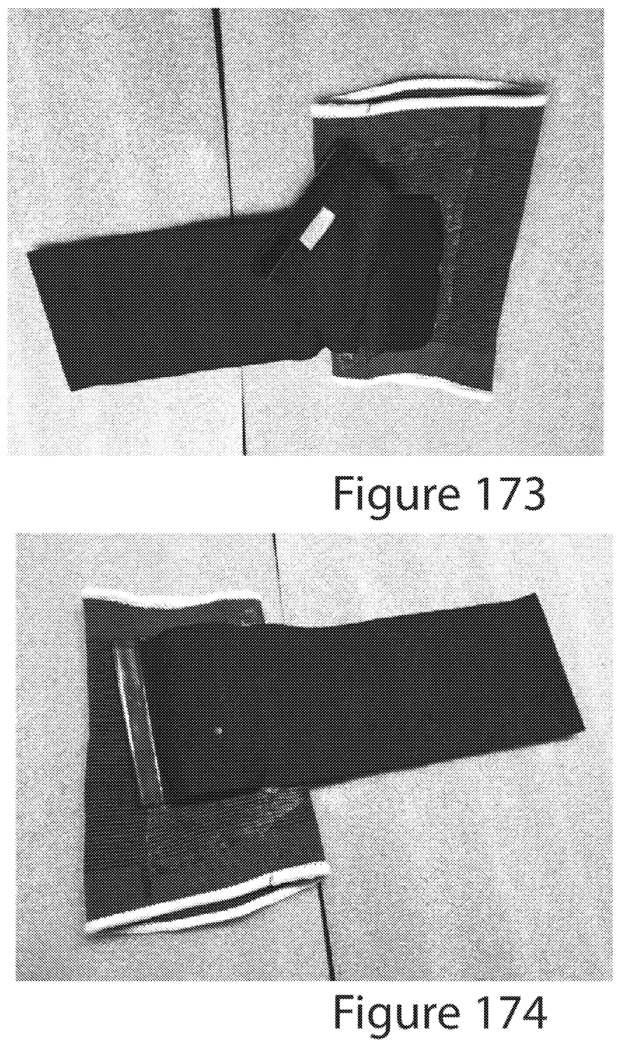

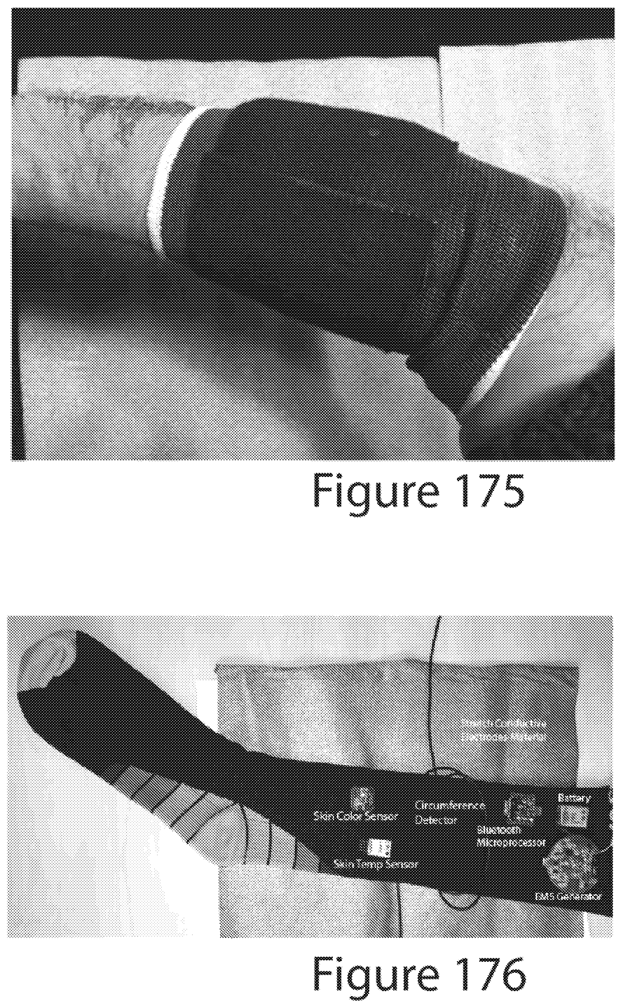

FIG. 173 is a photograph showing an embodiment of a contracture sleeve having a compression sleeve portion, foam urging block adjacent to electrodes disposed in use in face to face electrical contact with the forearm skin, and an clastic bandage for applying an urging force against the foam block to press the electrodes against the skin. FIG. 174 is a photograph showing an embodiment of a contracture sleeve having a compression sleeve portion and showing the clastic bandage for applying an urging force against the foam block to press the electrodes against the skin. FIG. 175 is a photograph showing an embodiment of a contracture sleeve having a compression sleeve portion located on the upper forearm of the patient, with a foam urging block adjacent to electrodes disposed in use in face to face electrical contact with the forearm skin, and an clastic bandage wrapped around tire forearm for applying an urging force against the foam block to press the electrodes against the skin. FIG. 176 is a photograph showing a configuration of a stocking having biometric detectors, microprocessor, batten and EMS signal generator.

The inventive digital therapeutic device and these example processes implemented as a software/hardware solution creates a drug/device combination therapy that puts the patient s own body into a real-time feedback loop. The embodiments described herein can be used for many types of diseases and conditions, and work with a large number of prescribed or over the counter drugs, herbal remedies, or other applications where an ingested chemical modifies a detectable biometric. These therapies available through the inventive digital therapeutic device can be used as complementary or alternatives to drugs and surgery, and typically can typically continue for as long as the target drug is prescribed for the patient, and/or be employed before or after the prescribed drug is taken as treatment by the patient.

The data detection, transmission, and storage described herein provide a detailed history of the patient's adherence to the prescribed course of drug therapy. The biometric parameters such as those described herein with regards to the embodiments can also be detected, logged and/or transmitted, enabling a detailed history of the patient's therapy, course of treatment, measured results of treatment, etc., and can be made available to improve the care given to the particular patient, and n the aggregate, provide significant data along with that of other patients, to assist in new drug discovery, treatment modifications, and a number of other advantages of the beneficial cycle created by detection, transmission, storage and analysis of biometric data taken directly from the patient during the course of drug therapy and/or other treatments.

Various modifications and adaptations to the foregoing exemplary embodiments of this invention may become apparent to those skilled in tire relevant arts in view of the foregoing description, when read in conjunction with the accompanying drawings. However, any and all modifications will still fall within the scope of the non-limiting and exemplary embodiments of this invention.

The embodiments described herein are intended to exemplary and non-limiting, the selection of biometric, environmental, or other measured conditions is not limited to a specific metric or multiple metrics described herein but will depend on the particular application and treatment, data collection, and/or other use of the detected metrics. Also, the treatments employed in any of the embodiments described herein is not limited to a specific treatment or action but will depend on the intended use and desired outcome of the combined detected metrics and applied treatments.

Furthermore, some of the features of the various non-limiting and exemplary embodiments of tins invention may be used to advantage without the corresponding use of other features. As such, the foregoing description should be considered as merely illustrative of the principles, teachings and exemplary embodiments of this invention, and not in limitation thereof.

Various modifications and adaptations to the foregoing exemplary embodiments of this invention may become apparent to those skilled in the relevant arts in view of the foregoing description, when read in conjunction with the accompanying drawings. However, any and all modifications will still fall within the scope of the non-limiting and exemplary embodiments of this invention.

The embodiments described herein are intended to exemplary and non-limiting, the selection of biometric, environmental, or other measured conditions is not limited to a specific metric or multiple metrics described herein but will depend on the particular application and treatment, data collection, and/or other use of the detected metrics. Also, the treatments employed in any of the embodiments described herein is not limited to a specific treatment or action but will depend on the intended use and desired outcome of the combined detected metrics and applied treatments.

Furthermore, some of the features of the various non-limiting and exemplary embodiments of this invention may be used to advantage without the corresponding use of other features. As such, the foregoing description should be considered as merely illustrative of the principles, teachings and exemplary embodiments of this invention, and not in limitation thereof.

1. A method, comprising: obtaining initial baseline biometric data using a biometric sensor having relatively higher accuracy, where the initial baseline biometric data includes a high accuracy detected biometric measurement;
detecting one or more biometric parameters, where the biometric parameters are dependent on at least one physiological change to a patient in response to a therapeutic treatment;
receiving by a microprocessor the high accuracy detected biometric measurement and the one or more biometric parameters;
applying probabilistic analysis to determine if at least one physiological change threshold has been exceeded dependent on the probabilistic analysis of the high accuracy detected biometric measurement and the two or more biometric parameters;
and activating an action depending on the determined exceeded said at least one physiological change.

2-3. (canceled)

4. A digital therapeutic device, comprising a wearable electronic therapeutic device having one or more biometric detectors each for detecting one or more biometric parameters, where the biometric parameters are dependent on at least one physiological change to a patient in response to a therapeutic treatment;
a microprocessor for receiving the one or more biometric parameters and applying probabilistic analysis to determine if at least one physiological change threshold has been exceeded dependent on the probabilistic analysis of the two or more biometric parameters;
and an activation circuit for activating an action depending on the determined exceeded said at least one physiological change.

5. The digital therapeutic device according to claim 4, wherein the therapeutic treatment includes an anticoagulant for treating a cardiovascular condition, and wherein the at least one physiological change includes an indication of a change in the cardiovascular condition.

6. The digital therapeutic device according to claim 4, wherein the action includes transmitting an alert, modifying the therapeutic treatment, and transmitting data dependent on at least one of the at least one physiological change, the one or more biometric parameters, the therapeutic treatment and the probabilistic analysis.

7. The digital therapeutic device according to claim 4, wherein the probabilistic analysis comprises determining from a data set of the one or more biometric parameters whether the data set is acceptable for deciding that the at least one physiological change threshold has been exceeded.

8. The digital therapeutic device according to claim 7, wherein the probabilistic analysis
further comprises applying a statistical weighting to each of the one or more biometric parameters, where the statistical weighting is dependent on a predetermined value of a ranking of importance in detecting each of the at least one physiological change for said each of the one or more biometric parameters relative to others of the one or more biometric parameters.

9. The digital therapeutic device according to claim 4, wherein at least one of the biometric values is determined from one or more water soluble molecules; and
on-demand sweat stimulator for stimulating the production of sweat by the patient and a sweat chemistry sensor for sensing the one or more water soluble molecules.

10. A digital therapeutic device, comprising:
a wearable electronic garment having at least one pair of electrodes for applying an electrical muscle stimulation signal through the skin of a patient to induce involuntary contractions in one or more muscles adjacent to a deep vein blood vessel, where the involuntary muscle contractions induce a squeezing action on the blood vessel and promote a flow of blood through the blood vessel in a direction towards a heart of the patient;
a biometric signal detector for detecting a biometric parameter indicative of the flow of blood through the blood vessel, wherein the biometric parameter is dependent on a therapeutic action of a pharmaceutical medicinal compound for inhibiting an initiation of coagulation of blood; and
a microprocessor for modifying the application of the electrical signal dependent on the detected biometric signal, wherein the applied electrical muscle stimulation signal is modified in response to the therapeutic action of the pharmaceutical medicinal compound.

11. The digital therapeutic device according to claim 10, wherein the biometric parameter is detectable dependent on at least one of skin temperature, skin color, blood flow, pulse, heartbeat, blood pressure, blood viscosity, skin tightness, swelling, blood chemistry, sweat chemistry, an electronic biomarker, a chemical biomarker, and electromyography.

12. The digital therapeutic device according to claim 10, wherein the applied electrical muscle stimulation signal is applied as a sequence of electrical signals through two or more pairs of electrodes for sequentially squeezing the blood vessel along a longitudinal axis of the blood vessel to promote the flow of blood through the blood vessel in the direction towards the heart determined by the sequential squeezing and one way vascular valves within the blood vessel.

13. The digital therapeutic device according to claim 10, wherein the biometric signal is dependent on heartbeat and the applied electrical muscle stimulation signal is modified to impart the squeezing action dependent on the heartbeat.

14. The digital therapeutic device according to claim 13, wherein the heartbeat biometric signal is detected as at least one of a biometric optical signal and an biometric electrical signal from at least one biometric detector in contact with a skin surface of the patient.

15. The digital therapeutic device according to claim 14, wherein the electrical muscle stimulation signal is applied to the at least one muscle through the skin surface from at least one electrode in contact with the skin surface, and wherein the biometric detector includes the at least one electrode that applies the electrical muscle stimulation signal used also for detecting the heartbeat from the biometric electrical signal.

16. The digital therapeutic device according to claim 10, wherein the biometric signal is dependent on surface vein blood flow and the applied electrical muscle stimulation signal is modified to impart the squeezing action dependent on the surface vein blood flow.

17. The digital therapeutic device according to claim 10, further comprising an elastic support, at least one electrode supportable by the elastic support and for applying stimulation electrical signals to skin of a user, and at least one urging member supportable by the elastic support adjacent to the at least one electrode for urging the at least one electrode towards the skin of the user.

18. An apparatus according to claim 10, further comprising a removably fixable dry electrode supportable by the elastic support and separate from the elastic support, wherein the dry electrode includes the at least one electrode.

19. An apparatus according to claim 10, wherein the dry electrode further includes the at least one urging member, and wherein the dry electrode and the elastic support interact to hold the at least one electrode in electrical contact with the skin of the user.

20. An apparatus according to claim 19, wherein the elastic support comprises an elastic fabric for applying a squeezing force against the dry electrode for cooperatively acting with the least one urging member for holding and urging the at least one electrode in electrical contact with the skin of the user.

21-95. (canceled)
